(19) United States
(12) Patent Application Publication
(10) Pub. No.: US 2023/0357188 A1
(43) Pub. Date: Nov. 9, 2023

(54) PHENANTHRENE ORGANIC COMPOUND AND USE THEREOF

(71) Applicants: Wuhan Tianma Microelectronics Co., Ltd., Wuhan (CN); Wuhan Tianma Microelectronics Co., Ltd. Shanghai Branch, Shanghai (CN)

(72) Inventors: Wei GAO, Wuhan (CN); Wenpeng DAI, Wuhan (CN); Lu ZHAI, Wuhan (CN); Yi LIU, Wuhan (CN)

(73) Assignees: Wuhan Tianma Microelectronics Co., Ltd., Wuhan (CN); Wuhan Tianma Microelectronics Co., Ltd. Shanghai Branch, Shanghai (CN)

(21) Appl. No.: 18/218,486

(22) Filed: Jul. 5, 2023

(30) Foreign Application Priority Data
Mar. 6, 2023 (CN) 202310233163.3

(51) Int. Cl. C07D 401/12; C07D 403/12; C07D 519/00; C07D 413/14; C07D 417/14; C07D 491/048; C07D 413/12; H10K 50/82; H10K 50/81; H10K 50/18; H10K 85/60

(52) U.S. Cl. CPC C07D 401/12; C07D 403/12; C07D 519/00; C07D 413/14; C07D 417/14; C07D 491/048; C07D 413/12; H10K 50/82; H10K 50/81; H10K 50/181; H10K 85/6572; H10K 85/656; H10K 85/615; C07B 2200/05

(57) ABSTRACT

Provided are a phenanthrene compound and use thereof, wherein the phenanthrene compound has the structure represented by Formula I. The phenanthrene organic compound contains a heteroarylamine structure, and at least one of Ar₂ and Ar₃ is selected from substituted or unsubstituted C2-C20 nitrogen-containing fused-ring heteroaryl having an electron-withdrawing property. All active sites of phenanthrene are substituted and passivated, and the phenanthrene has a stable chemical structure and high thermal stability. The phenanthrene compound thus has a high glass transition temperature and a high refractive index in the visible-light field, and when applied to the CPL layer of an organic electroluminescent device, can effectively improve the light extraction efficiency of the organic electroluminescent device, thereby improving the luminescence efficiency and service life of the organic electroluminescent device.

### CROSS-REFERENCE TO RELATED APPLICATION(S)

This application claims priority to Chinese Patent Application No. CN 202310233163.3 filed Mar. 6, 2023, the disclosure of which is incorporated herein by reference in its entirety.

### TECHNICAL FIELD

The present disclosure belongs to the field of organic electroluminescent materials, and relates to a phenanthrene organic compound and use thereof.

### BACKGROUND

According to the direction of light emitted by the organic light-emitting layer, the Organic Light Emitting Diode (OLED) displays can be divided into bottom-emitting OLED displays and top-emitting OLED displays. In the bottom-emitting OLED display, light is emitted toward a substrate, a reflective electrode is formed on the organic light-emitting layer, and a transparent electrode is formed under the organic light-emitting layer. If the OLED display is an active-matrix OLED display, since a thin-film transistor formed therein is partially opaque to light, the light-emitting area is reduced. In another aspect, in the top-emitting OLED display, a transparent electrode is formed on the organic light-emitting layer, and a reflective electrode is formed under the organic light-emitting layer. Therefore, light is emitted toward an opposite direction to a substrate, thereby increasing the light transmission area and improving the brightness.

The commonly used methods to improve the luminescence efficiency are to form structures such as folds, photonic crystals, and microlens arrays (MLAs) on the light emission surface of the substrate and to add a capping layer with a high refractive index on the semi-reflecting and semi-transmitting electrode with a low refractive index. The first two structures affect the angular distribution of the radiation spectrum of the OLED, the third structure is complex in manufacturing processes, but the use of the capping layer is simple in processes, can significantly improve the luminescence efficiency and thus is particularly received a lot of attention.

The materials of the capping layer are classified into two categories: inorganic materials and organic materials. When an OLED assembly is prepared by an evaporation method, to form a cover layer, the solution of a metal mask with high fineness needs to be used, but the metal mask has a problem that deformation caused by heat causes poor positioning accuracy. That is, the melting point of ZnSe is higher than 1100° C. (H. Riel. Phosphorescent top-emitting organic light-emitting devices with improved light outcoupling. Appl. Phys. Lett., 2003, 82, 466), and the mask with high fineness cannot be evaporated at an accurate position. Meanwhile, most inorganic substances have a high evaporation temperature and are not suitable for the mask with high fineness. The inorganic substance film forming method based on the sputtering method causes damage to the light-emitting device, and a cover layer using the inorganic substance as a constituent material thus cannot be used.

In view of the present situation in which the light extraction efficiency of the OLED device is low, one layer of a cover layer, i.e., a light extraction material, needs to be added to the device structure. According to the principle of optical absorption and refraction, the refractive index of this cover layer material should be as high as possible.

The current methods for improving the performance of the OLED light-emitting device include: reducing the drive voltage of the device, increasing the luminescence efficiency of the device, increasing the service life of the device, and the like. To achieve the continuous improvement of the performance of the OLED device, the structure and manufacturing process of the OLED device need to be innovated, and the photoelectric functional material of the OLED also needs to be continuously researched and innovated to create the OLED functional materials with better performance.

The current CPL materials are mainly hole transport layer materials and electron transport materials. The organic EL material involved below is known: for the cover layer for adjusting the refractive index, in a case where (8-hydroxyquinolinato)aluminium (hereinafter, simply referred to as Alq₃) is used, Alq₃ is generally used as a green light-emitting material or an electron-transport material but has relatively weak absorption near 450 nm when used in a blue light-emitting device. Therefore, in the case of the blue light-emitting device, the problem that the color purity is reduced exists.

It is therefore desirable in the art to develop cover layer materials with better performance.

### SUMMARY

The present disclosure is to provide a phenanthrene organic compound and use thereof.

A first aspect of the present disclosure is to provide a phenanthrene organic compound having the structure represented by Formula I:

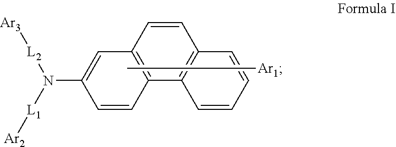

wherein Ar₁ is selected from substituted or unsubstituted C6-C20 aryl, substituted or unsubstituted C10-C20 fused-ring aryl, substituted or unsubstituted C2-C20 heteroaryl, or substituted or unsubstituted C4-C20 fused-ring heteroaryl;
L₁ and L₂ are independently selected from a single bond, substituted or unsubstituted C6-C20 arylene, substituted or unsubstituted C2-C20 heteroarylene, or substituted or unsubstituted C4-C20 fused-ring heteroarylene; and
Ar₂ and Ar₃ are independently selected from a substituted or unsubstituted C10-C20 fused-ring aromatic group or substituted or unsubstituted C4-C20 nitrogen-containing fused-ring heteroaryl having an electron-withdrawing property, and at least one of Ar₂ and Ar₃ is selected from substituted or unsubstituted C4-C20 nitrogen-containing fused-ring heteroaryl having an electron-withdrawing property.

In the present disclosure, C10-C20 may be C10, C12, C13, C14, C15, C16, C18 or C19, etc.
C6-C20 may be C6, C8, C10, C12, C13, C14, C15, C16, C18 or C20, etc.
C2-C20 may be C3, C5, C6, C8, C10, C12, C13, C14, C15, C16, C18 or C19, etc.
C4-C20 may be C5, C6, C8, C10, C12, C13, C14, C15, C16, C18 or C19, etc.

The phenanthrene organic compound of the present disclosure contains a heteroarylamine structure, and at least one of Ar₂ and Ar₃ is selected from substituted or unsubstituted C2-C20 nitrogen-containing fused-ring heteroaryl having an electron-withdrawing property. The active site of phenanthrene is passivated by substitution or steric hindrance, and the phenanthrene thus has a more stable chemical structure and higher long-term evaporation thermal stability. In this manner, the phenanthrene organic compound of the present disclosure has the following advantages: (1) the refractive index and light extraction efficiency are high; (2) no absorption exists in the wavelength regions of blue, green, and red, and the color purity is not decreased; (3) the difference of refractive indices measured in the wavelength regions of blue, green, and red is small; (4) the glass transition temperature and decomposition temperature are high, and the material can be evaporated but is not decomposed thermally; (5) the thin film made of the material has high stability, excellent durability and a long service life. The phenanthrene organic compound, when applied to the CPL layer of an organic electroluminescent device, can effectively improve the light extraction efficiency of the organic electroluminescent device, thereby improving the luminescence efficiency and service life of the organic electroluminescent device.

A second aspect of the present disclosure is to provide an organic electroluminescent material including the phenanthrene organic compound as described in the first aspect.

A third aspect of the present disclosure is to provide a capping layer material including the phenanthrene organic compound as described in the first aspect.

A fourth aspect of the present disclosure is to provide an electron blocking material including the phenanthrene organic compound as described in the first aspect.

A fifth aspect of the present disclosure is to provide an organic electroluminescent device including an anode, a cathode, and an organic thin-film layer disposed between the anode and the cathode, wherein the cathode is covered with a capping layer, and the capping layer includes the phenanthrene organic compound as described in the first aspect.

A sixth aspect of the present disclosure is to provide an organic electroluminescent device including an anode, a cathode, and an organic thin-film layer disposed between the anode and the cathode, wherein the organic thin-film layer includes the phenanthrene organic compound as described in the first aspect.

A seventh aspect of the present disclosure is to provide a display panel including the organic electroluminescent device as described in the sixth aspect.

An eighth aspect of the present disclosure is to provide an electronic device including the display panel as described in the seventh aspect.

### DETAILED DESCRIPTION

Technical solutions of the present disclosure are further described below through embodiments. It is to be understood by those skilled in the art that the embodiments described below are used for a better understanding of the present disclosure and are not to be construed as specific limitations to the present disclosure.

A first aspect of the present disclosure is to provide a phenanthrene organic compound having the structure represented by Formula I:

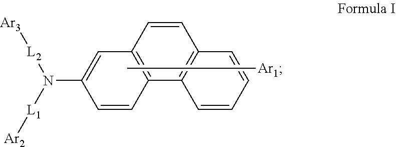

wherein Ar₁ is selected from substituted or unsubstituted C6-C20 aryl, substituted or unsubstituted C10-C20 fused-ring aryl, substituted or unsubstituted C2-C20 heteroaryl, or substituted or unsubstituted C4-C20 fused-ring heteroaryl;
L₁ and L₂ are independently selected from a single bond, substituted or unsubstituted C6-C20 arylene, substituted or unsubstituted C2-C20 heteroarylene, or substituted or unsubstituted C4-C20 fused-ring heteroarylene; and
Ar₂ and Ar₃ are independently selected from a substituted or unsubstituted C10-C20 fused-ring aromatic group or substituted or unsubstituted C4-C20 nitrogen-containing fused-ring heteroaryl having an electron-withdrawing property, and at least one of Ar₂ and Ar₃ is selected from substituted or unsubstituted C4-C20 nitrogen-containing fused-ring heteroaryl having an electron-withdrawing property.

In the present disclosure, C10-C20 may be C10, C12, C13, C14, C15, C16, C18 or C19, etc.
C6-C20 may be C6, C8, C10, C12, C13, C14, C15, C16, C18 or C20, etc.
C2-C20 may be C3, C5, C6, C8, C10, C12, C13, C14, C15, C16, C18 or C19, etc.
C4-C20 may be C5, C6, C8, C10, C12, C13, C14, C15, C16, C18 or C19, etc.

The phenanthrene organic compound of the present disclosure has a triarylamine structure, and the triarylamine structure is a special non-planar propeller-shaped molecular configuration. Compared with the planar molecular structure with a large conjugated system, in such a molecular configuration, the intermolecular interaction force is smaller, the required evaporation temperature is lower, and the long-term evaporation thermal stability of the material is higher.

At least one of Ar₂ and Ar₃ is selected from substituted or unsubstituted C2-C20 nitrogen-containing fused-ring heteroaryl having an electron-withdrawing property, and the phenanthrene organic compound of the present disclosure thus can effectively improve the molecular polarizability and the refractive index of materials.

Position 9 and position 10 of the phenanthrene group have high reactivity. When the phenanthrene group is located at the end group of the molecule, the exposed phenanthrene group located at the end group of the molecule is liable to collide with the phenanthrene group of an adjacent molecule during high-temperature evaporation, producing a reaction or causing molecular destruction. Compared with the molecular structure where the phenanthrene group is located at the end group, the phenanthrene in the phenanthrene compound of the present disclosure is linked to the chemical groups on both sides through position 2 and position 7, and position 9 and position 10 with high reactivity are effectively protected by the chemical groups on both sides so that the chemical structure of the phenanthrene is more stable and the long-term evaporation thermal stability is higher.

Compared with naphthalene, phenanthrene has a larger conjugated system and a better planar structure. Under high-temperature evaporation conditions, when the phenanthrene group is located at the end group of the molecule, due to stronger II-II interaction and stronger intermolecular force, the exposed phenanthrene group located at the end group of the molecule is liable to accumulate with the phenanthrene group of the adjacent molecule. To better evaporate the material on the substrate, a higher evaporation temperature is required to destroy the intermolecular force and even break intramolecular chemical bonds. As a result, the material is cracked, and more impurities are produced. Meanwhile, the material purity in the evaporation source is also continuously declined, the material purity in the thin film is declined, and more defects are produced, resulting in the deterioration of device performance such as luminescence efficiency and service life. The phenanthrene compound of the present disclosure puts the phenanthrene group inside the molecule, the intermolecular interaction force is reduced, the evaporation temperature is lowered, the long-term evaporation thermal stability of the material is improved, and defects are reduced, thereby significantly improving the device performance.

The phenanthrene compound of the present disclosure also has a high glass transition temperature and a high refractive index in the visible-light field, and when applied to the CPL layer of an organic electroluminescent device, can effectively improve the light extraction efficiency of the organic electroluminescent device, thereby improving the luminescence efficiency and service life of the organic electroluminescent device.

Preferably, Ar₁ in Formula I is selected from a substituted or unsubstituted C10-C20 fused aromatic ring group;
L₁ and L₂ are independently selected from a single bond or substituted or unsubstituted C6-C20 arylene; and
Ar₂ and Ar₃ are independently selected from a substituted or unsubstituted C10-C20 fused-ring aromatic group or substituted or unsubstituted C2-C20 nitrogen-containing fused-ring heteroaryl having an electron-withdrawing property, and at least one of Ar₂ and Ar₃ is selected from substituted or unsubstituted C2-C20 nitrogen-containing fused-ring heteroaryl having an electron-withdrawing property.

The fused aromatic ring group in the present disclosure includes, but is not limited to, naphthyl, fluorenyl, anthryl, indenyl, phenanthryl, pyrenyl, acenaphthenyl, triphenylene, chrysenyl, acenaphthylenyl or perylenyl, etc. The same expression hereinafter has the same meaning.

The nitrogen-containing fused-ring heteroaryl refers to a fused-ring heteroaryl containing N atoms and includes, but is not limited to, pyrazinyl, pyrimidinyl, pyridazinyl, triazinyl, quinolyl, isoquinolyl, quinoxalinyl, quinazolinyl, benzopyridazinyl, pyridopyridinyl, pyridopyrazinyl, imidazolyl, oxazolyl, thiazolyl, triazolyl, oxadiazolyl, thiadiazolyl, benzimidazolyl or o-phenanthrolinyl, etc.

In an embodiment, the substituent in the substituted group is selected from deuterium, tritium, halogen, cyano or adamantyl.

In an embodiment, L₁ and L₂ are independently selected from a single bond, phenylene, biphenylene, naphthylene, anthrylene, phenanthrylene, pyrenylene, fluoranthenylene, triphenylenylene, pyridylene, pyrimidinylene, triazinylene, furylene, thienylene, quinolylene, isoquinolylene, benzimidazolylene, benzoxazolylene, benzothiazolylene, dibenzofurylene, dibenzothienylene, thiazolylene, oxazolylene, oxadiazolylene, thiadiazolylene, benzofurylene, benzothienylene or phenanthrolinylene.

In an embodiment, L₁ and L₂ are selected from a single bond or phenylene.

In an embodiment, Ar₁ is selected from naphthyl, anthryl, phenanthryl, pyrenyl, fluoranthenyl or triphenylene, preferably naphthyl. Naphthalene can improve the molecular refractive index and has a smaller molecular weight, the evaporation temperature becomes lower, and the thermal stability of the material at high temperatures becomes higher. The larger the molecular weight of a fused ring is, the higher the evaporation temperature becomes and the worse long-term evaporation thermal stability becomes. Compared with other fused rings, naphthalene has lower chemical reactivity and better thermal and chemical stability.

In an embodiment, the C10-C20 fused-ring aromatic group in the definition of Ar₂ and Ar₃ is selected from naphthyl, anthryl, phenanthryl, pyrenyl, fluoranthenyl or triphenylene.

In an embodiment, the C4-C20 nitrogen-containing fused-ring heteroaryl having an electron-withdrawing property in the definition of Ar₂ and Ar₃ is selected from the structure represented by Formula II, Formula III or Formula IV:

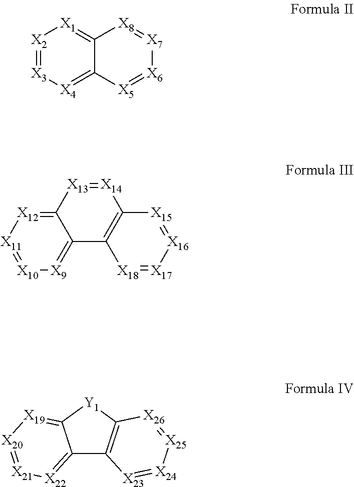

wherein X₁ to X₂₆ are independently selected from CR₁ or N;
at least one of X₁ to X₈ is selected from N;
at least one of X₉ to X₁₈ is selected from N;
at least one of X₁₉ to X₂₆ is selected from N;
Y₁ is selected from O or S;
wherein the linkage site of the group is any linkable position.

In an embodiment, at least one of Ar₂ and Ar₃ is selected from any one of the following nitrogen-containing fused-ring heteroaryl having an electron-withdrawing property:

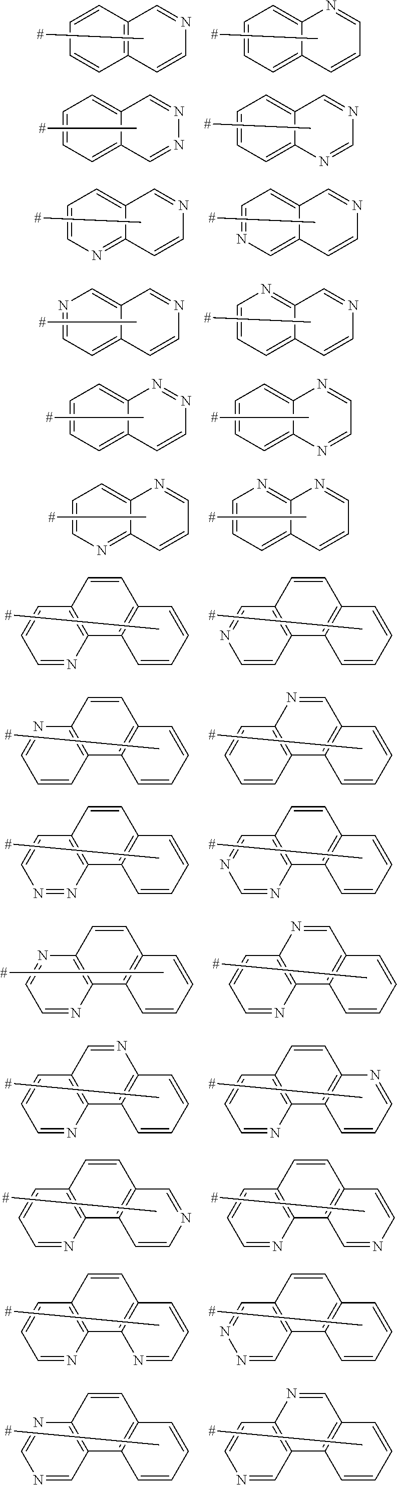

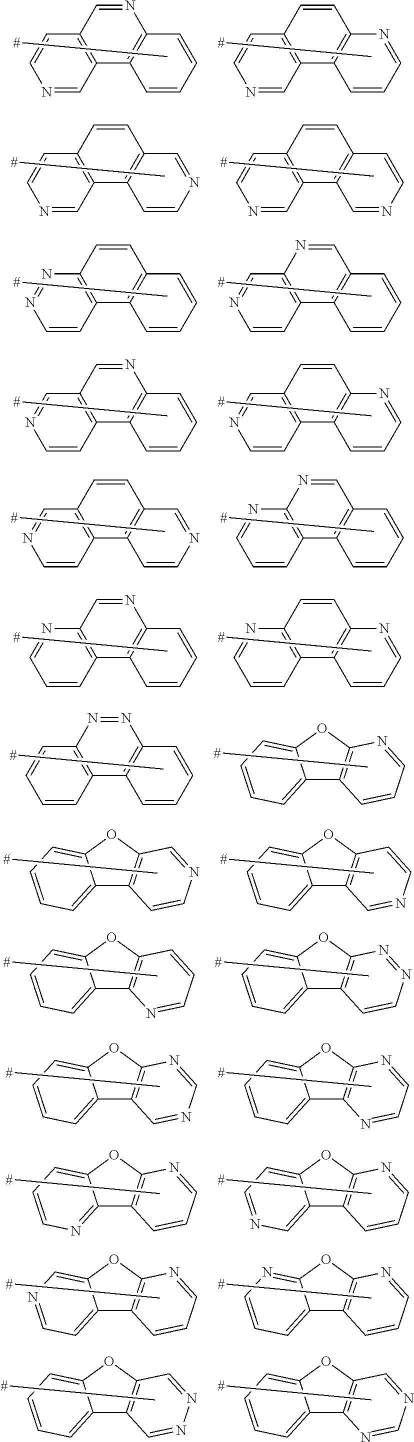

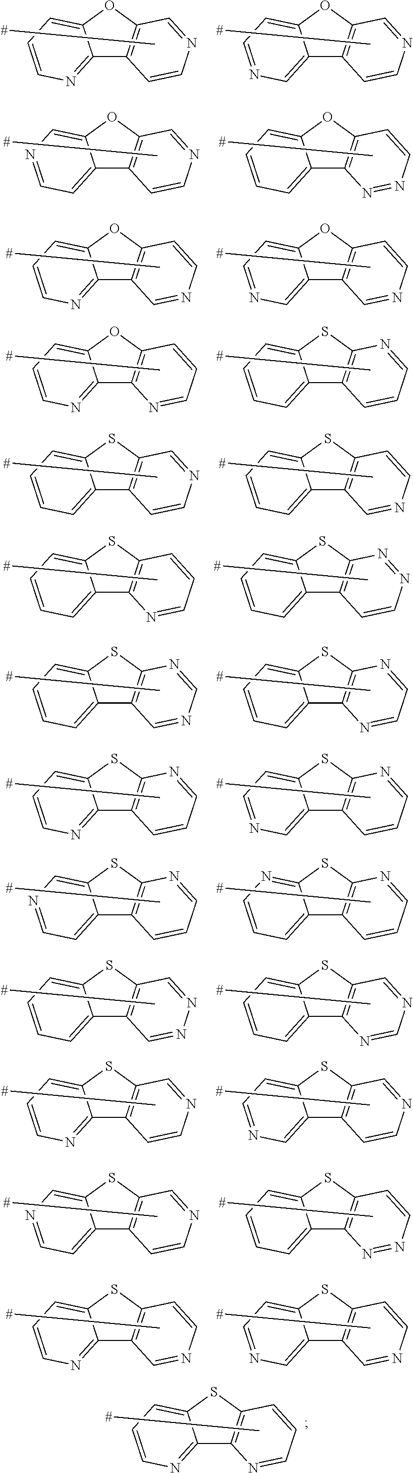

wherein # is a position where groups are joined.

In an embodiment, at least one of Are and Ar₃ is selected from the group represented by Formula V:

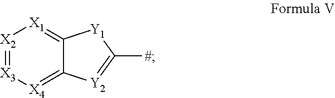

wherein X₁, X₂, X₃, and X₄ are independently selected from CR₁ or N;
Y₁ is selected from O, S, NR₂ or C(R₂)₂, and Y₂ is selected from N or CR₂;
at least one of X₁, X₂, X₃, X₄, Y₁, and Y₂ is N or NR₂;
R₁ and R₂ are selected from hydrogen, deuterium, C1-C10 alkyl or C6-C20 aryl.

In an embodiment, at least one of Ar₂ and Ar₃ is selected from any one of the following groups:

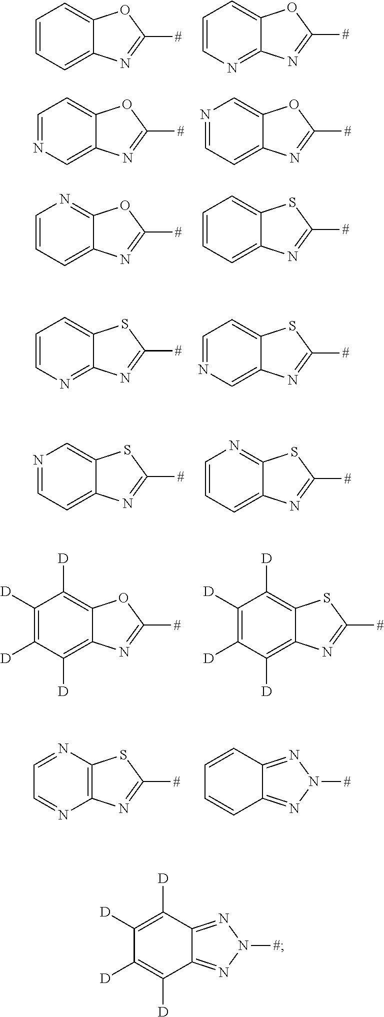

wherein D represents deuterium, and # is a position where groups are joined.

In an embodiment, at least one of Ar₂ and Ar₃ is selected from the group represented by Formula VI:

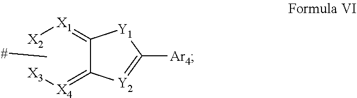

wherein X₁, X₂, X₃, and X₄ are independently selected from CR₁ or N;
Y₁ and Y₂ are independently selected from O, S or NR₂;
at least one of X₁, X₂, X₃, X₄, Y₁, and Y₂ is N or NR₂;
R₁ and R₂ are independently selected from hydrogen, deuterium or C6-C20 aryl.

In an embodiment, at least one of Ar₂ and Ar₃ is selected from any one of the following groups:

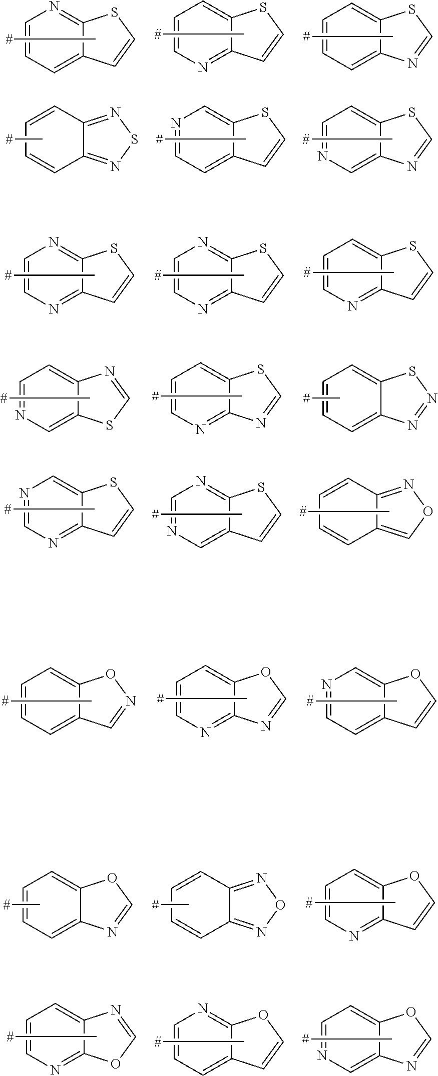

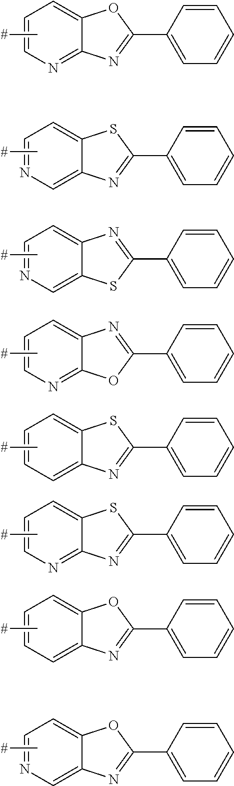

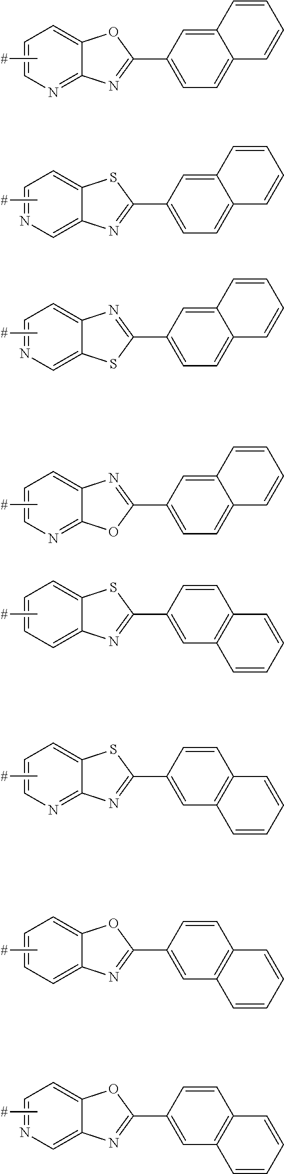

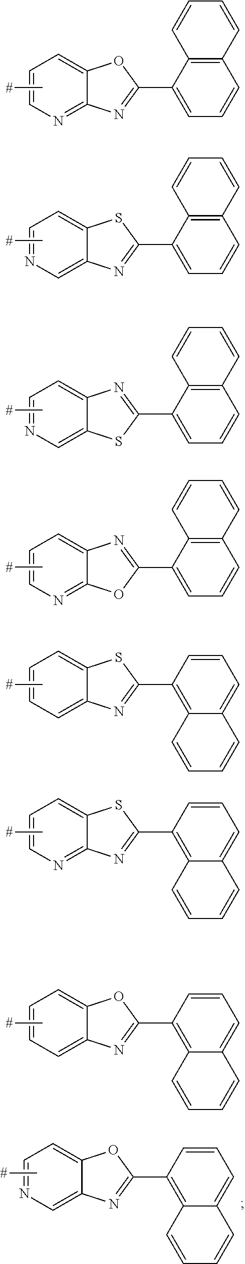

wherein # is a position where groups are joined.

In an embodiment, the phenanthrene organic compound has the structure represented by Formula II:

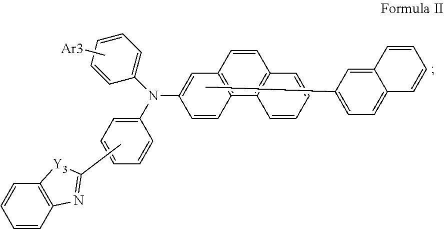

wherein Y₃ is selected from O or S;
Ar₃ is selected from any one of the following groups:

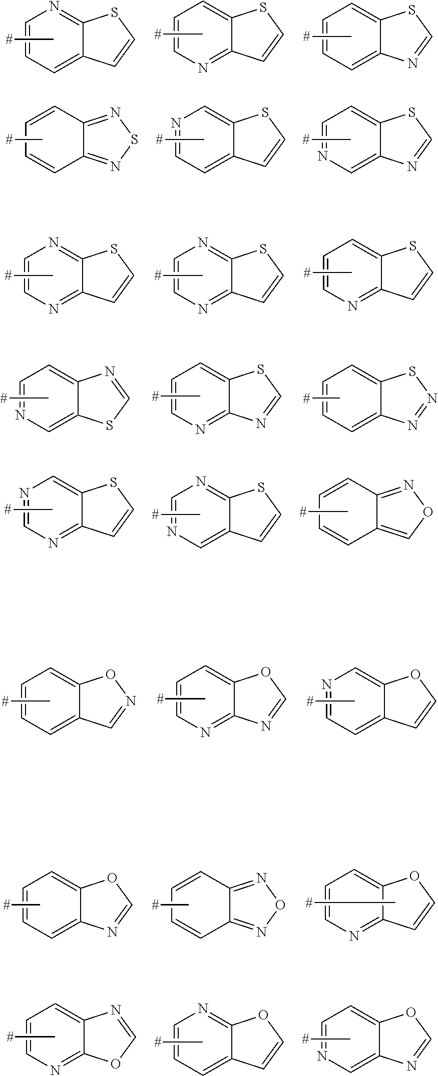

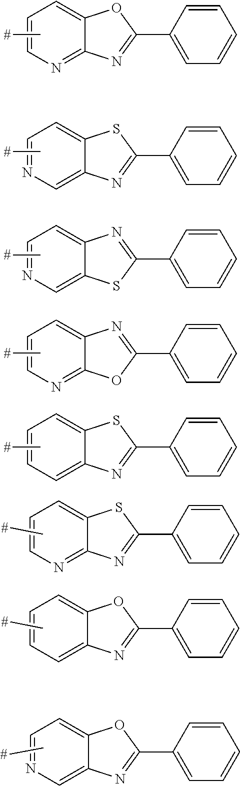

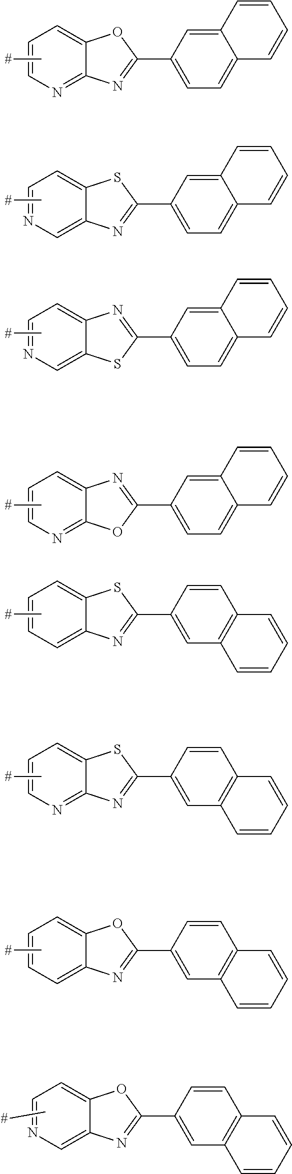

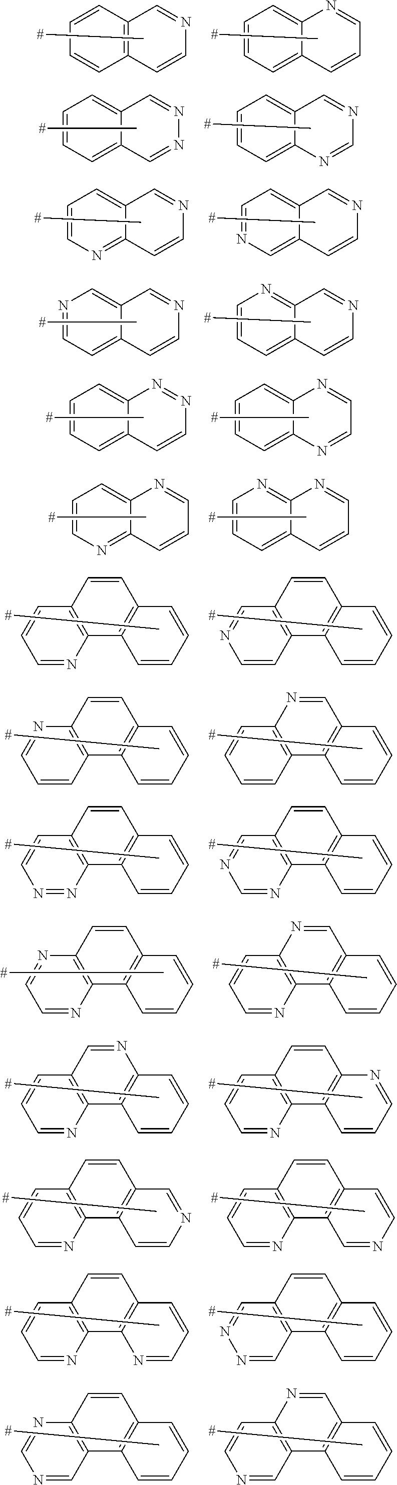

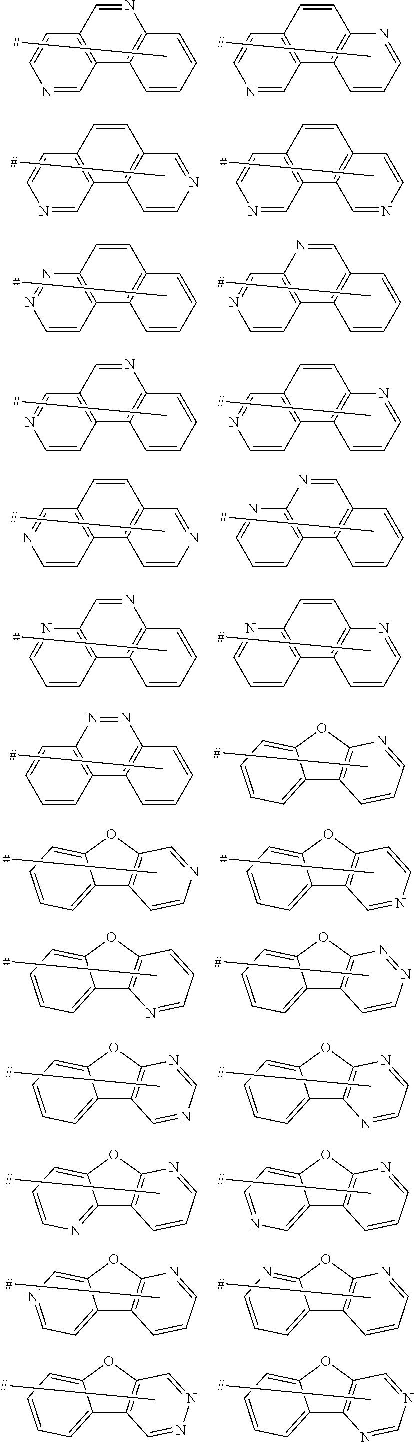

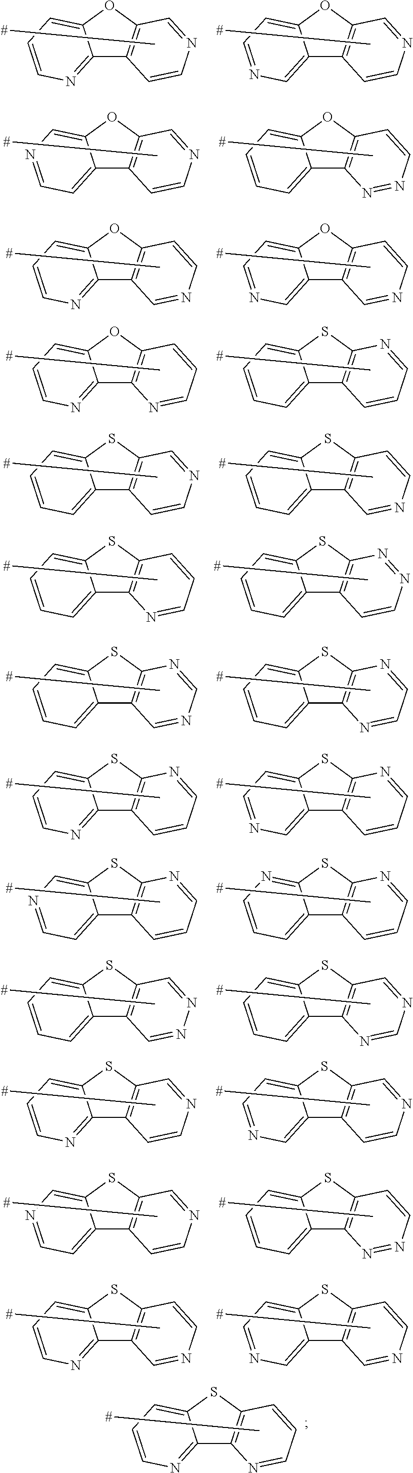

wherein # is a position where groups are joined.

When the compound having the structure represented by Formula II is selected, the benzoxazole group can improve the molecular polarizability and the refractive index of the molecule. The benzoxazole structure is simple in synthesis and low in cost, and benzoxazole has good thermal stability, chemical stability and photochemical stability.

In an embodiment, the phenanthrene organic compound is any one of the following compounds:

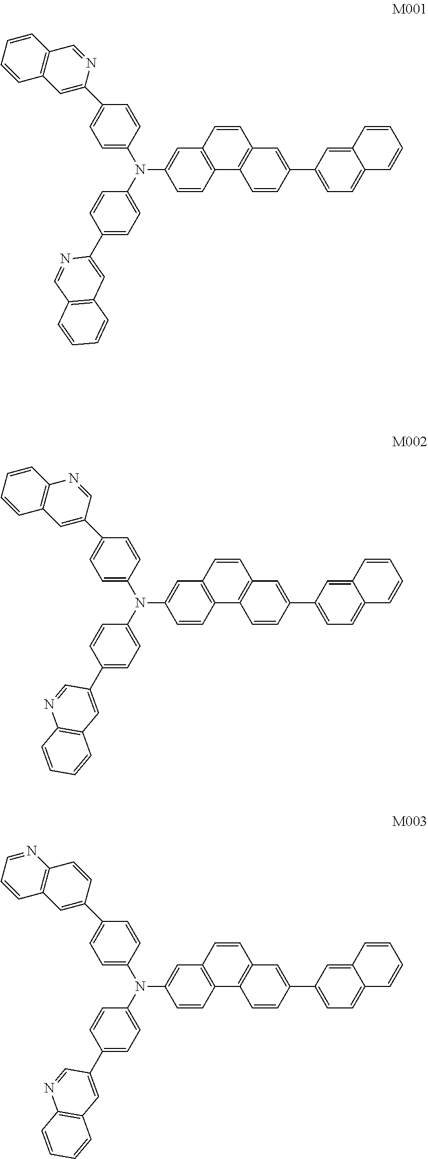

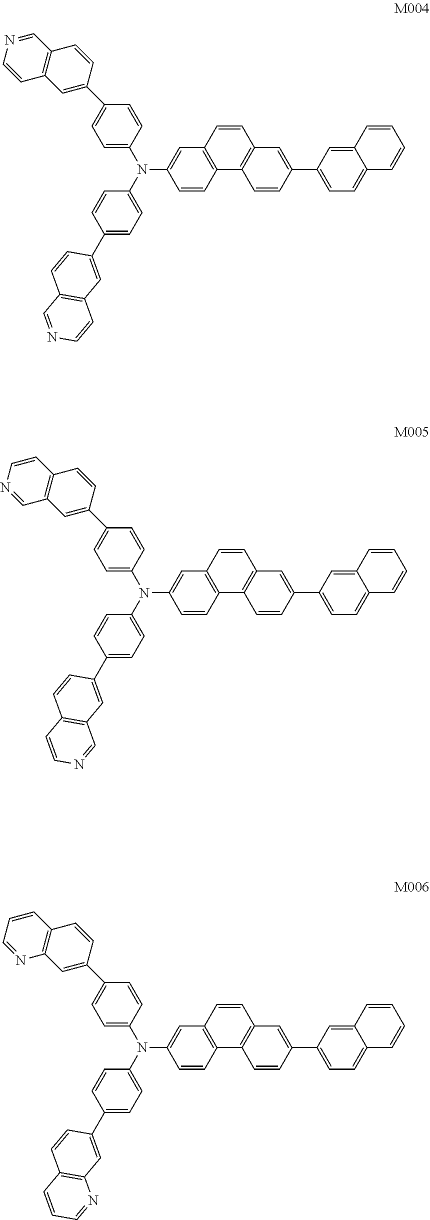

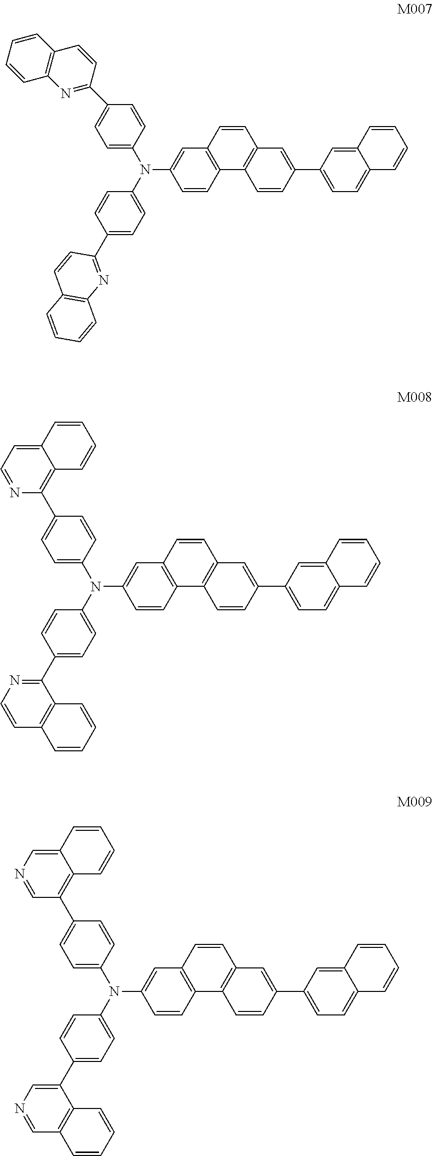

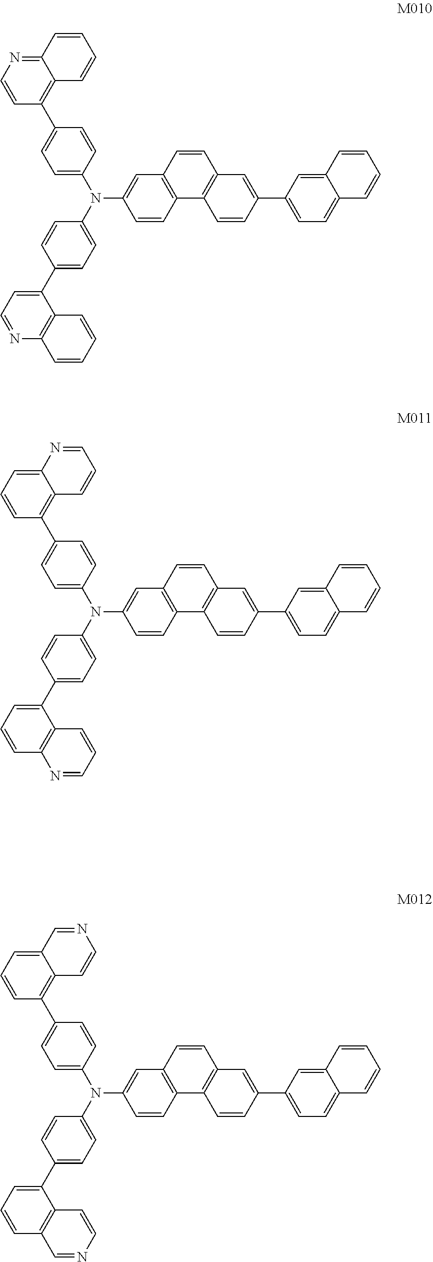

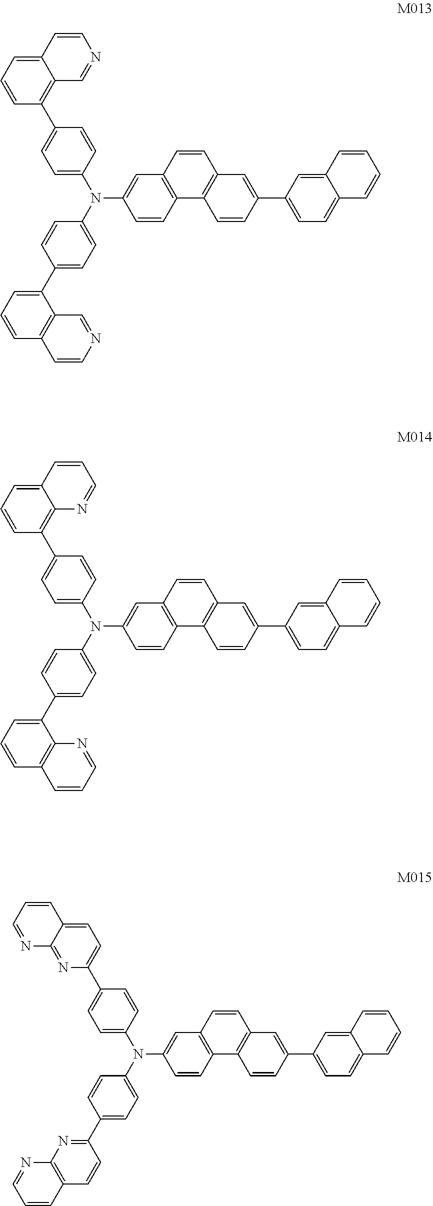

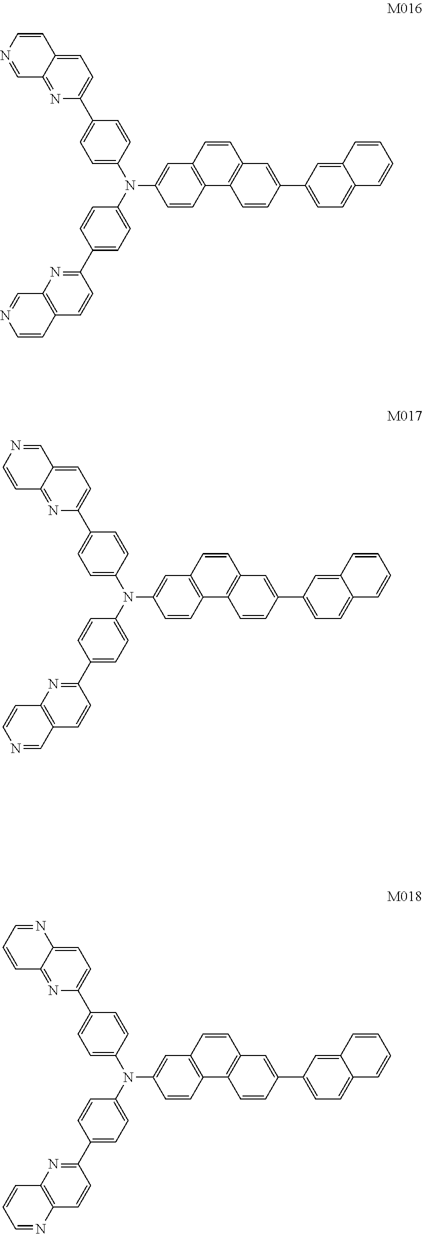

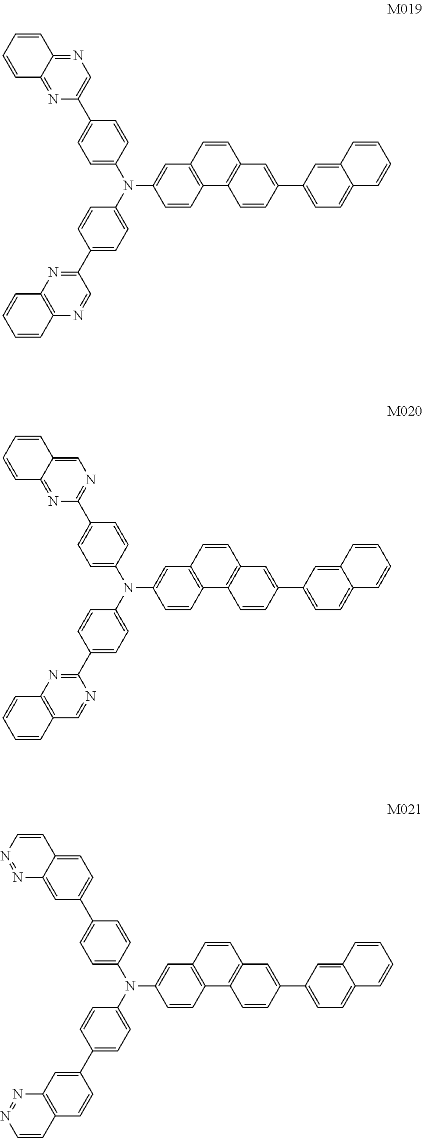

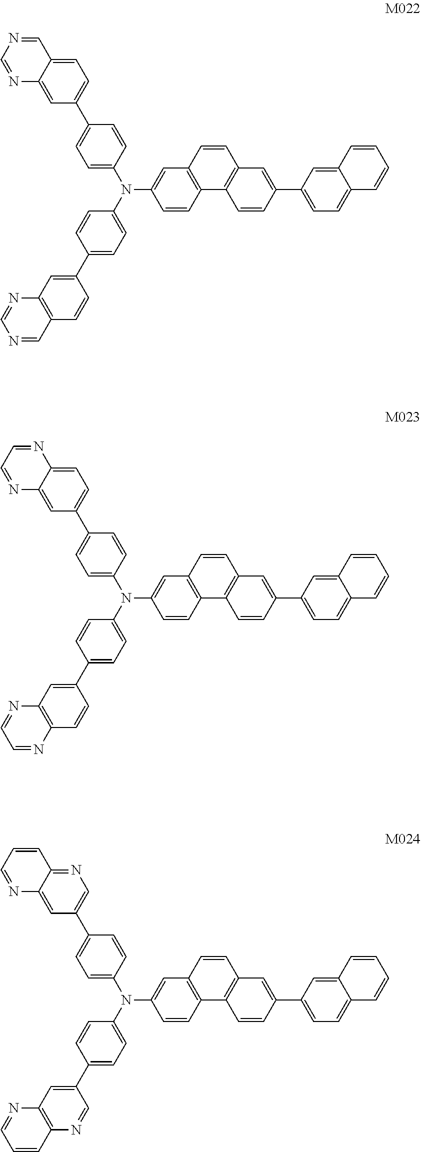

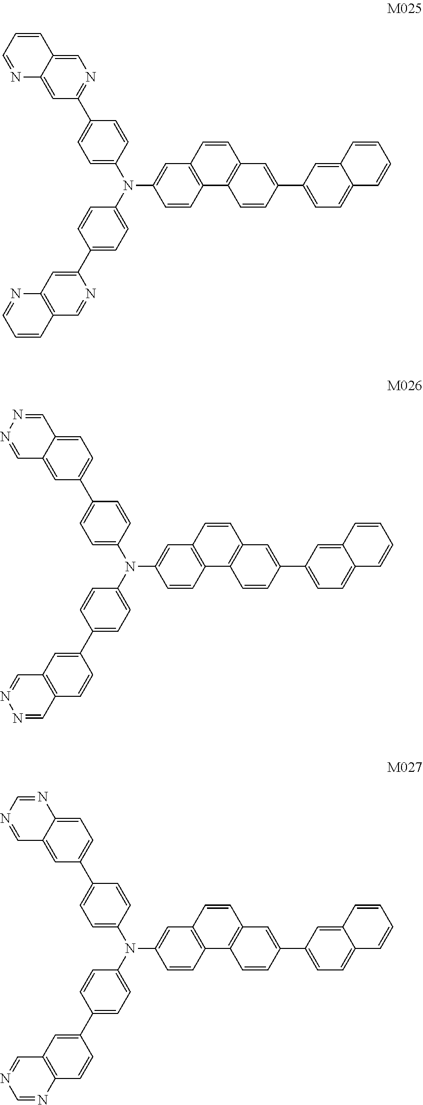

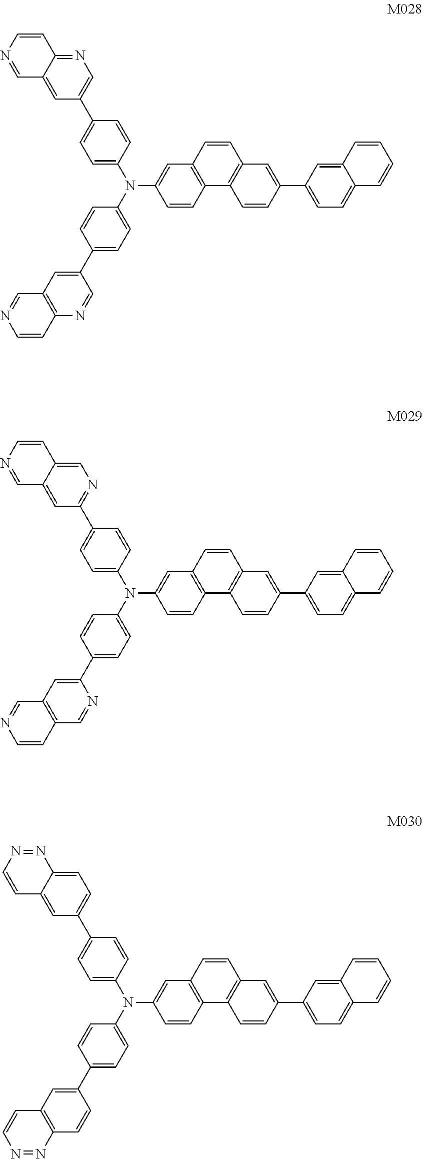

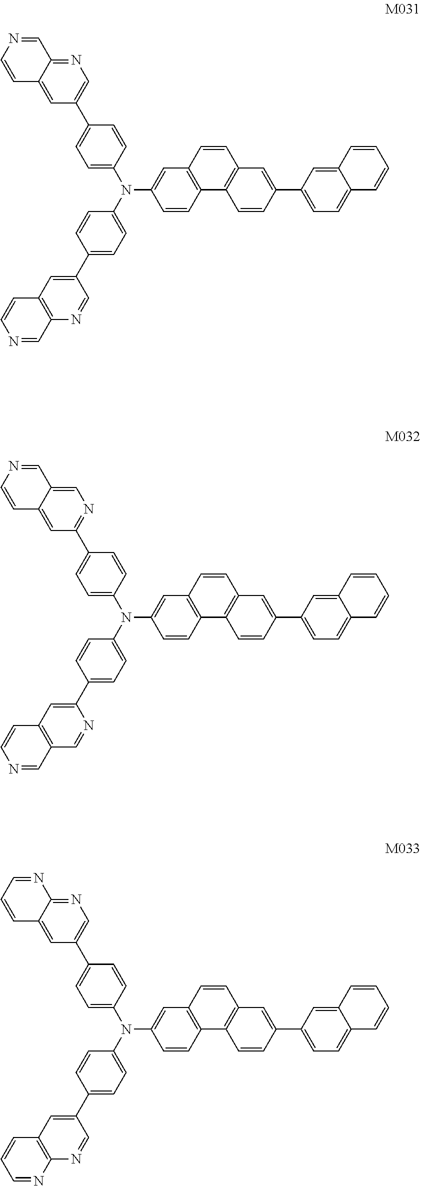

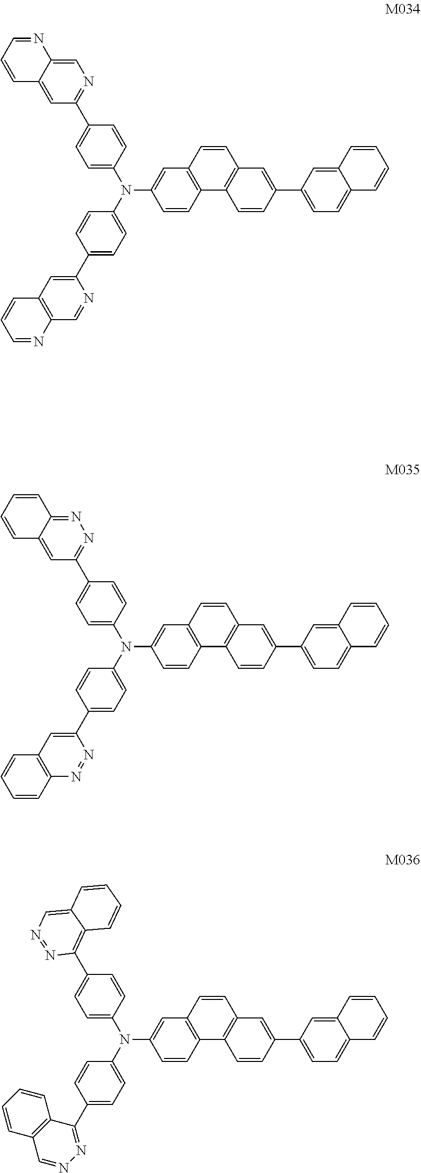

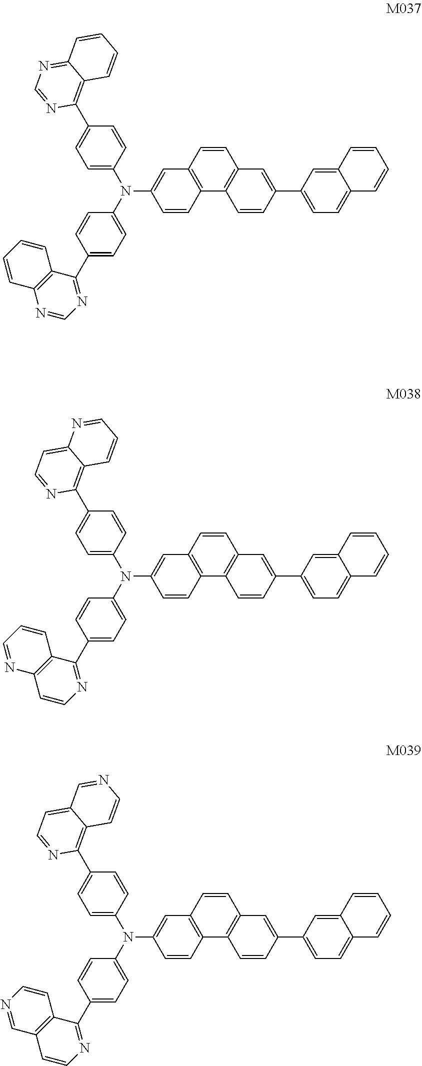

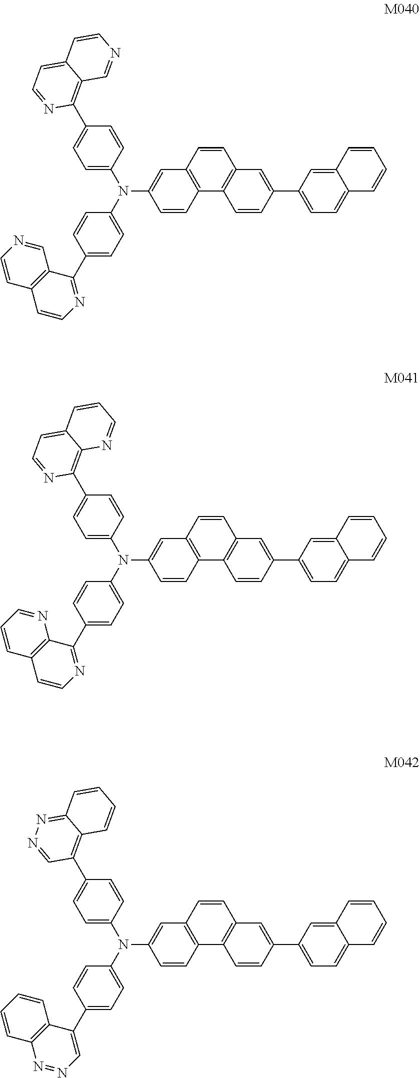

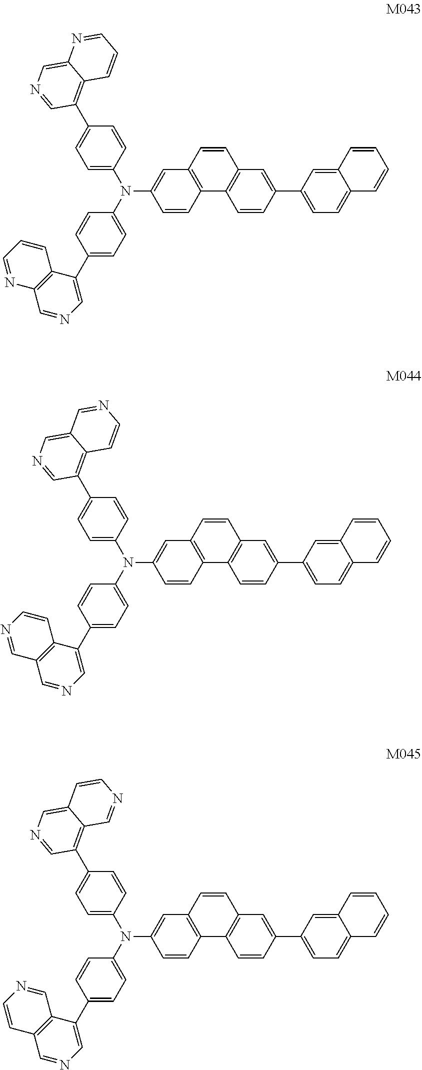

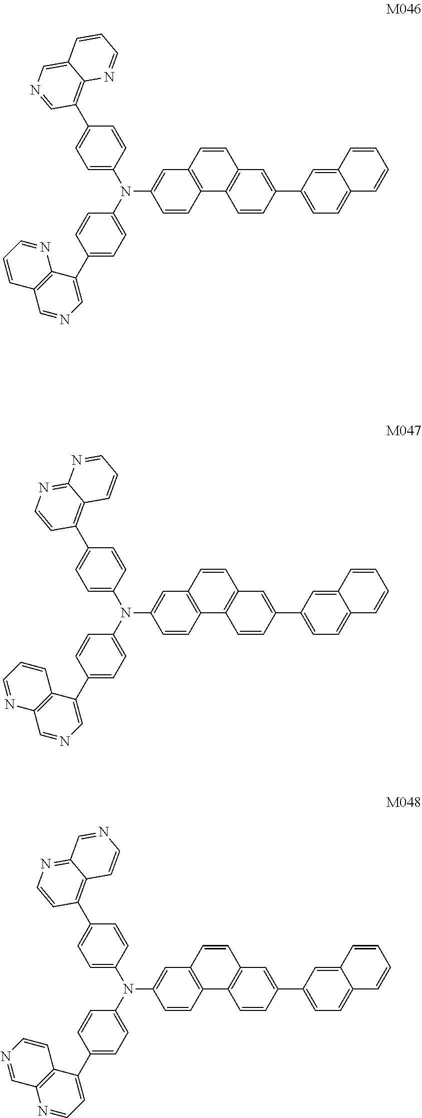

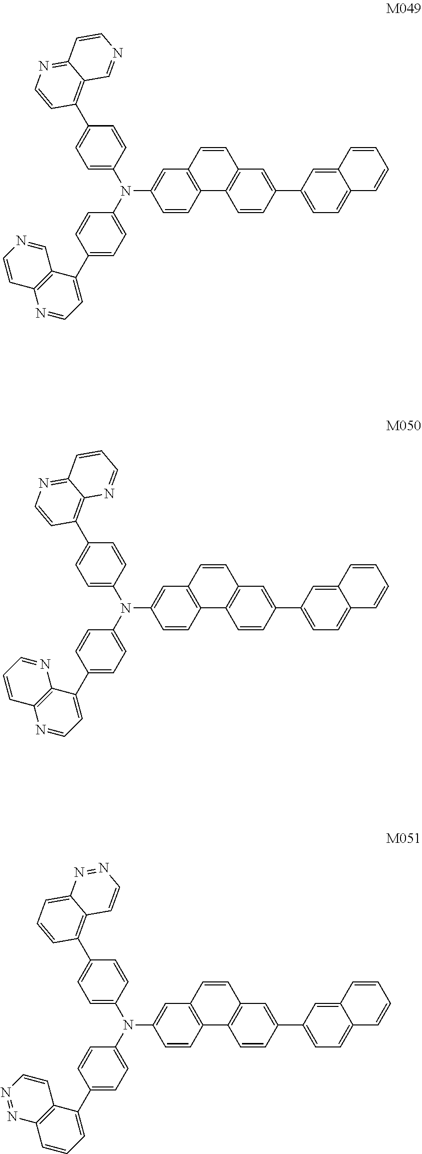

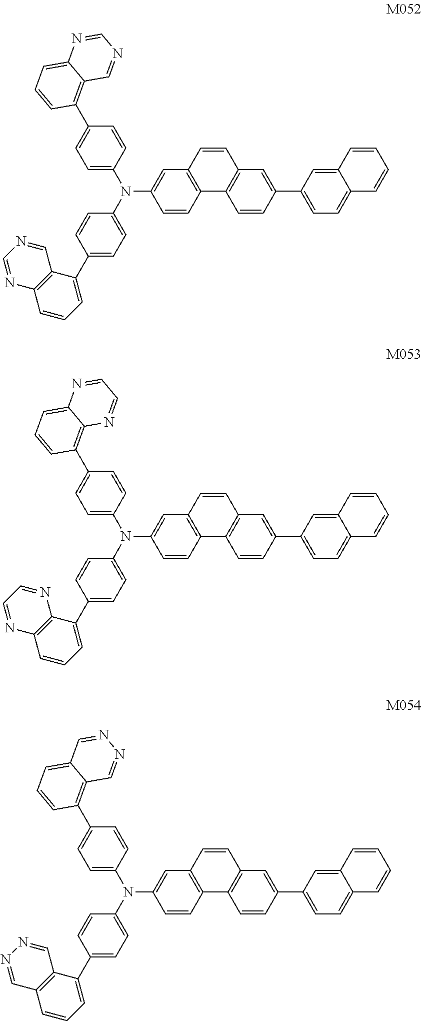

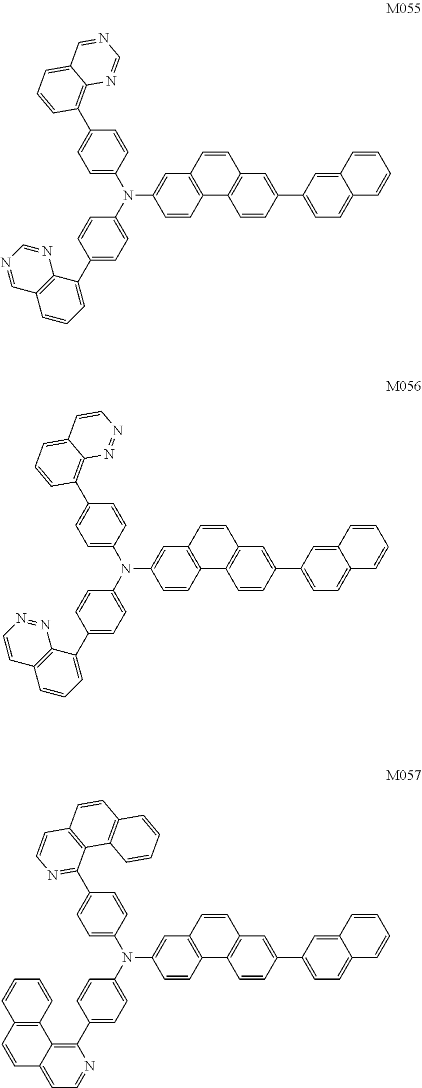

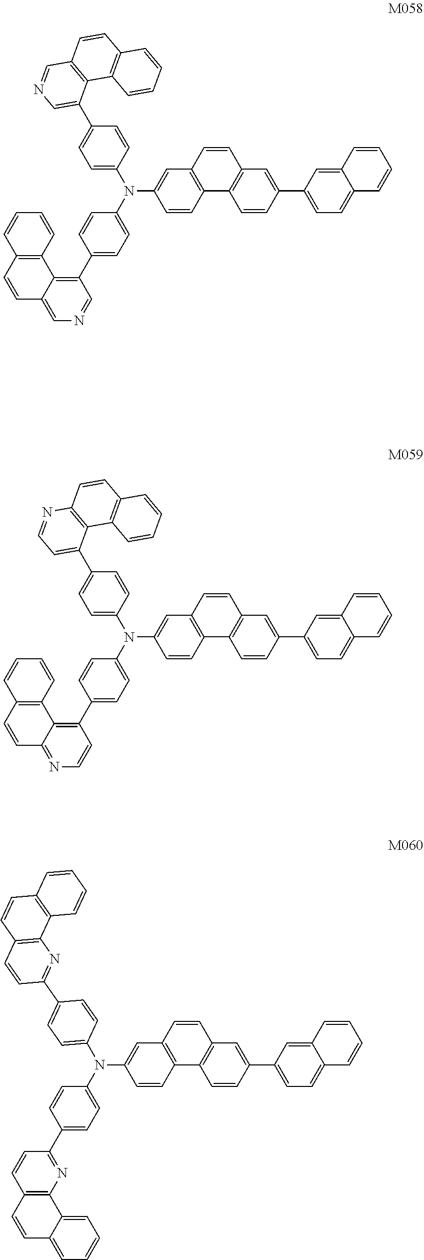

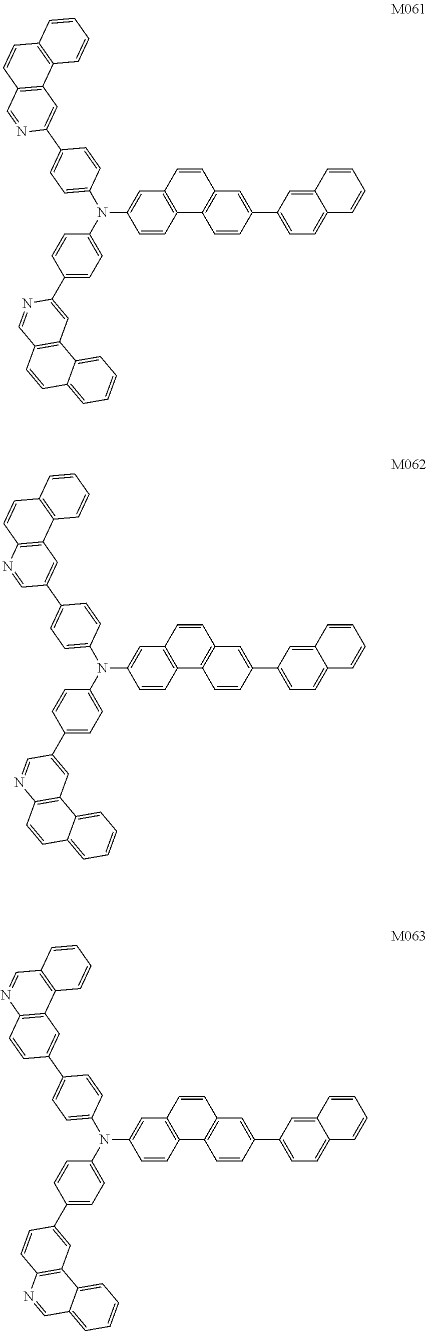

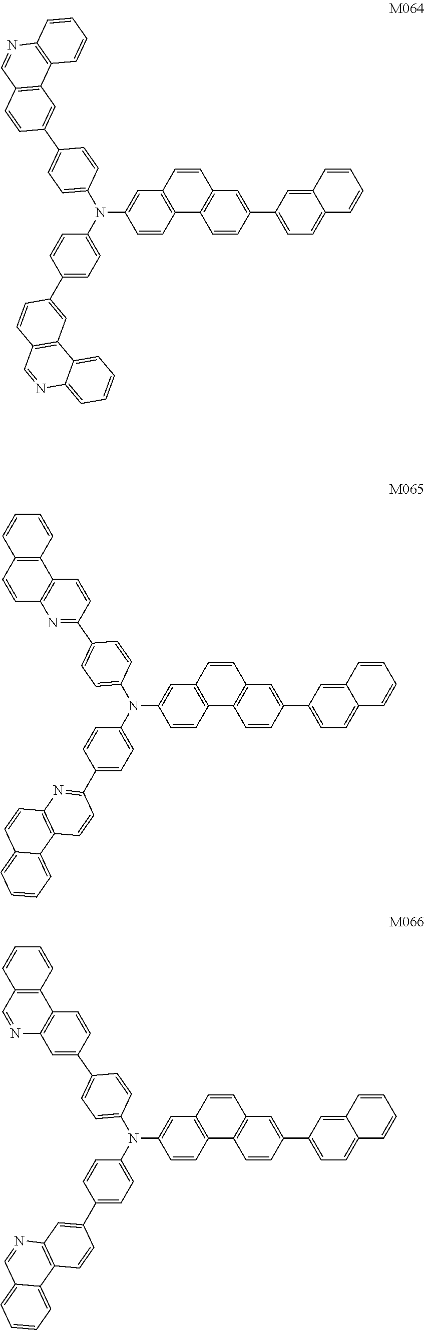

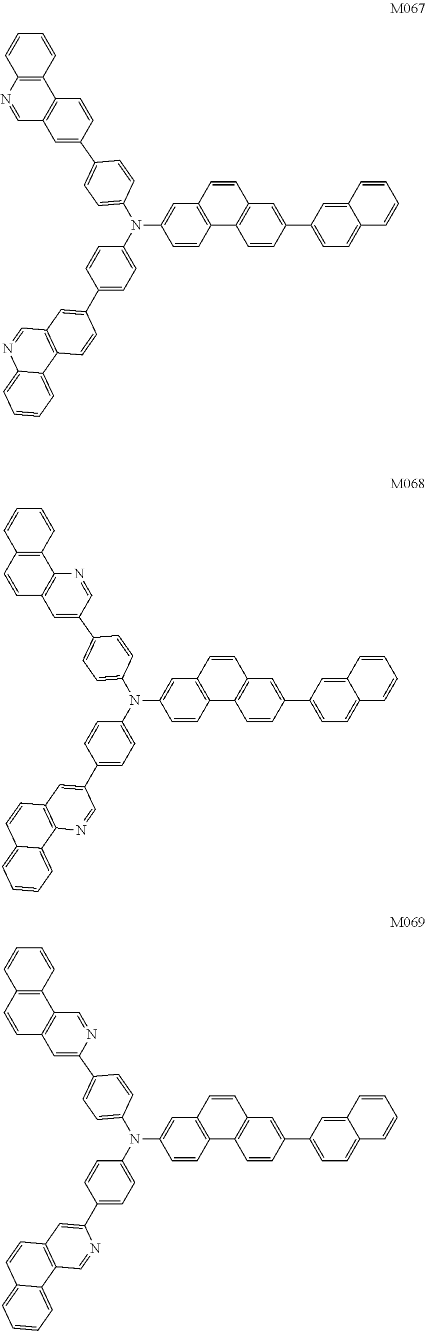

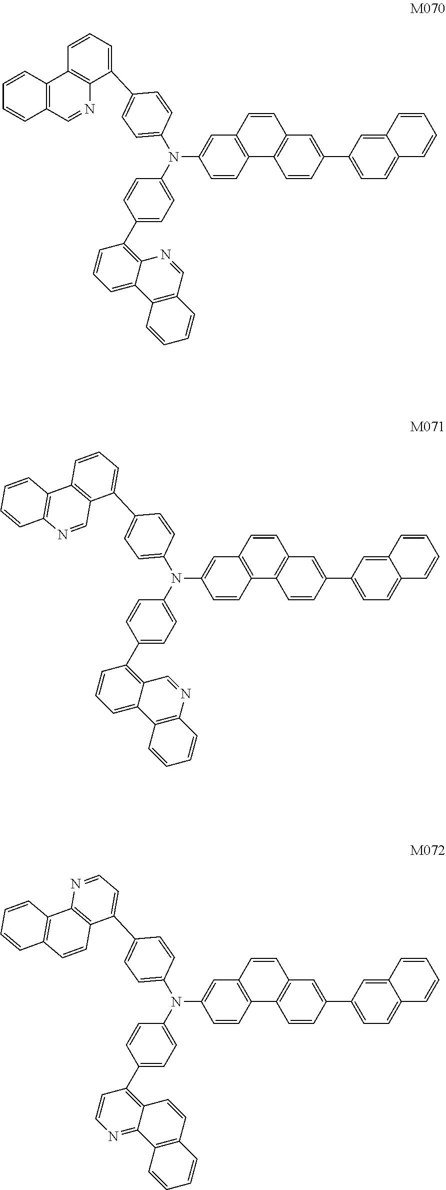

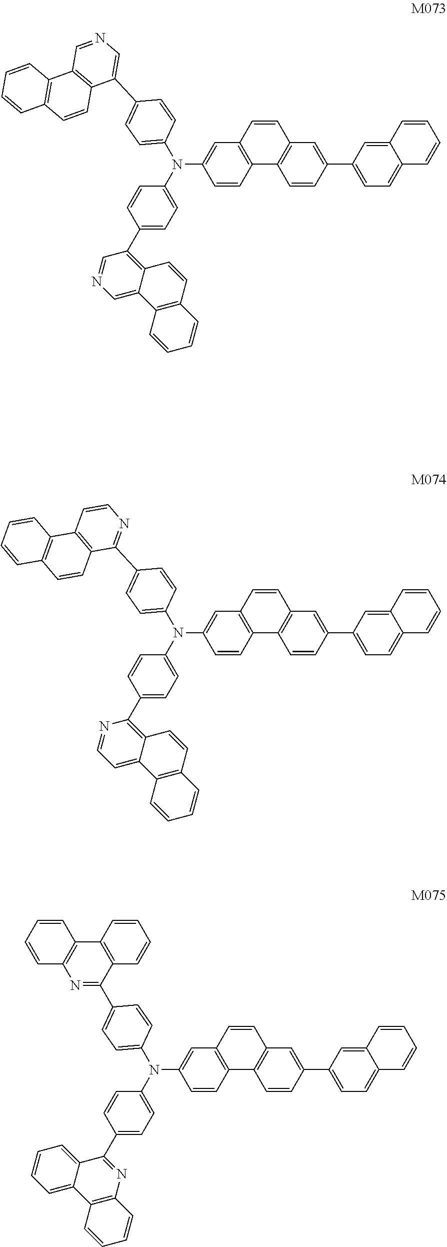

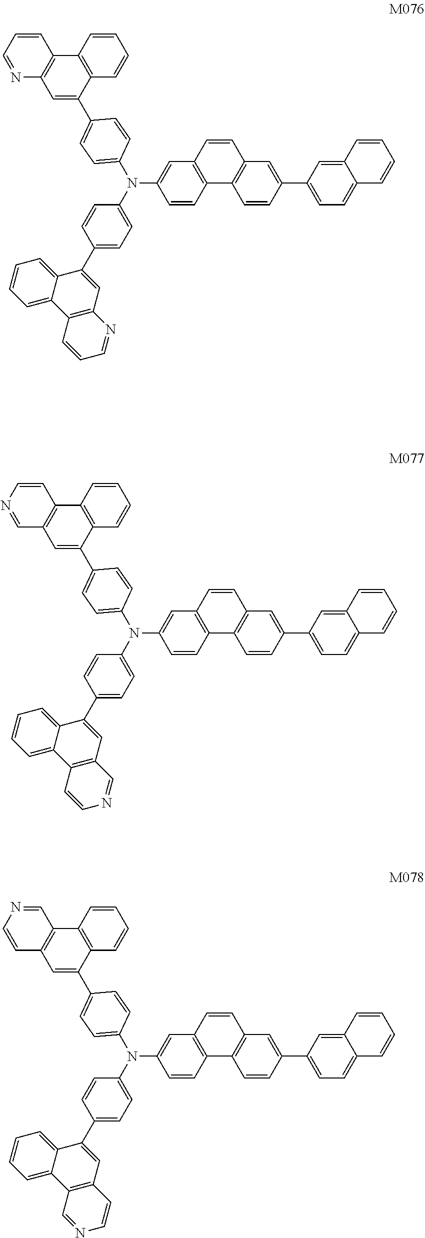

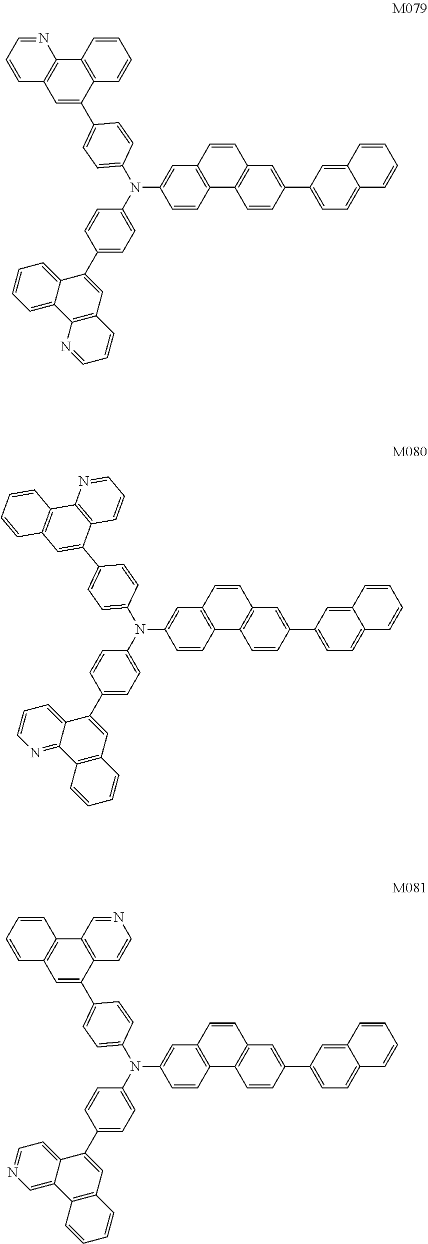

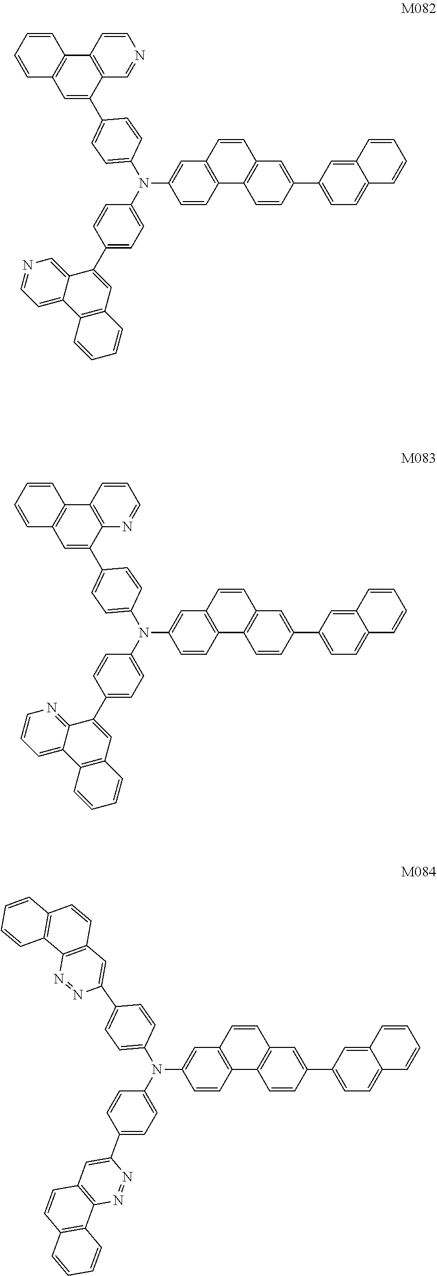

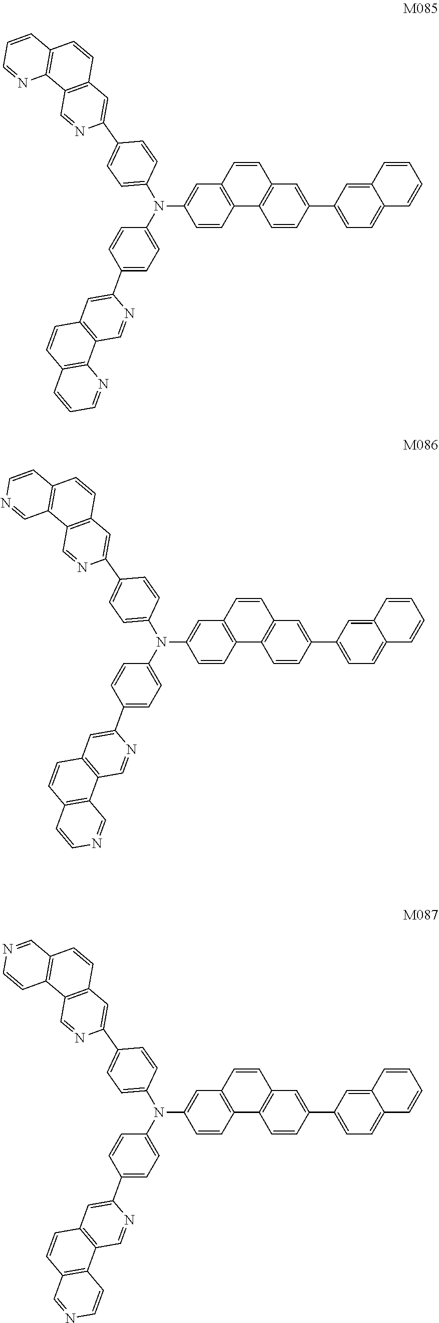

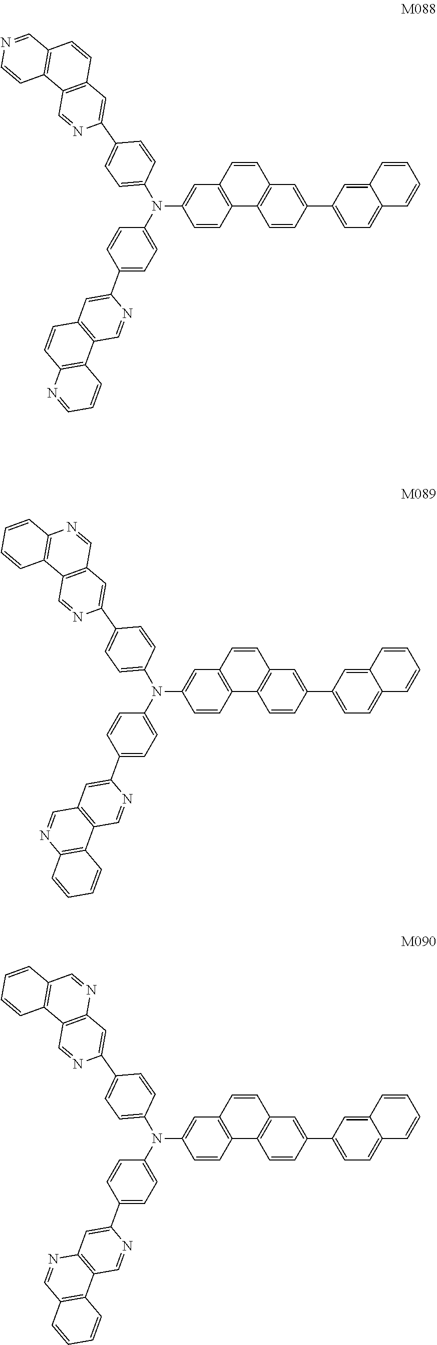

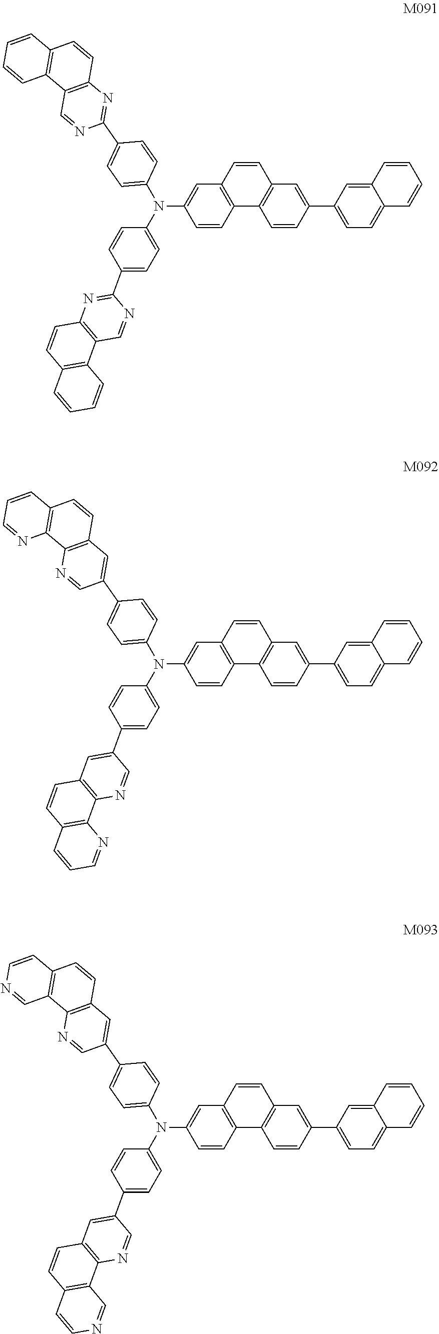

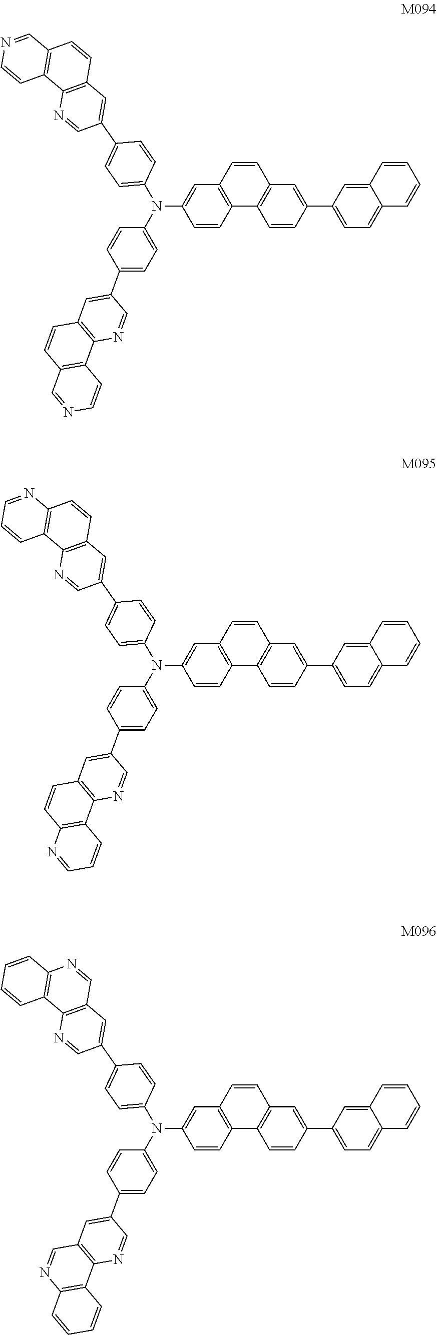

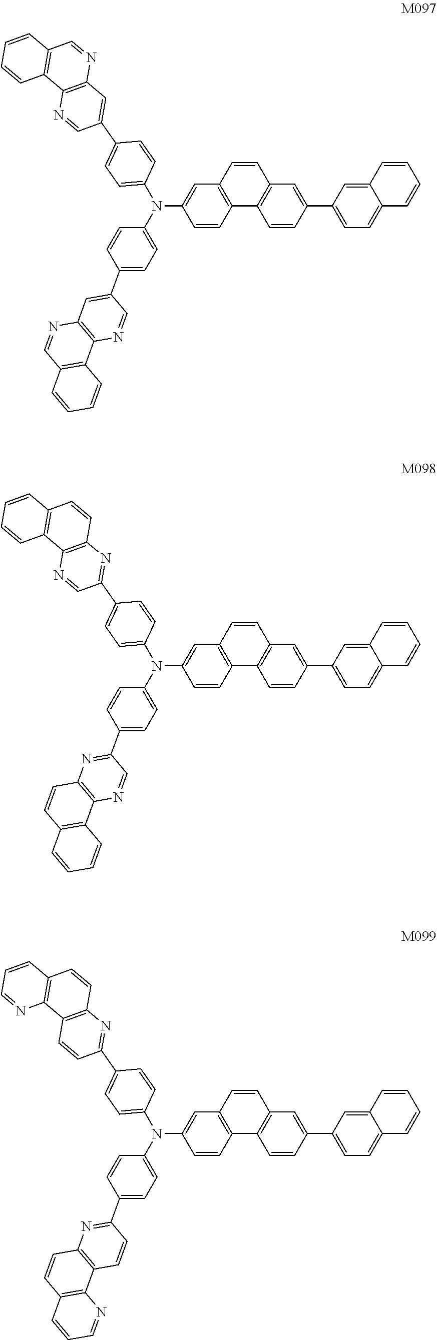

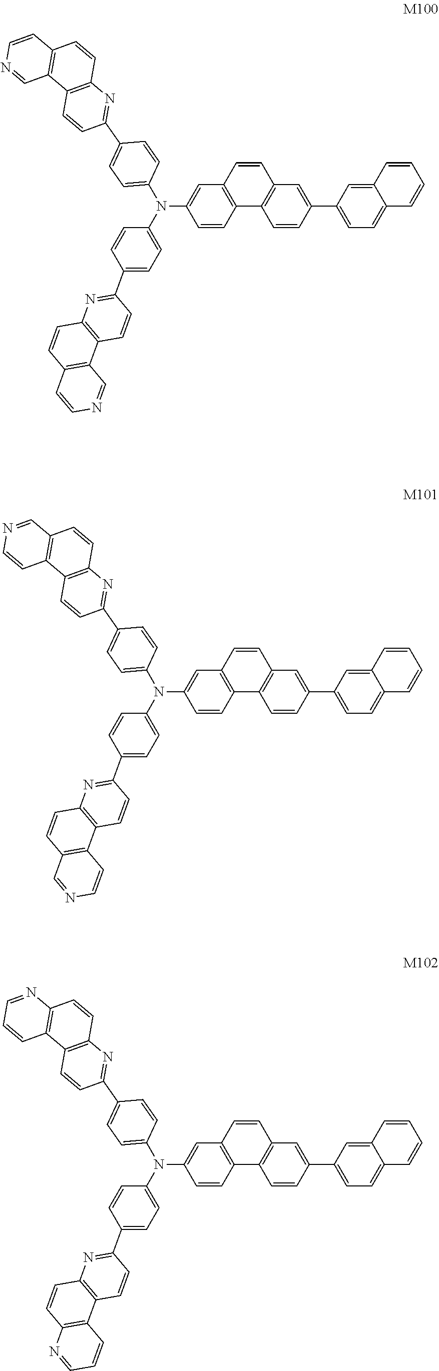

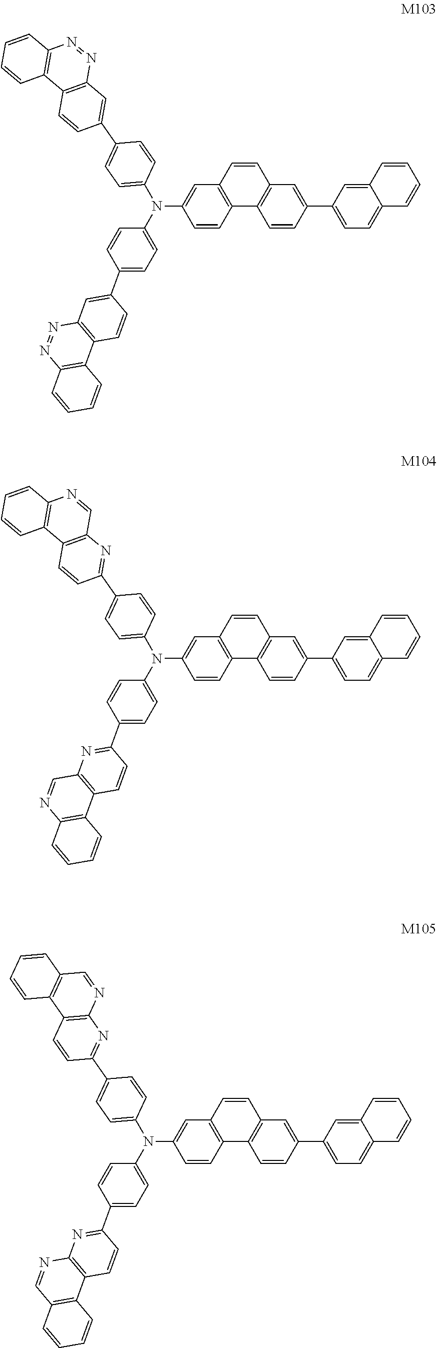

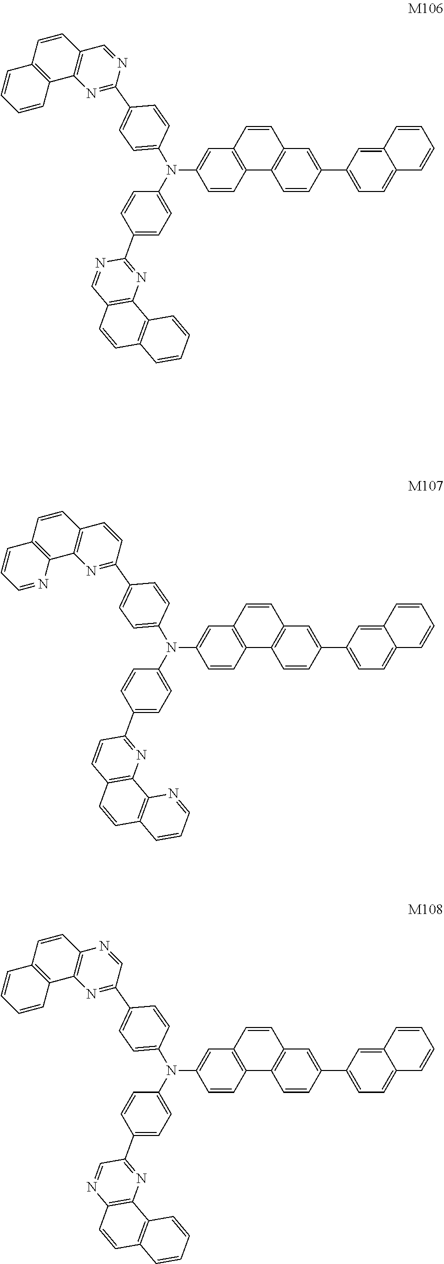

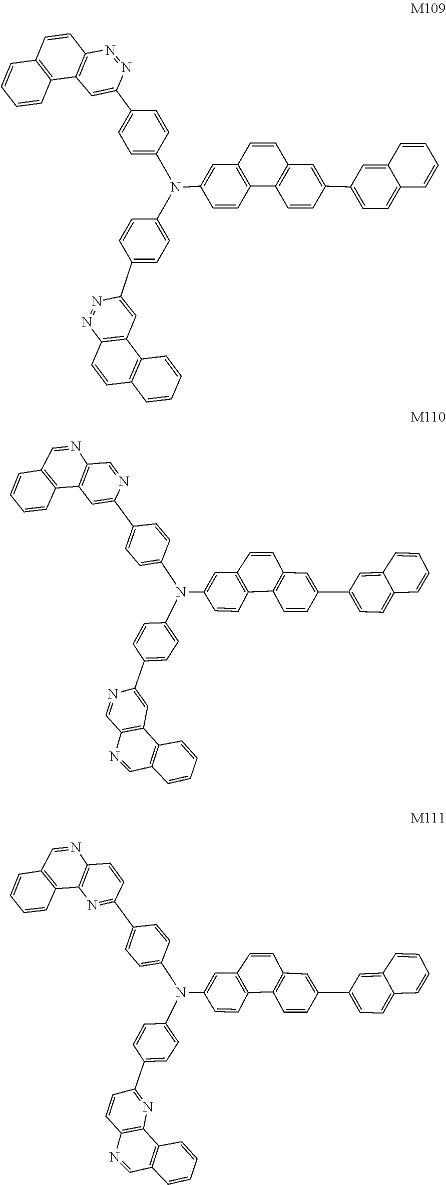

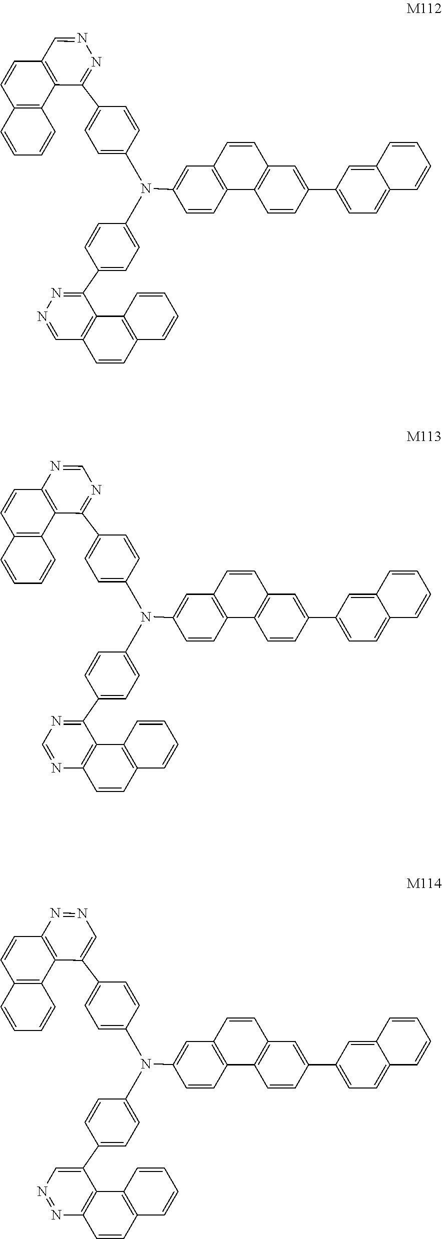

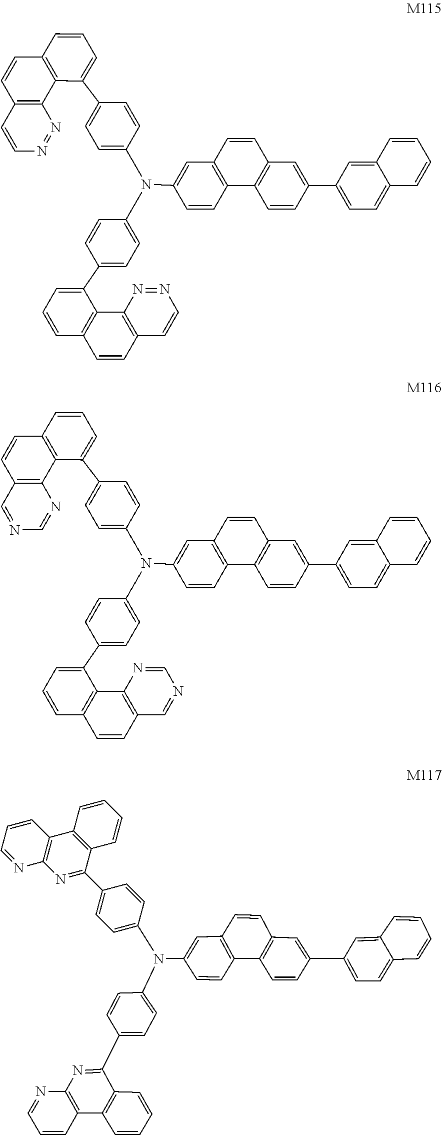

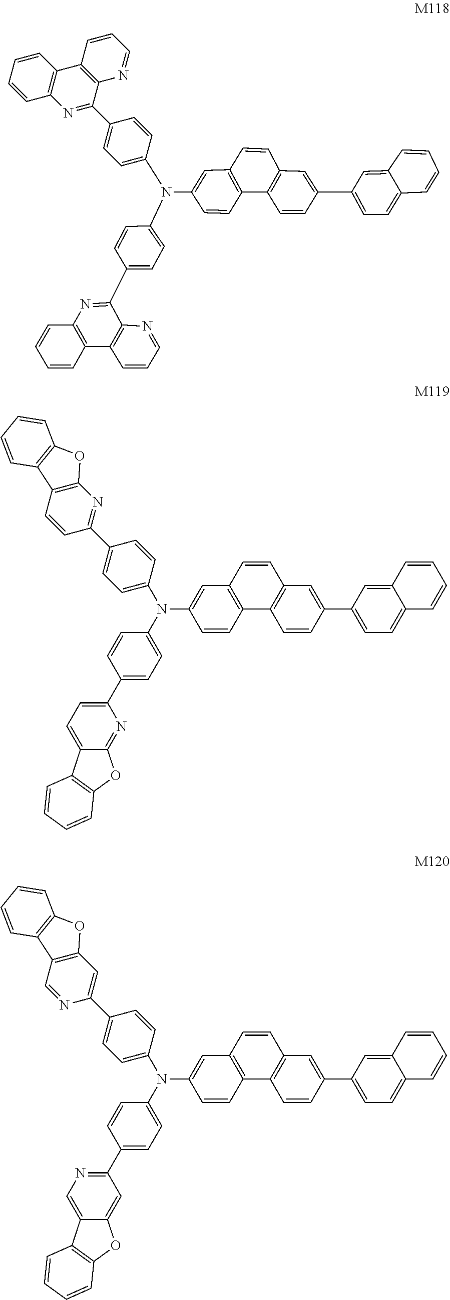

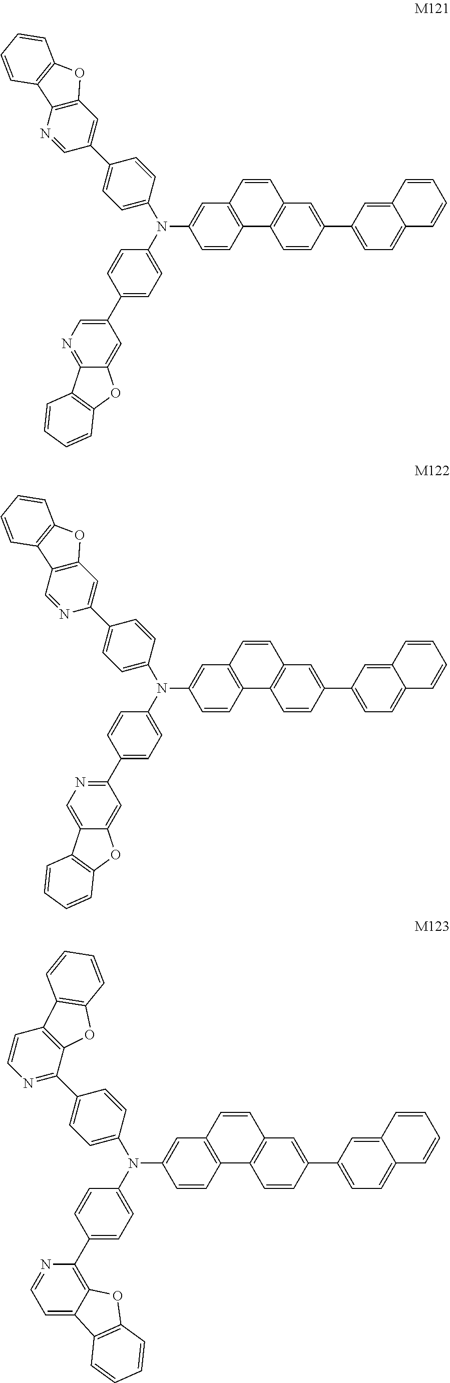

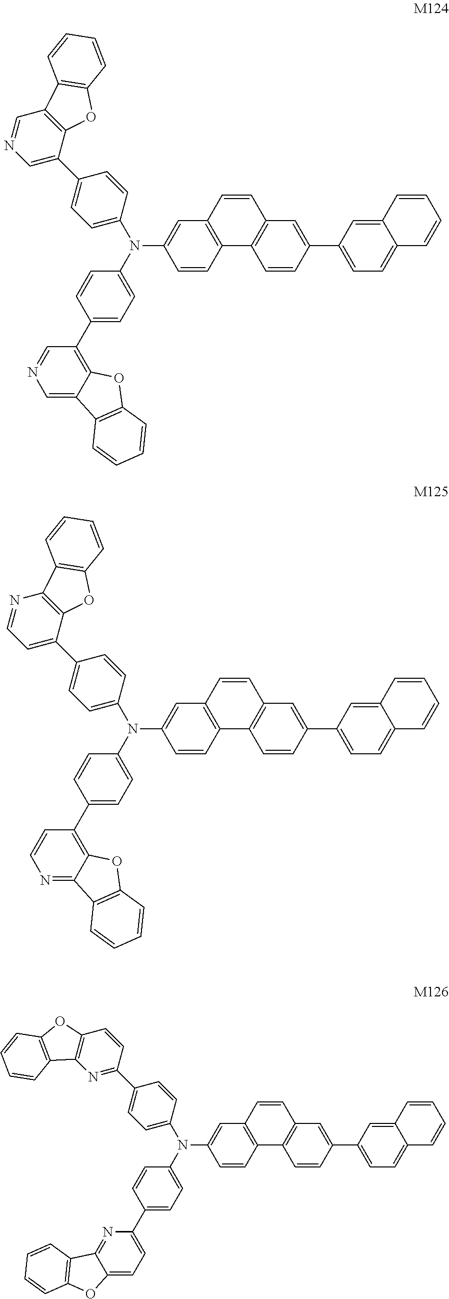

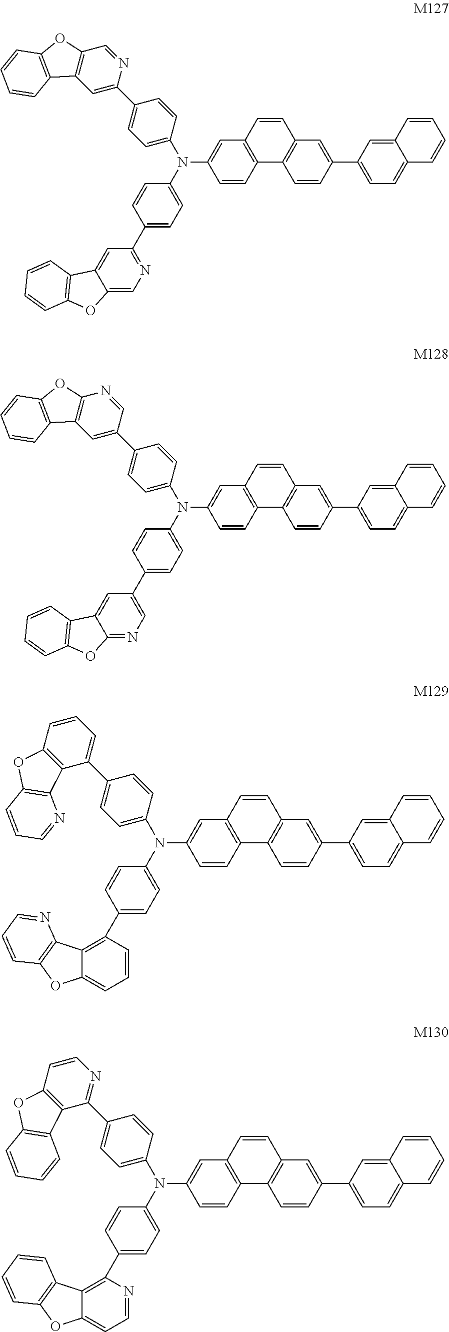

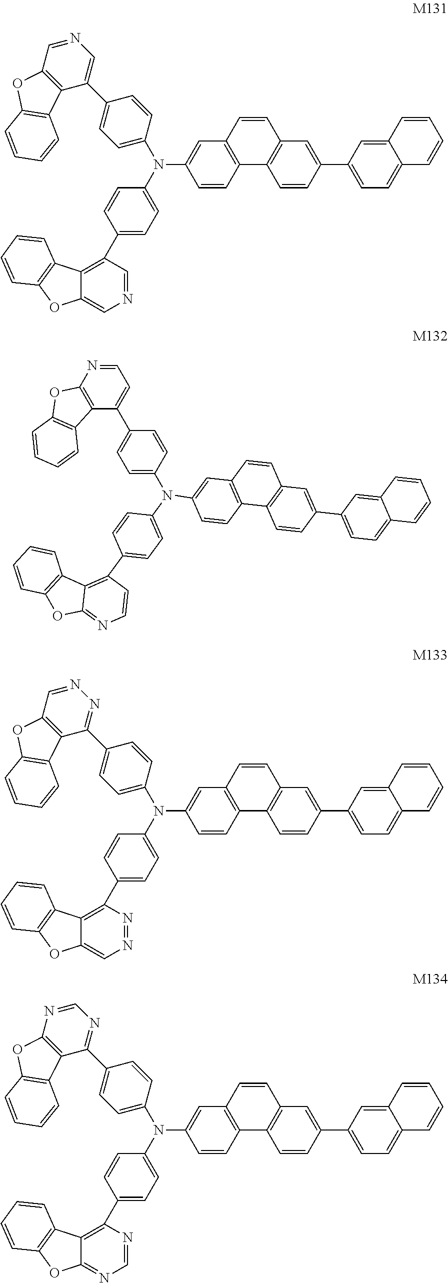

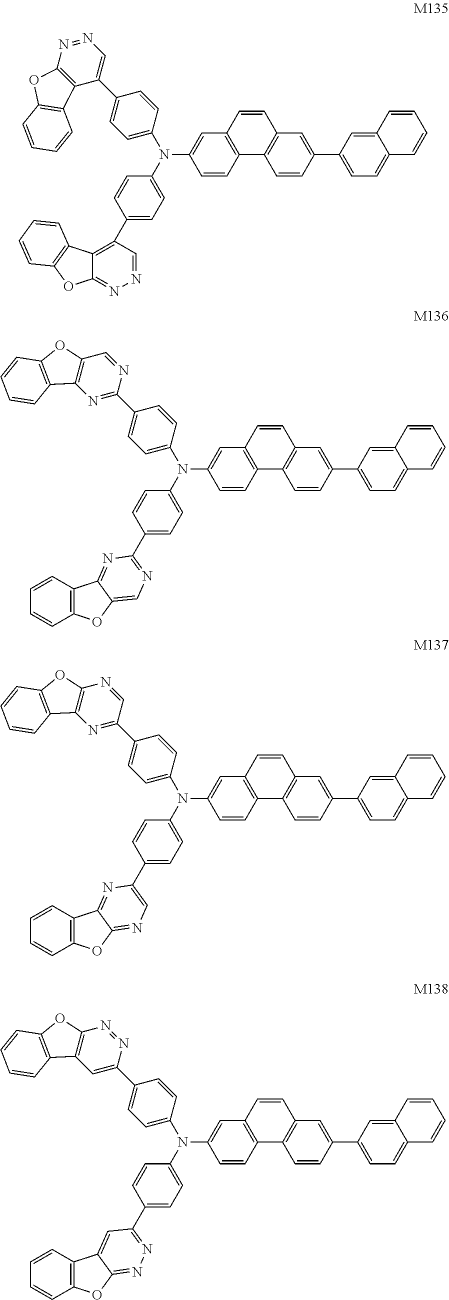

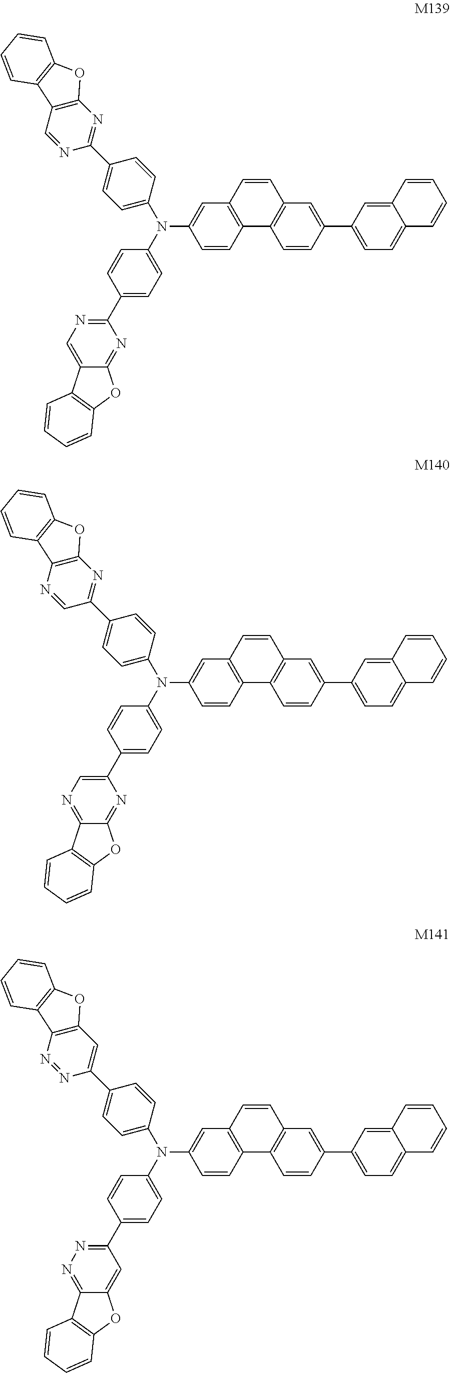

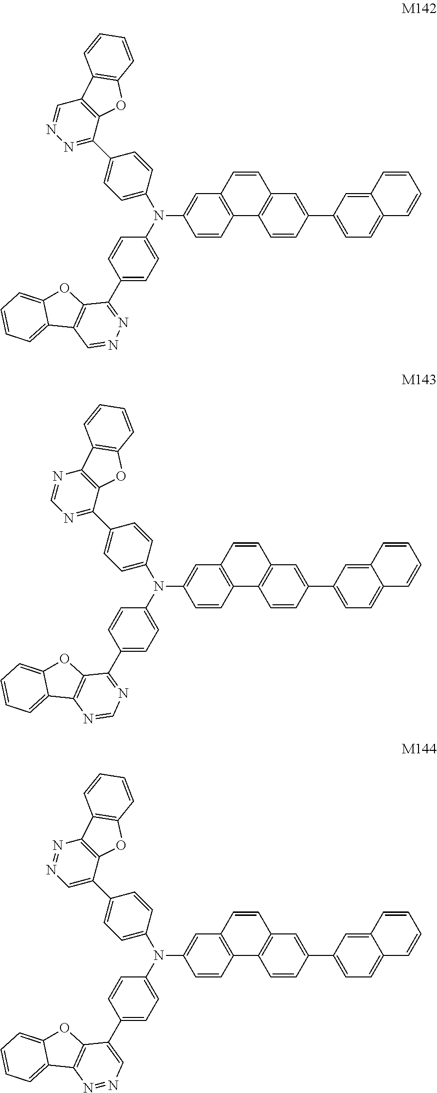

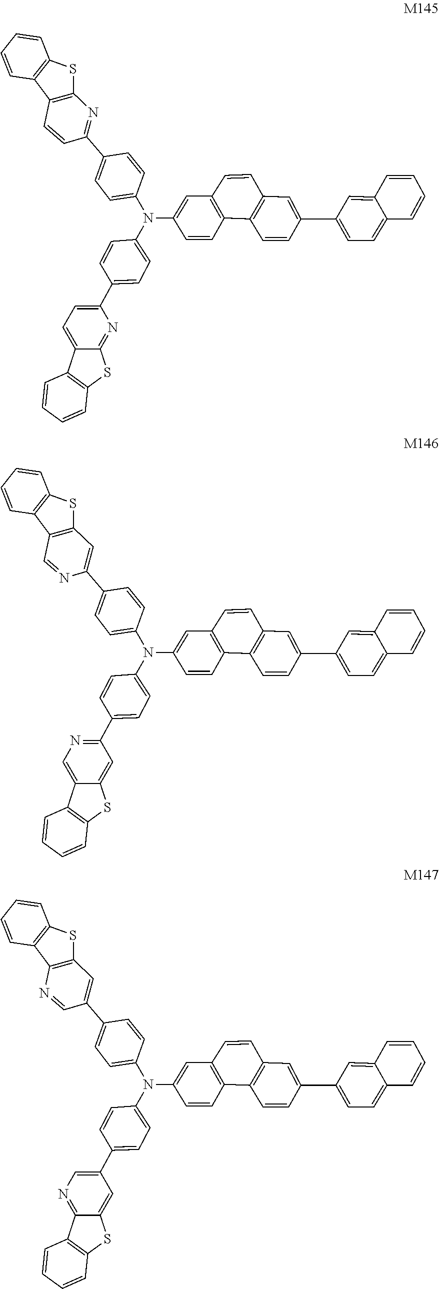

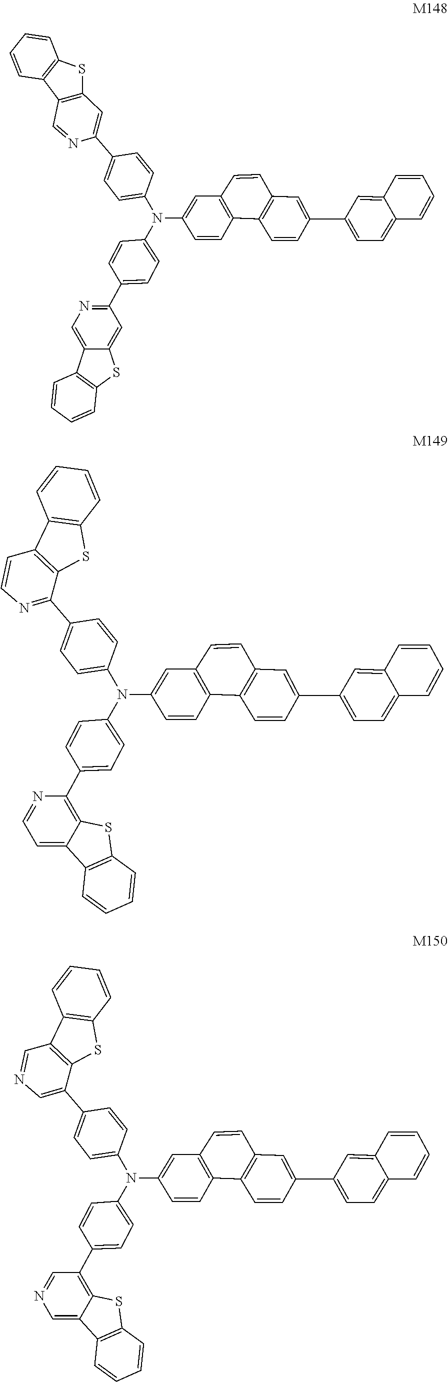

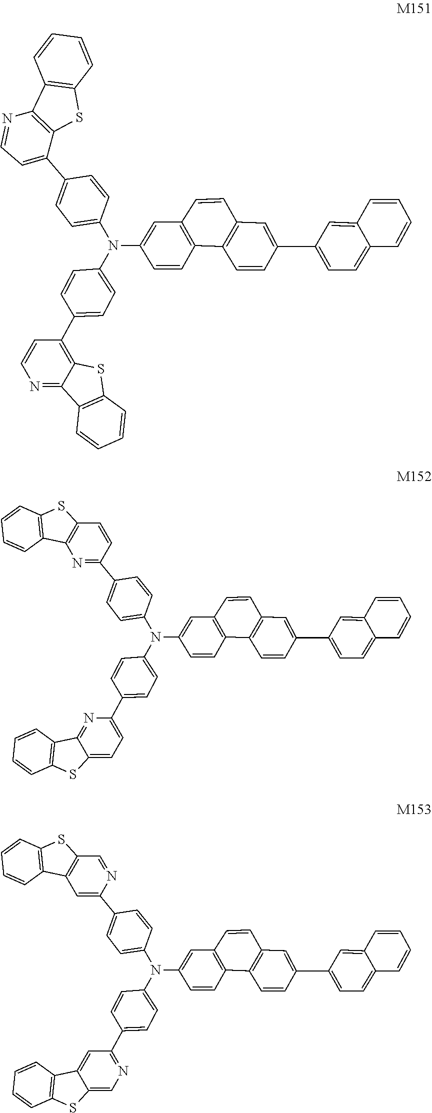

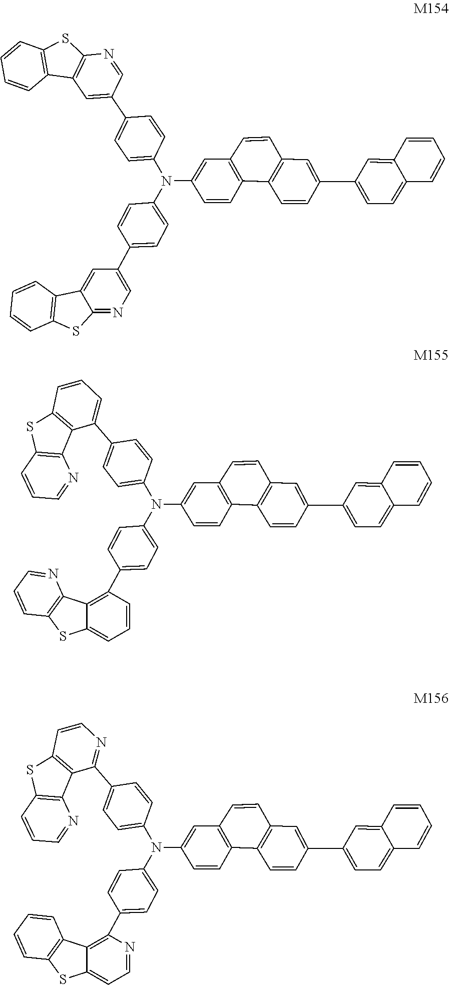

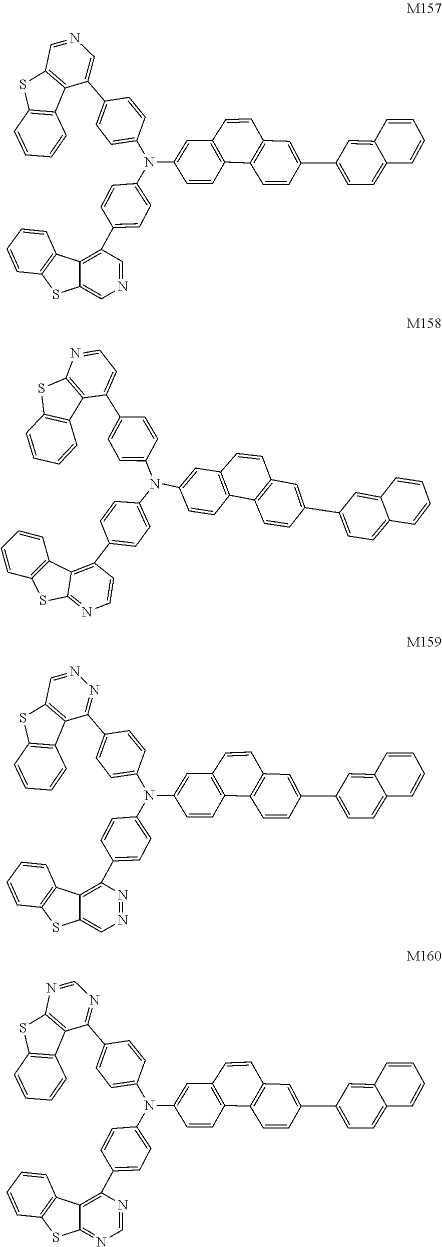

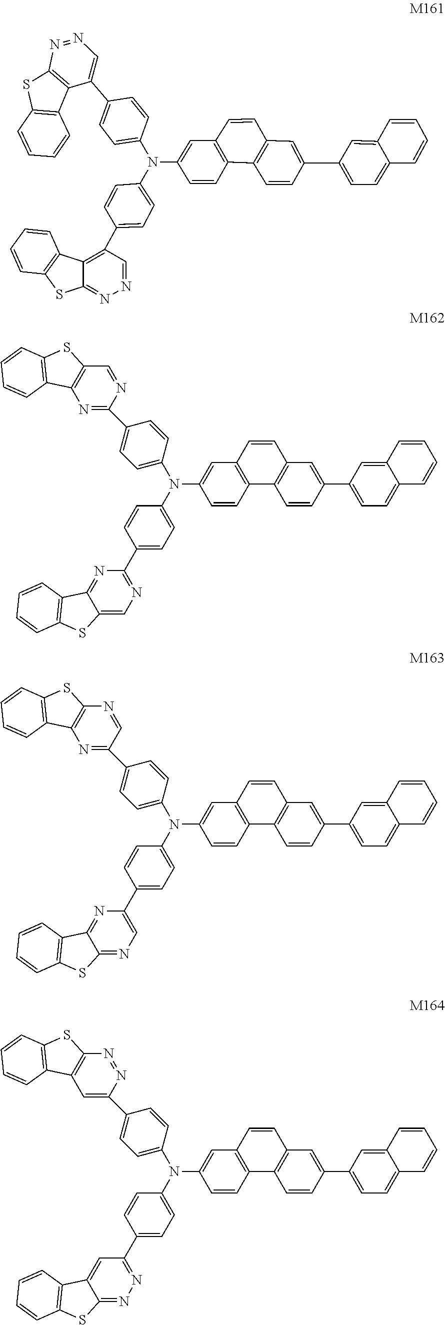

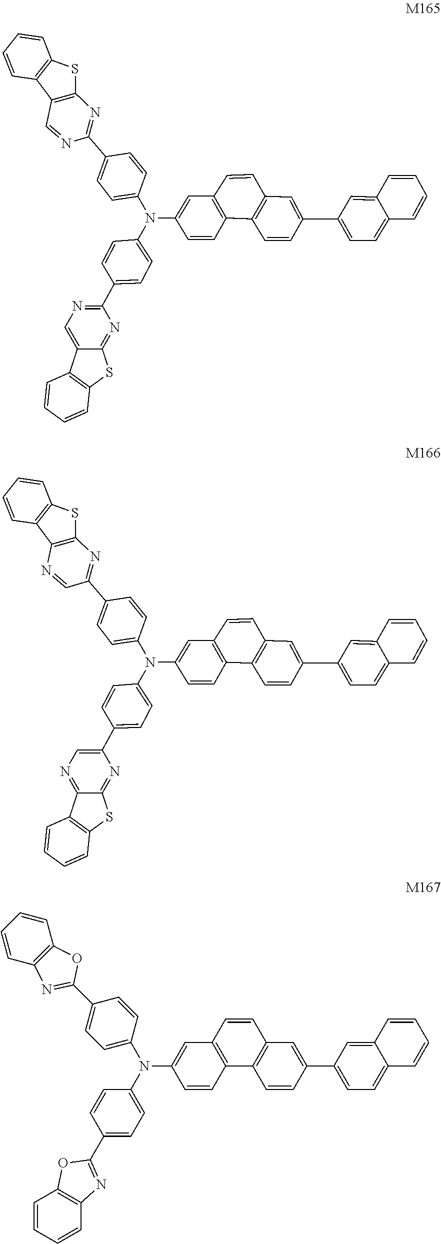

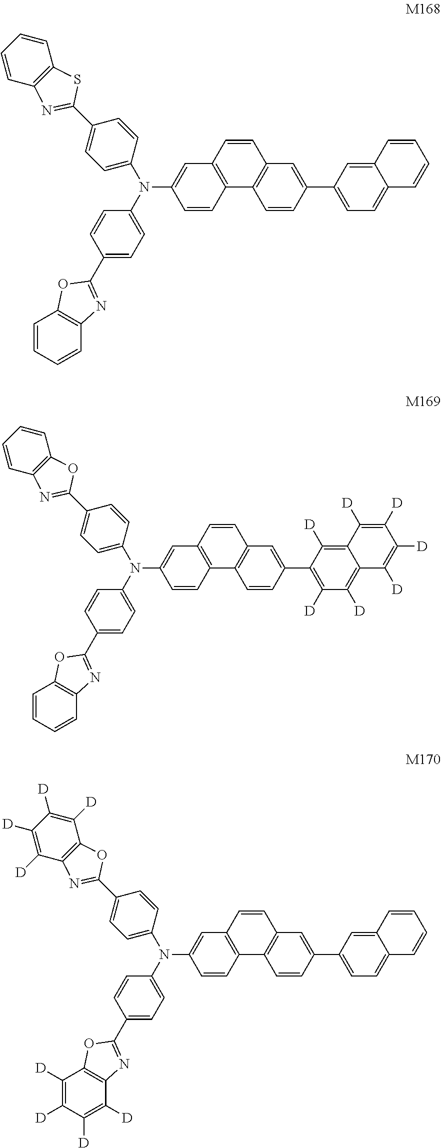

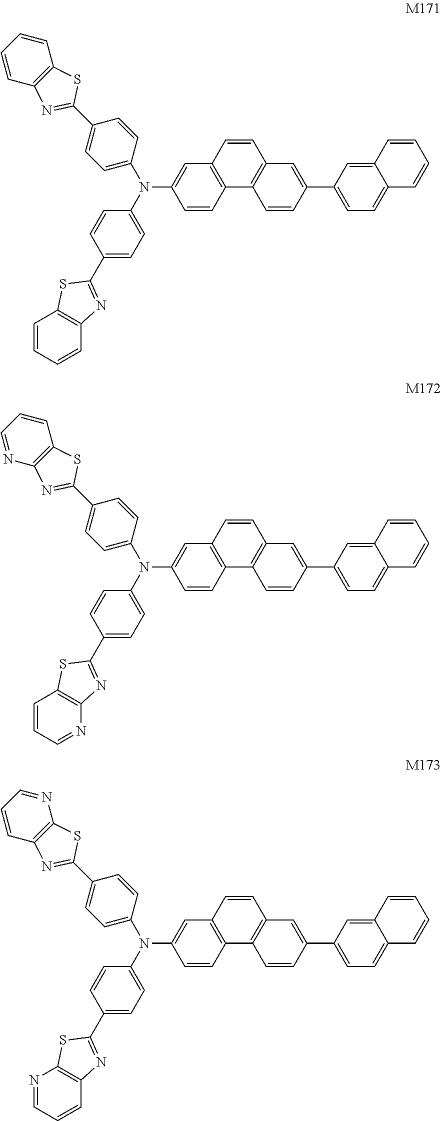

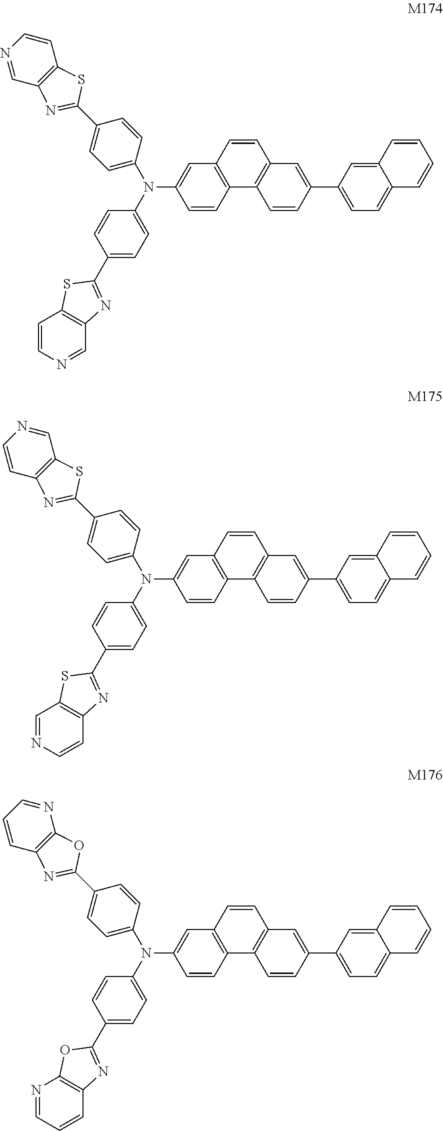

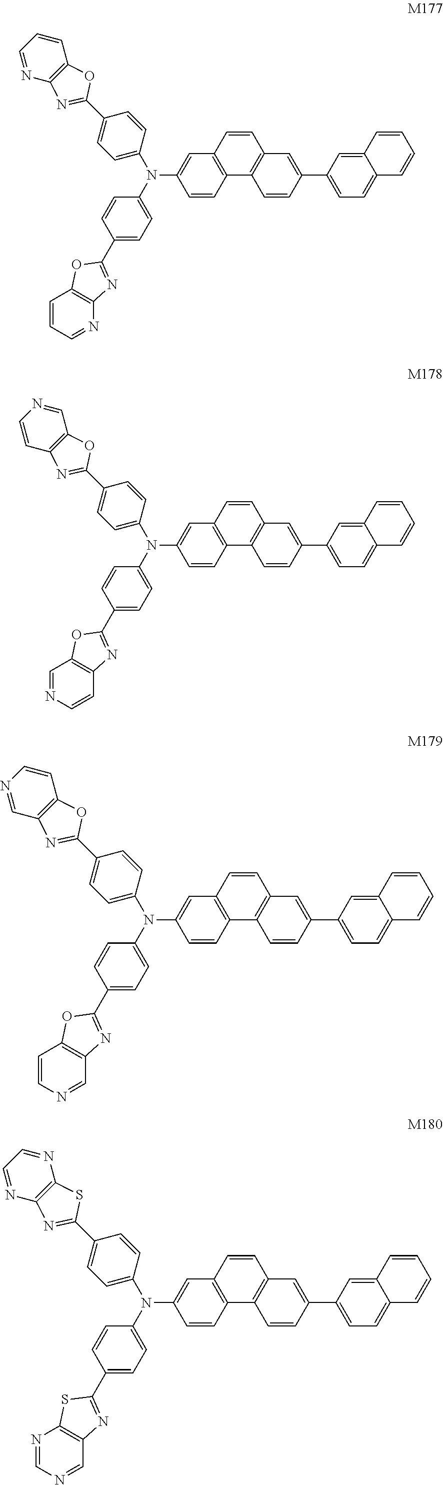

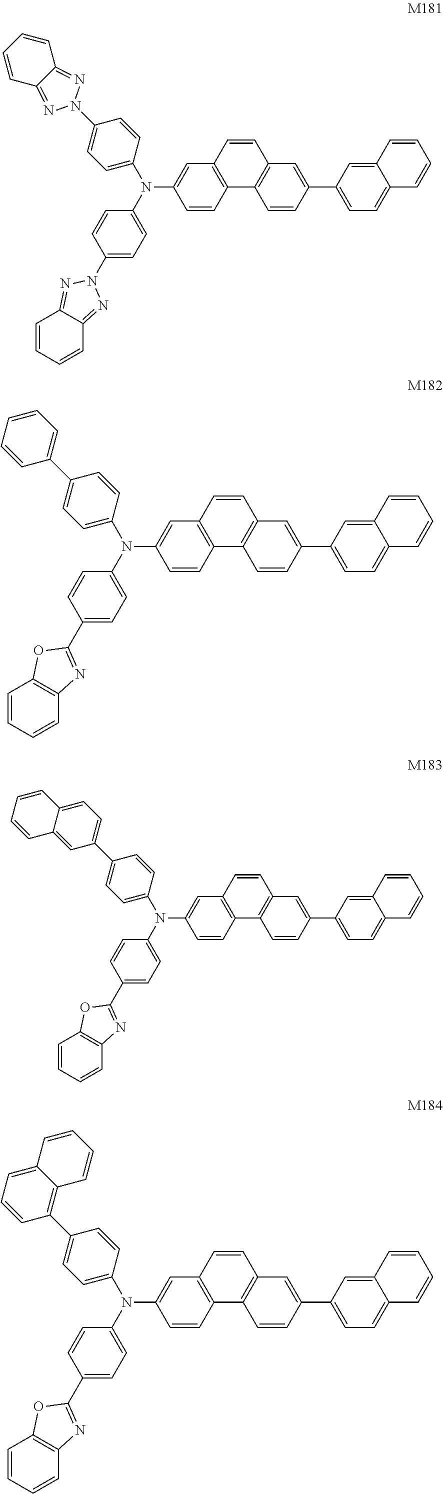

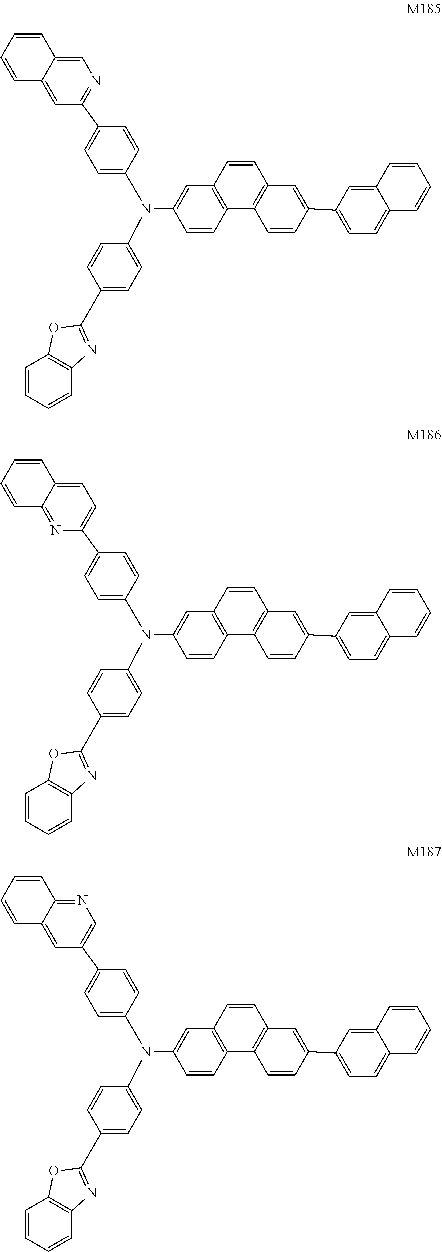

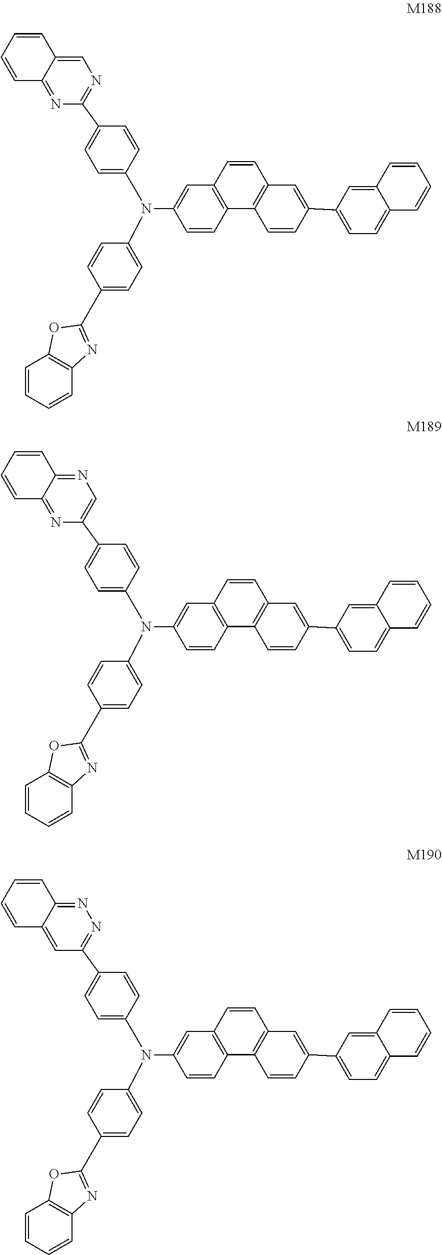

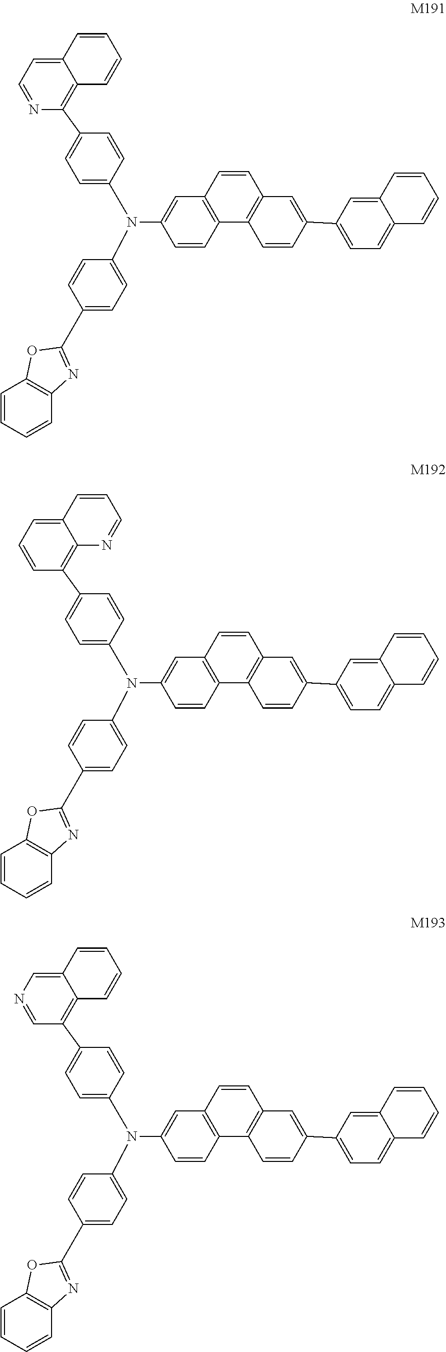

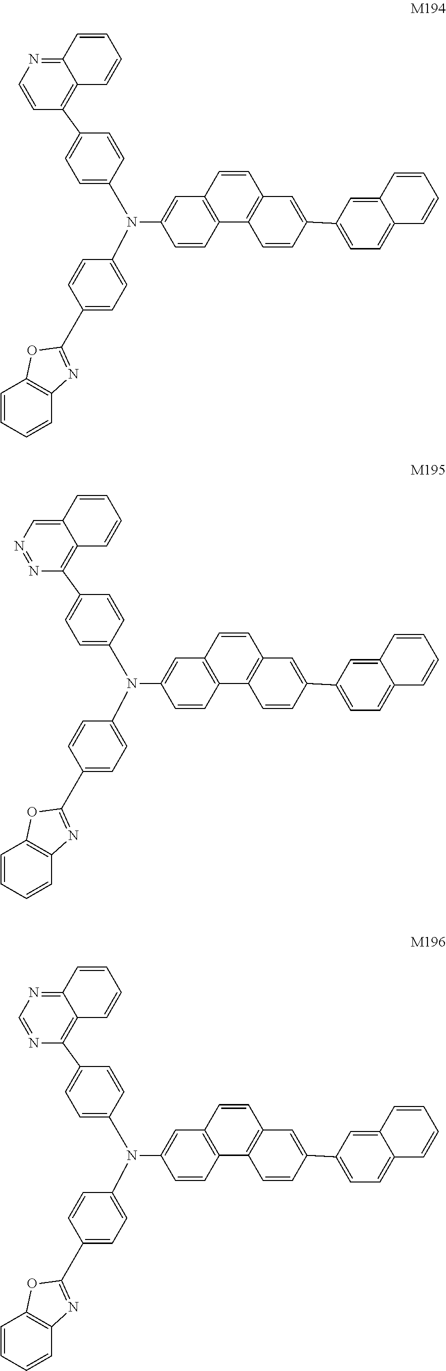

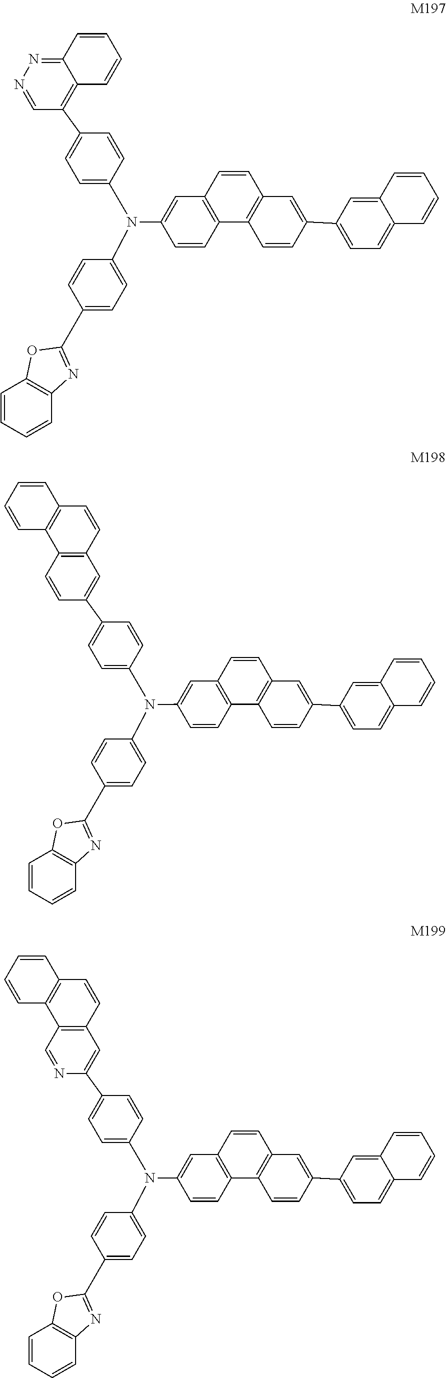

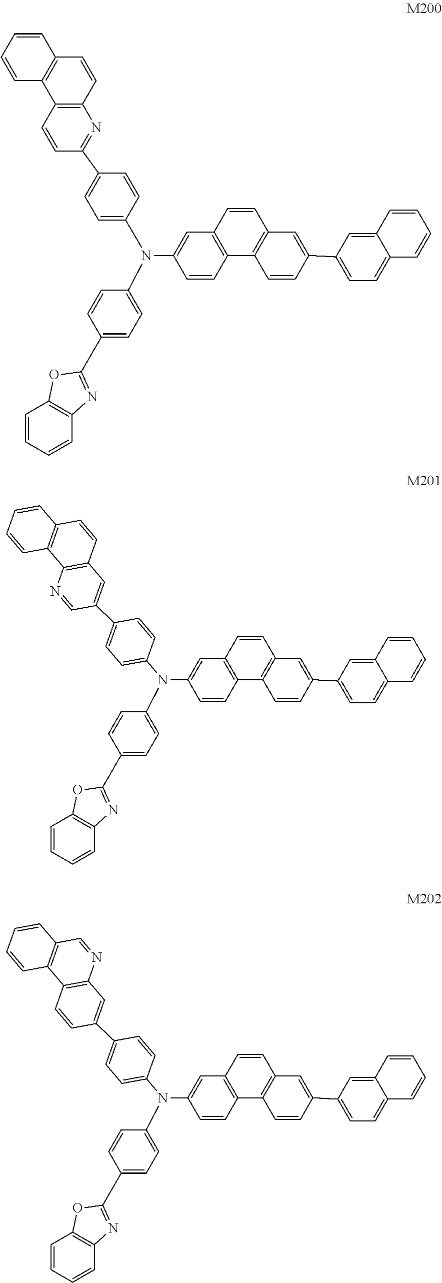

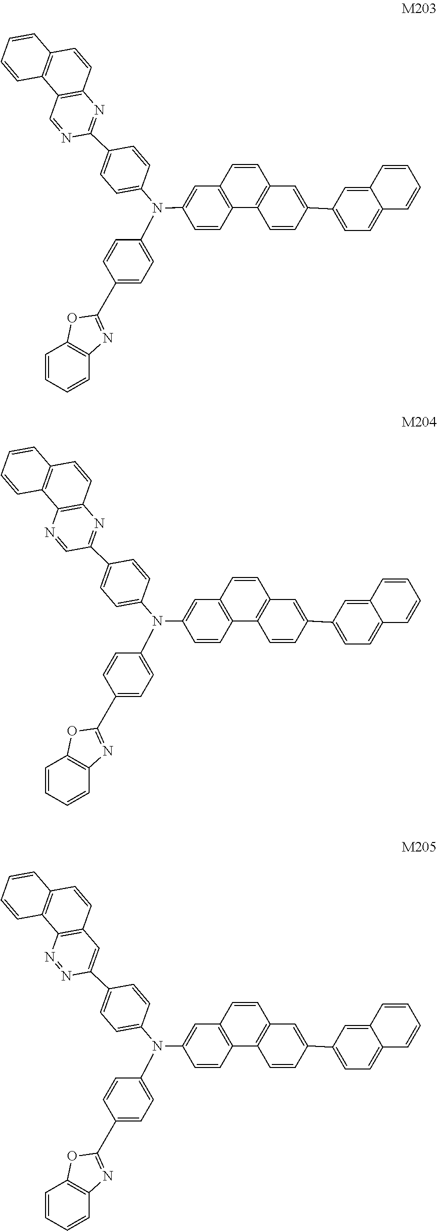

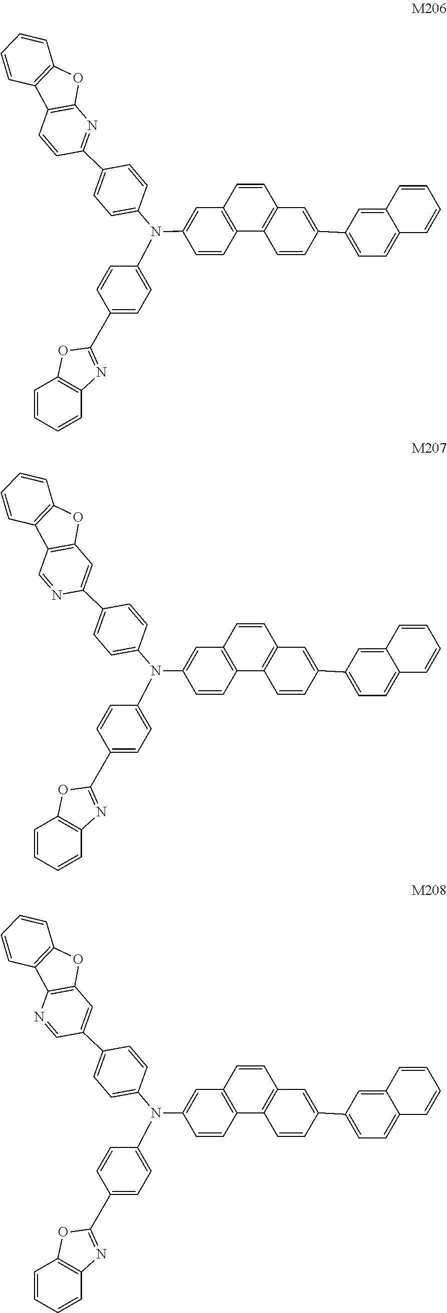

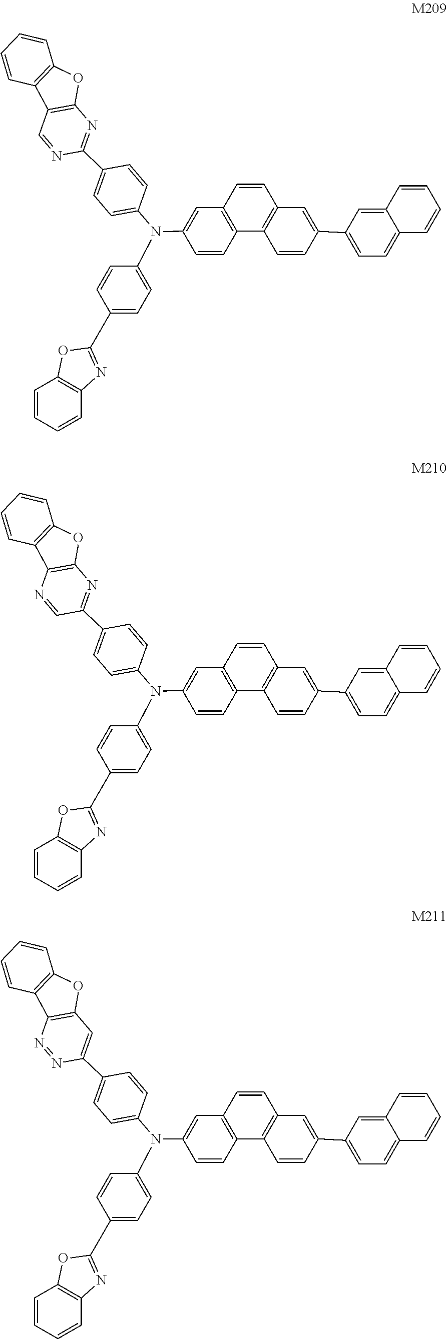

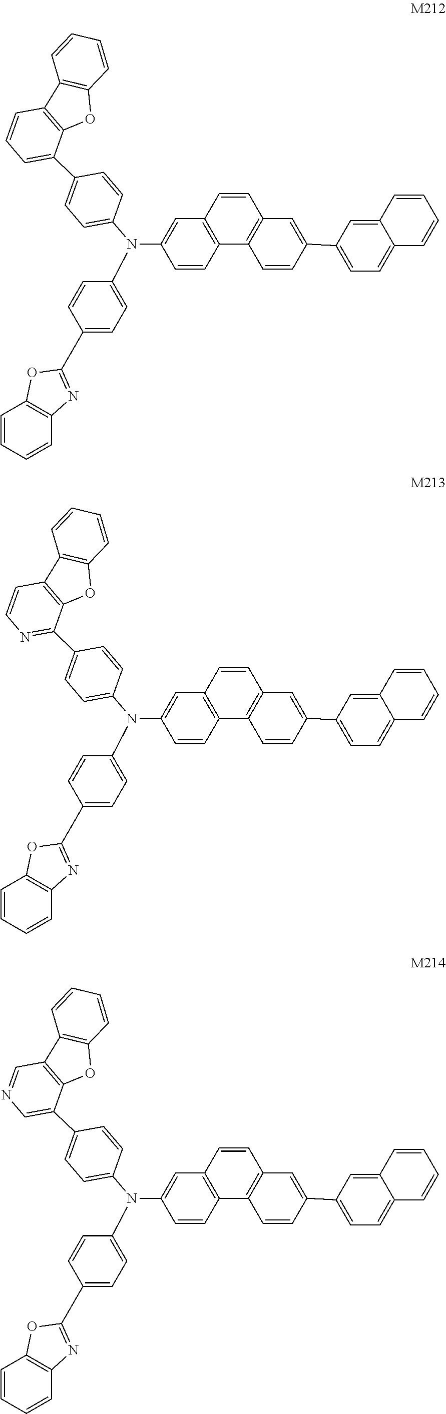

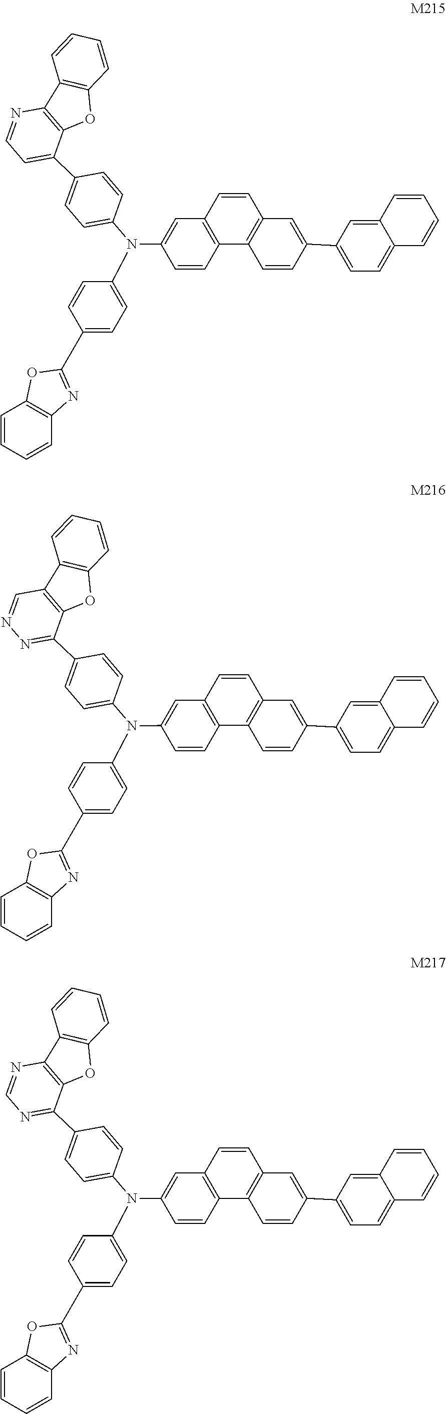

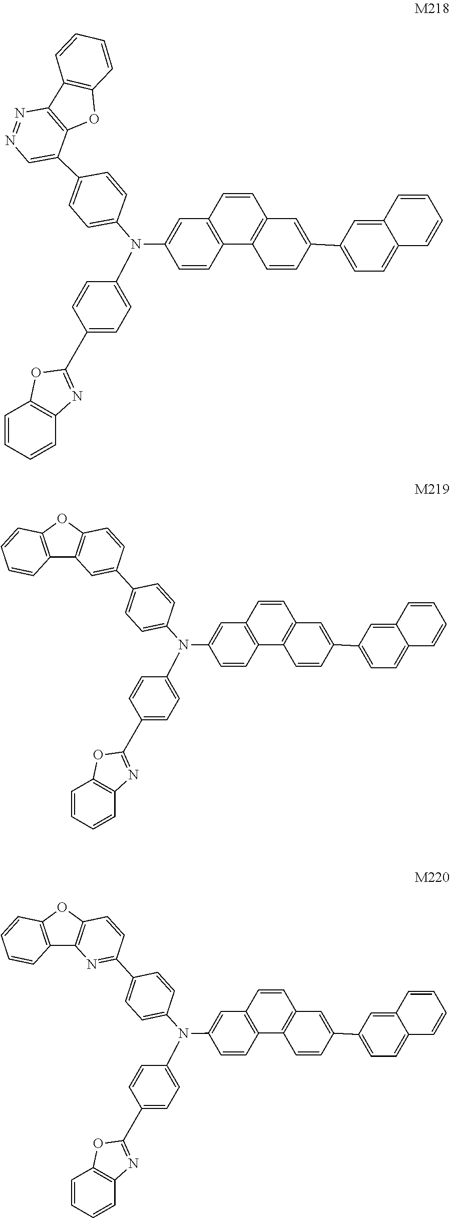

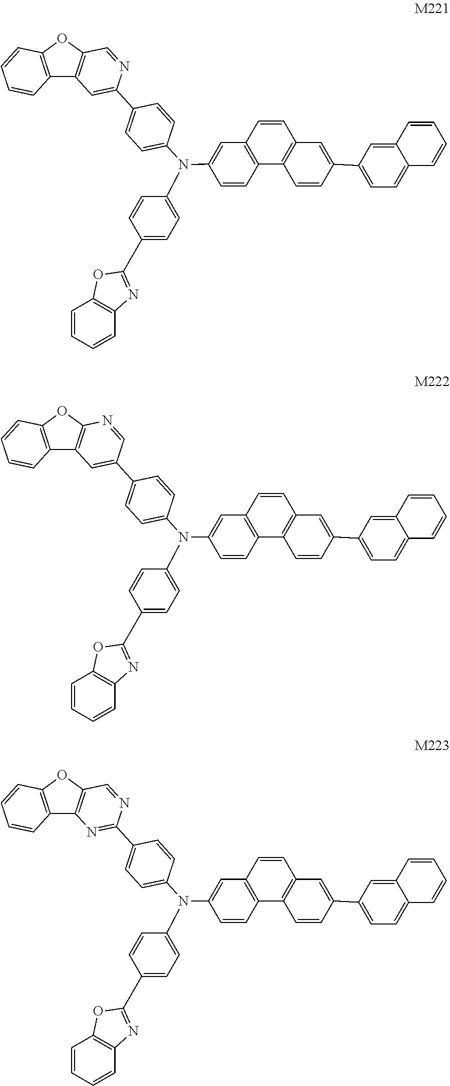

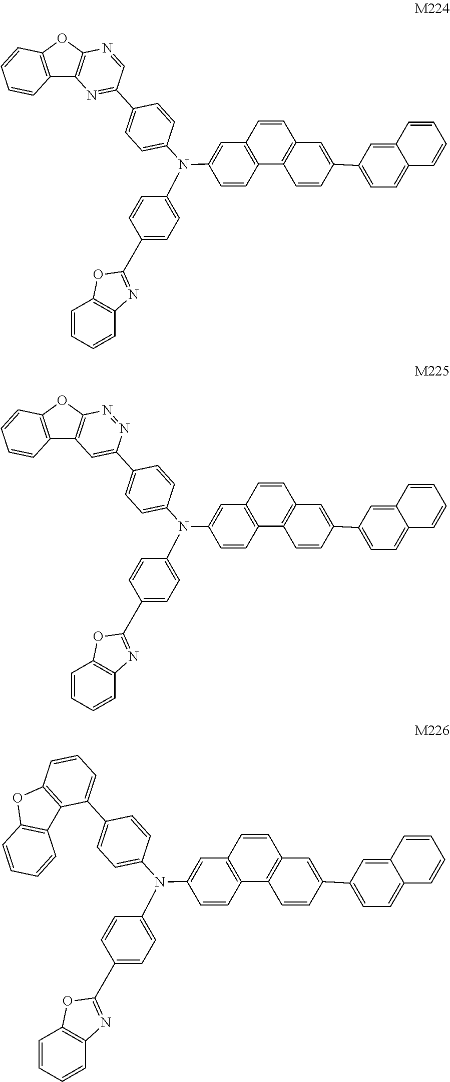

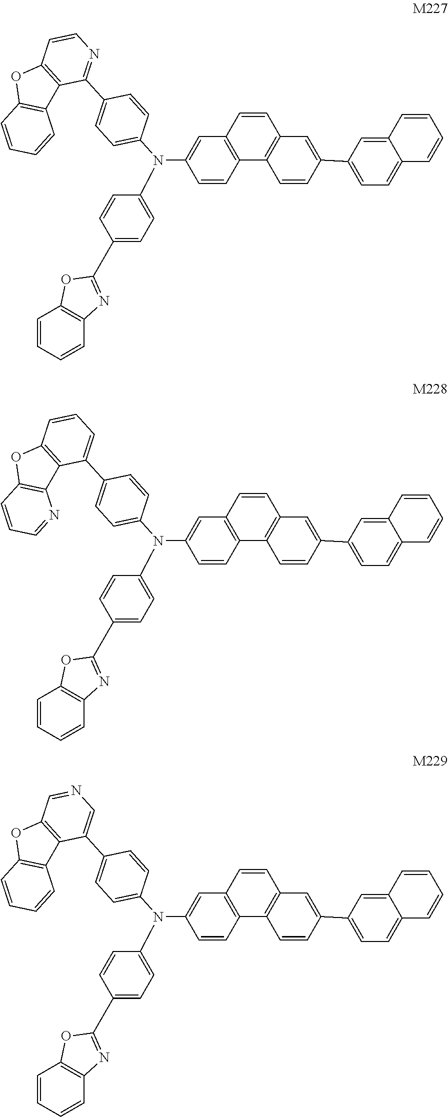

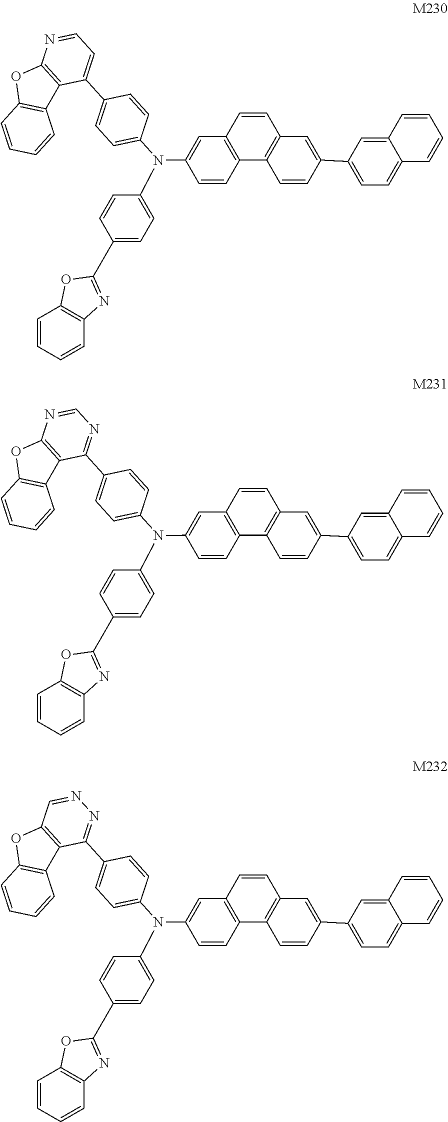

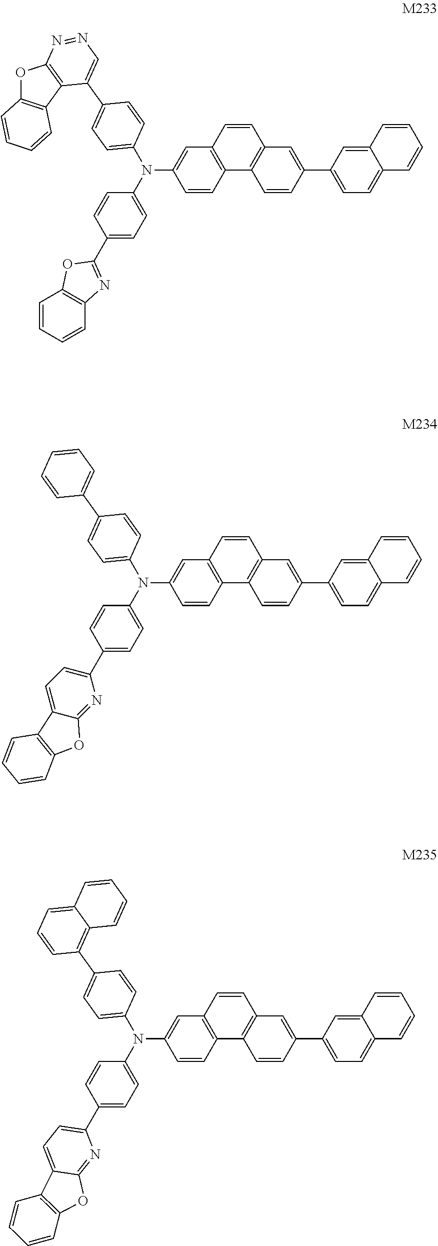

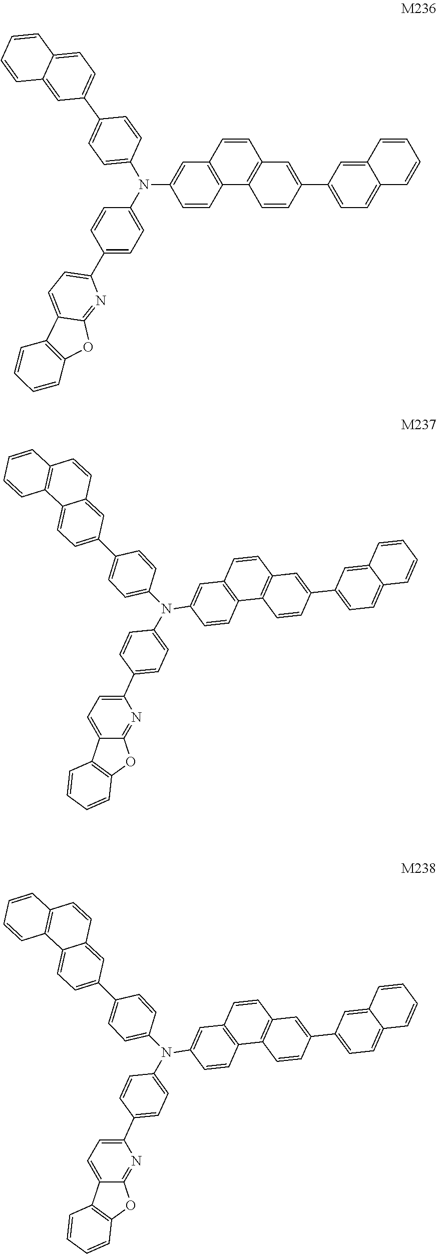

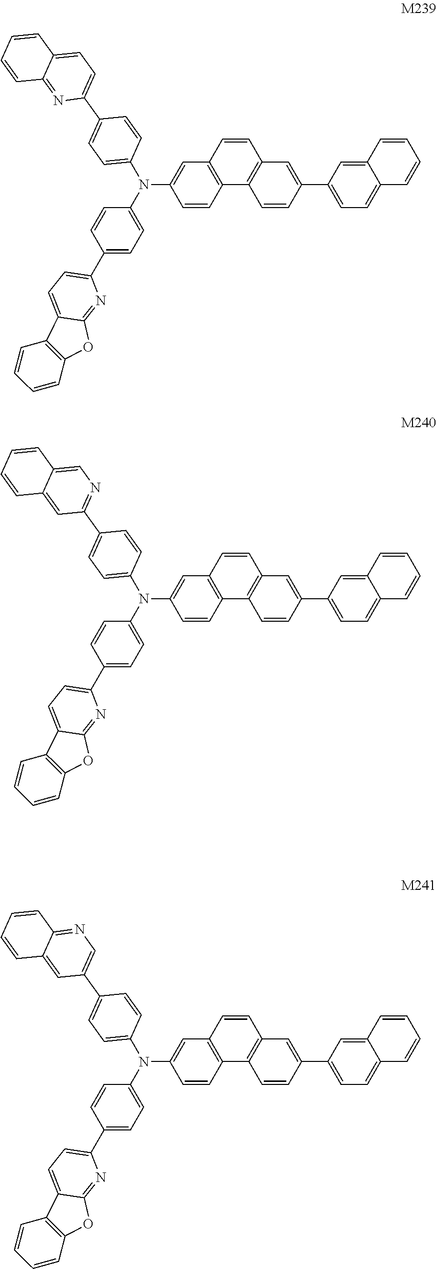

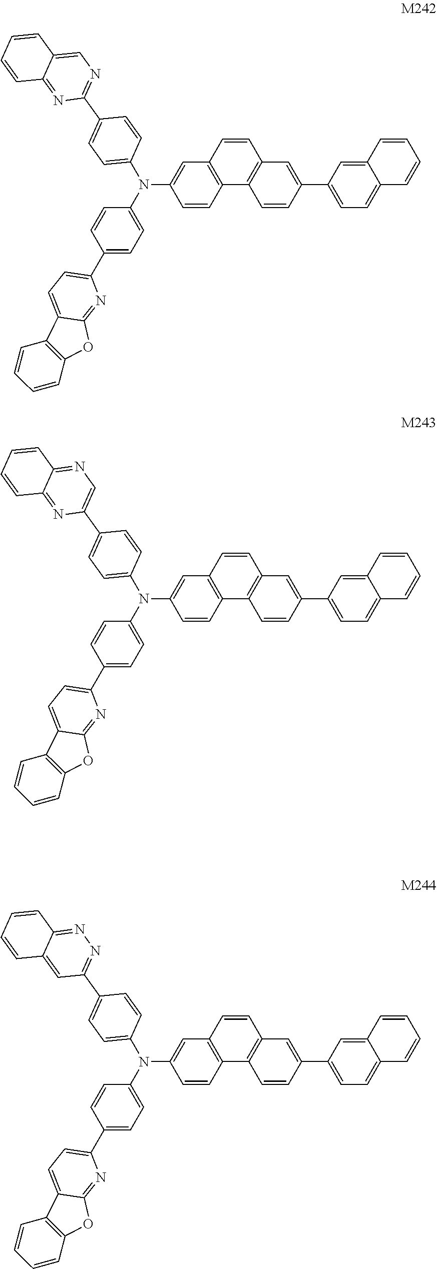

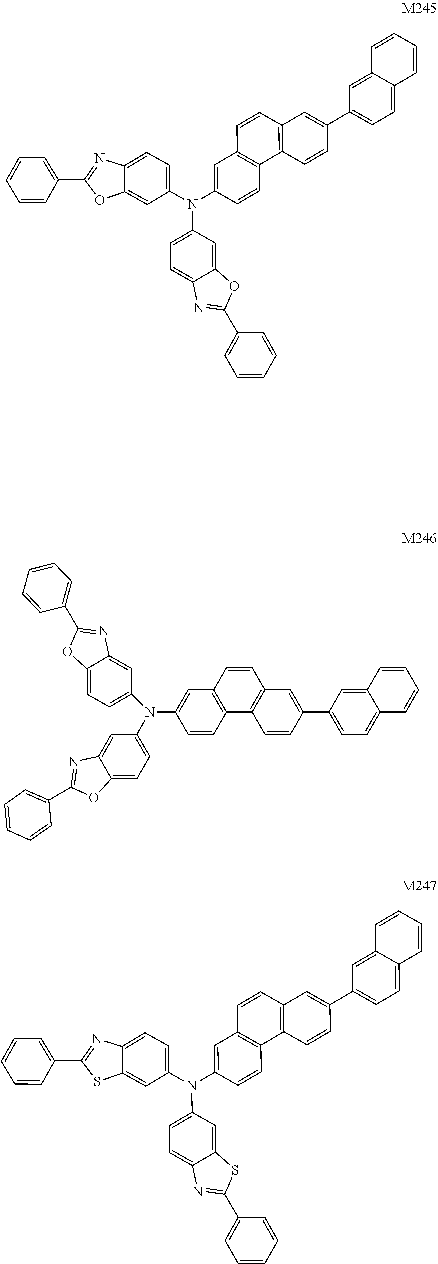

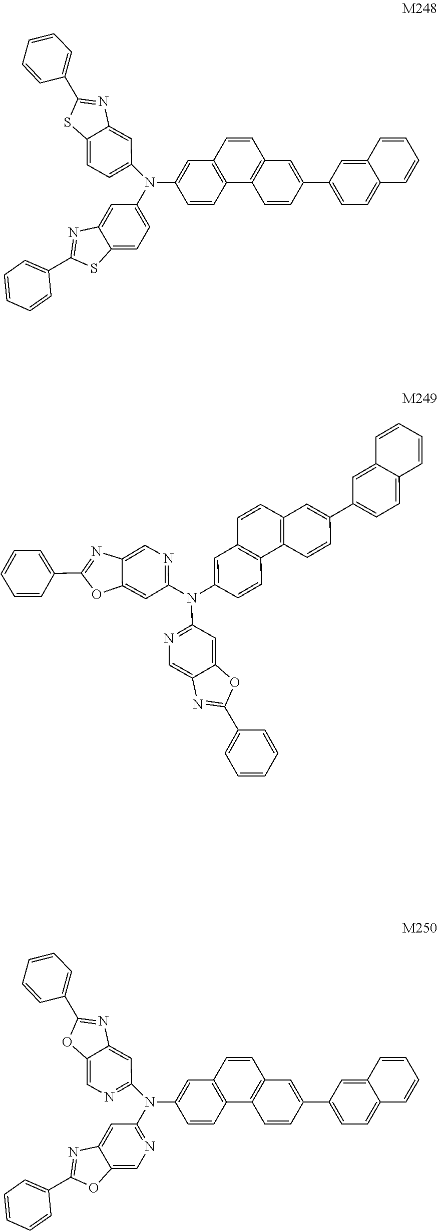

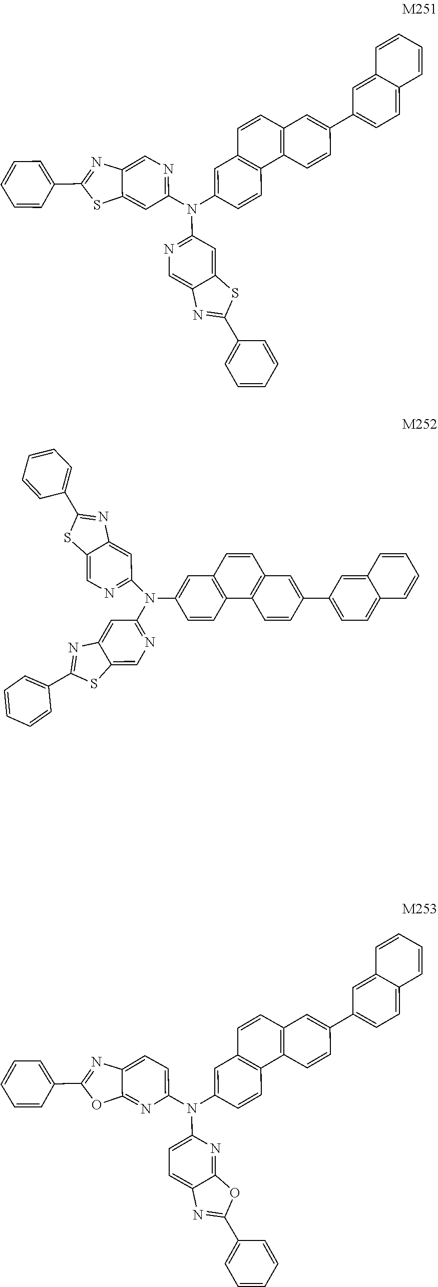

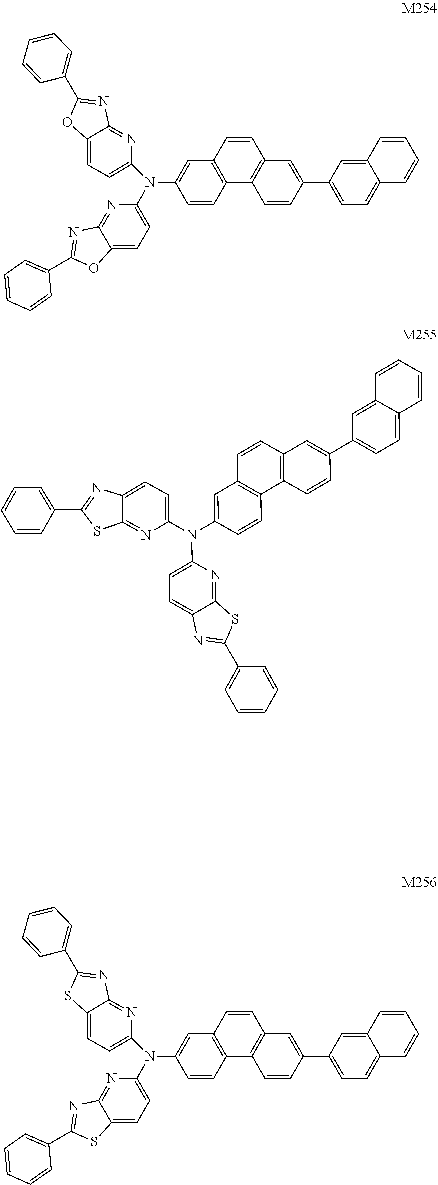

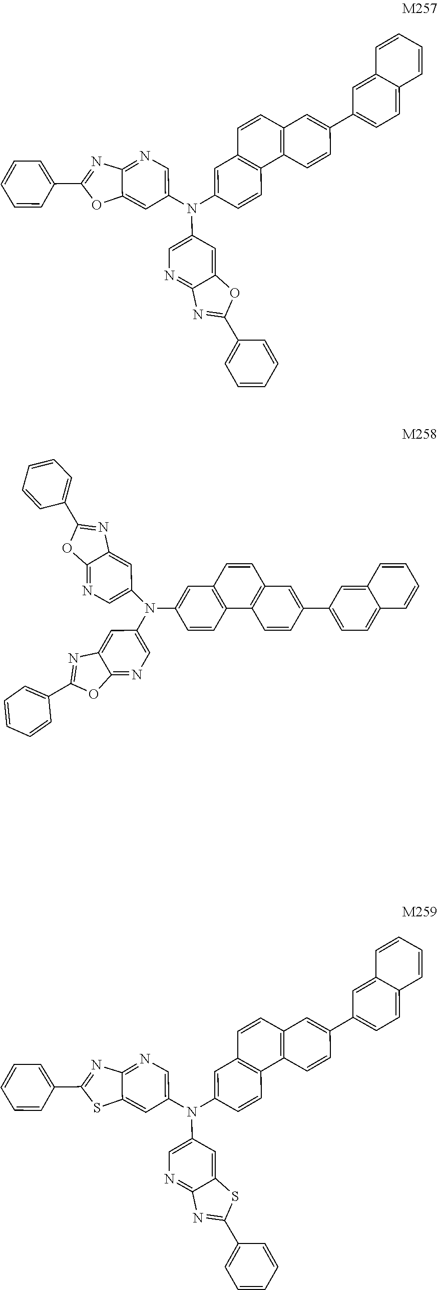

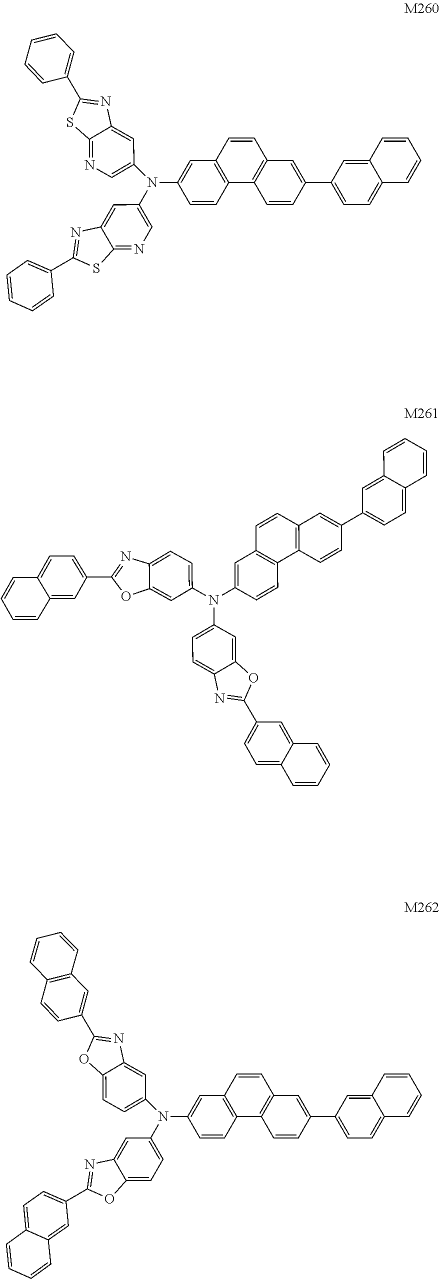

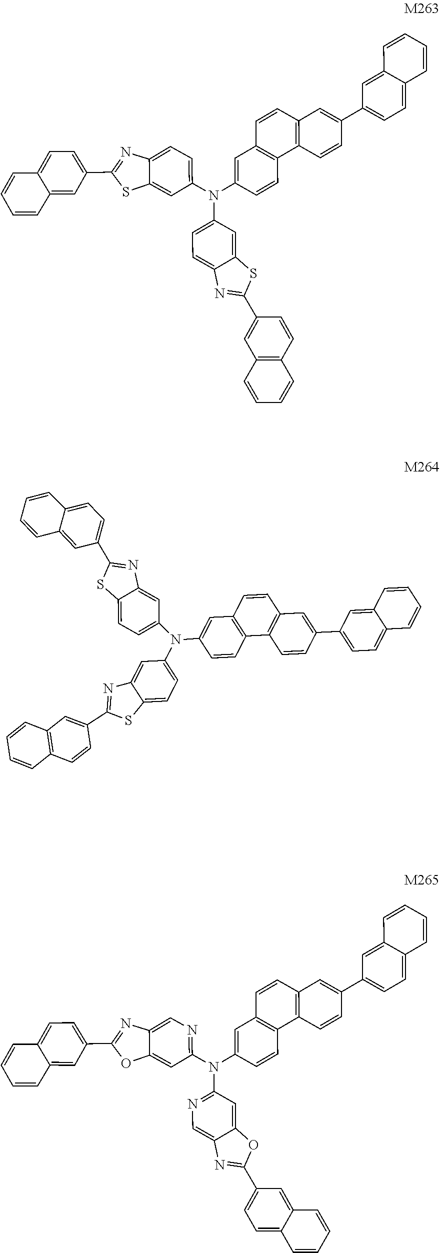

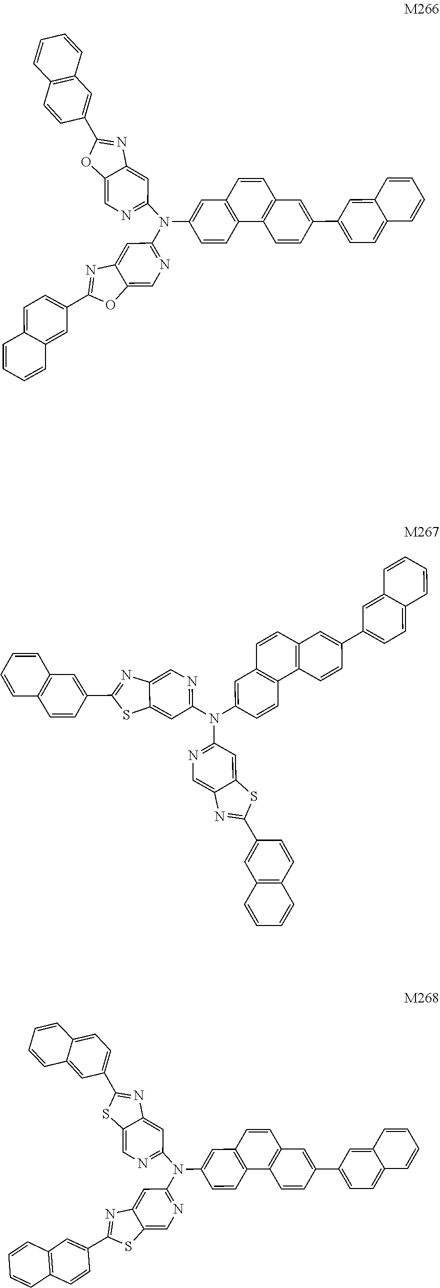

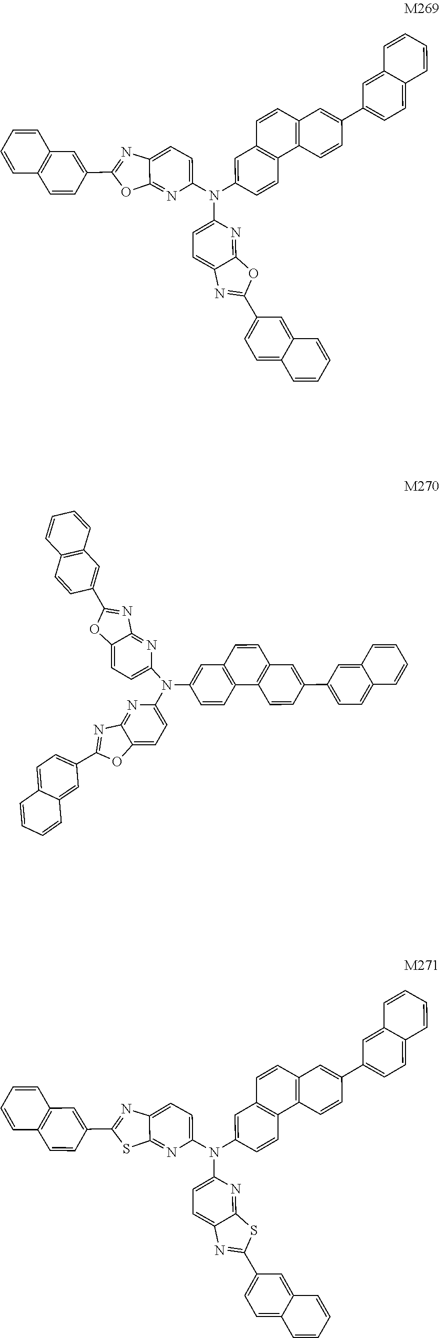

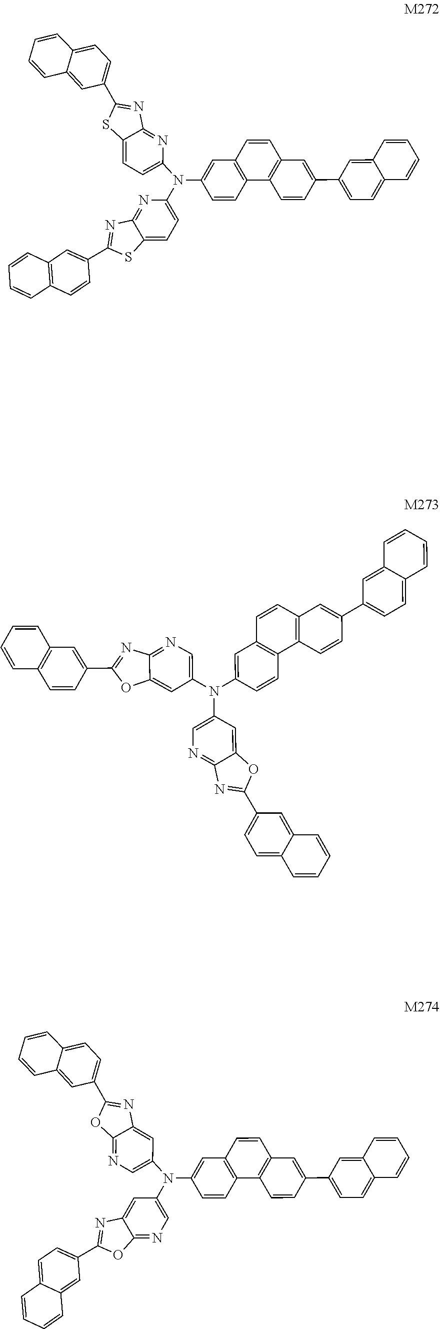

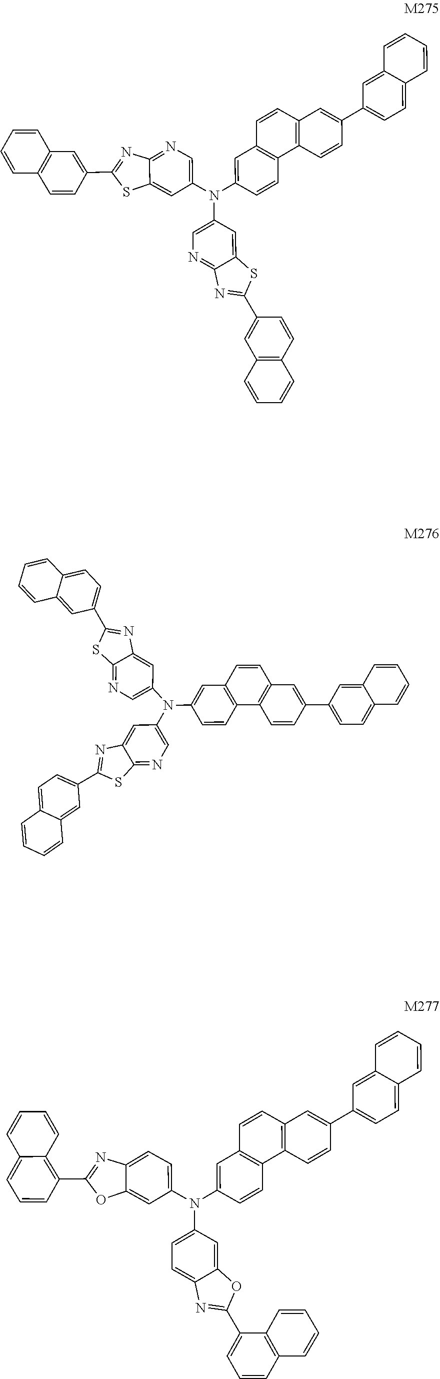

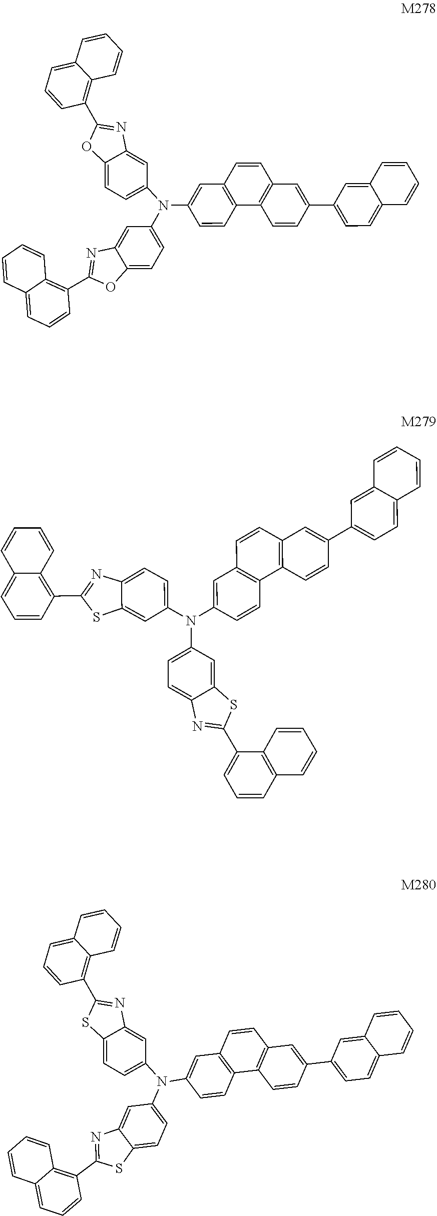

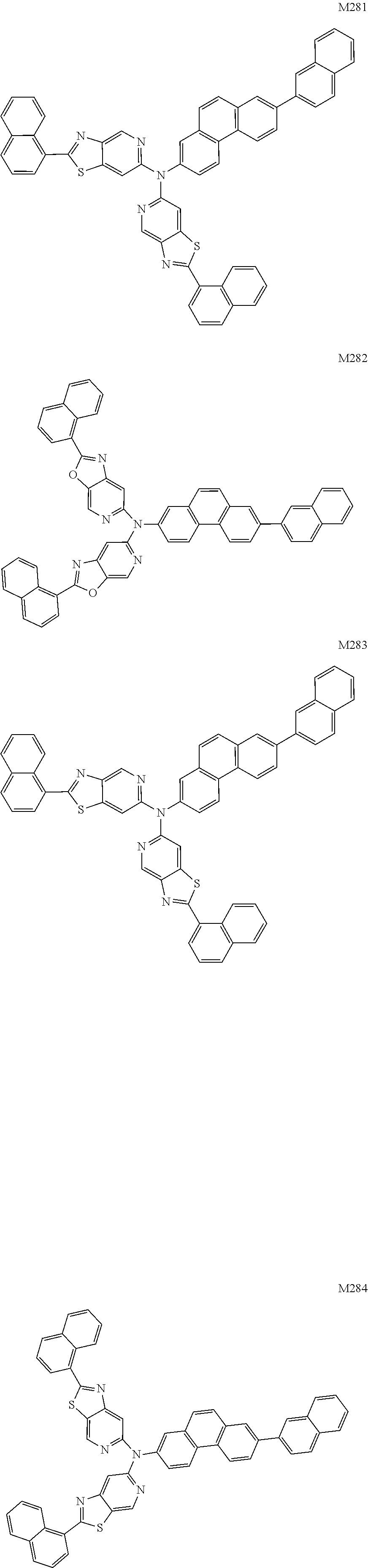

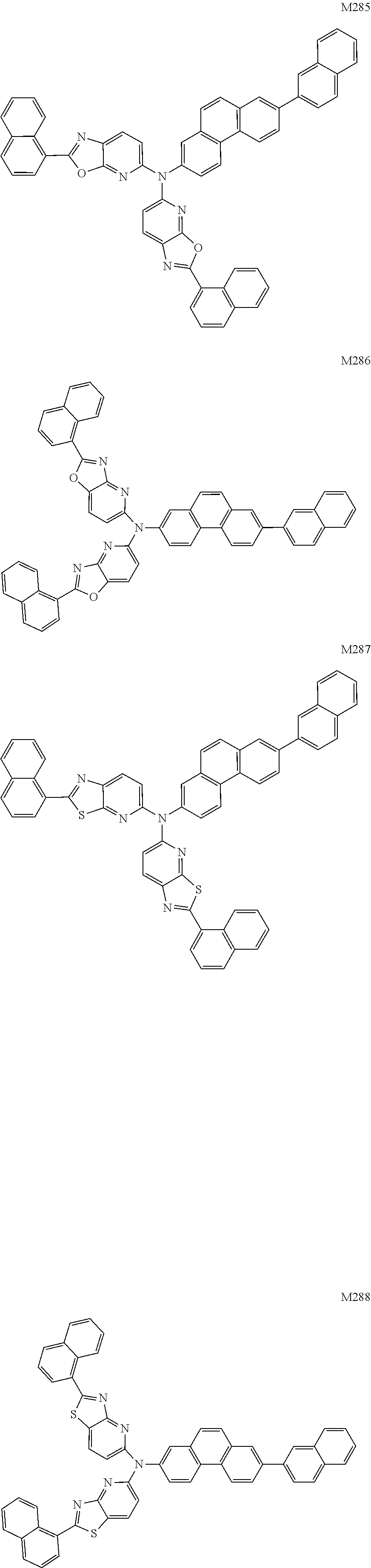

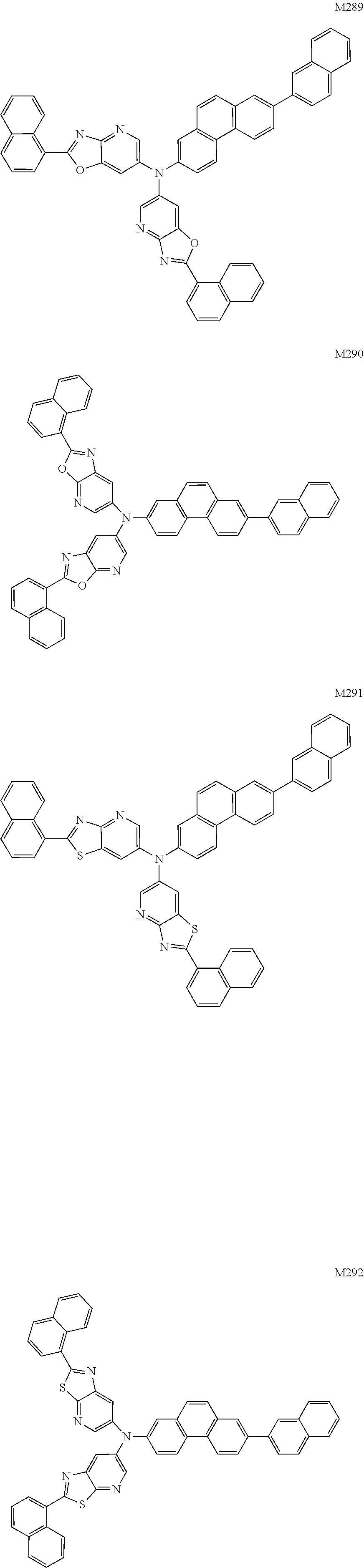

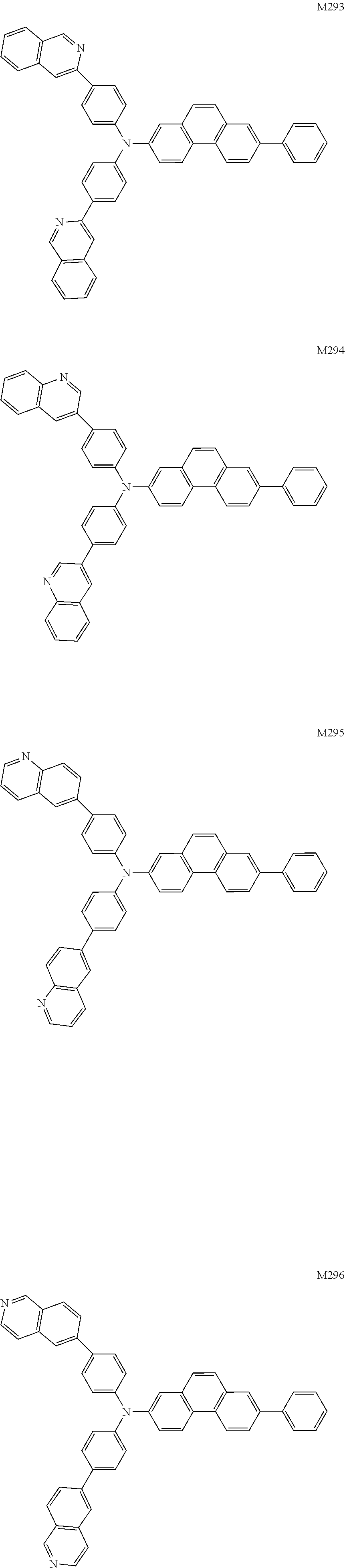

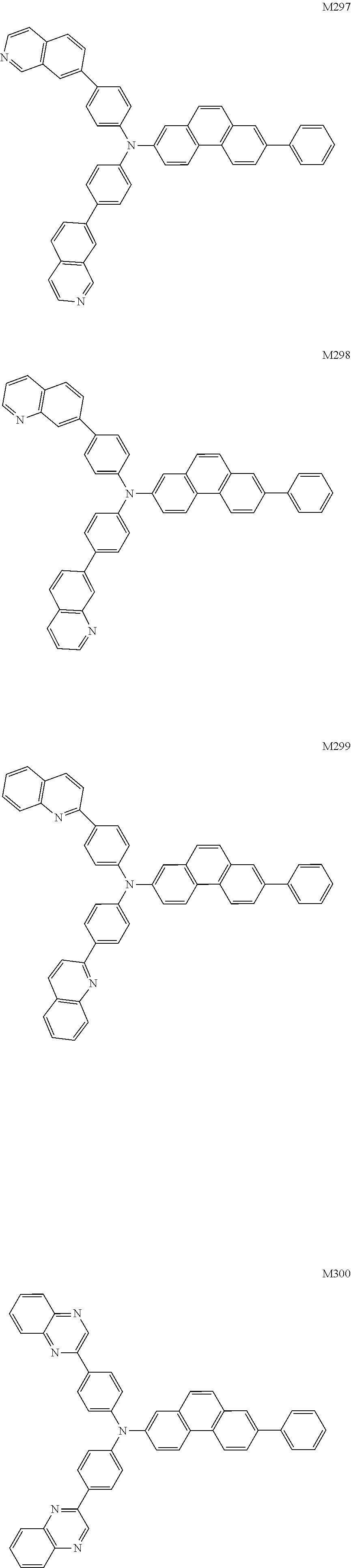

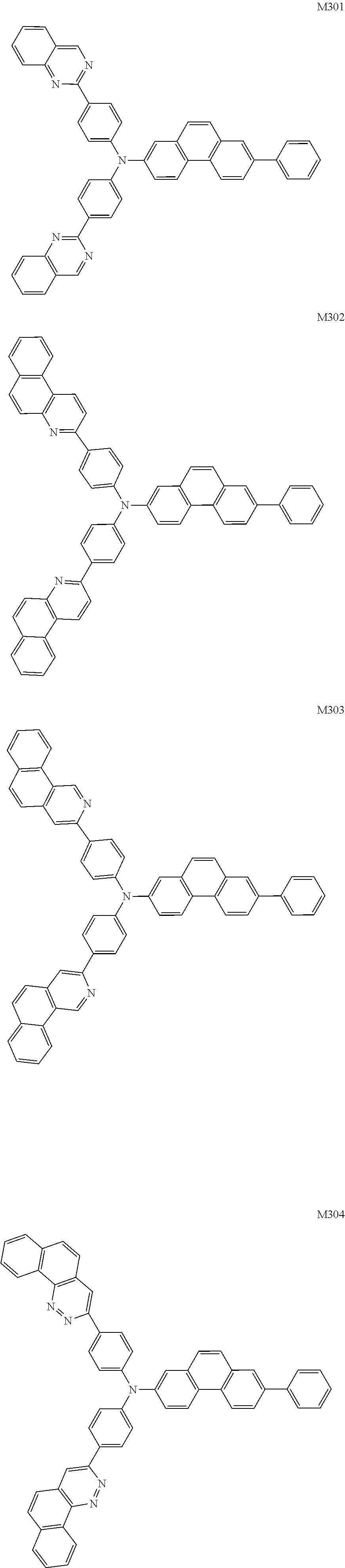

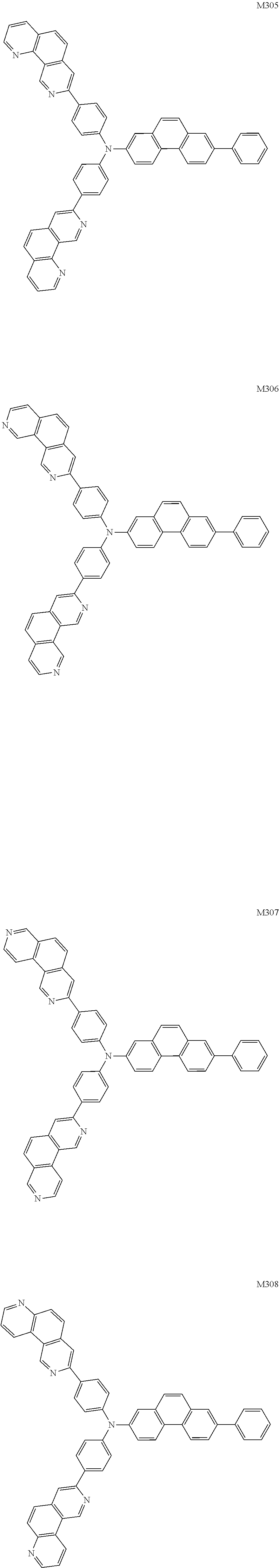

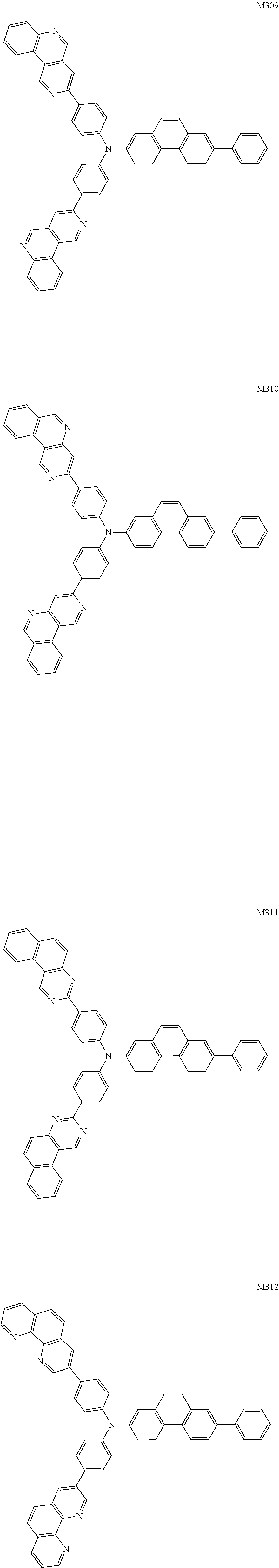

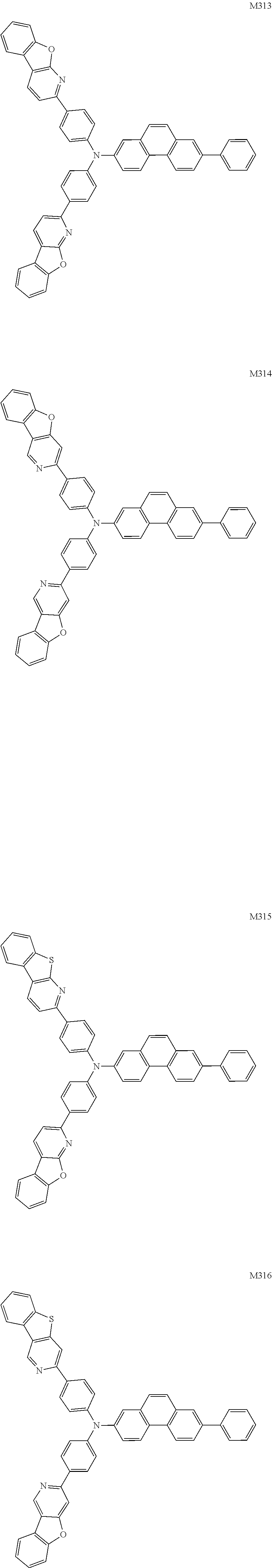

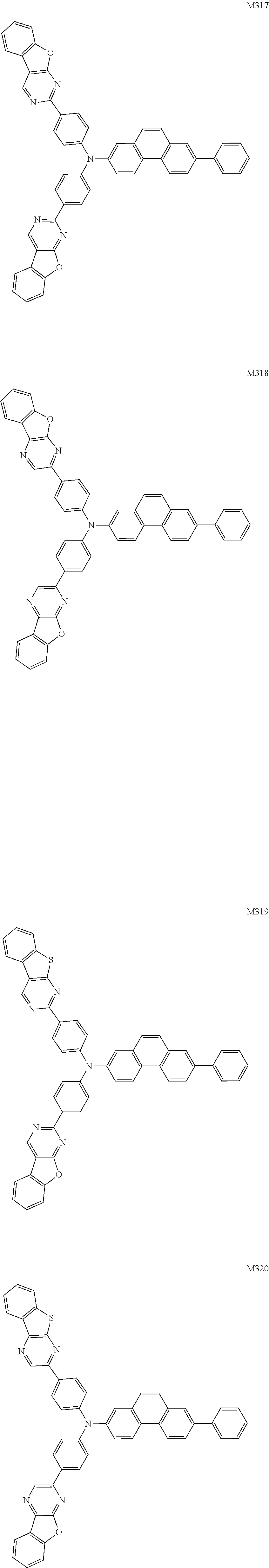

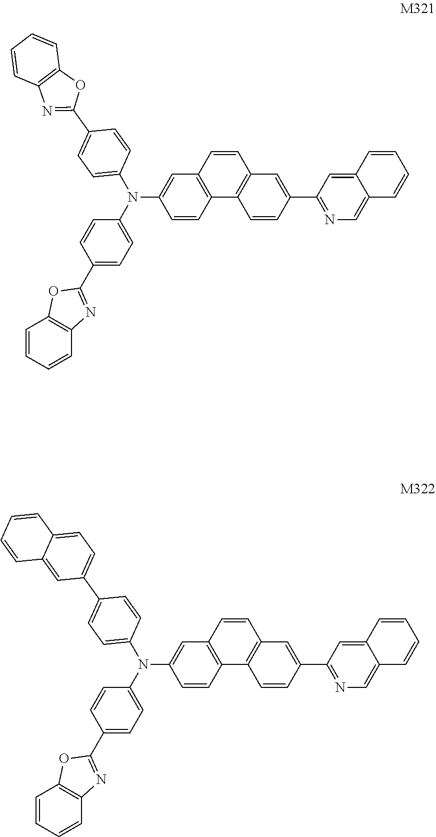

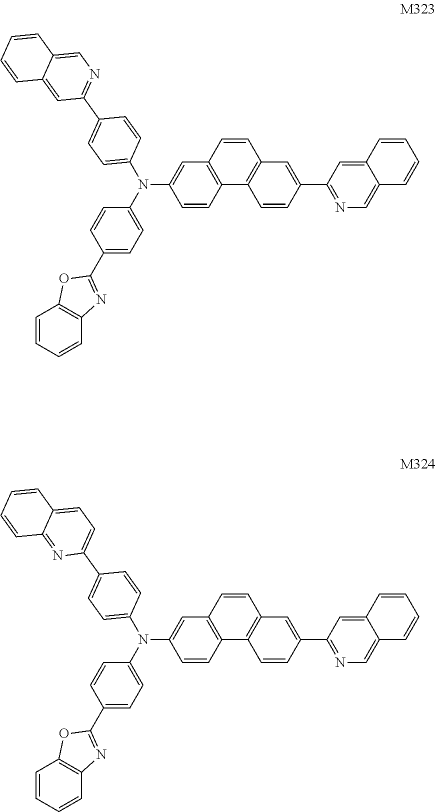

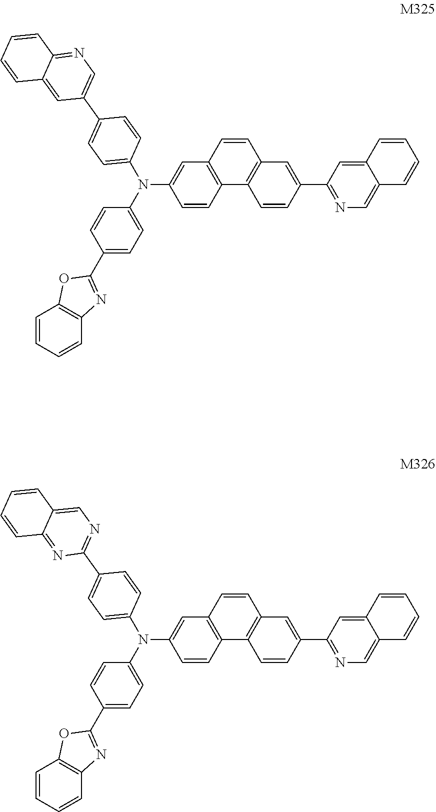

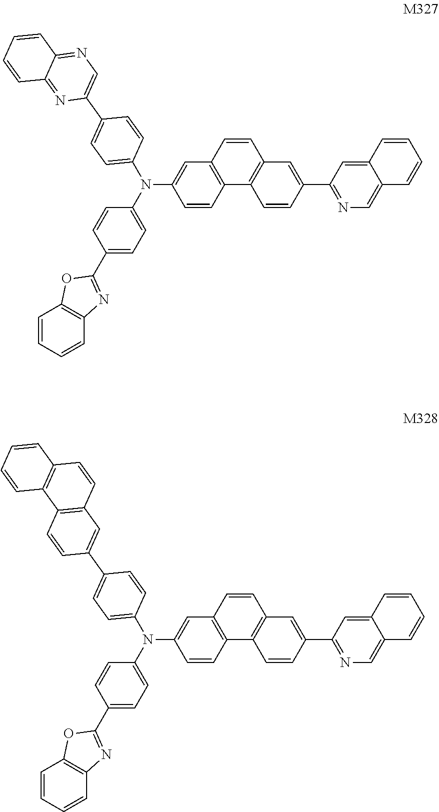

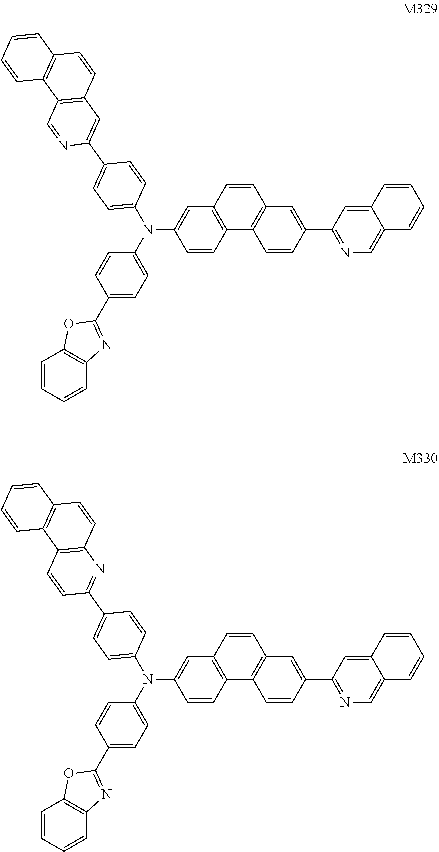

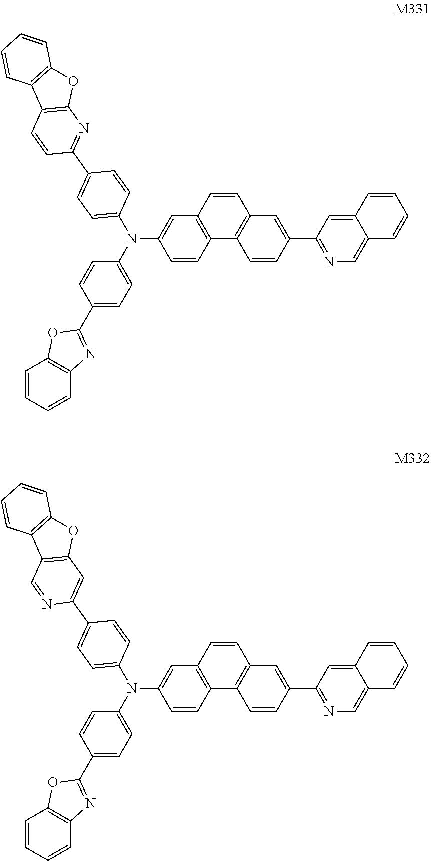

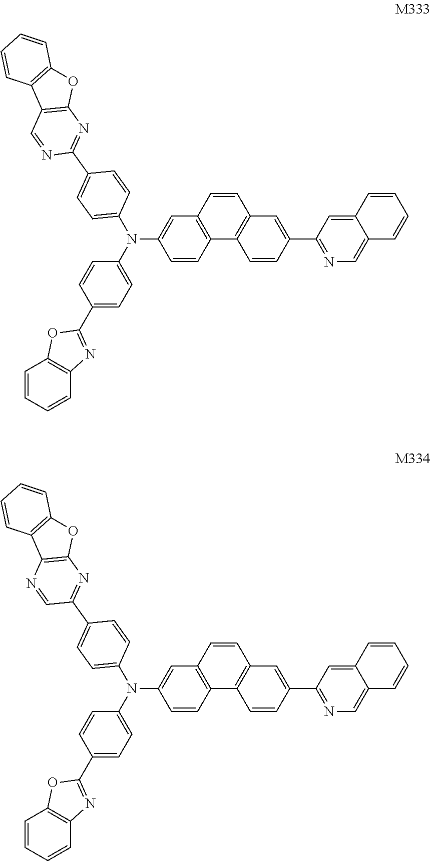

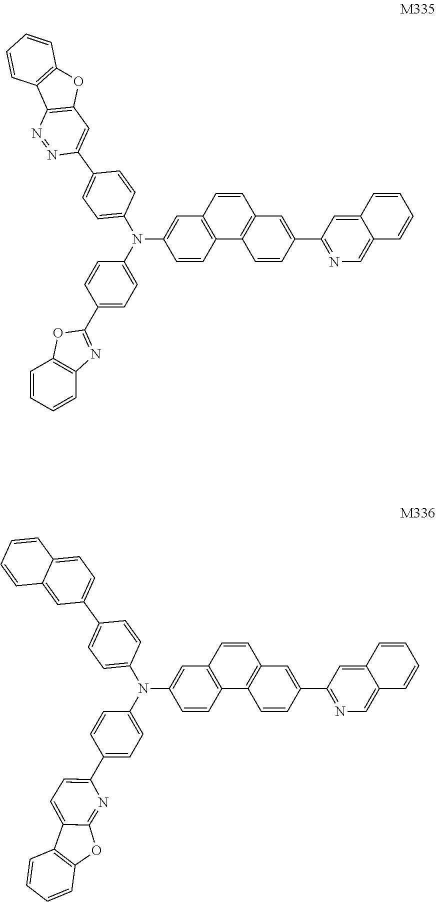

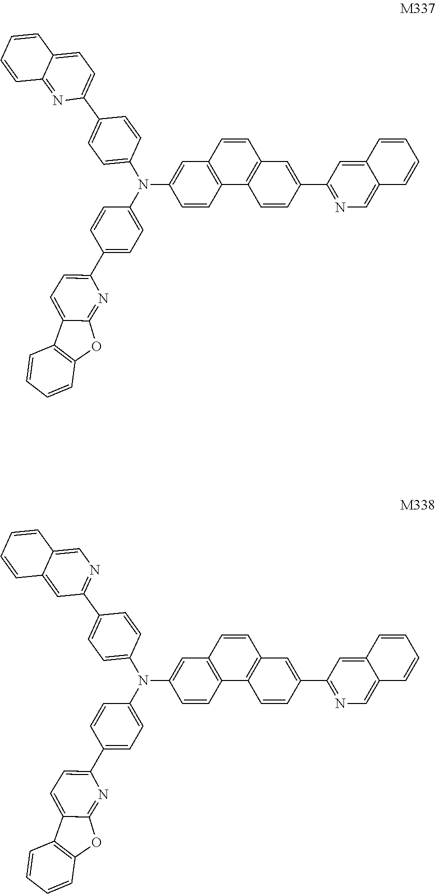

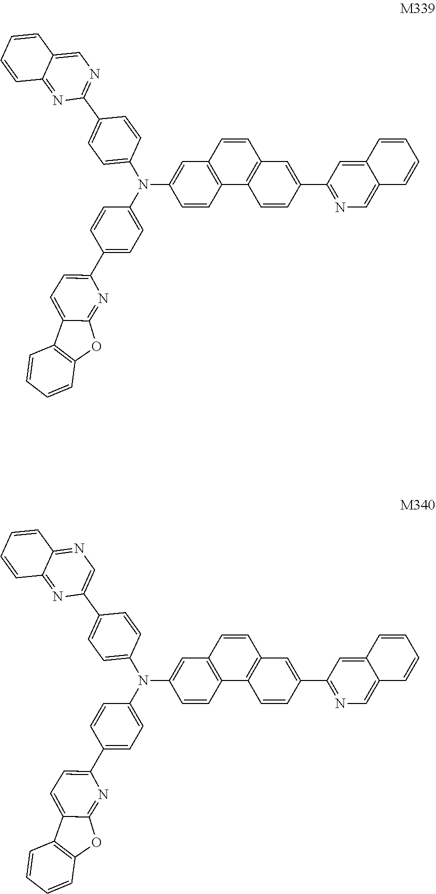

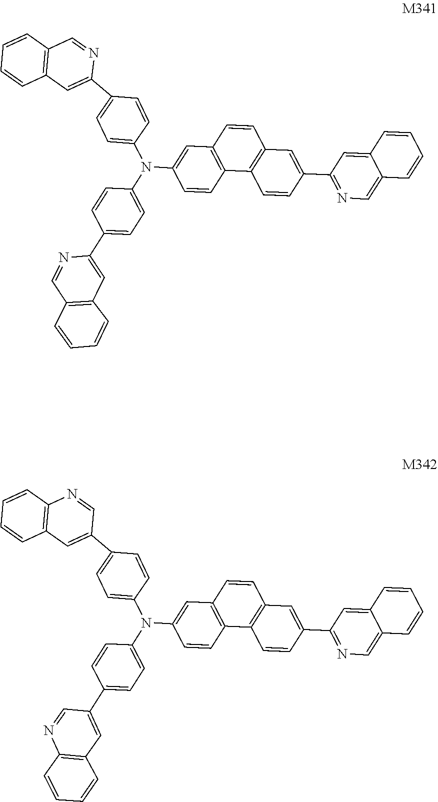

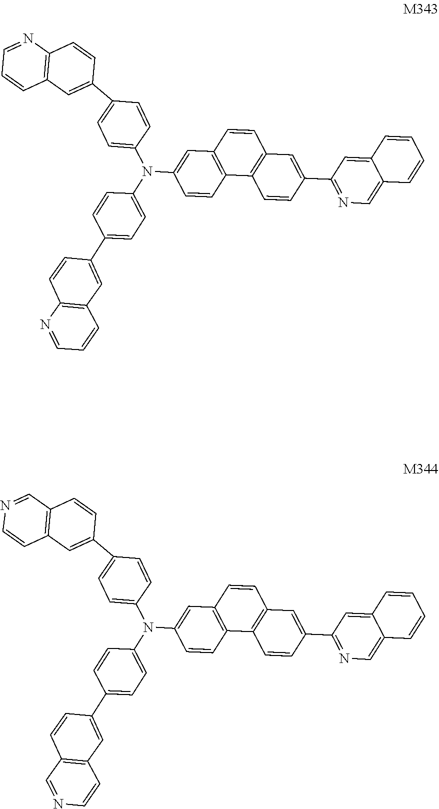

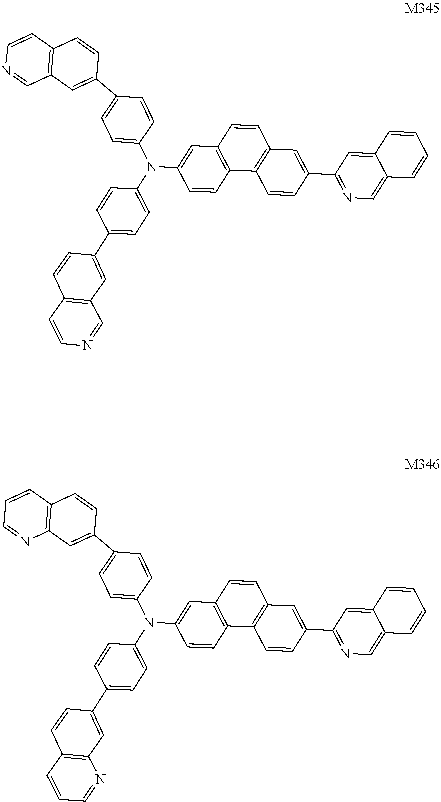

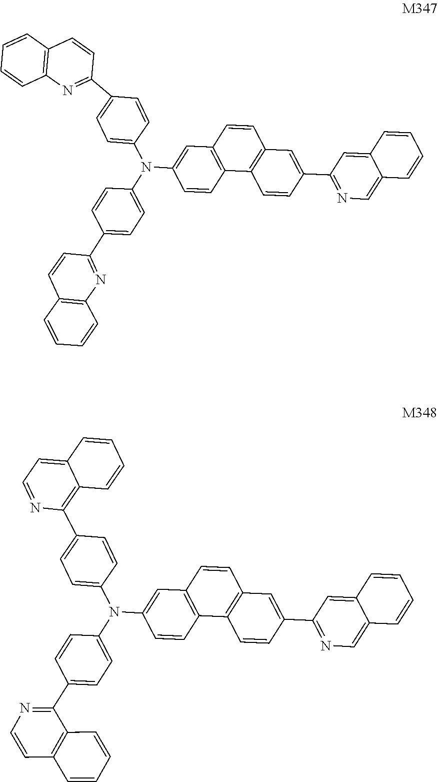

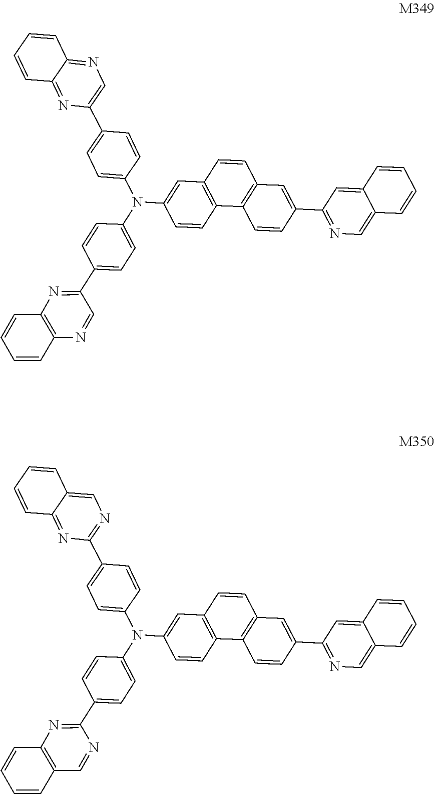

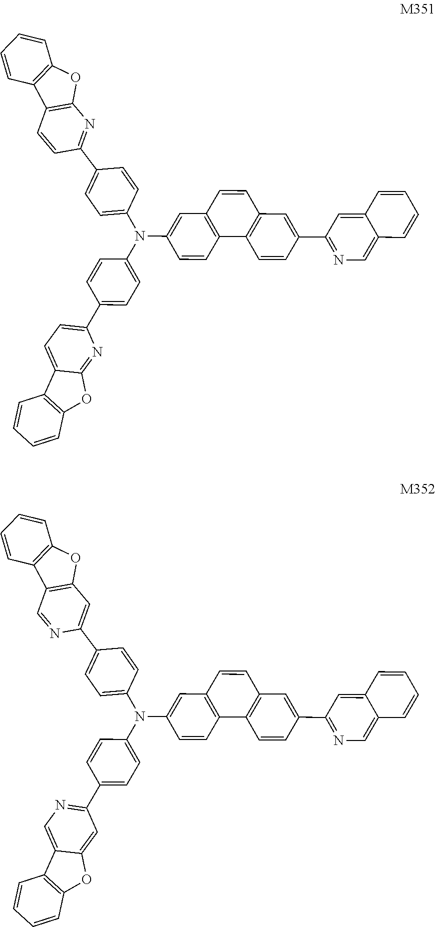

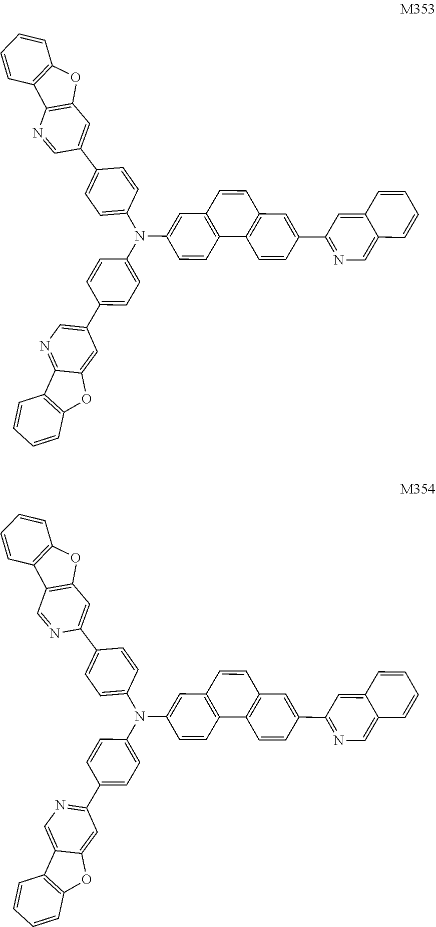

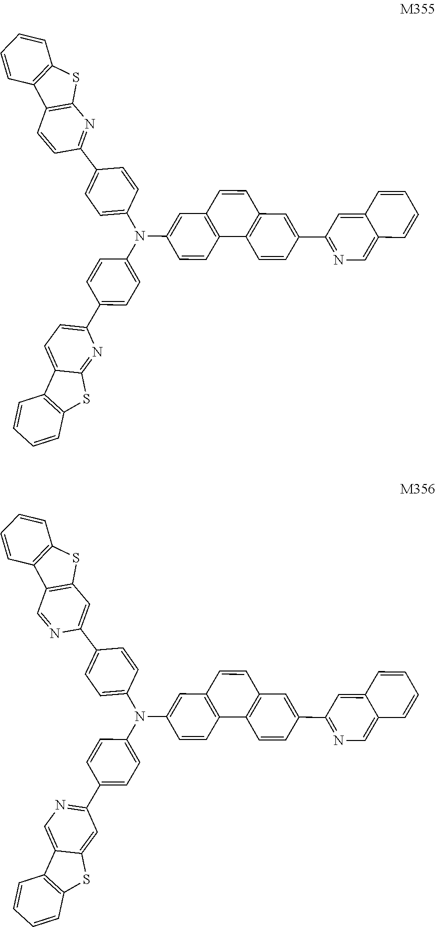

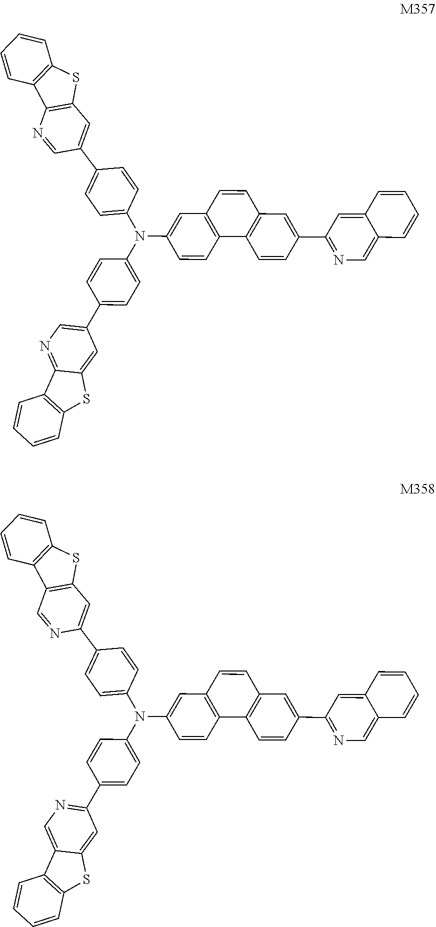

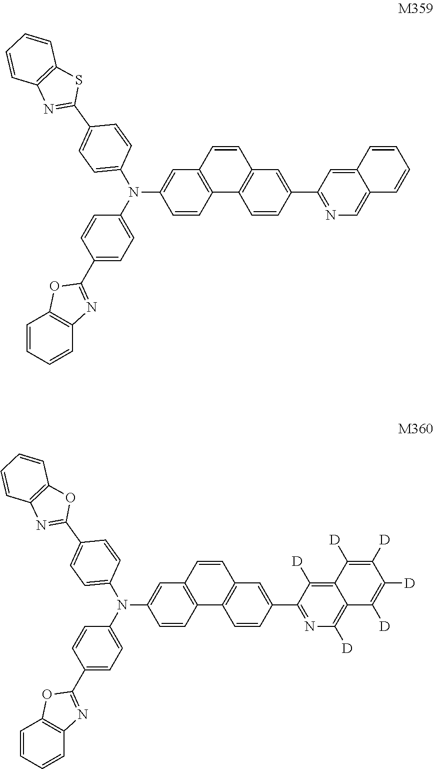

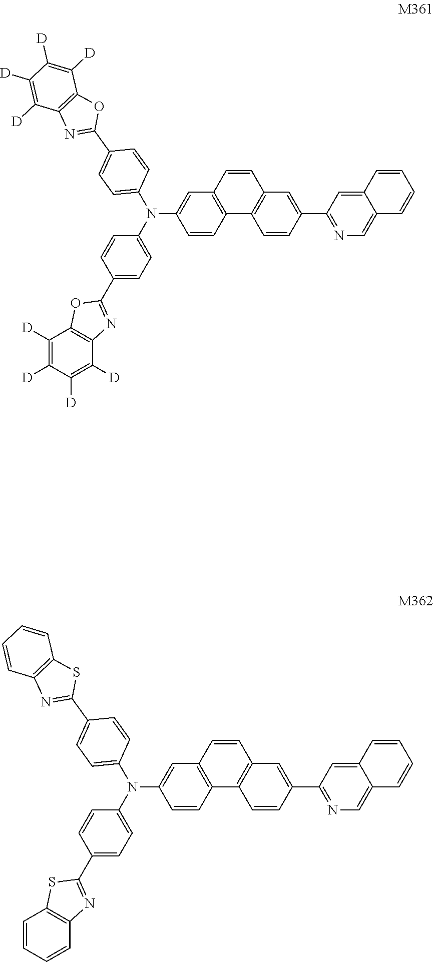

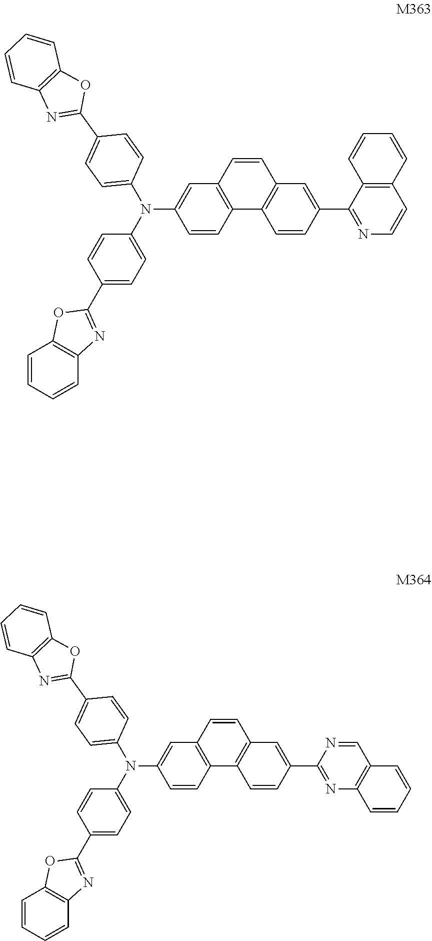

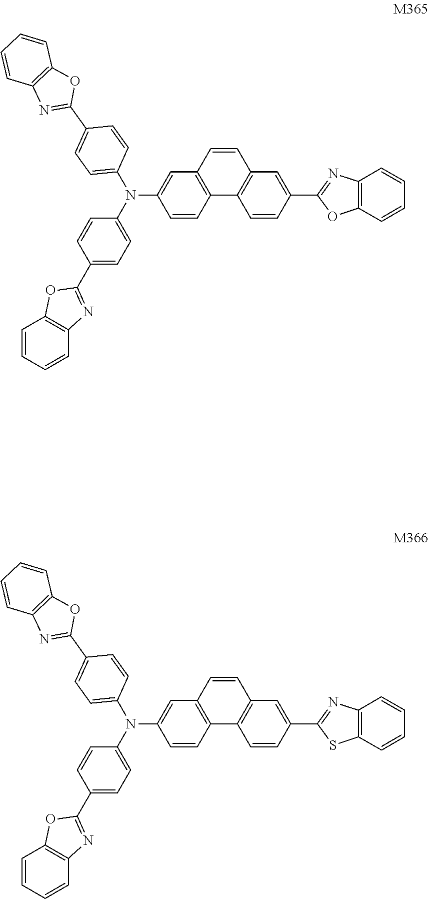

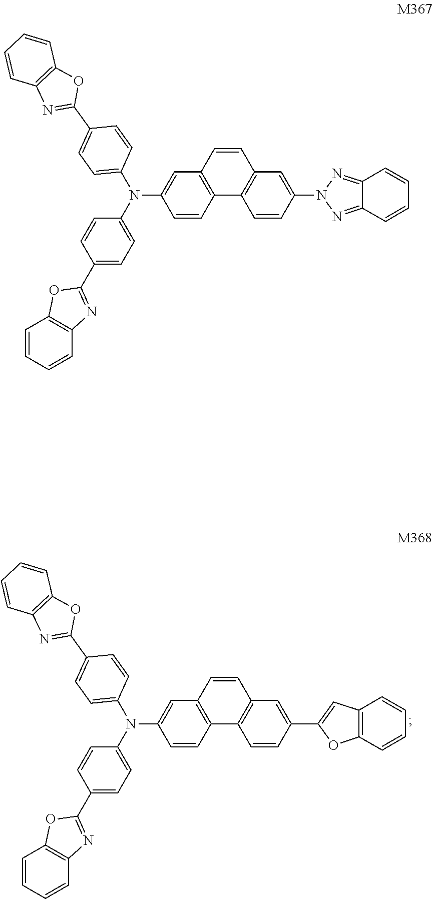

wherein D represents deuterium.

A second aspect of the present disclosure is to provide an organic electroluminescent material including the phenanthrene organic compound as described in the first aspect.

A third aspect of the present disclosure is to provide a capping layer material including the phenanthrene organic compound as described in the first aspect.

A fourth aspect of the present disclosure is to provide an electron blocking material including the phenanthrene organic compound as described in the first aspect.

A fifth aspect of the present disclosure is to provide an organic electroluminescent device including an anode, a cathode, and an organic thin-film layer disposed between the anode and the cathode, wherein the cathode is covered with a capping layer, and the capping layer includes the phenanthrene organic compound as described in the first aspect.

A sixth aspect of the present disclosure is to provide an organic electroluminescent device including an anode, a cathode, and an organic thin-film layer disposed between the anode and the cathode, wherein the organic thin-film layer includes the phenanthrene organic compound as described in the first aspect.

In the organic electroluminescent device provided by the present disclosure, the material of the anode may be a metal, a metal alloy, a metal oxide or a conductive polymer, wherein the metal includes copper, gold, silver, iron, chromium, nickel, manganese, palladium, platinum, etc., the metal alloy includes alloys formed of at least two of copper, gold, silver, iron, chromium, nickel, manganese, palladium, platinum, etc., the metal oxide includes indium oxide, zinc oxide, indium tin oxide (ITO), indium zinc oxide (IZO), etc., and the conductive polymer includes polyaniline, polypyrrole, poly(3-methylthiophene), etc. In addition to the above materials that facilitate hole injection and combinations thereof, the material of the anode further includes known materials suitable for use as the anode.

In the organic electroluminescent device, the material of the cathode may be a metal, a metal alloy or a multilayer metal material, wherein the metal includes aluminum, magnesium, silver, indium, tin, titanium, etc., the alloy includes alloys formed of at least two of aluminum, magnesium, silver, indium, tin and titanium, and the multilayer metal material includes LiF/Al, LiO₂/Al, BaF₂/Al, etc. In addition to the above materials that facilitate electron injection and combinations thereof, the material of the cathode further includes known materials suitable for use as the cathode.

In the organic electroluminescent device, the organic thin-film layer includes at least one light-emitting layer (EML) and may also include other functional layers such as any one or a combination of at least two of a hole injection layer (HIL), a hole transport layer (HTL), an electron blocking layer (EBL), a hole blocking layer (HBL), an electron transport layer (ETL) or an electron injection layer (EIL).

The organic electroluminescent device may be prepared by the following method: forming an anode on a transparent or opaque smooth substrate, forming organic thin layers on the anode, and forming a cathode on the organic thin layers. The organic thin layers may be formed by using known film forming methods such as evaporation, sputtering, spin coating, impregnation, and ion plating.

A seventh aspect of the present disclosure is to provide a display panel including the organic electroluminescent device as described in the sixth aspect.

An eighth aspect of the present disclosure is to provide an electronic device including the display panel as described in the seventh aspect.

Examples of the organic compound of the present disclosure are illustratively listed below.

### EXAMPLE 1

### Synthesis of Compound M100

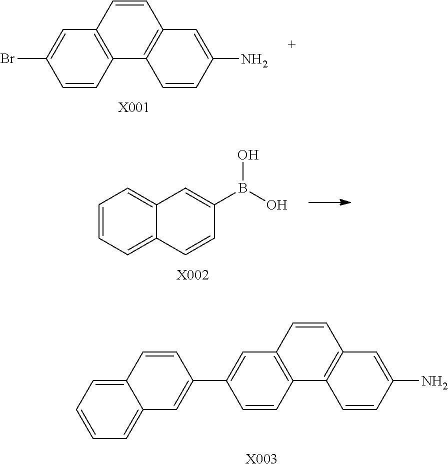

X001 (15.0 mmol), X002 (16.5 mmol), and K₂CO₃ (60 mmol) were weighed and added to a three-neck round-bottom flask that was equipped with a magnetic stir bar and was dried in a high-temperature oven. Toluene (45 mL) and H₂O (30 mL) were added. Then, the suspension was degassed and bubbled with nitrogen. After bubbling for 30 min, Pd(OAc)₂ (0.3 mmol) was added to the round-bottom flask, and the mixture was heated to reflux overnight with stirring. After the reaction was completed, the mixture was cooled to room temperature and extracted with ethyl acetate three times. The combined organic layer was washed with saturated brine and dried with anhydrous Na₂SO₄, and then volatile solvents were removed by distillation under reduced pressure to give the crude product. The crude product was purified by column chromatography to give the target compound X003 (13.8 mmol, 92%).

MALDI-TOF MS: C₂₄H₁₇N, m/z Calcd: 319.1, Found: 319.3.

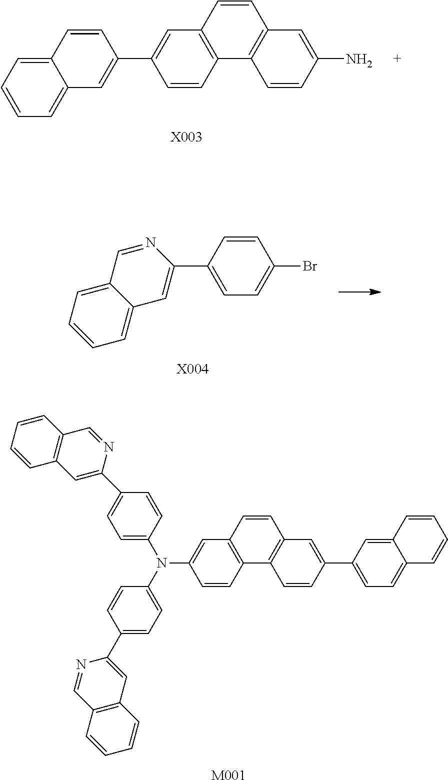 [SMILES: Nc1ccc2c(ccc3cc(-c4ccc5ccccc5c4)ccc32)c1 ; "X003" ; Brc1ccc(-c2cc3ccccc3cn2)cc1 ; "X004" ; c1ccc2cc(-c3ccc4c(ccc5cc(N(c6ccc(-c7cc8ccccc8cn7)cc6)c6ccc(-c7cc8ccccc8cn7)cc6)ccc54)c3)ccc2c1 ; "M001"]

X003 (5 mmol), X004 (10.5 mmol), and sodium tert-butoxide (33 mmol) were added to a 250 mL round-bottom flask. Pd(OAc)₂ (0.25 mmol) and tri-tert-butyl phosphine (0.25 mmol) were then added. The flask was evacuated and purged with introduced nitrogen three times, and 50 mL of toluene bubbled with nitrogen was added. Under the protection of nitrogen, the system was heated to reflux for 24 h. After the reaction was completed, the reaction system was cooled to room temperature and extracted with added dichloromethane and water three times. The organic phase was collected and dried with anhydrous Na₂SO₄, and the solvents in the organic phase were removed by a rotary evaporator. The concentrated reaction system was separated and purified by silica gel column chromatography with dichloromethane and petroleum ether in the ratio of 1:3 as the eluent. After column chromatography, the reaction system was purified by sublimation to give the product of 3.9 mmol as a white solid (with a yield of 73%).

MALDI-TOF MS: C₅₄H₃₅N₃, m/z Calcd: 725.3, Found: 725.3.

Elemental analysis: Calcd: C, 89.35; H, 4.86; N, 5.79, Found: C, 89.41; H, 4.83; N, 5.76.

### EXAMPLE 2

### Synthesis of Compound M20

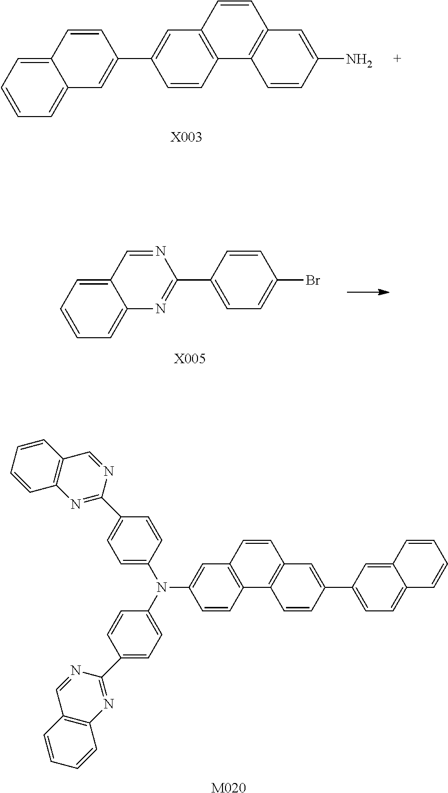 [SMILES: Nc1ccc2c(ccc3cc(-c4ccc5ccccc5c4)ccc32)c1 ; "X003" ; Brc1ccc(-c2ncc3ccccc3n2)cc1 ; "X005" ; c1ccc2cc(-c3ccc4c(ccc5cc(N(c6ccc(-c7ncc8ccccc8n7)cc6)c6ccc(-c7ncc8ccccc8n7)cc6)ccc54)c3)ccc2c1 ; "M020"]

Compound M020 was synthesized with reference to the synthesis method of Compound M001.

MALDI-TOF MS: C₅₂H₃₃N₅, m/z Calcd: 727.3, Found: 727.5.

Elemental analysis: Calcd: C, 85.81; H, 4.57; N, 9.62, Found: C, 85.86; H, 4.55; N, 9.59.

### EXAMPLE 3

### Synthesis of Compound M065

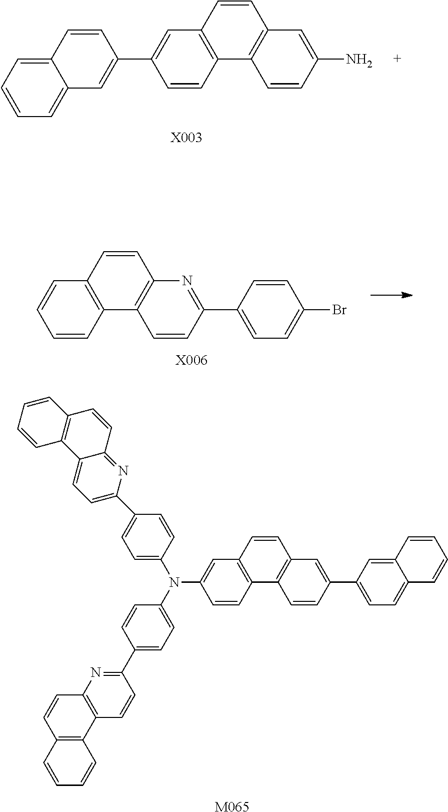

Compound M065 was synthesized with reference to the synthesis method of Compound M001.

MALDI-TOF MS: C₆₂H₃₉N₃, m/z Calcd: 825.3, Found: 825.4.

Elemental analysis: Calcd: C, 90.15; H, 4.76; N, 5.09, Found: C, 90.22; H, 4.73; N, 5.06.

### EXAMPLE 4

### Synthesis of Compound M092

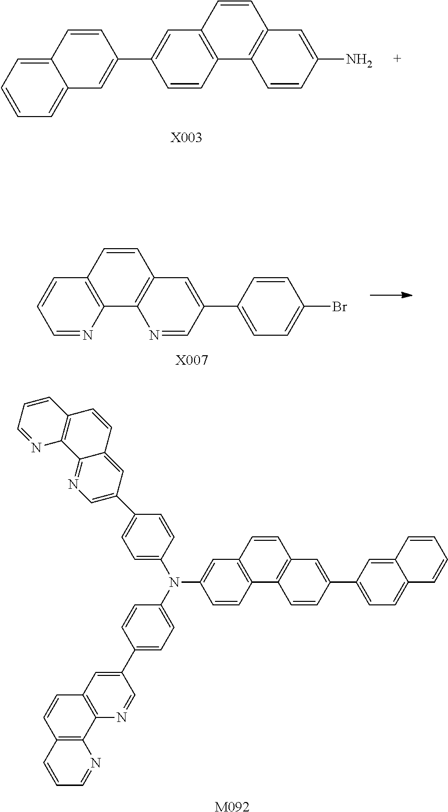

Compound M092 was synthesized with reference to the synthesis method of Compound M001.

MALDI-TOF MS: C₆₀H₃₇N₅, m/z Calcd: 827.3, Found: 827.5.

Elemental analysis: Calcd: C, 87.04; H, 4.50; N, 8.46, Found: C, 87.11; H, 4.47; N, 8.42.

### EXAMPLE 5

### Synthesis of Compound M119

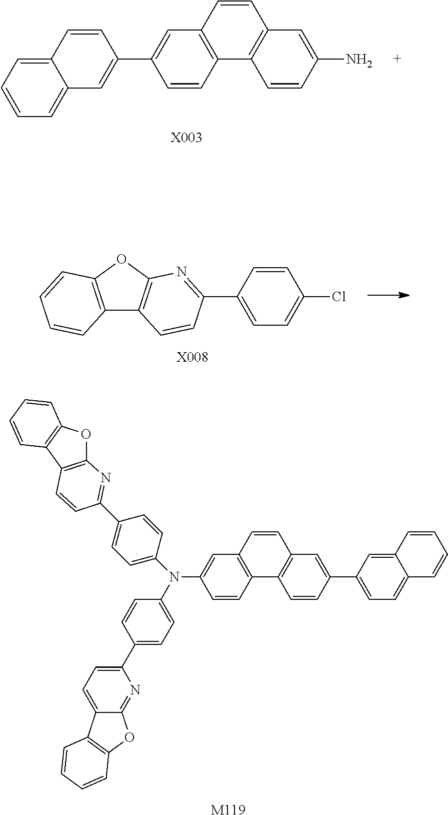

A stirrer was added to a 250 mL round-bottom flask, X003 (5 mmol), NiCl₂(dppf) (0.05 mmol), and 50 mL of toluene were added, then bubbling and purging were performed with nitrogen for 15 min, and i-PrMgCl (12 mmol, 3.0 M in 2-MeTHF) was added dropwise under a nitrogen atmosphere. After the addition was completed, the reaction mixture was stirred at room temperature for 30 min, a degassed toluene solution of X008 (15 mmol) (50 mL) was added, and the reaction mixture was stirred at 100° C. for 8 h and then quenched with 75 mL of saturated NH₄C₁ aqueous solution and 150 mL of H₂O. The reaction mixture was extracted with added dichloromethane three times. The organic phase was collected and dried with anhydrous Na₂SO₄, and the solvents in the organic phase were removed by a rotary evaporator. The concentrated reaction system was separated and purified by silica gel column chromatography with dichloromethane and petroleum ether as the eluent. After column chromatography, the reaction system was purified by sublimation to give the target compound M119 (4.1 mmol, with a yield of 82%).

MALDI-TOF MS: C₅₈H₃₅N₃O₂, m/z Calcd: 805.3, Found: 805.5.

Elemental analysis: Calcd: C, 86.44; H, 4.38; N, 5.21; O, 3.97, Found: C, 86.52; H, 4.36; N, 5.18; O, 3.94.

### EXAMPLE 6

### Synthesis of Compound M167

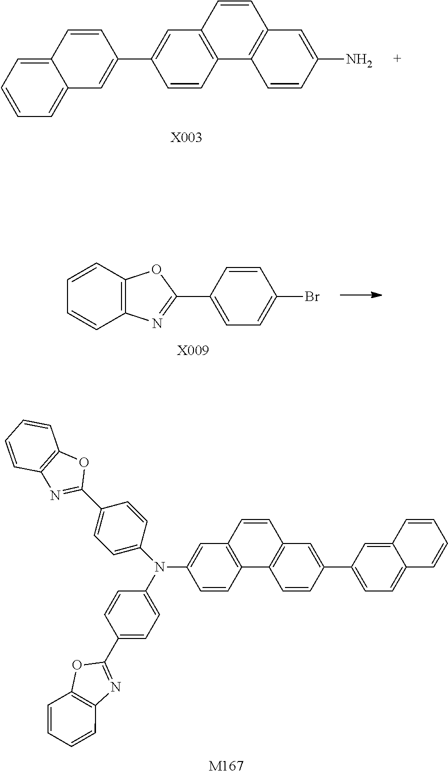 [SMILES: Nc1ccc2c(ccc3cc(-c4ccc5ccccc5c4)ccc32)c1 ; "X003" ; Brc1ccc(-c2nc3ccccc3o2)cc1 ; "X009" ; c1ccc2cc(-c3ccc4c(ccc5cc(N(c6ccc(-c7nc8ccccc8o7)cc6)c6ccc(-c7nc8ccccc8o7)cc6)ccc54)c3)ccc2c1 ; "M167"]

Compound M167 was synthesized with reference to the synthesis method of Compound M001.

MALDI-TOF MS: C₅₀H₃₁N₃O₂, m/z Calcd: 705.2, Found: 705.3.

Elemental analysis: Calcd: C, 85.09; H, 4.43; N, 5.95; O, 4.53, Found: C, 85.18; H, 4.40; N, 5.91; O, 4.51.

### EXAMPLE 7

### Synthesis of Compound M183

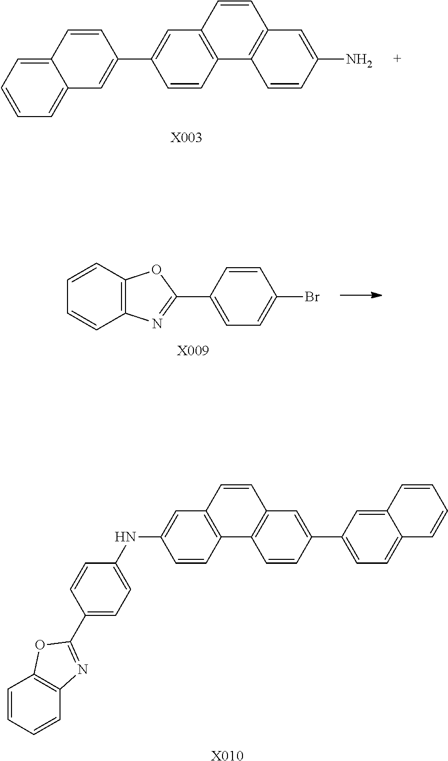

Compound X010 was synthesized with reference to the synthesis method of Compound M001.

MALDI-TOF MS: C₃₇H₂₄N₂O, m/z Calcd: 512.2, Found: 512.4.

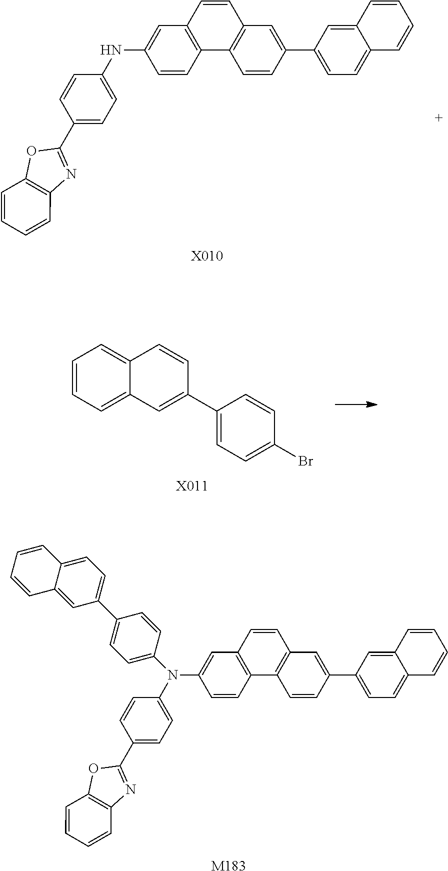 [SMILES: c1ccc2cc(-c3ccc4c(ccc5cc(Nc6ccc(-c7nc8ccccc8o7)cc6)ccc54)c3)ccc2c1 ; "X010" ; Brc1ccc(-c2ccc3ccccc3c2)cc1 ; "X011" ; c1ccc2cc(-c3ccc(N(c4ccc(-c5nc6ccccc6o5)cc4)c4ccc5c(ccc6cc(-c7ccc8ccccc8c7)ccc65)c4)cc3)ccc2c1 ; "M183"]

Compound M183 was synthesized with reference to the synthesis method of Compound M001.

MALDI-TOF MS: C₅₃H₃₄N₂O, m/z Calcd: 714.3, Found: 714.34.

Elemental analysis: Calcd: C, 89.05; H, 4.79; N, 3.92; O, 2.24, Found: C, 89.12; H, 4.77; N, 3.89; O, 2.22.

### EXAMPLE 8

### Synthesis of Compound M365

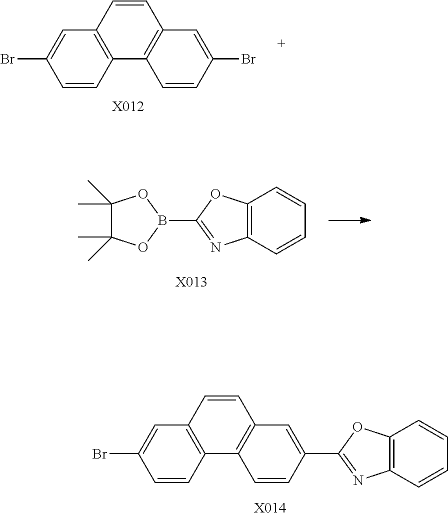

Under the protection of nitrogen, Compound X012 (5.0 mmol), X013 (5.1 mmol), [Pd₂(dba)₃]·CHCl₃ (0.1 mmol), and HP(tBu)₃·BF₄ (0.2 mmol) were weighed and added to a 500 mL two-neck flask. 200 mL of THF was added to the two-neck flask (with N₂ introduced for 15 min in advance to remove oxygen), then 20 mL of K₂CO₃ aqueous solution with the concentration of 1 M was added dropwise (with N₂ introduced for 15 min in advance to remove oxygen), and the resulting solution was stirred overnight at room temperature. After the reaction was completed, the solution was concentrated to 50 mL, 100 mL of deionized water was added, and a few drops of 2 M HCl were added, and the solution was extracted with dichloromethane. The organic phase was collected and dried with anhydrous Na₂SO₄. The solution obtained after drying was filtered, and the solvents were removed with a rotary evaporator to give the crude product. The crude product was separated and purified by silica gel column chromatography with dichloromethane and petroleum ether as the eluent, to give intermediate compound X014 (2.5 mmol, with a yield of 50%).

MALDI-TOF MS: C₂₁H₁₂BrNO, m/z Calcd: 373.0, Found: 373.1.

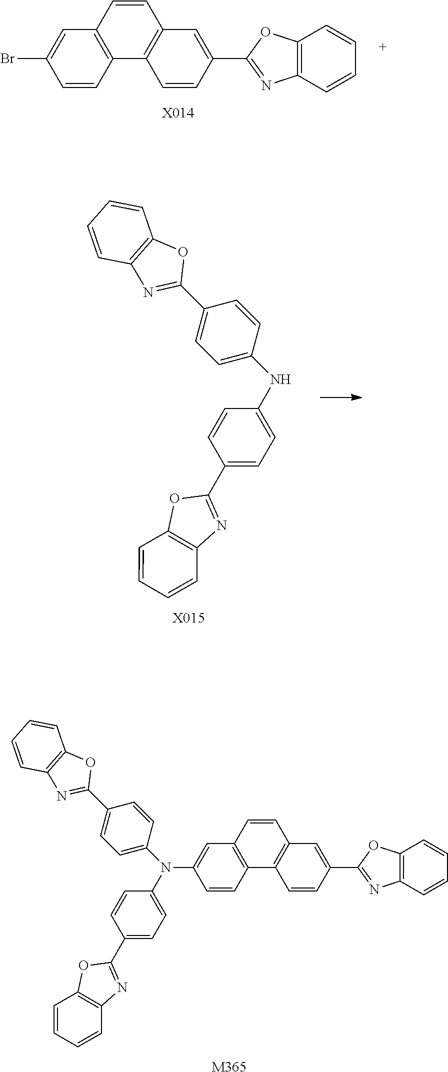

Compound M365 was synthesized with reference to the synthesis method of Compound M001.

MALDI-TOF MS: C₄₇H₂₈N₄O₃, m/z Calcd: 696.2, Found: 696.3.

Elemental analysis: Calcd: C, 81.02; H, 4.05; N, 8.04; O, 6.89, Found: C, 81.10; H, 4.02; N, 8.00; O, 6.86.

The refractive index data of the compounds are shown in Table 1.

TABLE 1

| | | Refractive | Refractive | Refractive |
|---|---|---|---|---|
| | | index | index | index |
| No. | Structure | 460 nm | 530 nm | 620 nm |
| M001 | 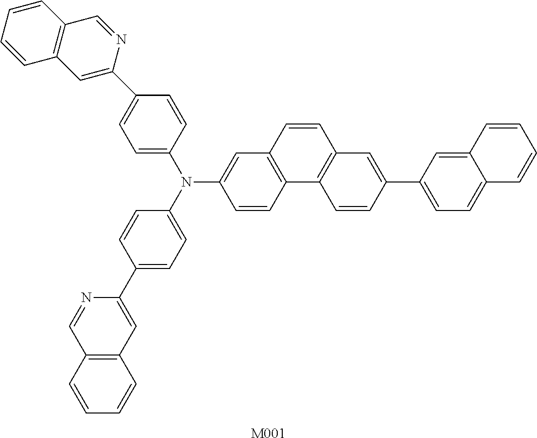 [SMILES: c1ccc2cc(-c3ccc4c(ccc5cc(N(c6ccc(-c7cc8ccccc8cn7)cc6)c6ccc(-c7cc8ccccc8cn7)cc6)ccc54)c3)ccc2c1 ; "M001"] | 2.32 | 2.18 | 2.10 |
| M007 | 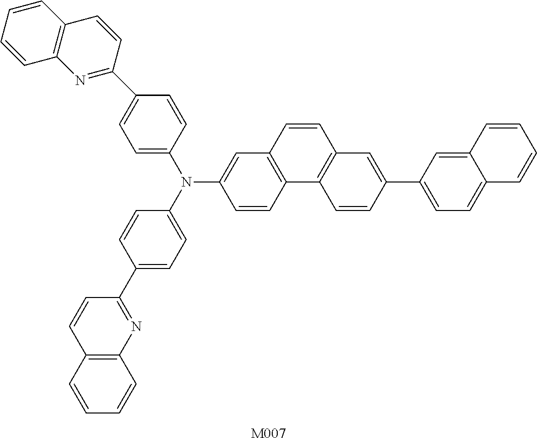 [SMILES: c1ccc2cc(-c3ccc4c(ccc5cc(N(c6ccc(-c7ccc8ccccc8n7)cc6)c6ccc(-c7ccc8ccccc8n7)cc6)ccc54)c3)ccc2c1 ; "M007"] | 2.30 | 2.17 | 2.09 |
| M019 | 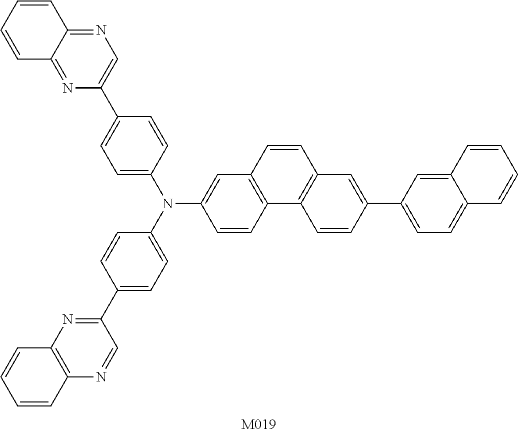 [SMILES: c1ccc2cc(-c3ccc4c(ccc5cc(N(c6ccc(-c7cnc8ccccc8n7)cc6)c6ccc(-c7cnc8ccccc8n7)cc6)ccc54)c3)ccc2c1 ; "M019"] | 2.34 | 2.19 | 2.10 |
| M020 | 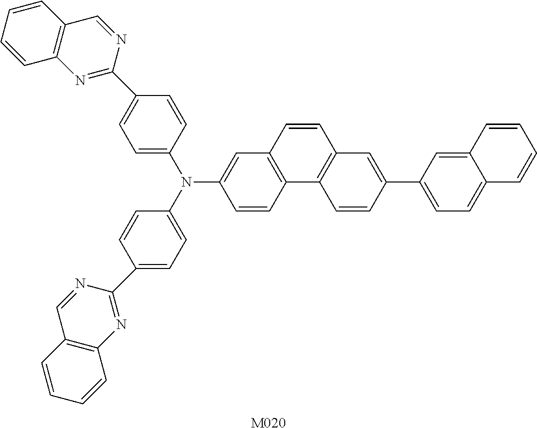 [SMILES: c1ccc2cc(-c3ccc4c(ccc5cc(N(c6ccc(-c7ncc8ccccc8n7)cc6)c6ccc(-c7ncc8ccccc8n7)cc6)ccc54)c3)ccc2c1 ; "M020"] | 2.35 | 2.20 | 2.11 |
| M037 | 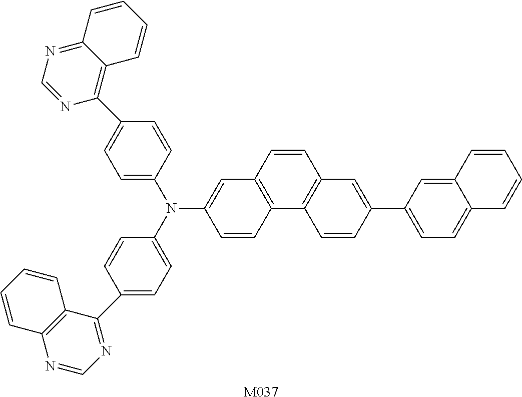 [SMILES: c1ccc2cc(-c3ccc4c(ccc5cc(N(c6ccc(-c7ncnc8ccccc78)cc6)c6ccc(-c7ncnc8ccccc78)cc6)ccc54)c3)ccc2c1 ; "M037"] | 2.18 | 2.08 | 2.02 |
| M065 | 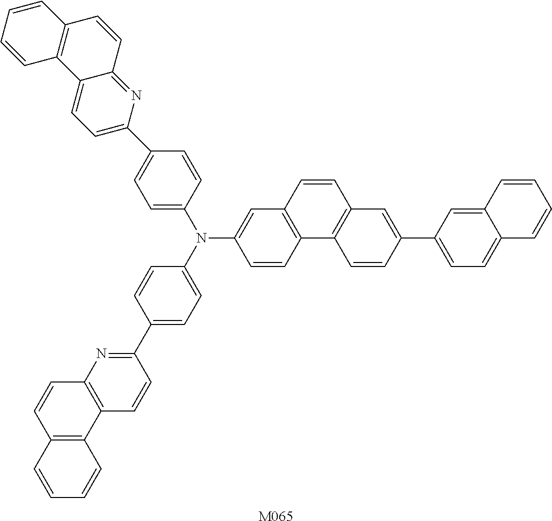 [SMILES: c1ccc2cc(-c3ccc4c(ccc5cc(N(c6ccc(-c7ccc8c(ccc9ccccc98)n7)cc6)c6ccc(-c7ccc8c(ccc9ccccc98)n7)cc6)ccc54)c3)ccc2c1 ; "M065"] | 2.40 | 2.24 | 2.15 |
| M069 | 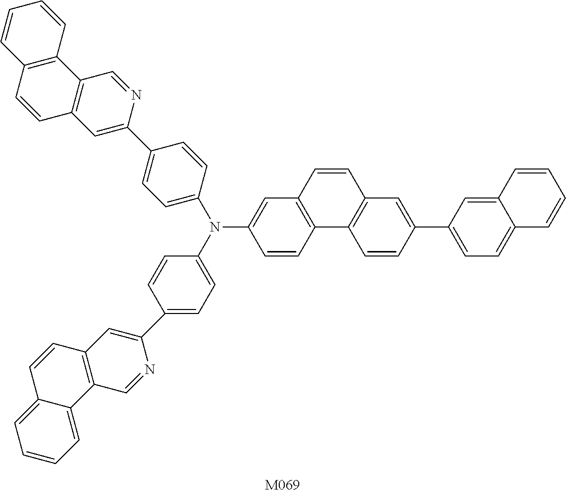 [SMILES: c1ccc2cc(-c3ccc4c(ccc5cc(N(c6ccc(-c7cc8ccc9ccccc9c8cn7)cc6)c6ccc(-c7cc8ccc9ccccc9c8cn7)cc6)ccc54)c3)ccc2c1 ; "M069"] | 2.40 | 2.24 | 2.15 |
| M075 | 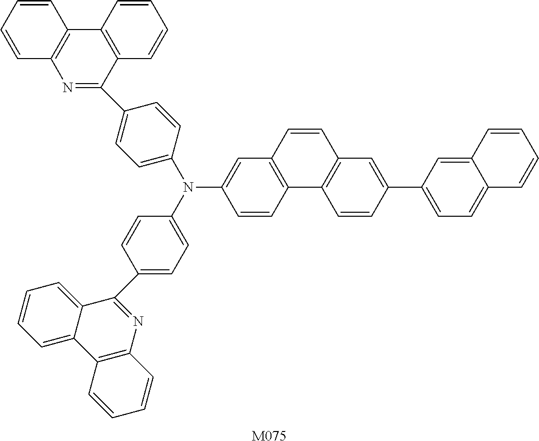 [SMILES: c1ccc2cc(-c3ccc4c(ccc5cc(N(c6ccc(-c7nc8ccccc8c8ccccc78)cc6)c6ccc(-c7nc8ccccc8c8ccccc78)cc6)ccc54)c3)ccc2c1 ; "M075"] | 2.21 | 2.11 | 2.04 |
| M091 | 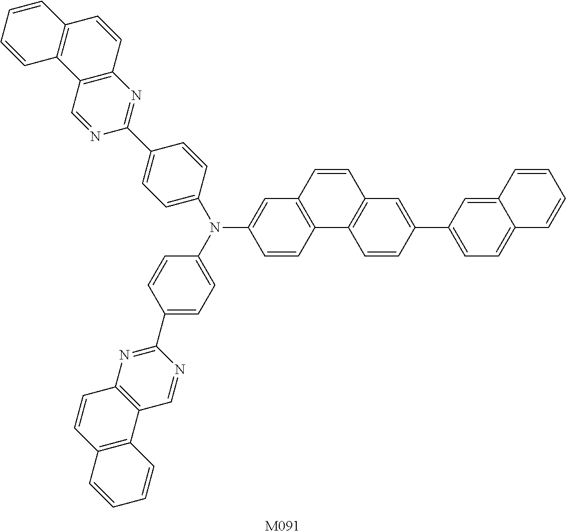 [SMILES: c1ccc2cc(-c3ccc4c(ccc5cc(N(c6ccc(-c7ncc8c(ccc9ccccc98)n7)cc6)c6ccc(-c7ncc8c(ccc9ccccc98)n7)cc6)ccc54)c3)ccc2c1 ; "M091"] | 2.44 | 2.26 | 2.16 |
| M092 | 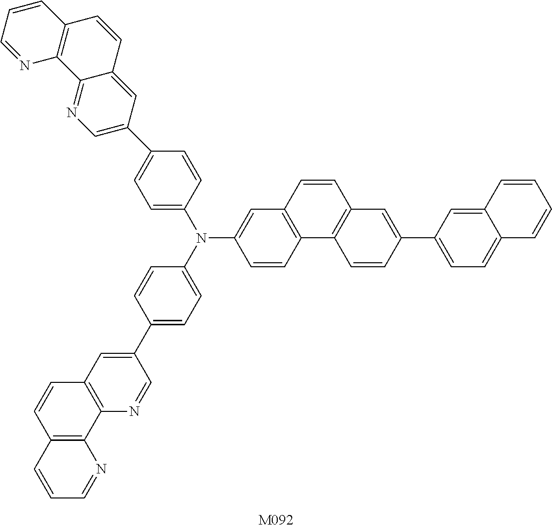 [SMILES: c1ccc2cc(-c3ccc4c(ccc5cc(N(c6ccc(-c7cnc8c(ccc9cccnc98)c7)cc6)c6ccc(-c7cnc8c(ccc9cccnc98)c7)cc6)ccc54)c3)ccc2c1 ; "M092"] | 2.29 | 2.17 | 2.10 |
| M119 | 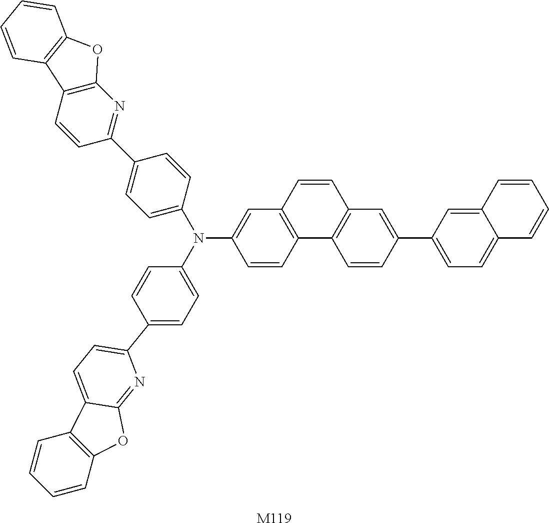 [SMILES: c1ccc2cc(-c3ccc4c(ccc5cc(N(c6ccc(-c7ccc8c(n7)oc7ccccc78)cc6)c6ccc(-c7ccc8c(n7)oc7ccccc78)cc6)ccc54)c3)ccc2c1 ; "M119"] | 2.39 | 2.23 | 2.14 |
| M122 | 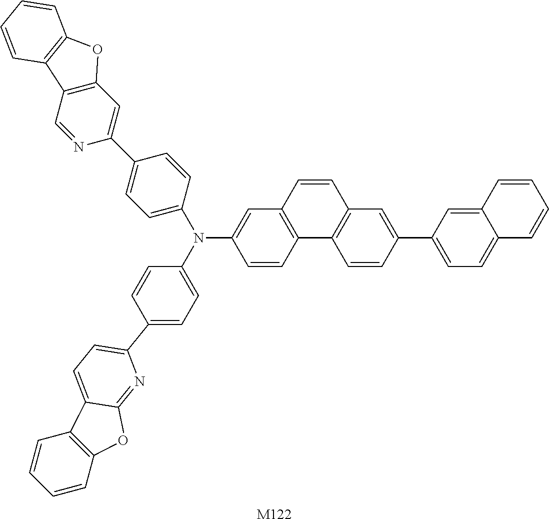 [SMILES: c1ccc2cc(-c3ccc4c(ccc5cc(N(c6ccc(-c7cc8oc9ccccc9c8cn7)cc6)c6ccc(-c7ccc8c(n7)oc7ccccc78)cc6)ccc54)c3)ccc2c1 ; "M122"] | 2.37 | 2.22 | 2.13 |
| M136 | 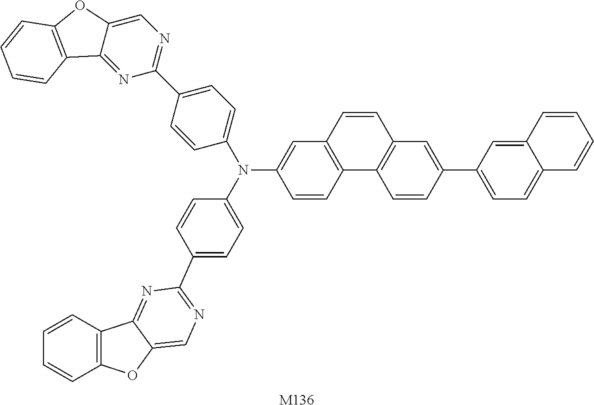 [SMILES: c1ccc2cc(-c3ccc4c(ccc5cc(N(c6ccc(-c7ncc8oc9ccccc9c8n7)cc6)c6ccc(-c7ncc8oc9ccccc9c8n7)cc6)ccc54)c3)ccc2c1 ; "M136"] | 2.26 | 2.14 | 2.07 |
| M139 | 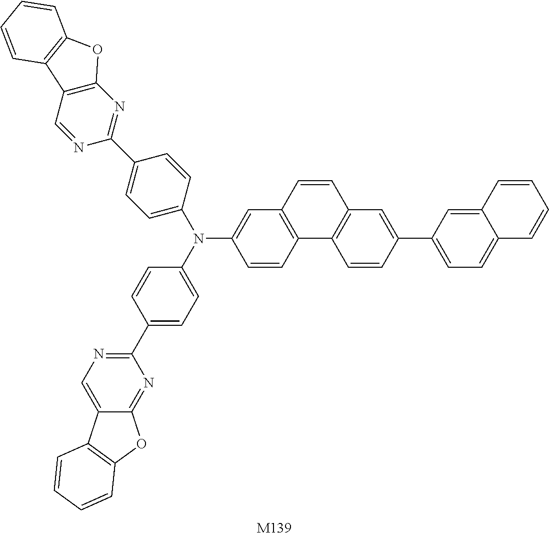 [SMILES: c1ccc2cc(-c3ccc4c(ccc5cc(N(c6ccc(-c7ncc8c(n7)oc7ccccc78)cc6)c6ccc(-c7ncc8c(n7)oc7ccccc78)cc6)ccc54)c3)ccc2c1 ; "M139"] | 2.44 | 2.26 | 2.15 |
| M145 | 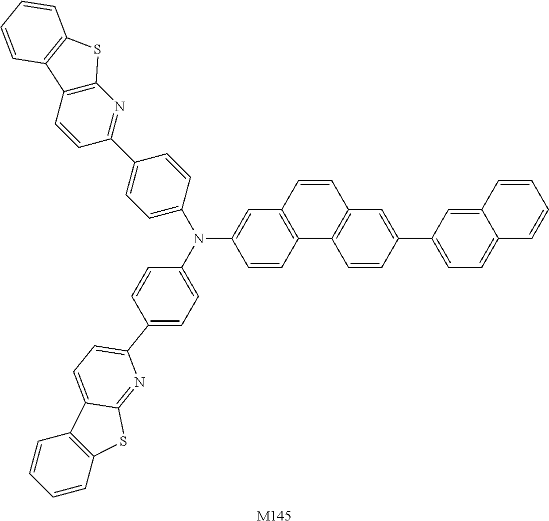 [SMILES: c1ccc2cc(-c3ccc4c(ccc5cc(N(c6ccc(-c7ccc8c(n7)sc7ccccc78)cc6)c6ccc(-c7ccc8c(n7)sc7ccccc78)cc6)ccc54)c3)ccc2c1 ; "M145"] | 2.38 | 2.22 | 2.13 |
| M165 | 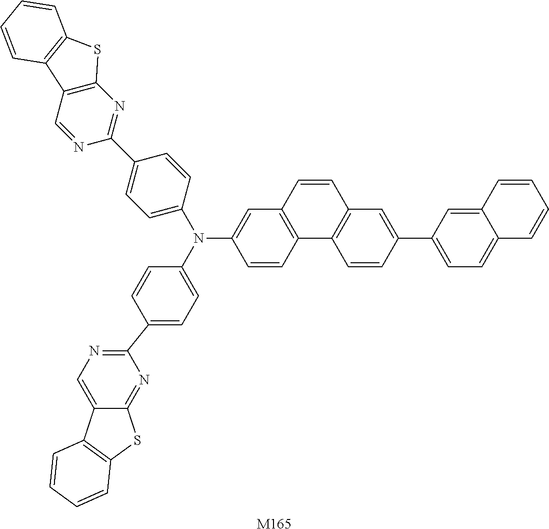 [SMILES: c1ccc2cc(-c3ccc4c(ccc5cc(N(c6ccc(-c7ncc8c(n7)sc7ccccc78)cc6)c6ccc(-c7ncc8c(n7)sc7ccccc78)cc6)ccc54)c3)ccc2c1 ; "M165"] | 2.44 | 2.26 | 2.16 |
| M167 | 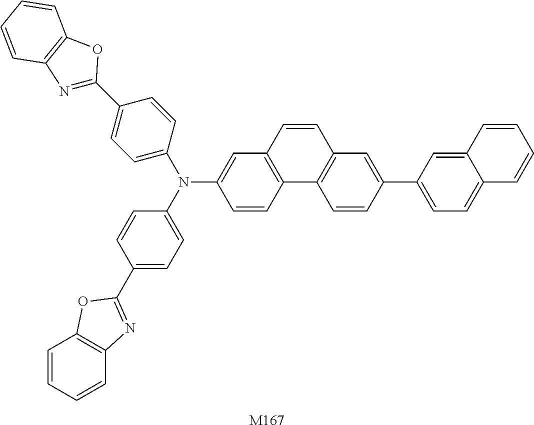 [SMILES: c1ccc2cc(-c3ccc4c(ccc5cc(N(c6ccc(-c7nc8ccccc8o7)cc6)c6ccc(-c7nc8ccccc8o7)cc6)ccc54)c3)ccc2c1 ; "M167"] | 2.29 | 2.16 | 2.08 |
| M171 | 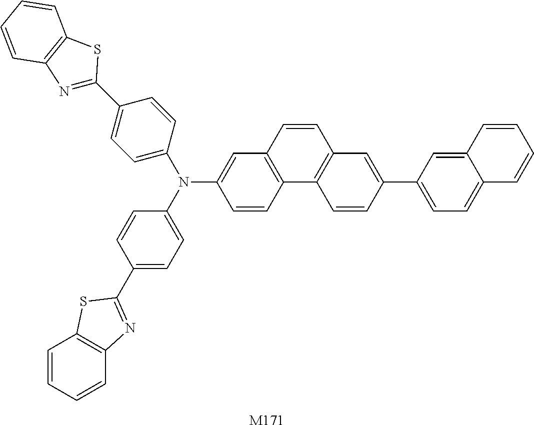 [SMILES: c1ccc2cc(-c3ccc4c(ccc5cc(N(c6ccc(-c7nc8ccccc8s7)cc6)c6ccc(-c7nc8ccccc8s7)cc6)ccc54)c3)ccc2c1 ; "M171"] | 2.34 | 2.19 | 2.10 |
| M181 | 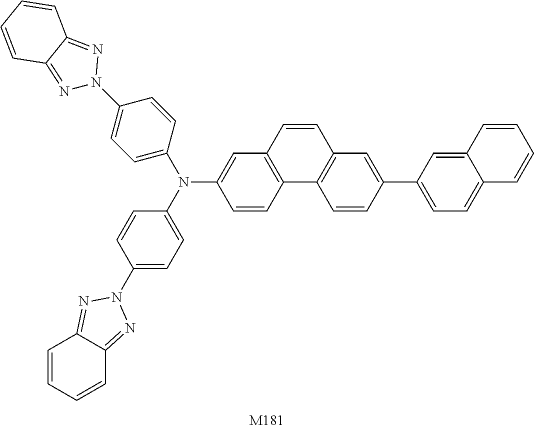 [SMILES: c1ccc2cc(-c3ccc4c(ccc5cc(N(c6ccc(-n7nc8ccccc8n7)cc6)c6ccc(-n7nc8ccccc8n7)cc6)ccc54)c3)ccc2c1 ; "M181"] | 2.29 | 2.15 | 2.07 |
| M183 | 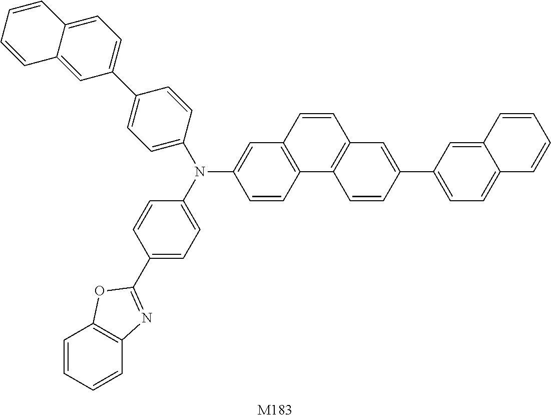 [SMILES: c1ccc2cc(-c3ccc(N(c4ccc(-c5nc6ccccc6o5)cc4)c4ccc5c(ccc6cc(-c7ccc8ccccc8c7)ccc65)c4)cc3)ccc2c1 ; "M183"] | 2.27 | 2.14 | 2.07 |
| M185 | 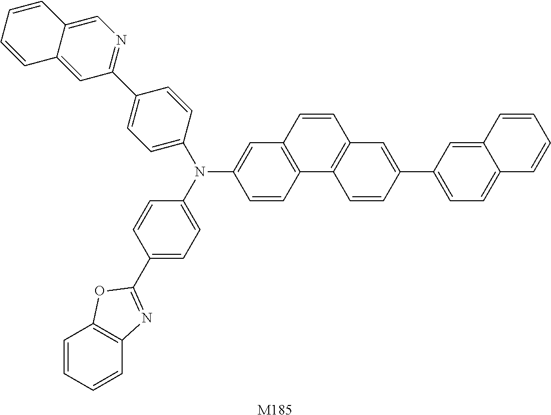 [SMILES: c1ccc2cc(-c3ccc4c(ccc5cc(N(c6ccc(-c7cc8ccccc8cn7)cc6)c6ccc(-c7nc8ccccc8o7)cc6)ccc54)c3)ccc2c1 ; "M185"] | 2.31 | 2.17 | 2.09 |
| M188 | 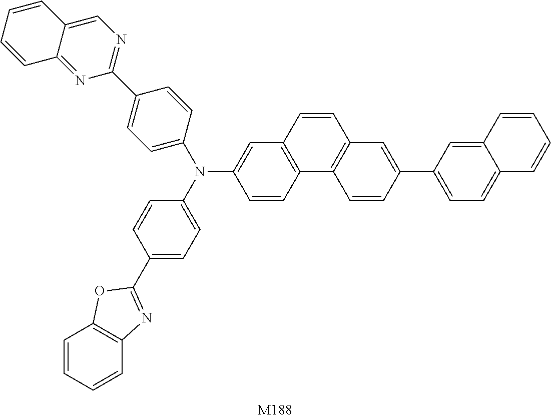 [SMILES: c1ccc2cc(-c3ccc4c(ccc5cc(N(c6ccc(-c7ncc8ccccc8n7)cc6)c6ccc(-c7nc8ccccc8o7)cc6)ccc54)c3)ccc2c1 ; "M188"] | 2.32 | 2.18 | 2.09 |
| M200 | 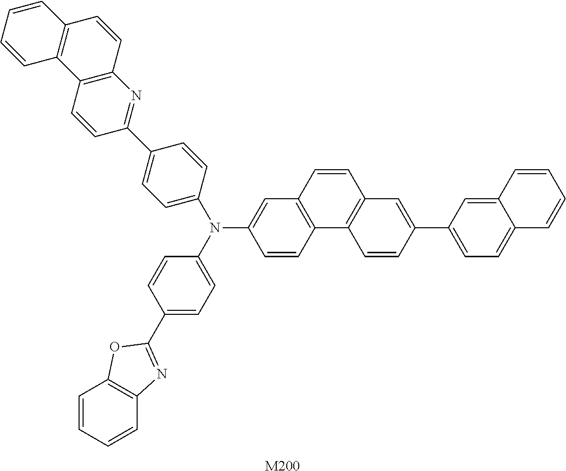 [SMILES: c1ccc2cc(-c3ccc4c(ccc5cc(N(c6ccc(-c7ccc8c(ccc9ccccc98)n7)cc6)c6ccc(-c7nc8ccccc8o7)cc6)ccc54)c3)ccc2c1 ; "M200"] | 2.35 | 2.20 | 2.12 |
| M206 | 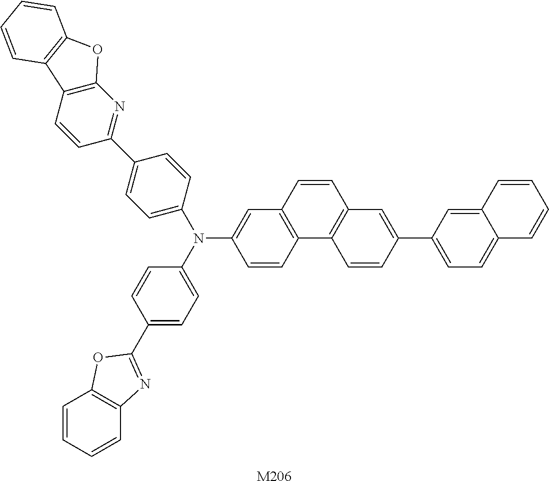 [SMILES: c1ccc2cc(-c3ccc4c(ccc5cc(N(c6ccc(-c7ccc8c(n7)oc7ccccc78)cc6)c6ccc(-c7nc8ccccc8o7)cc6)ccc54)c3)ccc2c1 ; "M206"] | 2.35 | 2.20 | 2.11 |
| M236 | 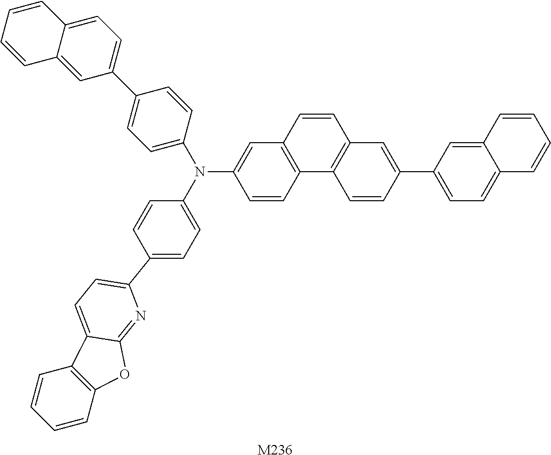 [SMILES: c1ccc2cc(-c3ccc(N(c4ccc(-c5ccc6c(n5)oc5ccccc56)cc4)c4ccc5c(ccc6cc(-c7ccc8ccccc8c7)ccc65)c4)cc3)ccc2c1 ; "M236"] | 2.32 | 2.18 | 2.10 |
| M240 | 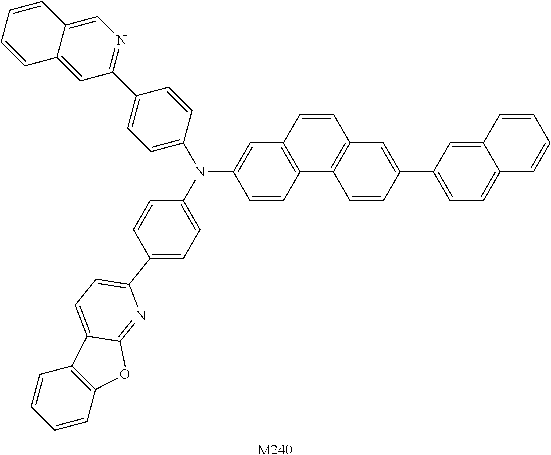 [SMILES: c1ccc2cc(-c3ccc4c(ccc5cc(N(c6ccc(-c7cc8ccccc8cn7)cc6)c6ccc(-c7ccc8c(n7)oc7ccccc78)cc6)ccc54)c3)ccc2c1 ; "M240"] | 2.36 | 2.21 | 2.12 |
| M246 | 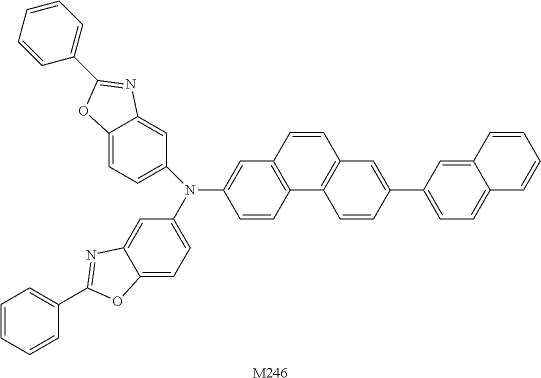 [SMILES: c1ccc(-c2nc3cc(N(c4ccc5c(ccc6cc(-c7ccc8ccccc8c7)ccc65)c4)c4ccc5oc(-c6ccccc6)nc5c4)ccc3o2)cc1 ; "M246"] | 2.23 | 2.12 | 2.05 |
| M262 | 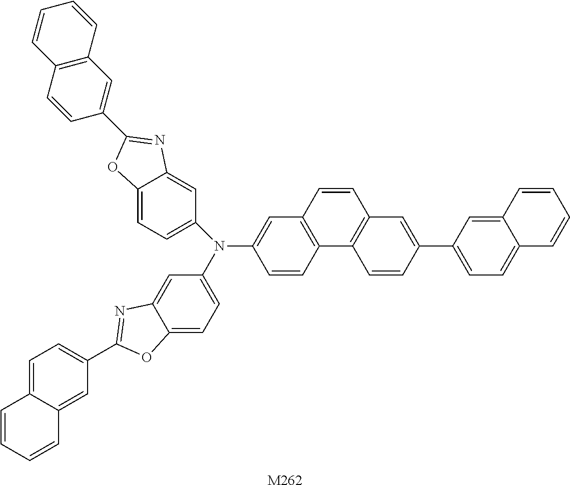 [SMILES: c1ccc2cc(-c3ccc4c(ccc5cc(N(c6ccc7oc(-c8ccc9ccccc9c8)nc7c6)c6ccc7oc(-c8ccc9ccccc9c8)nc7c6)ccc54)c3)ccc2c1 ; "M262"] | 2.31 | 2.18 | 2.10 |
| M293 | 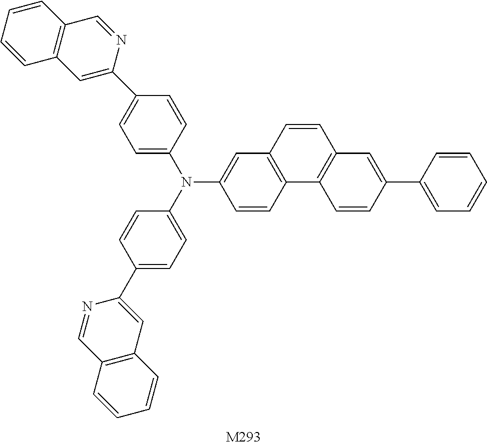 [SMILES: c1ccc(-c2ccc3c(ccc4cc(N(c5ccc(-c6cc7ccccc7cn6)cc5)c5ccc(-c6cc7ccccc7cn6)cc5)ccc43)c2)cc1 ; "M293"] | 2.26 | 2.14 | 2.06 |
| M301 | 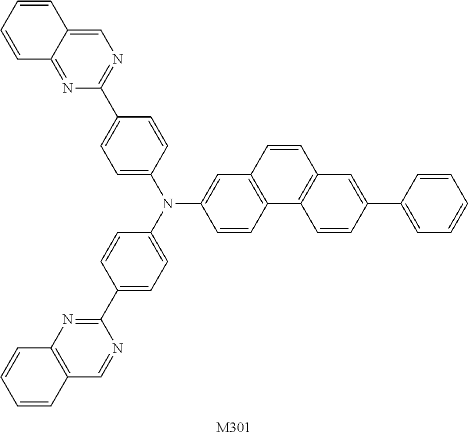 [SMILES: c1ccc(-c2ccc3c(ccc4cc(N(c5ccc(-c6ncc7ccccc7n6)cc5)c5ccc(-c6ncc7ccccc7n6)cc5)ccc43)c2)cc1 ; "M301"] | 2.30 | 2.16 | 2.08 |
| M313 | 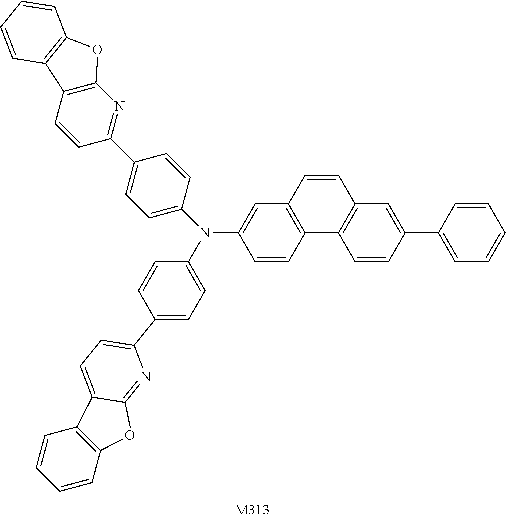 [SMILES: c1ccc(-c2ccc3c(ccc4cc(N(c5ccc(-c6ccc7c(n6)oc6ccccc67)cc5)c5ccc(-c6ccc7c(n6)oc6ccccc67)cc5)ccc43)c2)cc1 ; "M313"] | 2.34 | 2.19 | 2.10 |
| M321 | 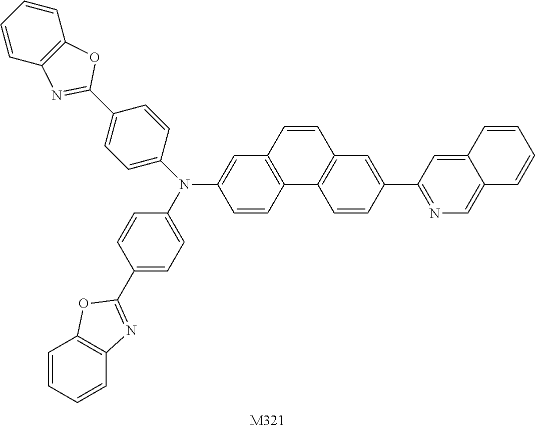 [SMILES: c1ccc2cc(-c3ccc4c(ccc5cc(N(c6ccc(-c7nc8ccccc8o7)cc6)c6ccc(-c7nc8ccccc8o7)cc6)ccc54)c3)ncc2c1 ; "M321"] | 2.33 | 2.19 | 2.10 |
| M331 | 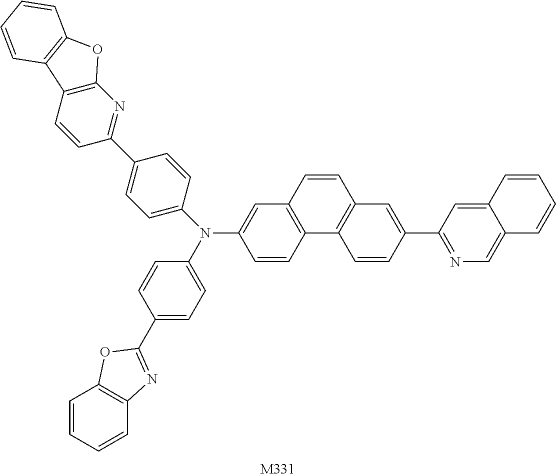 [SMILES: c1ccc2cc(-c3ccc4c(ccc5cc(N(c6ccc(-c7ccc8c(n7)oc7ccccc78)cc6)c6ccc(-c7nc8ccccc8o7)cc6)ccc54)c3)ncc2c1 ; "M331"] | 2.39 | 2.22 | 2.13 |
| M341 | 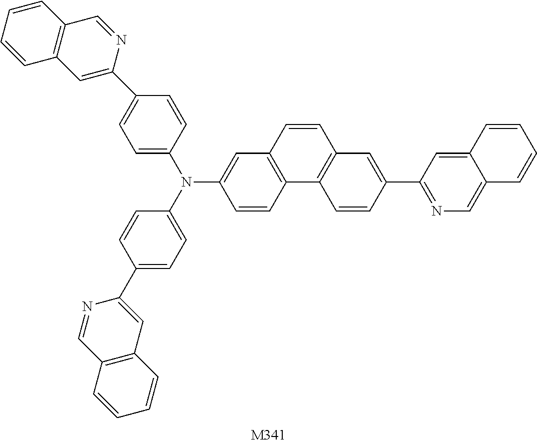 [SMILES: c1ccc2cc(-c3ccc(N(c4ccc(-c5cc6ccccc6cn5)cc4)c4ccc5c(ccc6cc(-c7cc8ccccc8cn7)ccc65)c4)cc3)ncc2c1 ; "M341"] | 2.36 | 2.21 | 2.12 |
| M365 | 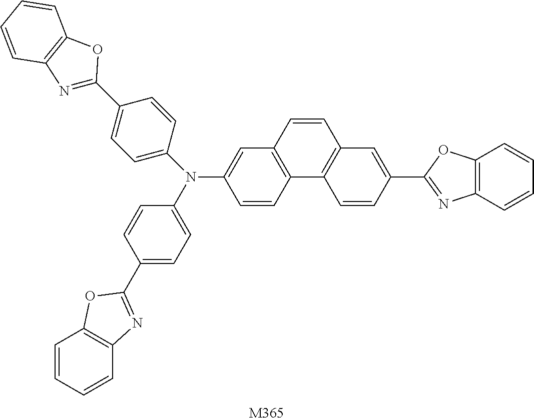 [SMILES: c1ccc2oc(-c3ccc(N(c4ccc(-c5nc6ccccc6o5)cc4)c4ccc5c(ccc6cc(-c7nc8ccccc8o7)ccc65)c4)cc3)nc2c1 ; "M365"] | 2.31 | 2.17 | 2.08 |
| M367 | 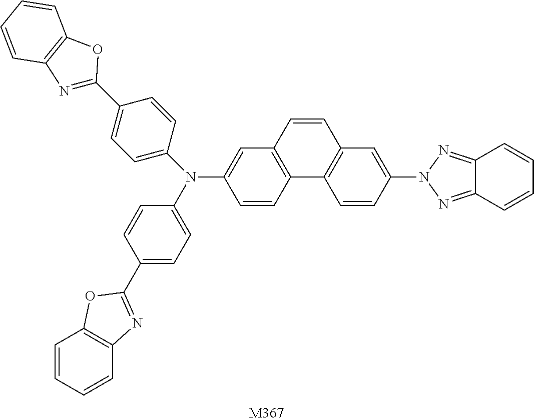 [SMILES: c1ccc2nn(-c3ccc4c(ccc5cc(N(c6ccc(-c7nc8ccccc8o7)cc6)c6ccc(-c7nc8ccccc8o7)cc6)ccc54)c3)nc2c1 ; "M367"] | 2.31 | 2.16 | 2.08 |
| Ref1 | 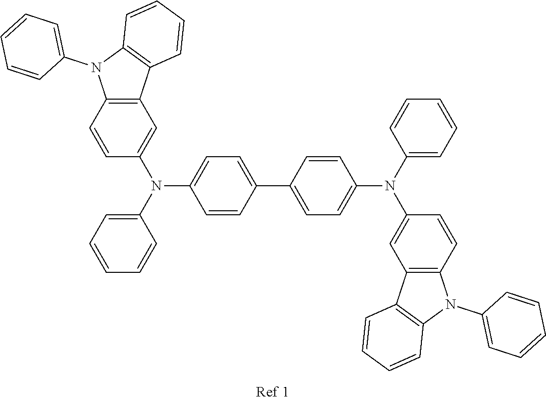 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1] | 2.03 | 1.95 | 1.90 |
| Ref2 | 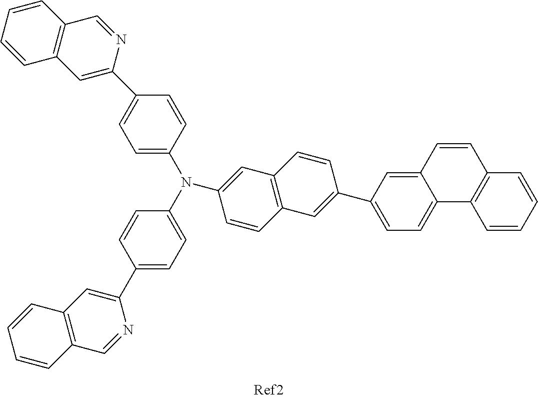 [SMILES: c1ccc2cc(-c3ccc(N(c4ccc(-c5cc6ccccc6cn5)cc4)c4ccc5cc(-c6ccc7c(ccc8ccccc87)c6)ccc5c4)cc3)ncc2c1] | 2.33 | 2.18 | 2.10 |
| Ref3 | 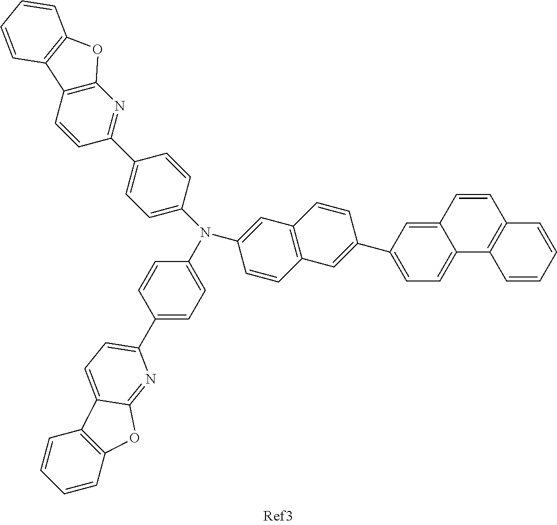 [SMILES: c1ccc2c(c1)ccc1cc(-c3ccc4cc(N(c5ccc(-c6ccc7c(n6)oc6ccccc67)cc5)c5ccc(-c6ccc7c(n6)oc6ccccc67)cc5)ccc4c3)ccc12] | 2.40 | 2.23 | 2.14 |
| Ref4 | 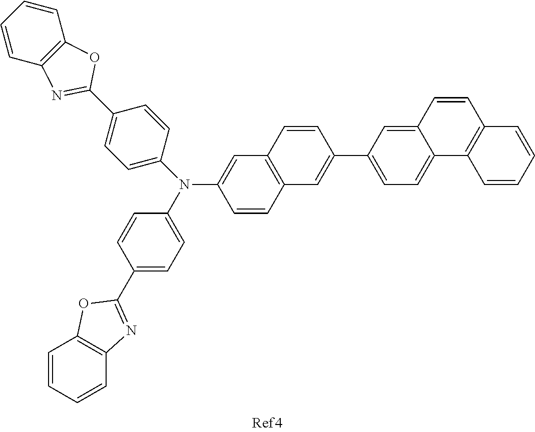 [SMILES: c1ccc2c(c1)ccc1cc(-c3ccc4cc(N(c5ccc(-c6nc7ccccc7o6)cc5)c5ccc(-c6nc7ccccc7o6)cc5)ccc4c3)ccc12] | 2.30 | 2.16 | 2.09 |
| Ref5 | 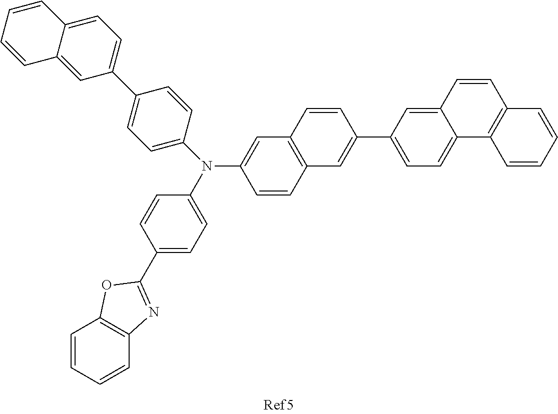 [SMILES: c1ccc2cc(-c3ccc(N(c4ccc(-c5nc6ccccc6o5)cc4)c4ccc5cc(-c6ccc7c(ccc8ccccc87)c6)ccc5c4)cc3)ccc2c1] | 2.28 | 2.14 | 2.08 |
| Ref6 | 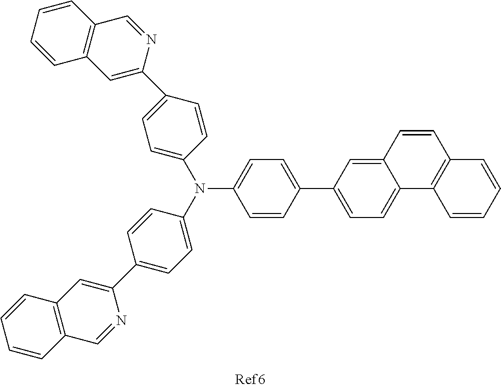 [SMILES: c1ccc2cc(-c3ccc(N(c4ccc(-c5ccc6c(ccc7ccccc76)c5)cc4)c4ccc(-c5cc6ccccc6cn5)cc4)cc3)ncc2c1] | 2.27 | 2.14 | 2.07 |

As can be seen from the refractive index data in Table 1, the compound of the present disclosure has a higher refractive index over the entire visible wavelength range compared with the cover layer material Refl commonly used in the industry at present. It is concluded that in blue, green and red light devices, higher luminescence efficiency can be achieved by using the above materials as the capping layer materials in organic electroluminescent devices.

### APPLICATION EXAMPLE 1A

This application example provides an organic electroluminescent device. The present disclosure provides a structure diagram of the organic electroluminescent device, the organic electroluminescent device includes a substrate, an anode, a hole injection layer, a first hole transport layer, a second hole transport layer, an electron transport layer, an electron injection layer, a cathode 8 and a capping layer which are stacked in sequence.

The structure of an OLED blue light device is: ITO (10 nm)/Compound 1:Compound 2 (with the mass ratio of 3:97) (5 nm)/Compound 3 (100 nm)/Compound 4 (5 nm)/Compound 5:Compound 6 (with the mass ratio of 97:3) (30 nm)/Compound 7 (5 nm)/Compound 8:Compound 9 (with the mass ratio of 1:1) (30 nm)/Mg:Ag (with the mass ratio of 10:90, 10 mass % Mg) (10 nm)/M001 (70 nm).

The organic electroluminescent device was prepared by the steps below.
1) A glass substrate was cut into 50 mm×50 mm×0.7 mm, sonicated in isopropanol and deionized water for 30 min separately, and exposed under ozone for about 10 min for cleaning to give the substrate 1. The obtained glass substrate having the 10 nm ITO anode was installed on a vacuum deposition device.
2) The hole injection layer material Compound 2 and a p-doped material Compound 1 were co-deposited at a doping ratio of 3% (mass ratio) by means of vacuum evaporation on the ITO anode 2 as the hole injection layer 3 with a thickness of 5 nm.
3) The hole transport layer material Compound 3 was deposited by means of vacuum evaporation on the hole injection layer 3 as the first hole transport layer 4 with a thickness of 100 nm.
4) The hole transport material Compound 4 was deposited by means of vacuum evaporation on the first hole transport layer 4 as the second hole transport layer 5 with a thickness of 5 nm.
5) One layer of light-emitting layer 6 with a thickness of 30 nm was deposited by means of vacuum evaporation on the second hole transport layer 5, wherein Compound 5 serving as a host material was doped with Compound 6 serving as a doped material at a ratio of 3% (mass ratio).
6) The electron transport material Compound 7 was deposited by means of vacuum evaporation on the light-emitting layer 6 as the electron transport layer 7 with a thickness of 5 nm.
7) The electron transport materials Compound 8 and Compound 9 were co-deposited by means of vacuum evaporation on the electron transport layer 7 at a doping mass ratio of 1:1 as the electron injection layer 8 with a thickness of 30 nm.
8) The magnesium-silver electrode was deposited by means of vacuum evaporation on the electron injection layer 8 at an Mg—Ag ratio of 1:9 as the cathode 9 with a thickness of 10 nm.
(9) Compound M001 was deposited by means of vacuum evaporation on the cathode 9 as the capping layer 10 with a thickness of 70 nm.

The structures of compounds used in the organic electroluminescent device are as follows:

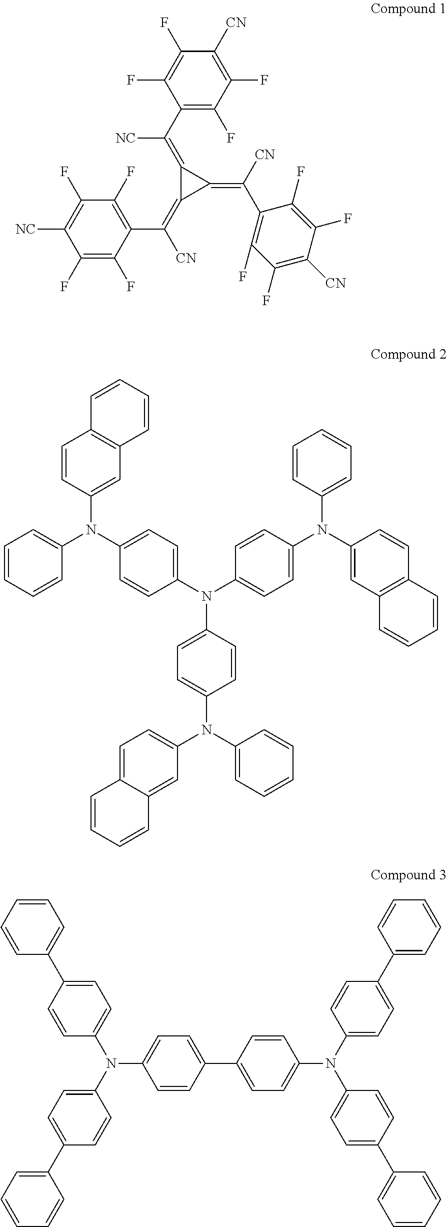 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

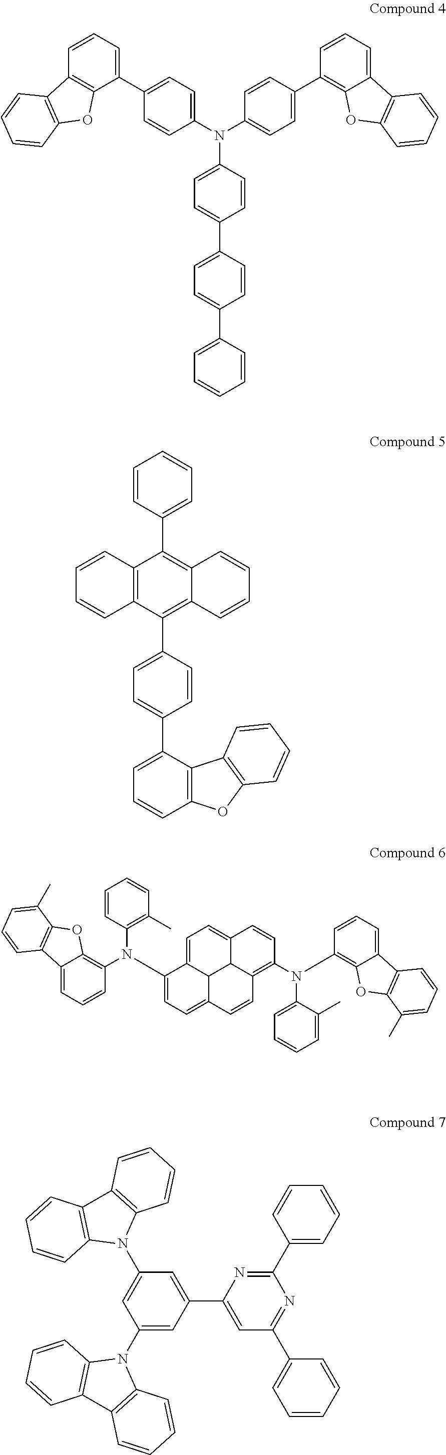 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2c3ccccc3c(-c3ccc(-c4cccc5oc6ccccc6c45)cc3)c3ccccc23)cc1 ; Cc1ccccc1N(C1=CC=C2C=CC3=C(N(c4ccccc4C)c4cccc5c4oc4c(C)cccc45)C=CC4=CC=C1C2C43)c1cccc2c1oc1c(C)cccc12 ; c1ccc(-c2cc(-c3cc(-n4c5ccccc5c5ccccc54)cc(-n4c5ccccc5c5ccccc54)c3)nc(-c3ccccc3)n2)cc1]

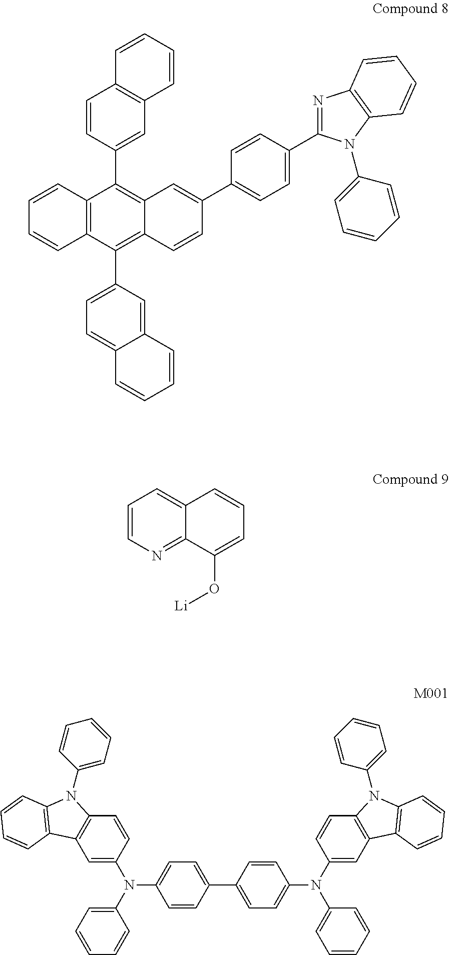

### Application Example 1B

The preparation method was the same as that of Application Example 1A, but the following device structure was adopted.

The structure of an OLED green light device is: ITO (10 nm)/Compound 1:Compound 2 (with the mass ratio of 3:97) (5 nm)/Compound 3 (140 nm)/Compound 4 (5 nm)/CBP:Ir(ppy)₃ (with the mass ratio of 9:1) (40 nm)/Compound 7 (5 nm)/Compound 8:Compound 9 (with the mass ratio of 1:1) (30 nm)/Mg:Ag (with the mass ratio of 10:90, 10 mass % Mg) (10 nm)/M001 (70 nm).

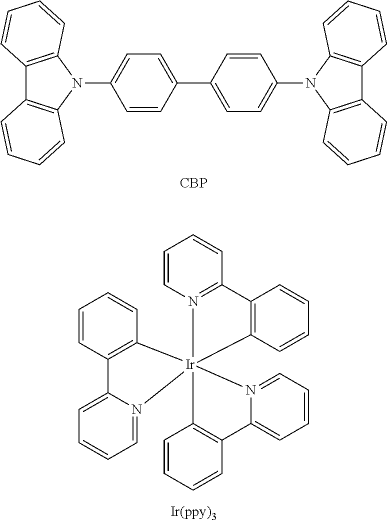

### Application Example 1C

The preparation method was the same as that of Application Example 1A, but the following device structure was adopted.

The structure of an OLED red light device is: ITO (10 nm)/Compound 1:Compound 2 (with the mass ratio of 3:97) (5 nm)/Compound 3 (190 nm)/Compound 4 (5 nm)/CBP:Ir(piq)₂(acac) (with the mass ratio of 96:4) (40 nm)/Compound 7 (5 nm)/Compound 8:Compound 9 (with the mass ratio of 1:1) (30 nm)/Mg:Ag (with the mass ratio of 10:90, 10 mass % Mg) (10 nm)/M001 (70 nm).

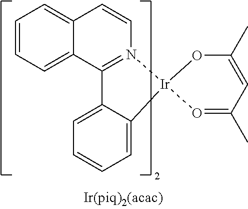

Application Examples 2 (A, B, and C)-72 (A, B, and C) differ from Examples 1 (A, B, and C) in that Compound M001 was replaced with the compounds shown in Table 3.

### Comparative Example 1

This comparative example differs from Application Example 1 in that the organic compound M001 in step (8) was replaced with an equal amount of comparative compound Refl, and other preparation steps were the same.

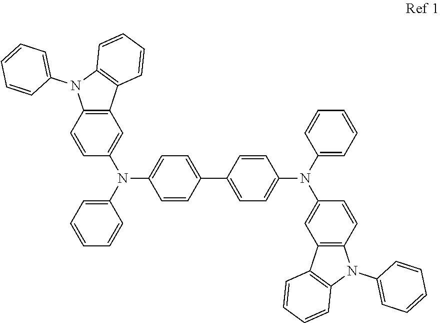 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

### Comparative Example 2

This comparative example differs from Application Example 1A/Application Example 1B/Application Example 1C in that the organic compound M001 in step (8) was replaced with an equal amount of comparative compound Ref2, and other preparation steps were the same.

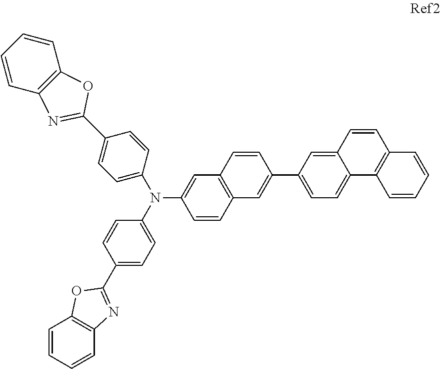 [SMILES: c1ccc2c(c1)ccc1cc(-c3ccc4cc(N(c5ccc(-c6nc7ccccc7o6)cc5)c5ccc(-c6nc7ccccc7o6)cc5)ccc4c3)ccc12]

### Comparative Example 3

This comparative example differs from Application Example 1A/Application Example 1B/Application Example 1C in that the organic compound M001 in step (8) was replaced with an equal amount of comparative compound Ref3, and other preparation steps were the same.

### Comparative Example 4

This comparative example differs from Application Example 1A/Application Example 1B/Application Example 1C in that the organic compound M001 in step (8) was replaced with an equal amount of comparative compound Ref4, and other preparation steps were the same.

### Comparative Example 5

This comparative example differs from Application Example 1A/Application Example 1B/Application Example 1C in that the organic compound M001 in step (8) was replaced with an equal amount of comparative compound Ref5, and other preparation steps were the same.

### Comparative Example 6

This comparative example differs from Application Example 1A/Application Example 1B/Application Example 1C in that the organic compound M001 in step (8) was replaced with an equal amount of comparative compound Ref6, and other preparation steps were the same.

Performance evaluation of organic electroluminescent devices:

The currents of the organic electroluminescent device at different voltages were tested by the Keithley 2365A digital nanovoltmeter, and then the currents were divided by the luminescence area to obtain the current densities of the organic electroluminescent device at different voltages. The brightness and radiant energy flux densities of the organic electroluminescent device at different voltages were tested by the Konicaminolta CS-2000 spectroradiometer. According to the current densities and brightness of the organic electroluminescent device at different voltages, the working drive voltage and current efficiency (Cd/A) of the organic electroluminescent device at the same current density (10 mA/cm²) were obtained. The lifetime (under the testing condition of 50 mA/cm²) was obtained by measuring the time when the brightness of the organic electroluminescent device reached 95% of its initial brightness. Specific data is shown in Table 2.

TABLE 2
Device performance data list

| | | Blue light | Green light | Red light |
|---|---|---|---|---|
| | | current | current | current |
| | | efficiency | efficiency | efficiency |
| | | (referenced | (referenced | (referenced |
| | | to the | to the | to the |
| | CPL | comparative | comparative | comparative |
| No. | material | device 1) | device 1) | device 1) |
| Application Example | M001 | 107% | 114% | 114% |
| 1A/1B/1C | | | | |
| Application Example | M007 | 107% | 114% | 114% |
| 2A/2B/2C | | | | |
| Application Example | M019 | 107% | 114% | 114% |
| 3A/3B/3C | | | | |
| Application Example | M020 | 107% | 114% | 114% |
| 4A/4B/4C | | | | |
| Application Example | M037 | 105% | 109% | 110% |
| 5A/5B/5C | | | | |
| Application Example | M065 | 107% | 115% | 115% |
| 6A/6B/6C | | | | |
| Application Example | M069 | 107% | 115% | 115% |
| 7A/7B/7C | | | | |
| Application Example | M075 | 105% | 109% | 109% |
| 8A/8B/8C | | | | |
| Application Example | M091 | 108% | 116% | 116% |
| 9A/9B/9C | | | | |
| Application Example | M092 | 107% | 114% | 114% |
| 10A/10B/10C | | | | |
| Application Example | M119 | 107% | 114% | 115% |
| 11A/11B/11C | | | | |
| Application Example | M122 | 107% | 114% | 114% |
| 12A/12B/12C | | | | |
| Application Example | M136 | 106% | 112% | 112% |
| 13A/13B/13C | | | | |
| Application Example | M139 | 108% | 116% | 116% |
| 14A/14B/14C | | | | |
| Application Example | M145 | 107% | 115% | 115% |
| 15A/15B/15C | | | | |
| Application Example | M165 | 108% | 116% | 116% |
| 16A/16B/16C | | | | |
| Application Example | M167 | 107% | 113% | 114% |
| 17A/17B/17C | | | | |
| Application Example | M171 | 107% | 114% | 114% |
| 18A/18B/18C | | | | |
| Application Example | M181 | 107% | 113% | 113% |
| 19A/19B/19C | | | | |
| Application Example | M183 | 106% | 112% | 112% |
| 20A/20B/20C | | | | |
| Application Example | M185 | 107% | 114% | 114% |
| 21A/21B/21C | | | | |
| Application Example | M188 | 107% | 114% | 114% |
| 22A/22B/22C | | | | |
| Application Example | M200 | 107% | 114% | 115% |
| 23A/23B/23C | | | | |
| Application Example | M206 | 107% | 114% | 114% |
| 24A/24B/24C | | | | |
| Application Example | M236 | 107% | 114% | 114% |
| 25A/25B/25C | | | | |
| Application Example | M240 | 107% | 115% | 115% |
| 26A/26B/26C | | | | |
| Application Example | M246 | 106% | 111% | 112% |
| 27A/27B/27C | | | | |
| Application Example | M262 | 107% | 114% | 114% |
| 28A/28B/28C | | | | |
| Application Example | M293 | 106% | 112% | 112% |
| 29A/29B/29C | | | | |
| Application Example | M301 | 106% | 113% | 114% |
| 30A/30B/30C | | | | |
| Application Example | M313 | 107% | 114% | 114% |
| 31A/31B/31C | | | | |
| Application Example | M321 | 107% | 114% | 114% |
| 32A/32B/32C | | | | |
| Application Example | M331 | 107% | 115% | 115% |
| 33A/33B/33C | | | | |
| Application Example | M341 | 107% | 115% | 115% |
| 34A/34B/34C | | | | |
| Application Example | M365 | 107% | 114% | 114% |
| 35A/35B/35C | | | | |
| Application Example | M367 | 107% | 113% | 114% |
| 36A/36B/36C | | | | |
| Comparative | Ref1 | 100% | 100% | 100% |
| Example 1A/1B/1C | | | | |
| Comparative | Ref2 | 107% | 114% | 114% |
| Example 2A/2B/2C | | | | |
| Comparative | Ref3 | 107% | 114% | 115% |
| Example 3A/3B/3C | | | | |
| Comparative | Ref4 | 107% | 113% | 114% |
| Example 4A/4B/4C | | | | |
| Comparative | Ref5 | 106% | 112% | 112% |
| Example 5A/5B/5C | | | | |
| Comparative | Ref6 | 106% | 112% | 113% |
| Example 6A/6B/6C | | | | |

Note:
In the table, the blue light current efficiency corresponding to Application Example 1A/1B/1C refers to the current efficiency of Application Example 1A, the green light current efficiency refers to the current efficiency of Application Example 1B, the red light current efficiency refers to the current efficiency of Application Example 1C, and so on.

As can be seen from the above Examples and Comparative Example 1, compared with the commercial conventional capping layer material Compound Refl, the compounds provided by the present disclosure achieve higher luminescence efficiency when applied to blue light, green light and red light devices, where the blue light luminescence efficiency is improved by 5%-8%, the green light efficiency is improved by 9%-16%, and the red light efficiency is improved by 9%-16%. It shows that the compounds of the present disclosure, when used as the capping layer material, have excellent light extraction capability and can effectively improve the luminescence efficiency of the organic electroluminescent device.

### Example 9

To further verify the long-term evaporation thermal stability of the phenanthrene compound of the present disclosure, the material M001 was placed in an ampoule tube, evacuated to 1×10⁻⁴ Pa and sintered at a high temperature. After the material was heated at 400° C. for 200 h, the sealed ampoule tube was broken to take out the material M001. The purity before and after heating was tested by HPLC.

The material Ref2 was placed in an ampoule tube, evacuated to 1×104 Pa and sintered at a high temperature. After the material was heated at 400° C. for 200 h, the sealed ampoule tube was broken to take out the material Ref2. The purity before and after heating was tested by HPLC.

### Example 10

With reference to Example 9, M001 was replaced with M119 and Ref2 was replaced with Ref3.

### Example 11

With reference to Example 9, M001 was replaced with M167 and Ref2 was replaced with Ref4.

### Example 12

With reference to Example 9, M001 was replaced with M183 and Ref2 was replaced with Ref5.

### Example 13

With reference to Example 9, M001 was replaced with M293 and Ref2 was replaced with Ref6.

TABLE 3
Material purity data table before and after heating

| | Purity | Purity |
|---|---|---|
| Material | before | after |
| name | heating | heating |
| M001 | 99.9% | 94.9% |
| Ref2 | 99.9% | 75.4% |
| M119 | 99.9% | 92.7% |
| Ref3 | 99.9% | 68.1% |
| M167 | 99.9% | 96.0% |
| Ref4 | 99.9% | 82.3% |
| M183 | 99.9% | 95.6% |
| Ref5 | 99.9% | 78.8% |
| M293 | 99.9% | 98.8% |
| Ref6 | 99.9% | 90.4% |

As can be seen from the data in Table 3, on the premise of the same purity before heating, compared with the material Ref2/Ref3/Ref4/Ref5/Ref6, the purity of the compound M001/M119/M167/M183/M293 of the present disclosure after heating is significantly higher than the purity of the material Ref2/Ref3/Ref4/Ref5/Ref6 after heating at the same temperature and for the same heating time. The compounds of the present disclosure make no significant difference in the material refractive index and device performance by merely exchanging the connection modes of the phenanthrene group and the naphthalene group, but significantly improve and promote the long-term evaporation thermal stability of the material. In a case where the current mainstream processing technology of the OLED is long-term evaporation in a vacuum environment, the present disclosure has important practical significance and mass production value.

The applicant has stated that although the phenanthrene organic compound of the present disclosure and use thereof are described through the preceding embodiments, the present disclosure is not limited to the preceding embodiments, which means that the implementation of the present disclosure does not necessarily depend on the preceding embodiments. It should be apparent to those skilled in the art that any improvements made to the present disclosure, equivalent replacements of raw materials of the product, additions of adjuvant ingredients, selections of specific methods, etc. in the present disclosure all fall within the protection scope and the disclosure scope of the present disclosure.

What is claimed is:

1. A phenanthrene organic compound, having the structure represented by Formula I: 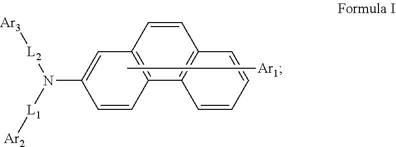
wherein Ar₁ is selected from substituted or unsubstituted C6-C20 aryl, substituted or unsubstituted C10-C20 fused-ring aryl, substituted or unsubstituted C2-C20 heteroaryl, or substituted or unsubstituted C4-C20 fused-ring heteroaryl;
L₁ and L₂ are independently selected from a single bond, substituted or unsubstituted C6-C20 arylene, substituted or unsubstituted C2-C20 heteroarylene, or substituted or unsubstituted C4-C20 fused-ring heteroarylene; and
Ar₂ and Ar₃ are independently selected from a substituted or unsubstituted C10-C20 fused-ring aromatic group or substituted or unsubstituted C4-C20 nitrogen-containing fused-ring heteroaryl having an electron-withdrawing property, and at least one of Ar₂ and Ar₃ is selected from substituted or unsubstituted C4-C20 nitrogen-containing fused-ring heteroaryl having an electron-withdrawing property.

2. The phenanthrene organic compound according to claim 1, wherein Ar₁ is selected from a substituted or unsubstituted C10-C20 fused aromatic ring group;
L₁ and L₂ are independently selected from a single bond or substituted or unsubstituted C6-C20 arylene; and
Ar₂ and Ar₃ are independently selected from a substituted or unsubstituted C10-C20 fused-ring aromatic group or substituted or unsubstituted C2-C20 nitrogen-containing fused-ring heteroaryl having an electron-withdrawing property, and at least one of Ar₂ and Ar₃ is selected from substituted or unsubstituted C2-C20 nitrogen-containing fused-ring heteroaryl having an electron-withdrawing property.

3. The phenanthrene organic compound according to claim 1, wherein the substituent in the substituent group is selected from deuterium, tritium, halogen, cyano or adamantyl.

4. The phenanthrene organic compound according to claim 1, wherein L₁ and L₂ are independently selected from a single bond, phenylene, biphenylene, naphthylene, anthrylene, phenanthrylene, pyrenylene, fluoranthenylene, triphenylenylene, pyridylene, pyrimidinylene, triazinylene, furylene, thienylene, quinolylene, isoquinolylene, benzimidazolylene, benzoxazolylene, benzothiazolylene, dibenzofurylene, dibenzothienylene, thiazolylene, oxazolylene, oxadiazolylene, thiadiazolylene, benzofurylene, benzothienylene or phenanthrolinylene.

5. The phenanthrene organic compound according to claim 4, wherein L₁ and L₂ are selected from a single bond or phenylene.

6. The phenanthrene organic compound according to claim 1, wherein Ar₁ is selected from naphthyl, anthryl, phenanthryl, pyrenyl, fluoranthenyl or triphenylene.

7. The phenanthrene organic compound according to claim 1, wherein the C10-C20 fused-ring aromatic group in the definition of Ar₂ and Ar₃ is selected from naphthyl, anthryl, phenanthryl, pyrenyl, fluoranthenyl or triphenylene.

8. The phenanthrene organic compound according to claim 1, wherein the C4-C20 nitrogen-containing fused-ring heteroaryl having an electron-withdrawing property in the definition of Ar₂ and Ar₃ is selected from the structure represented by Formula II, Formula III or Formula IV: 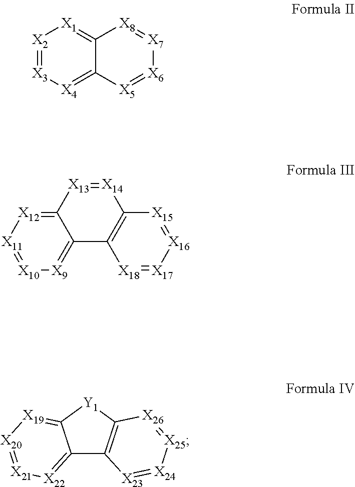
wherein X₁ to X₂₆ are independently selected from CR₁ or N;
at least one of X₁ to X₈ is selected from N;
at least one of X₉ to X₁₈ is selected from N;
at least one of X₁₉ to X₂₆ is selected from N;
Y₁ is selected from O or S;
wherein the linkage site of the group is any linkable position.

9. The phenanthrene organic compound according to claim 8, wherein at least one of Ar₂ and Ar₃ is selected from any one of the following nitrogen-containing fused-ring heteroaryl having an electron-withdrawing property: 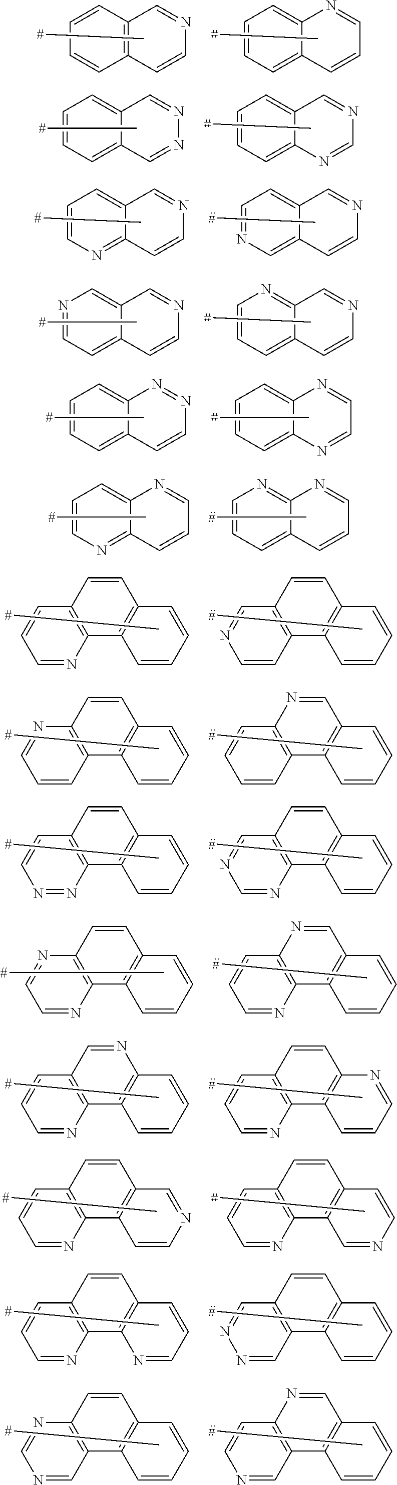 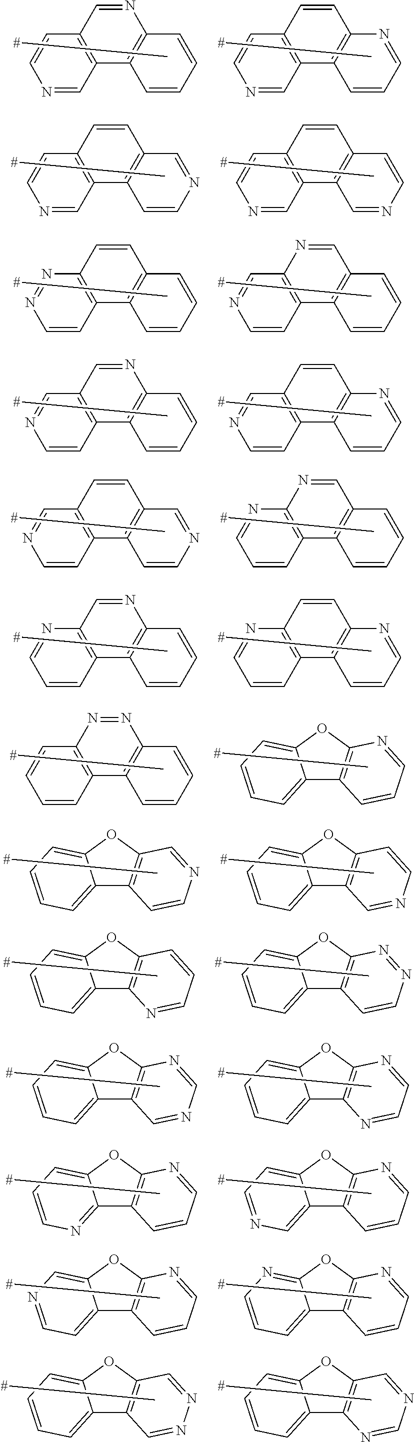 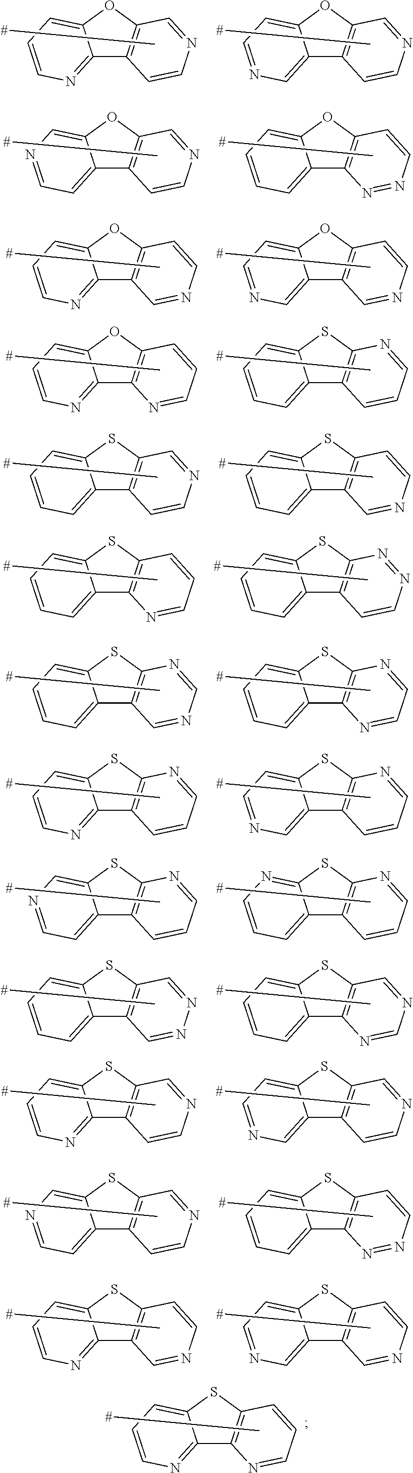
wherein # is a position where groups are joined.

10. The phenanthrene organic compound according to claim 1, wherein at least one of Ar₂ and Ar₃ is selected from the group represented by Formula V: 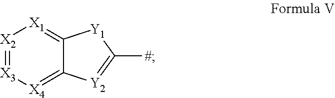
wherein X₁, X₂, X₃, and X₄ are independently selected from CR₁ or N;
Y₁ is selected from O, S, NR₂ or C(R₂)₂, and Y₂ is selected from N or CR₂;
at least one of X₁, X₂, X₃, X₄, Y₁, and Y₂ is N or NR₂; and
R₁ and R₂ are selected from hydrogen, deuterium, C1-C10 alkyl or C6-C20 aryl.

11. The phenanthrene organic compound according to claim 10, wherein at least one of Ar₂ and Ar₃ is selected from any one of the following groups: 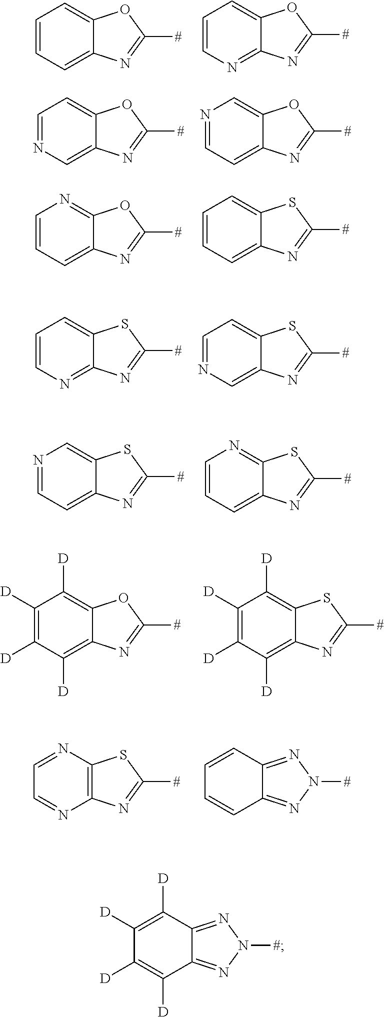
wherein D represents deuterium, and # is a position where groups are joined.

12. The phenanthrene organic compound according to claim 1, wherein at least one of Ar₂ and Ar₃ is selected from the group represented by Formula VI: 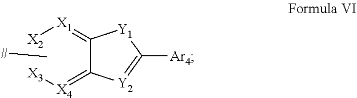
wherein X₁, X₂, X₃, and X₄ are independently selected from CR₁ or N;
Y₁ and Y₂ are independently selected from O, S or NR₂;
at least one of X₁, X₂, X₃, X₄, Y₁, and Y₂ is N or NR₂; and
R₁ and R₂ are independently selected from hydrogen, deuterium or C6-C20 aryl.

13. The phenanthrene organic compound according to claim 12, wherein at least one of Ar₂ and Ar₃ is selected from any one of the following groups: 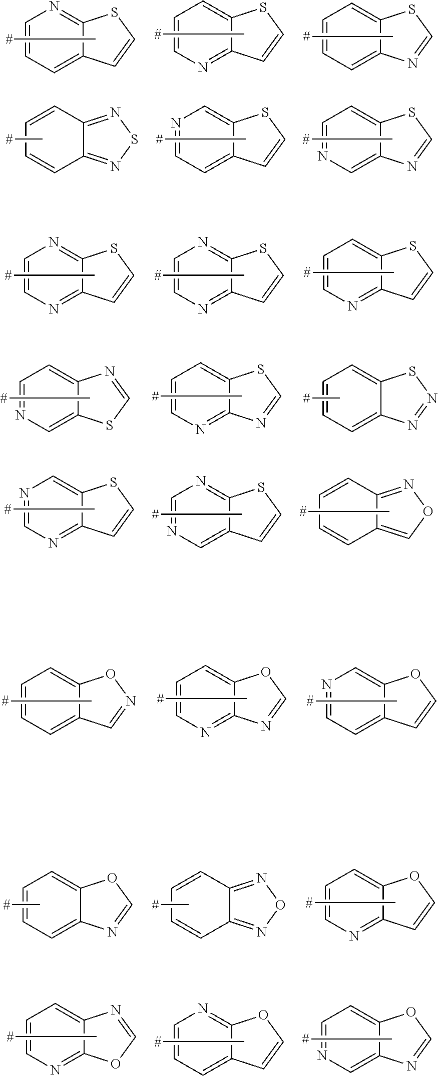 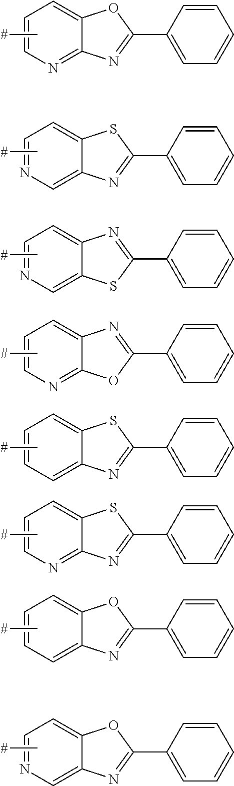 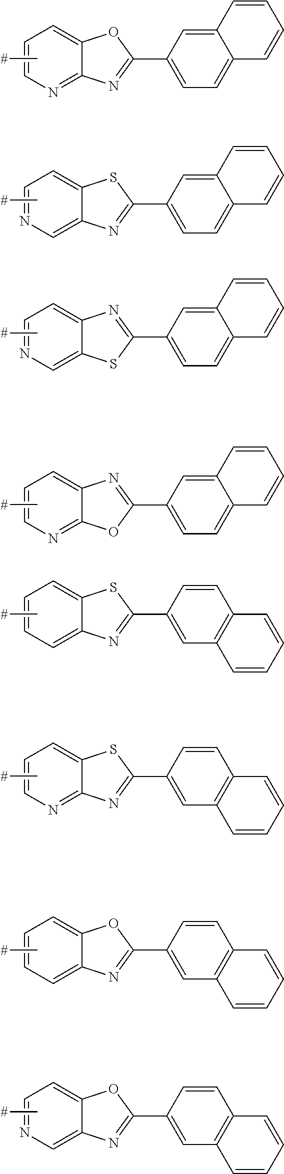 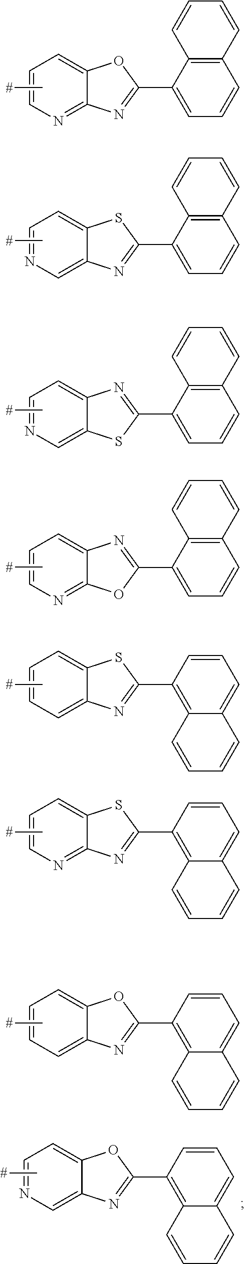
wherein # is a position where groups are joined.

14. The phenanthrene organic compound according to claim 1, wherein the phenanthrene organic compound has the following structure: 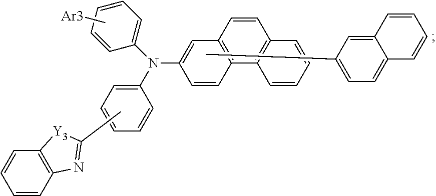
wherein Y₃ is selected from O or S;
Ar₃ is selected from any one of the following groups: 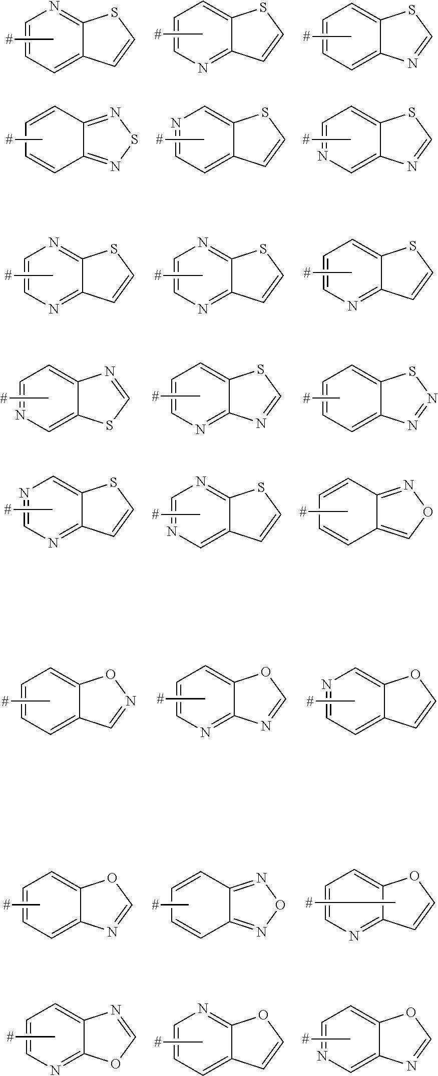 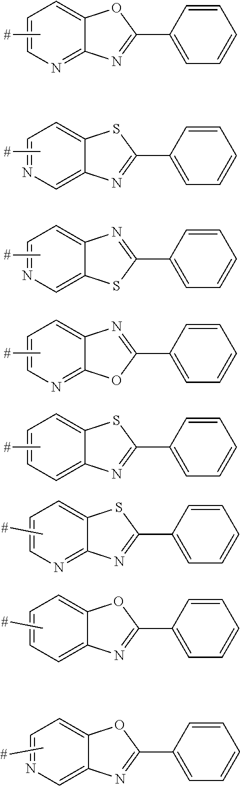 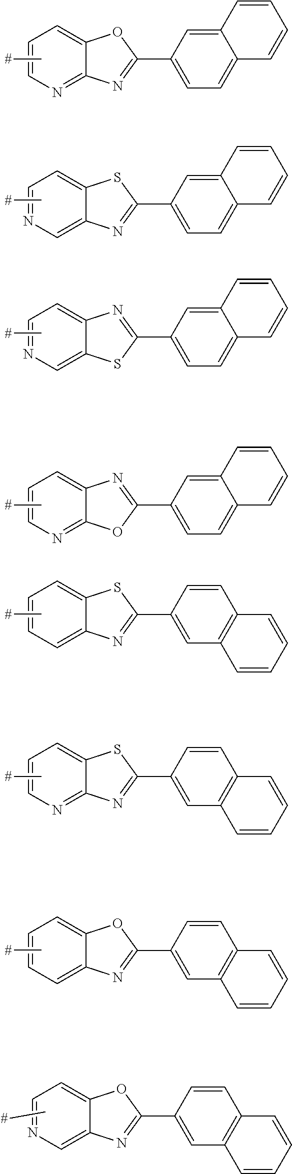 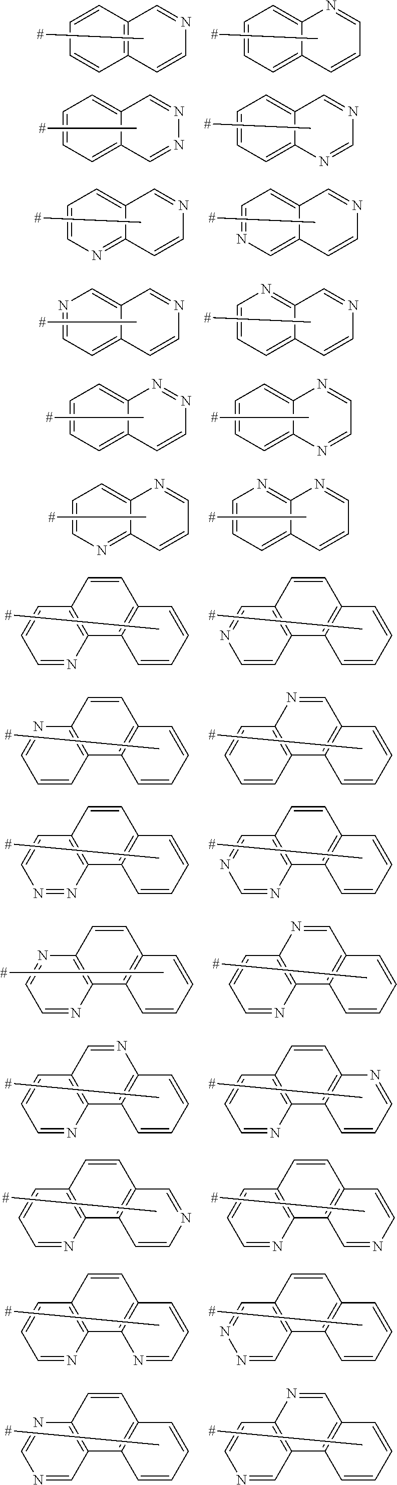 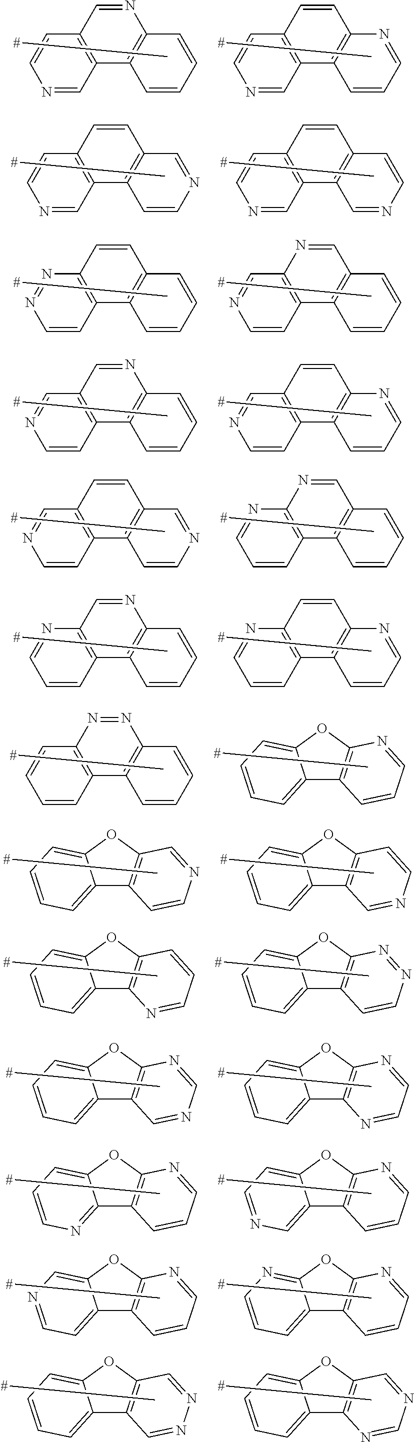 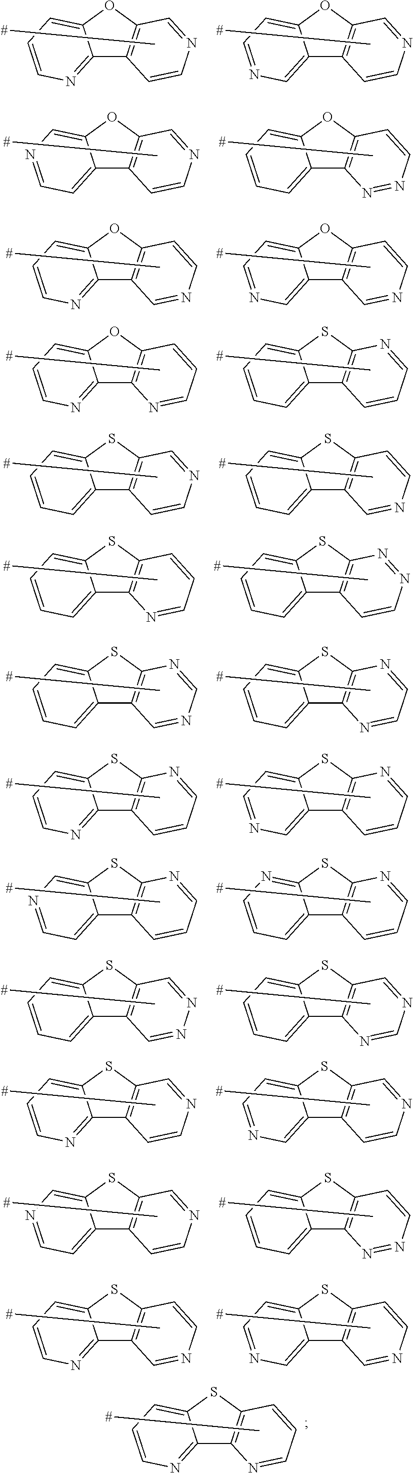
wherein # is a position where groups are joined.

15. The phenanthrene organic compound according to claim 1, wherein the phenanthrene organic compound is any one of the following compounds: 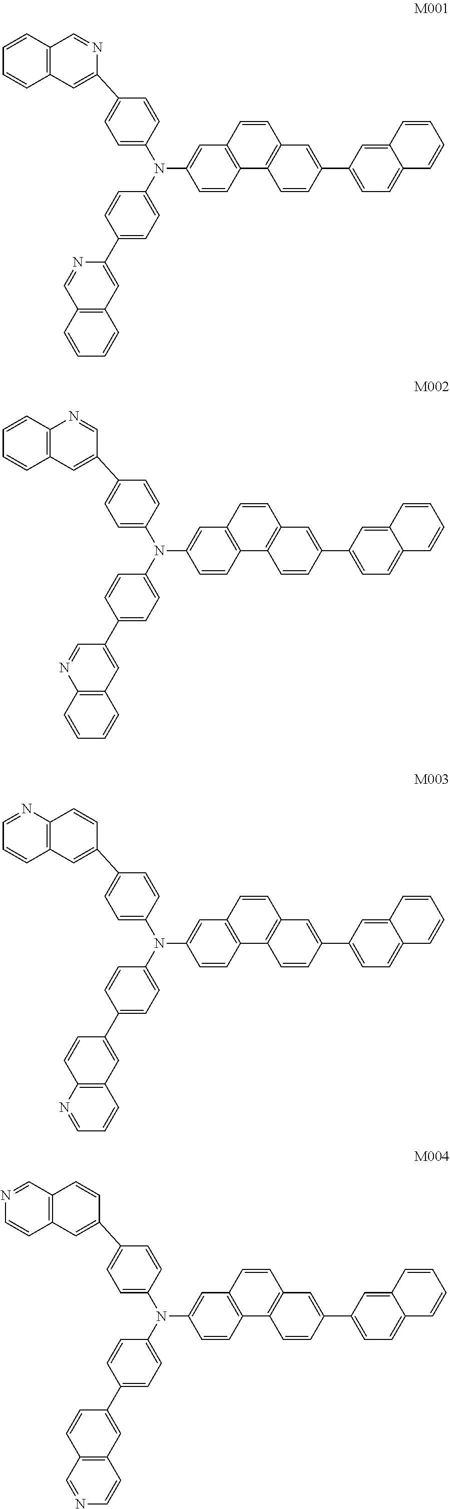 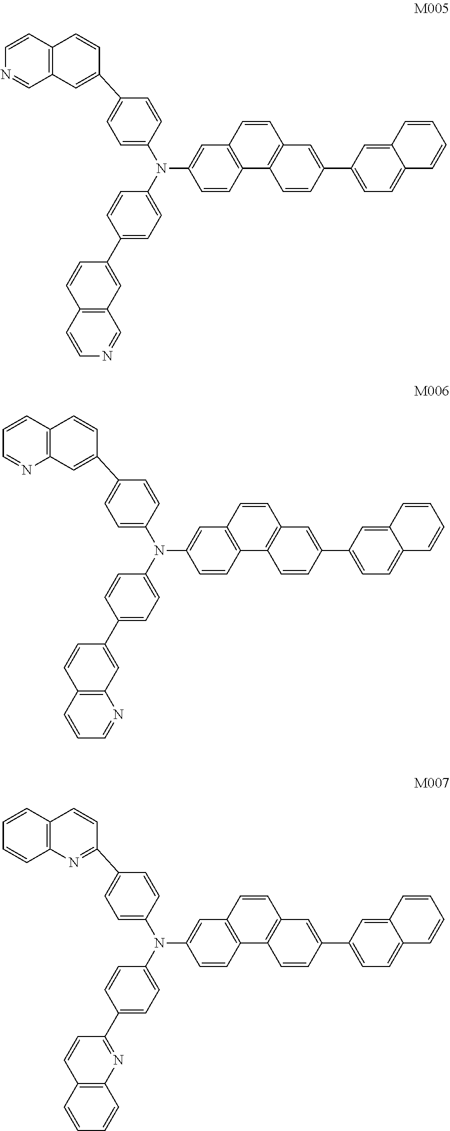 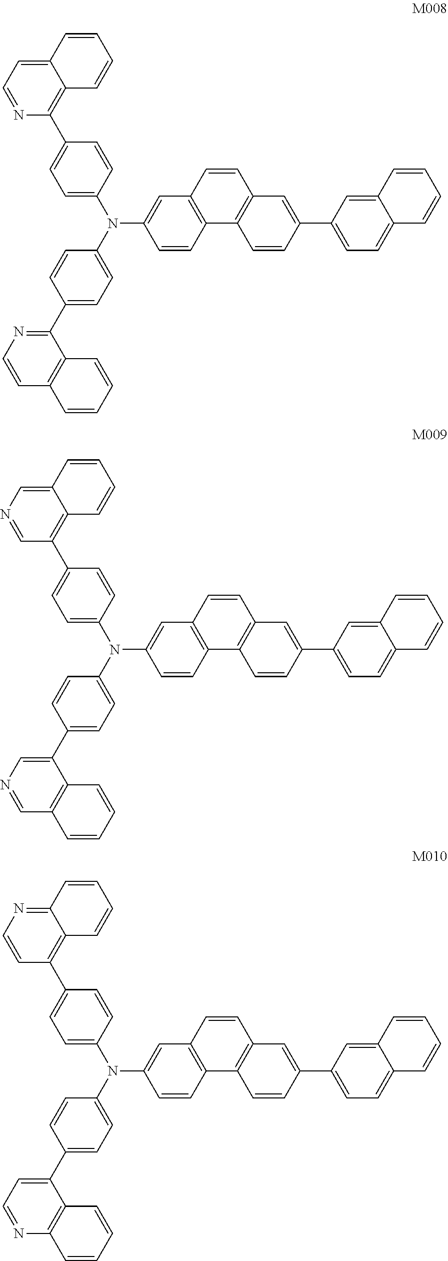 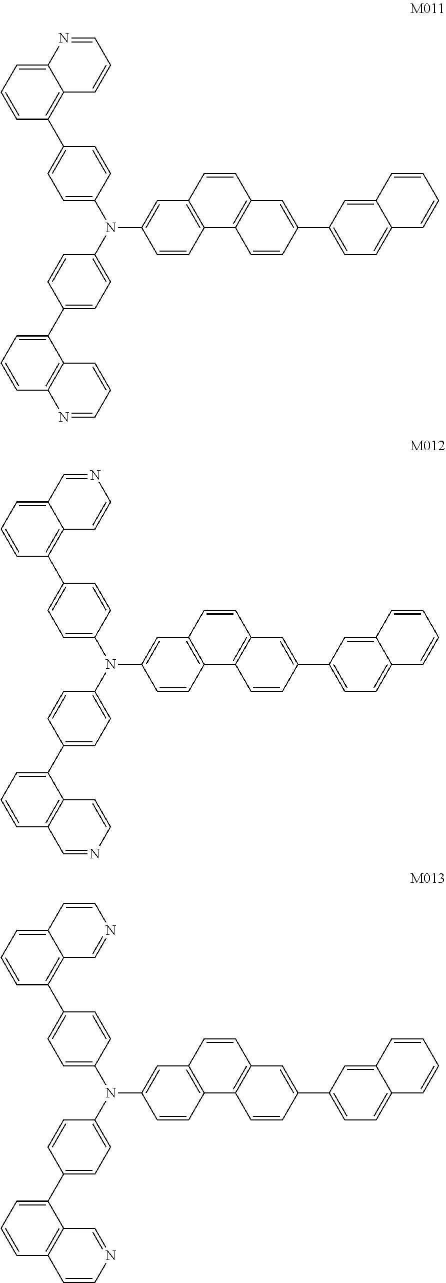 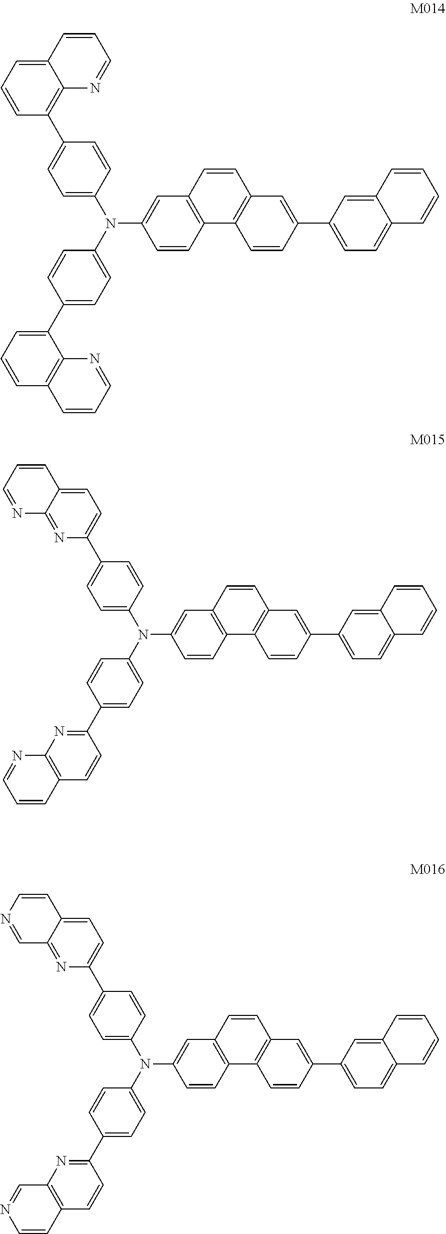 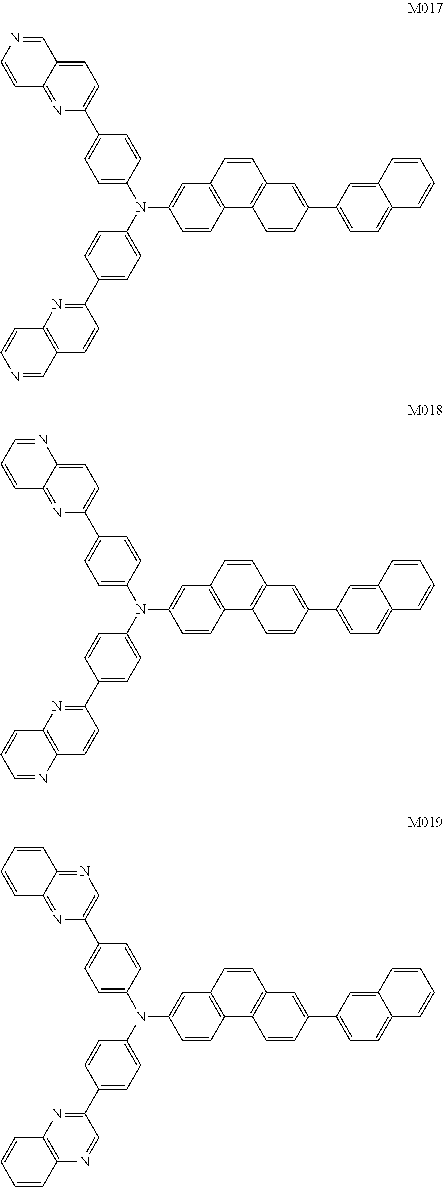 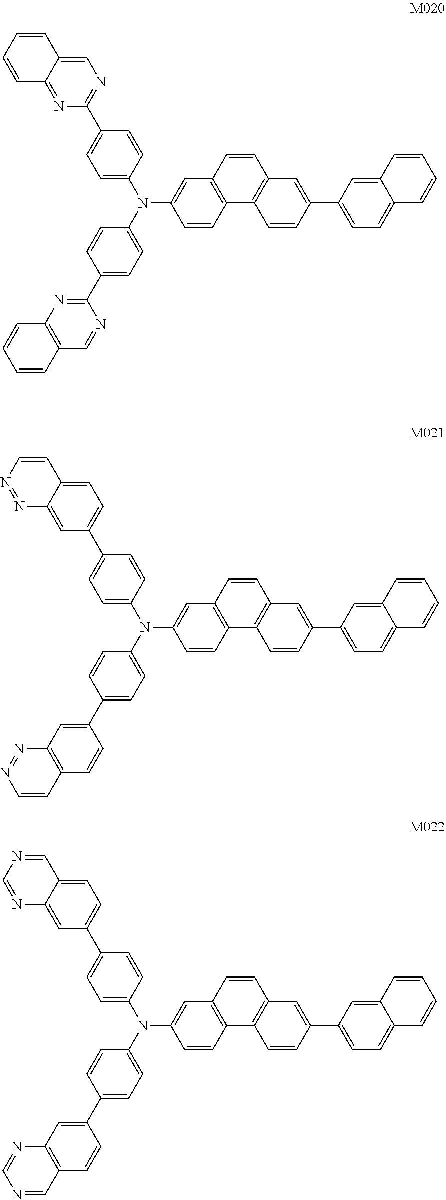 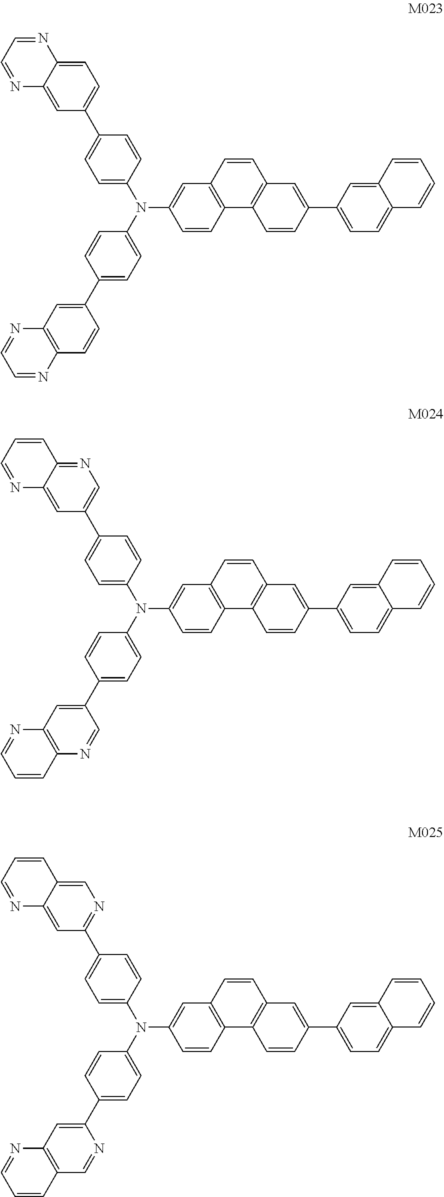 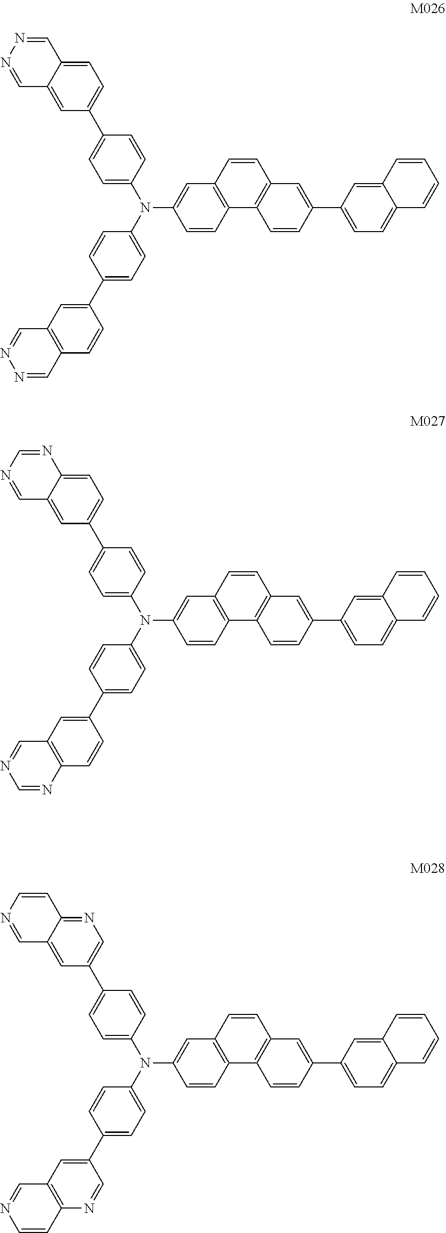 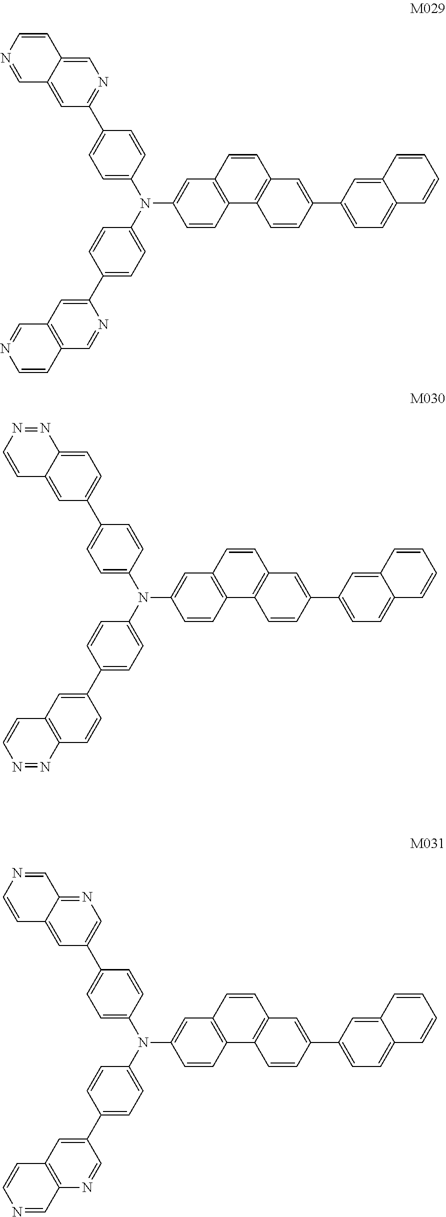 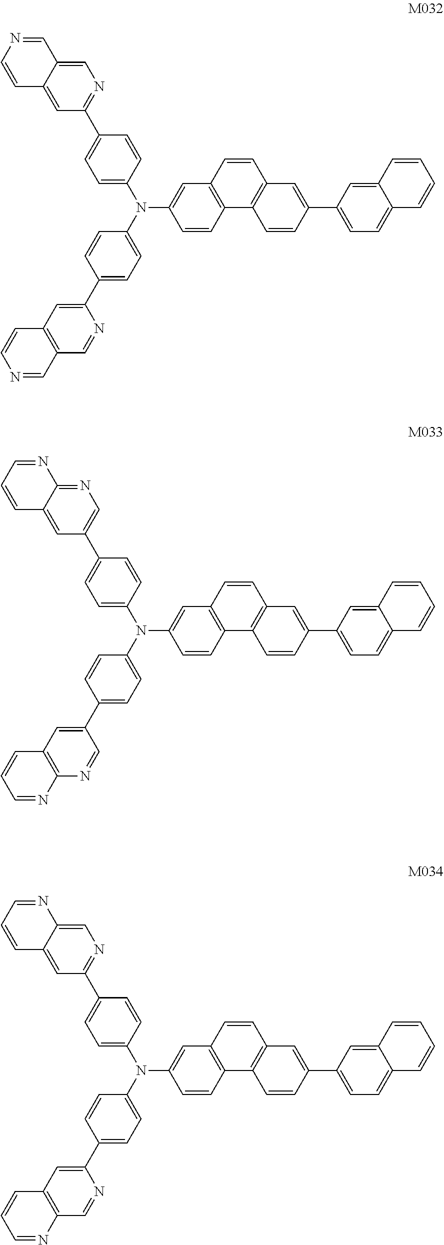 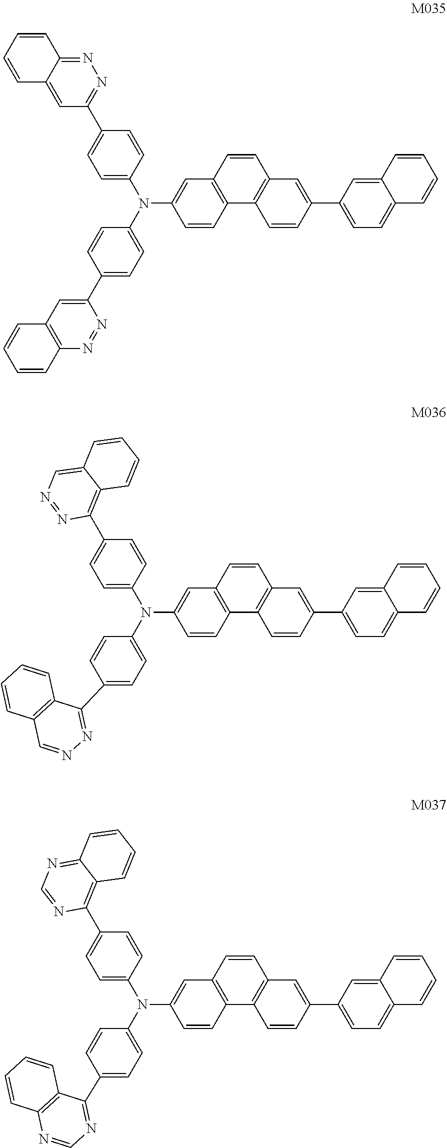 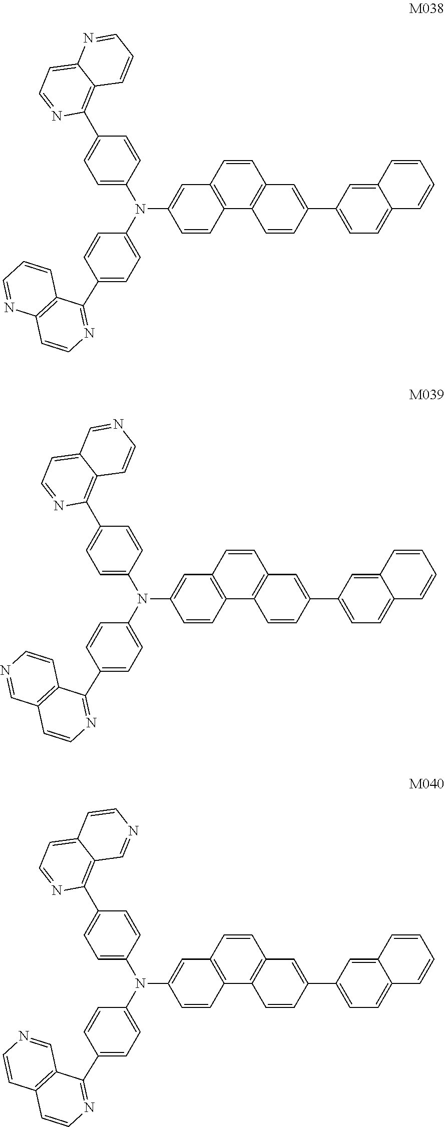 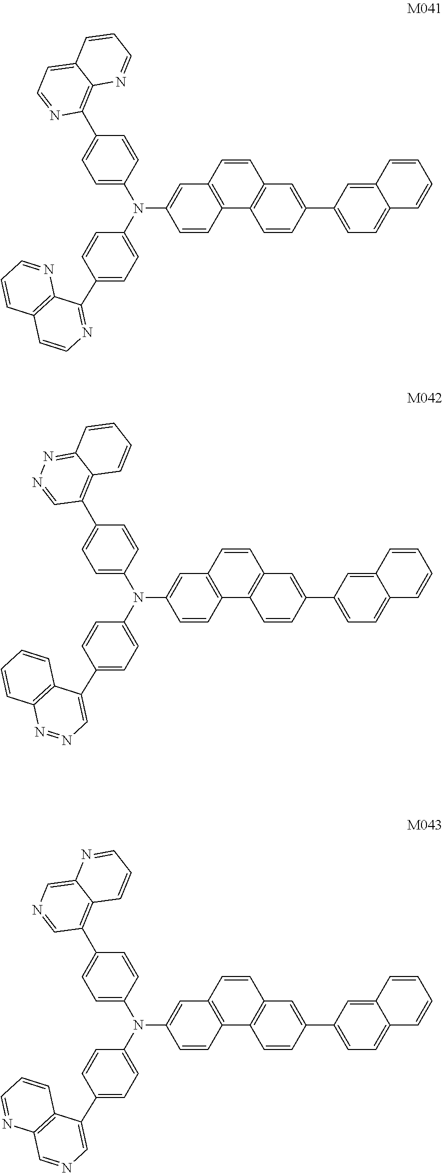 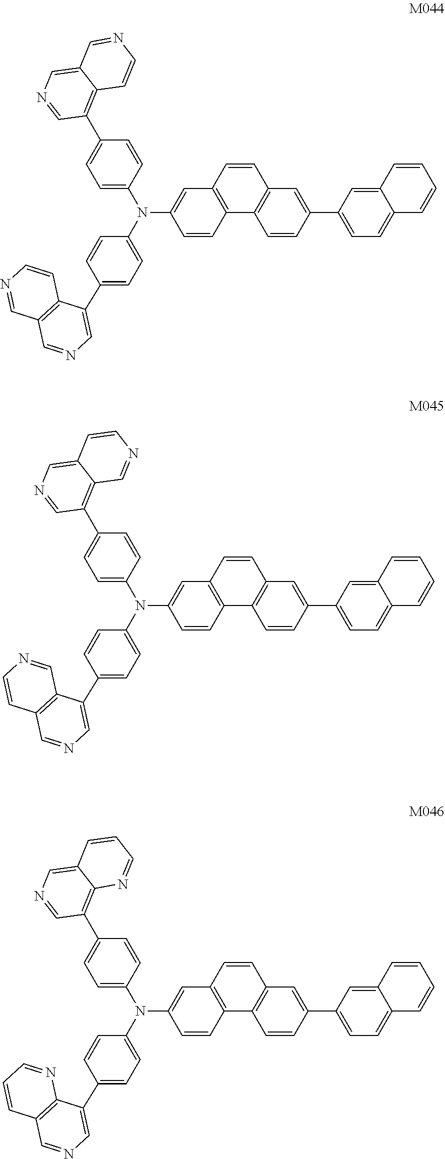 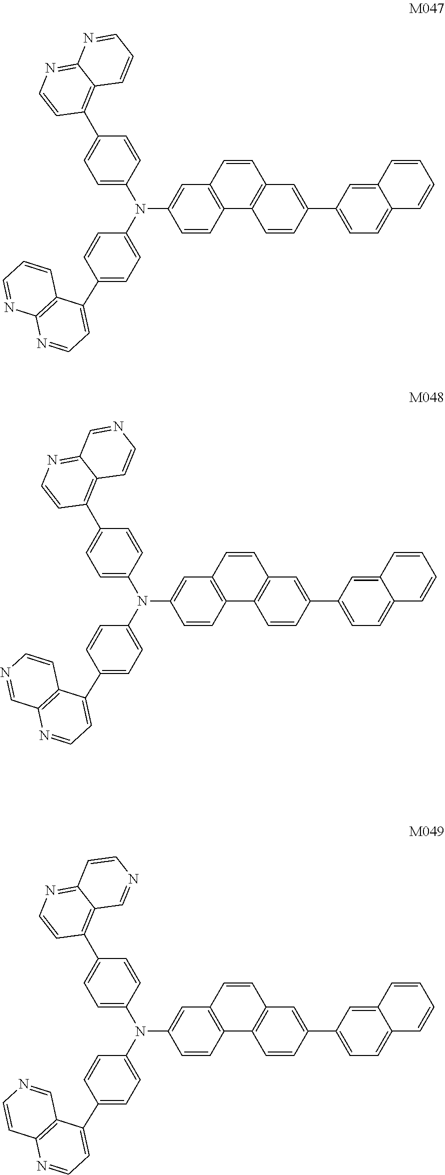 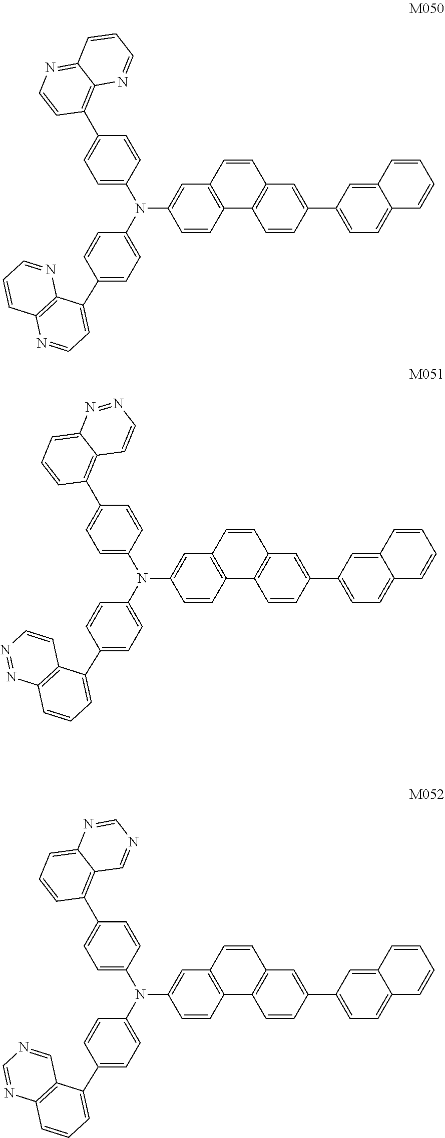 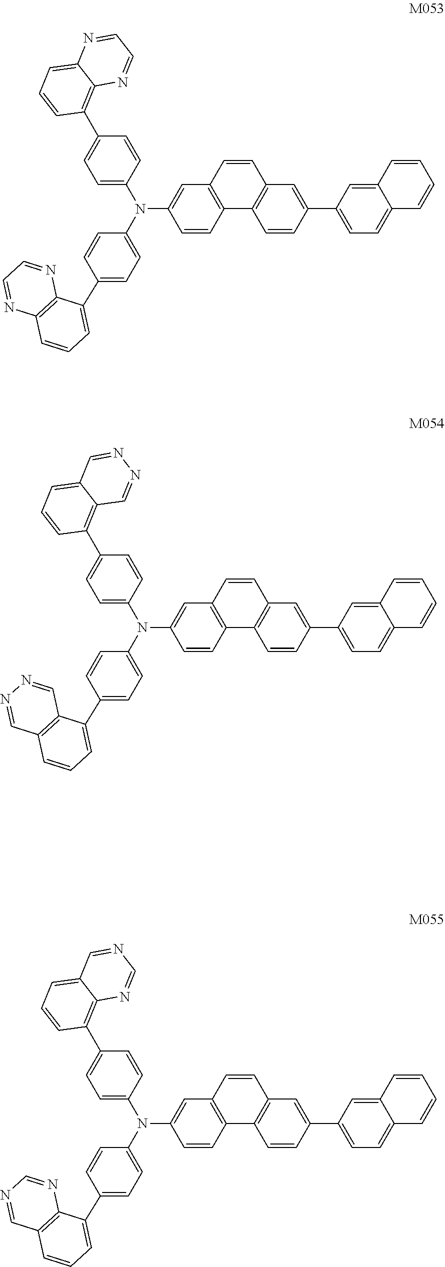 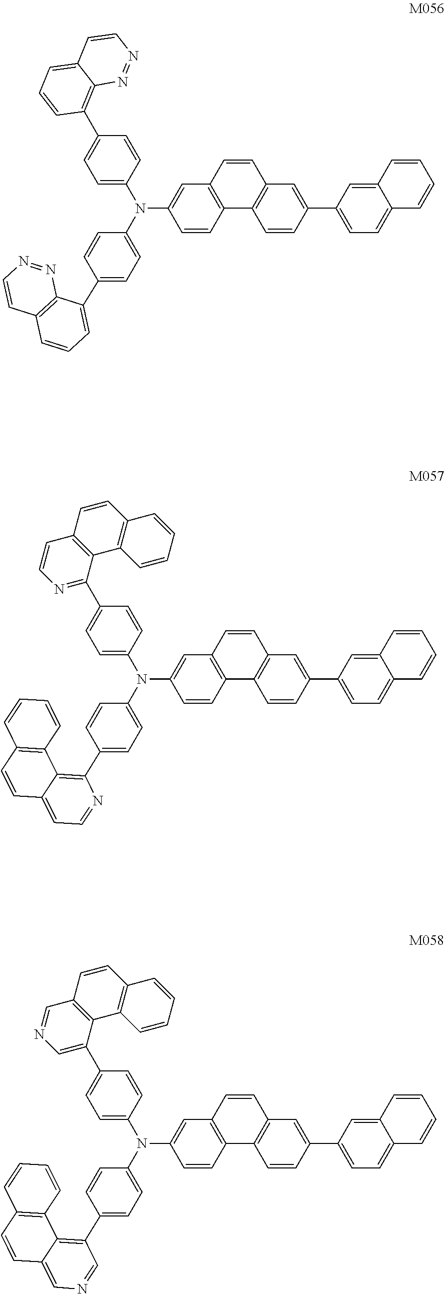 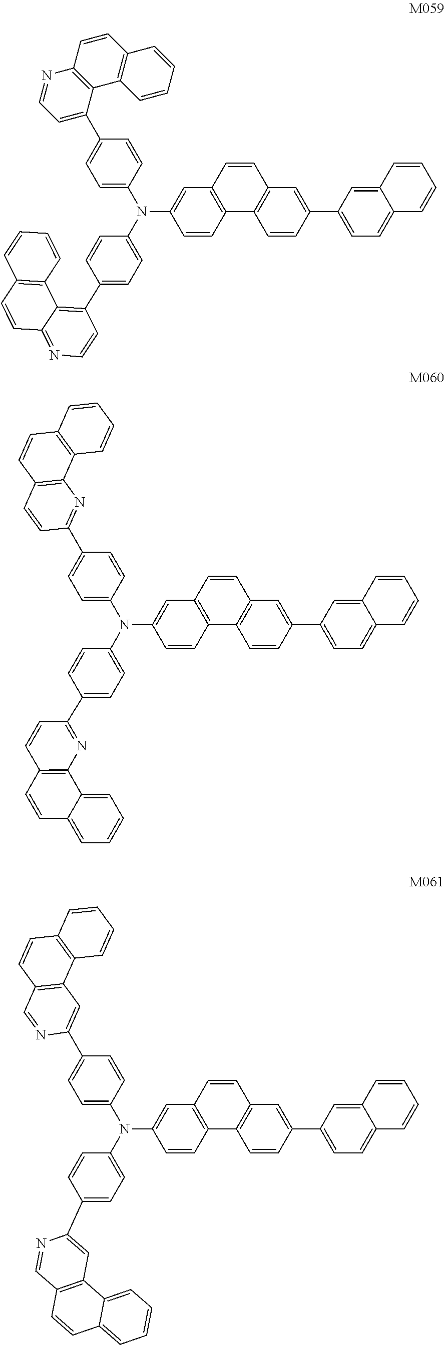 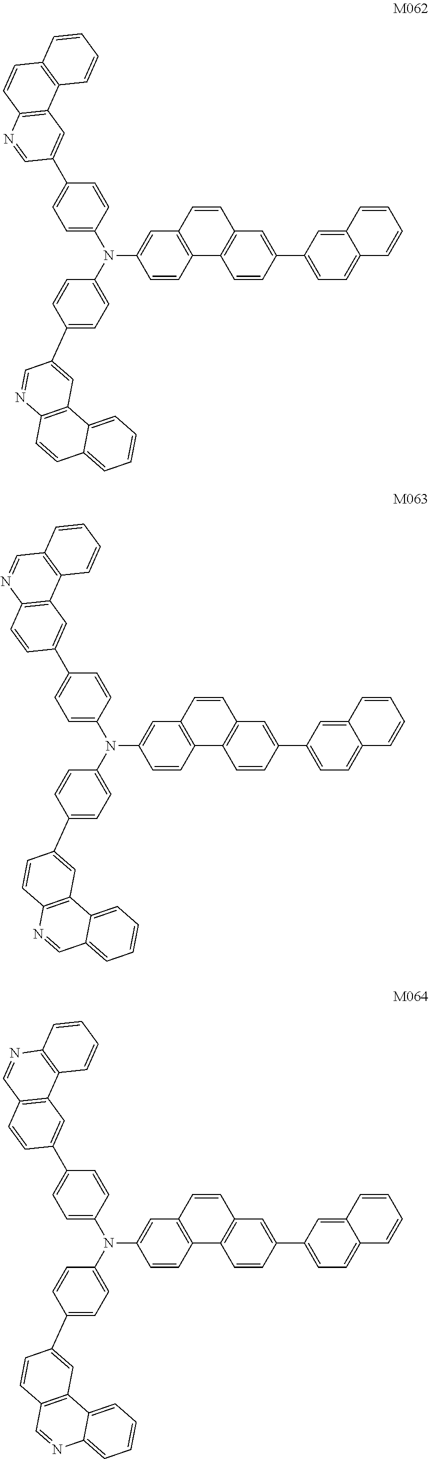 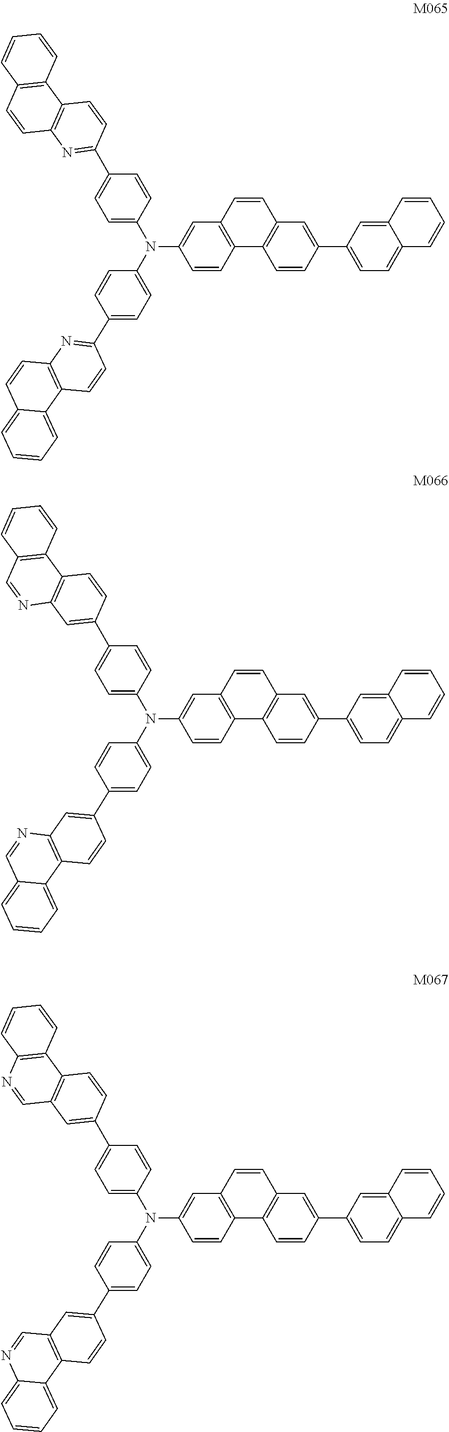 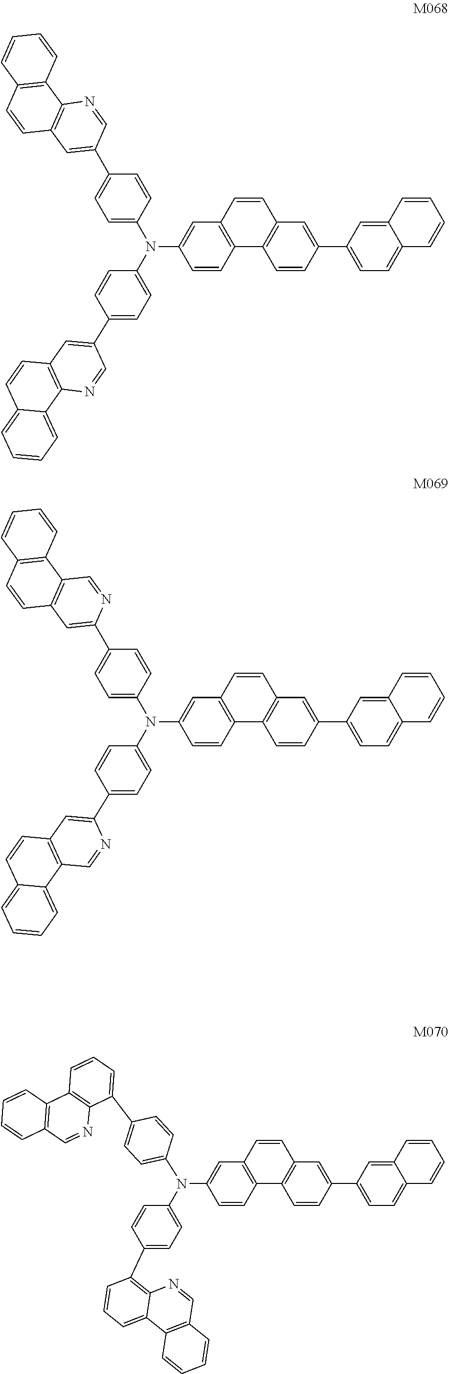 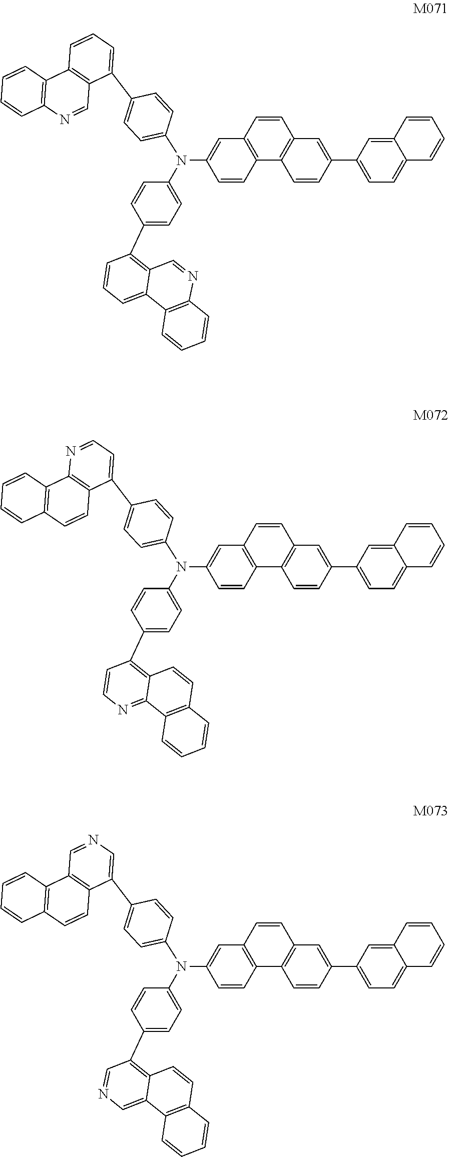 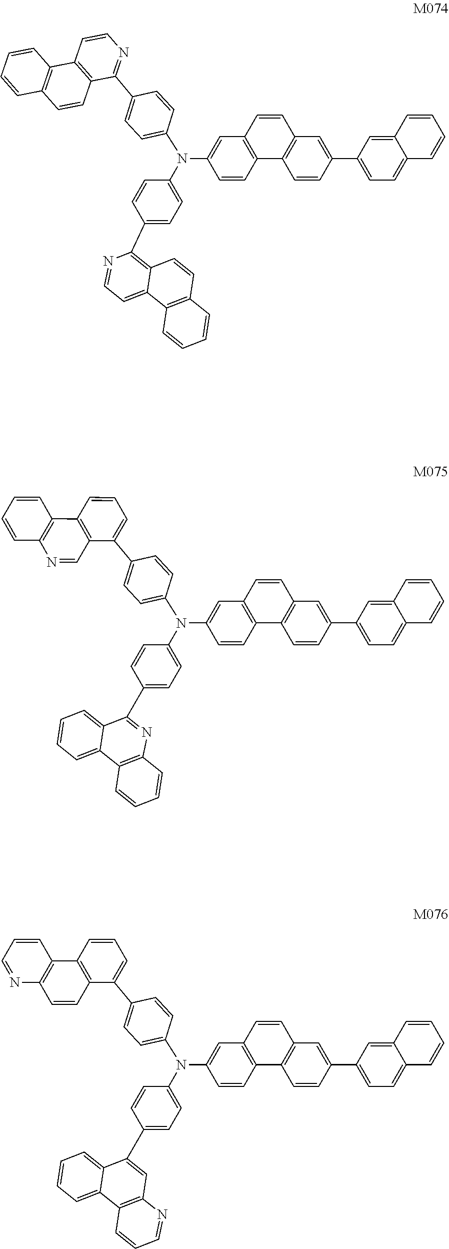 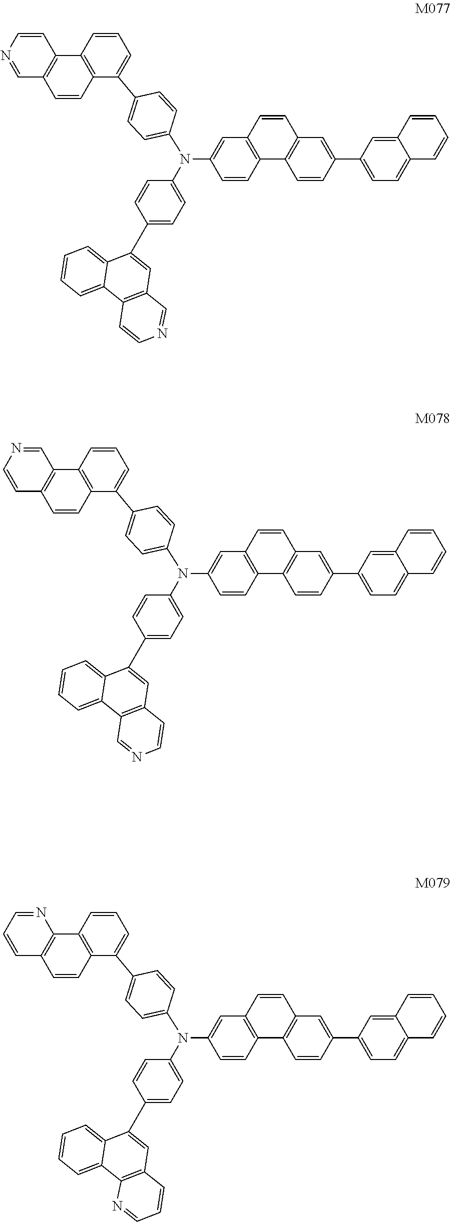 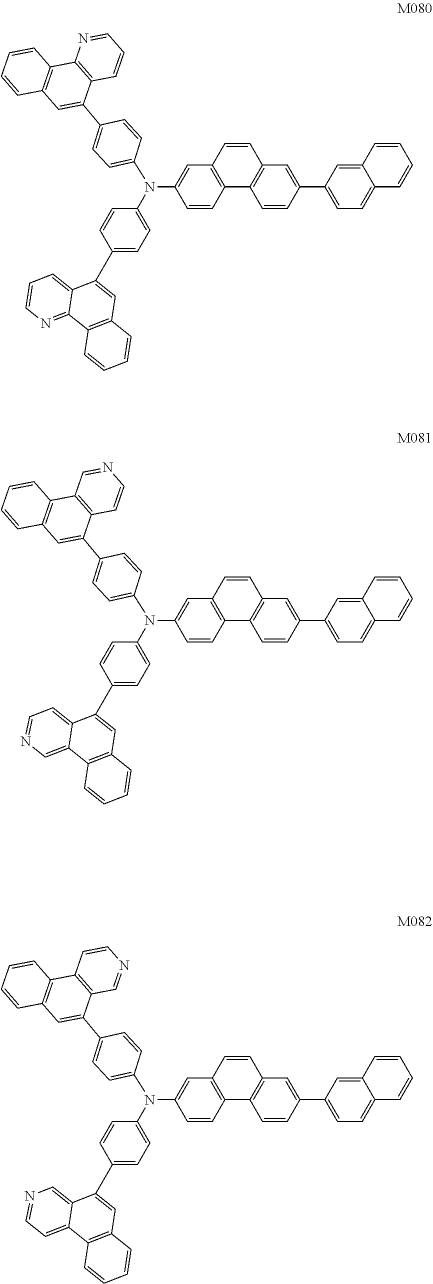 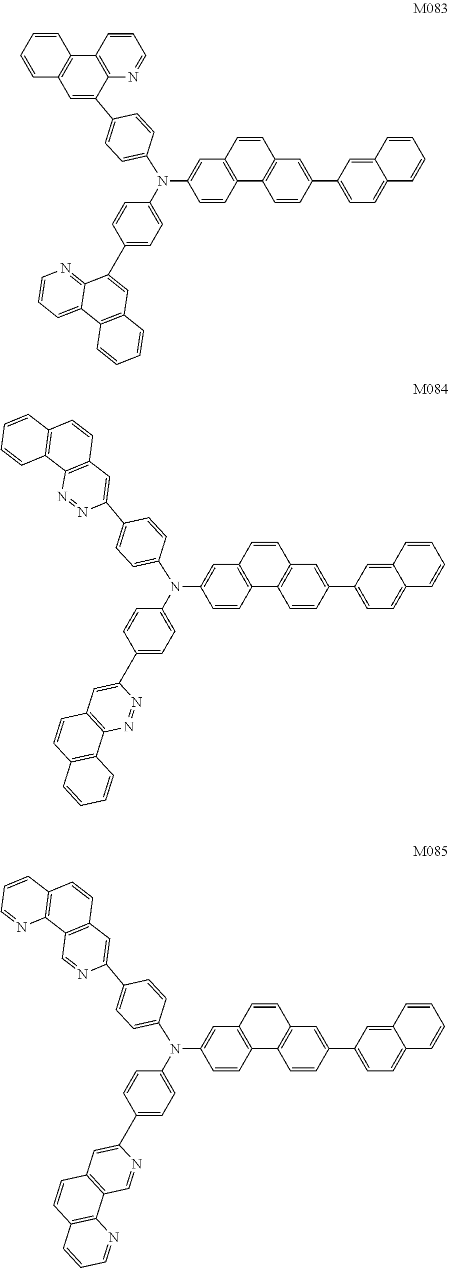 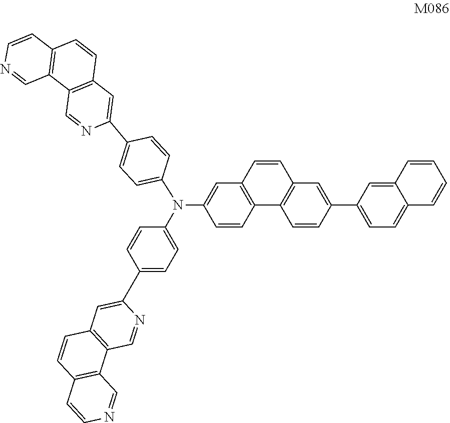 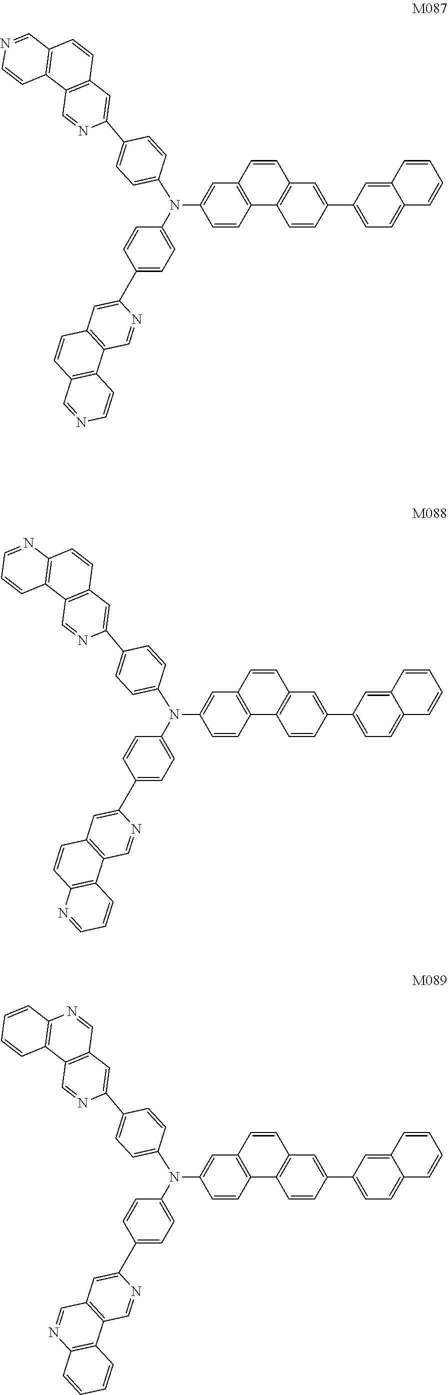 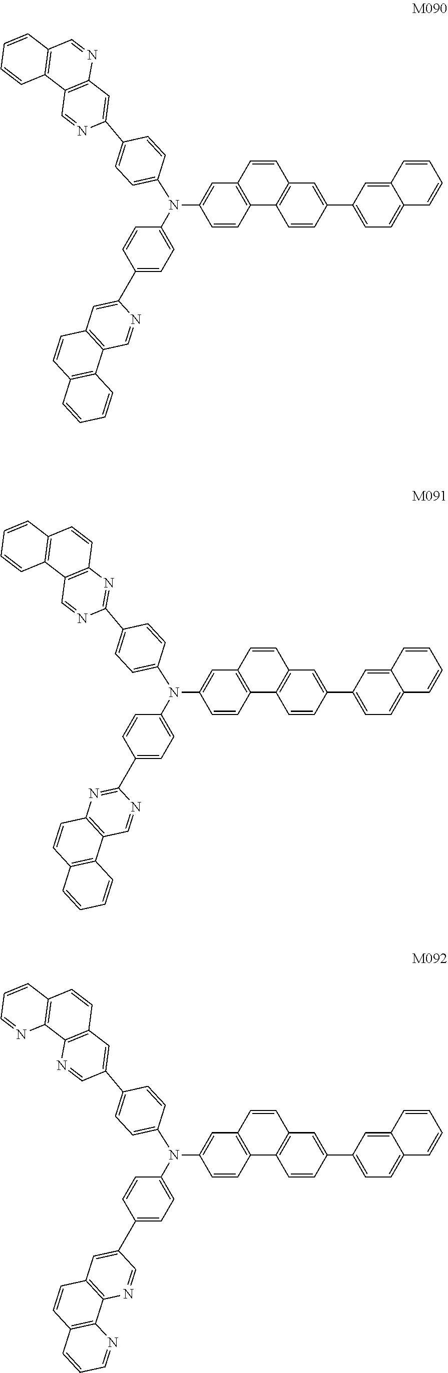 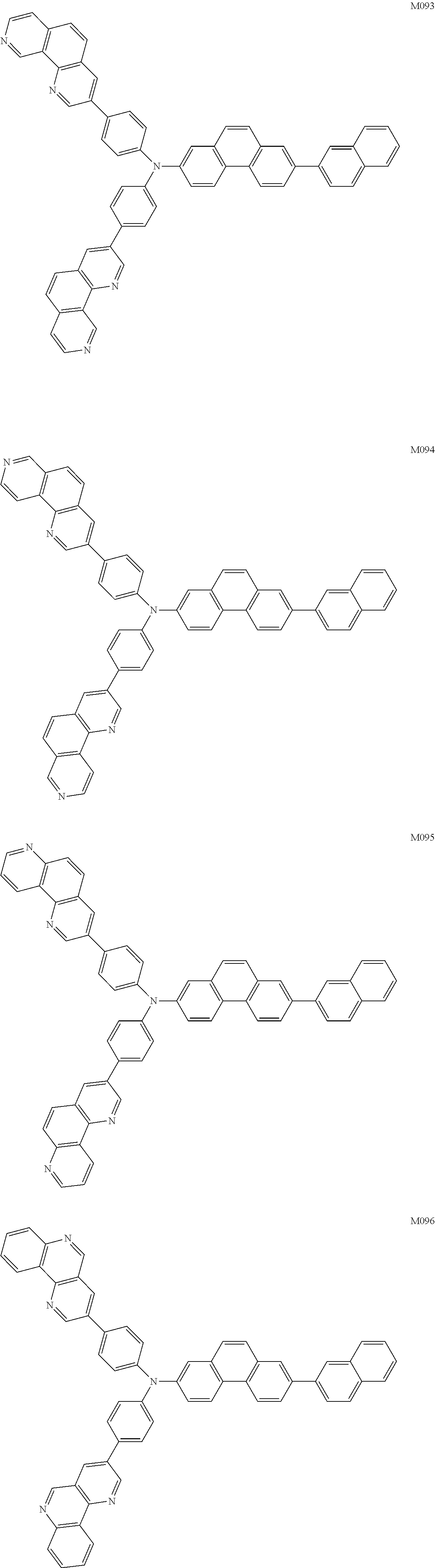 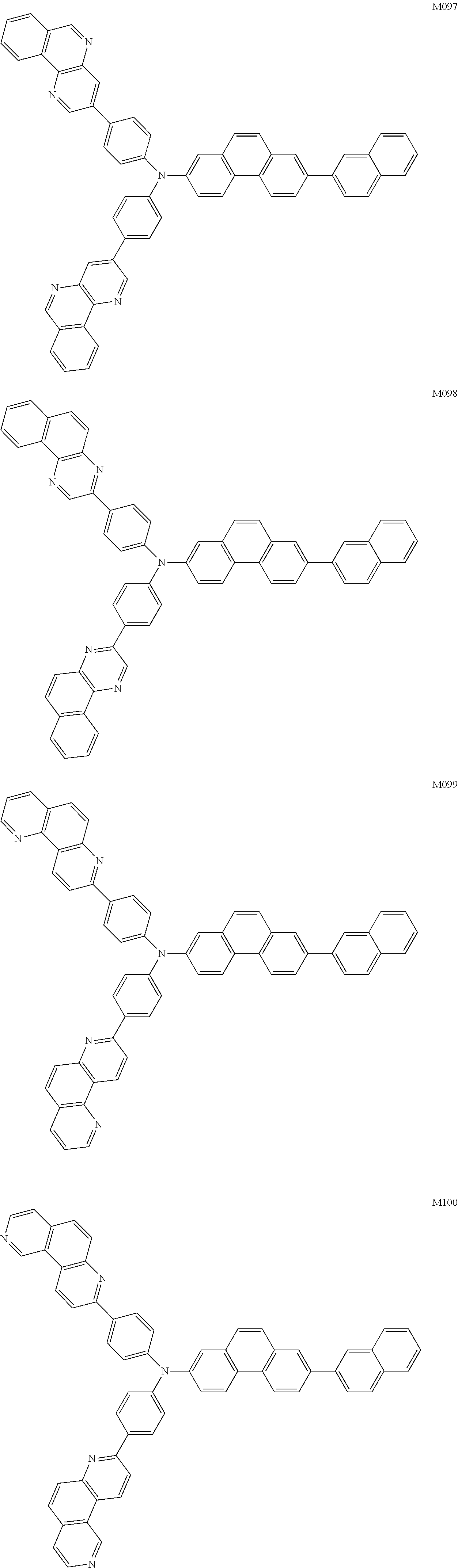 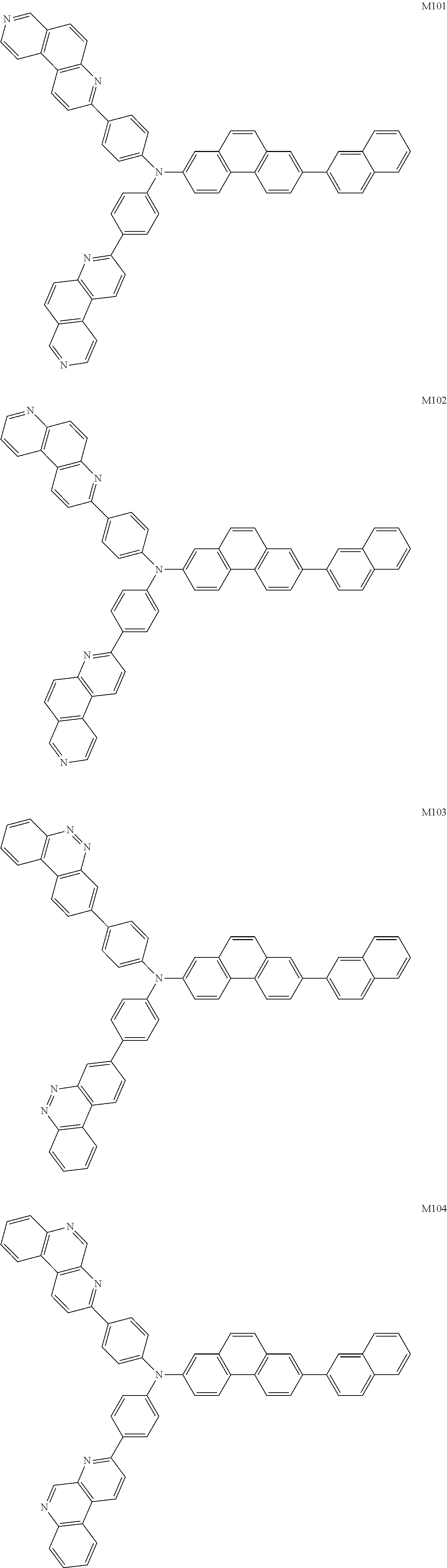 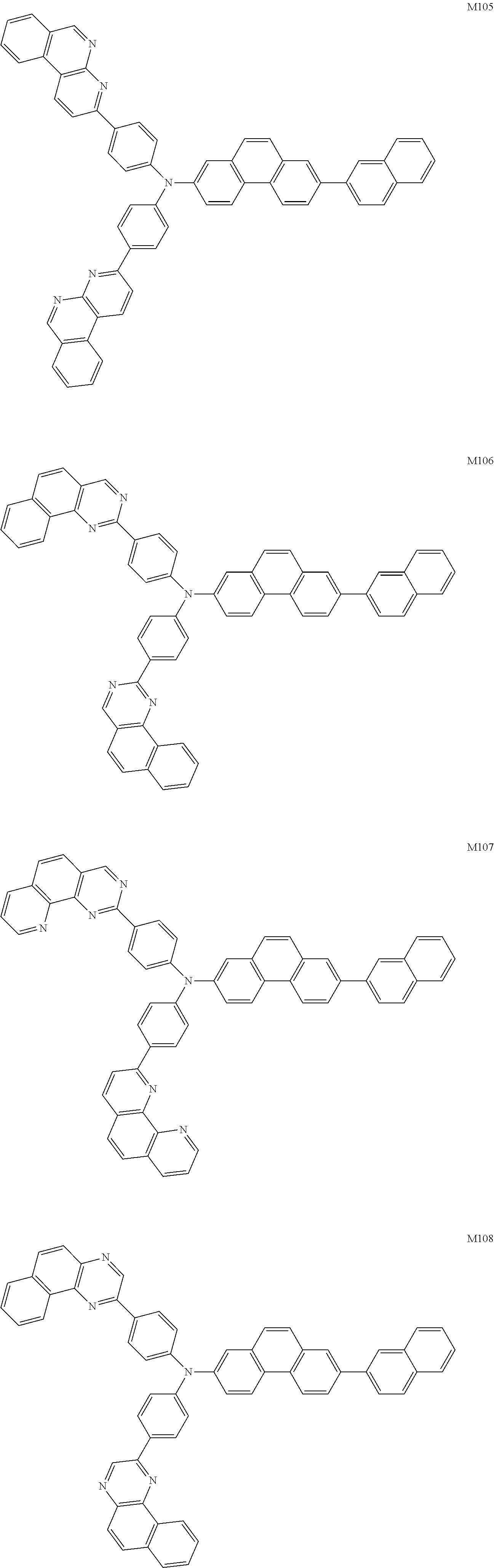 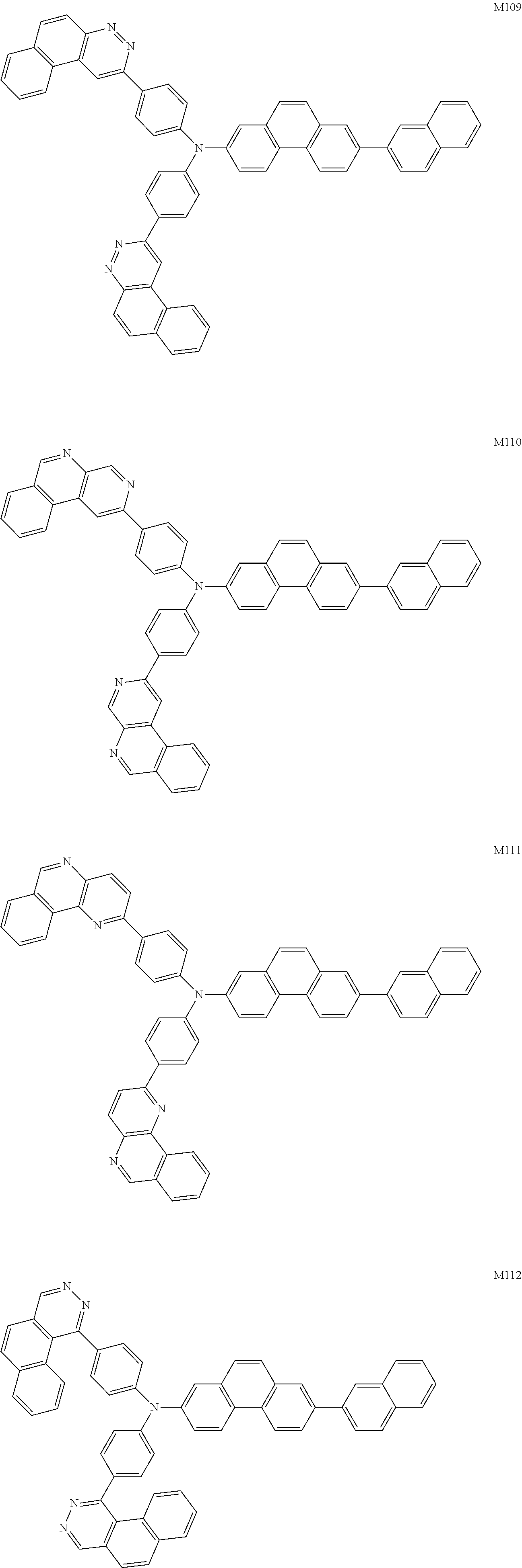 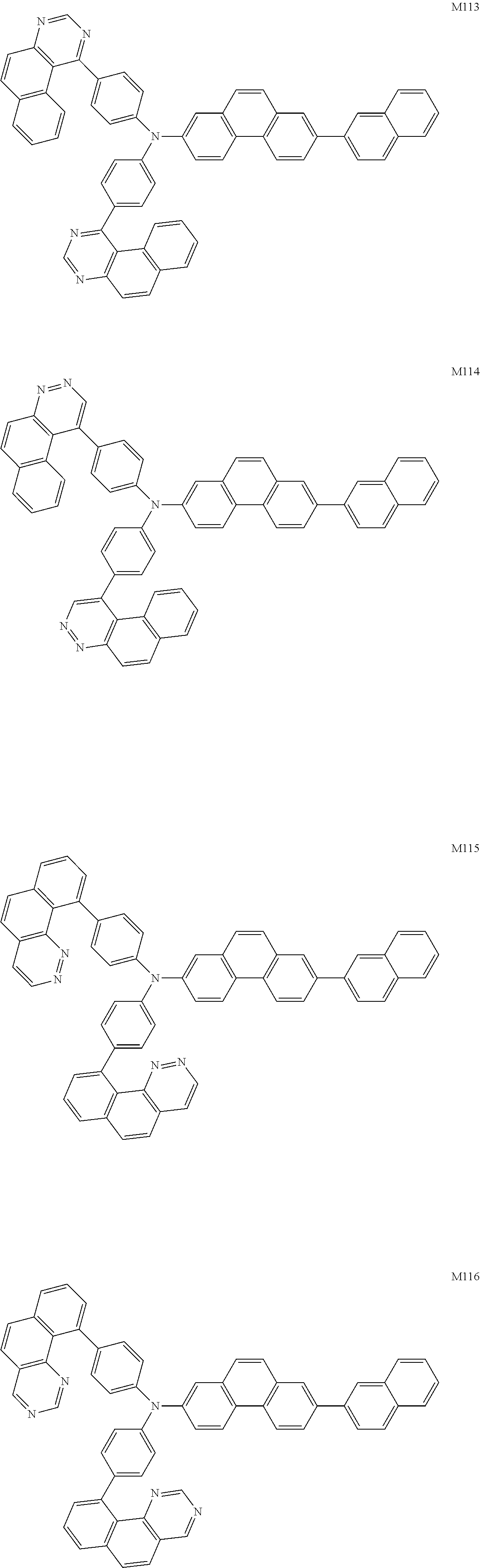 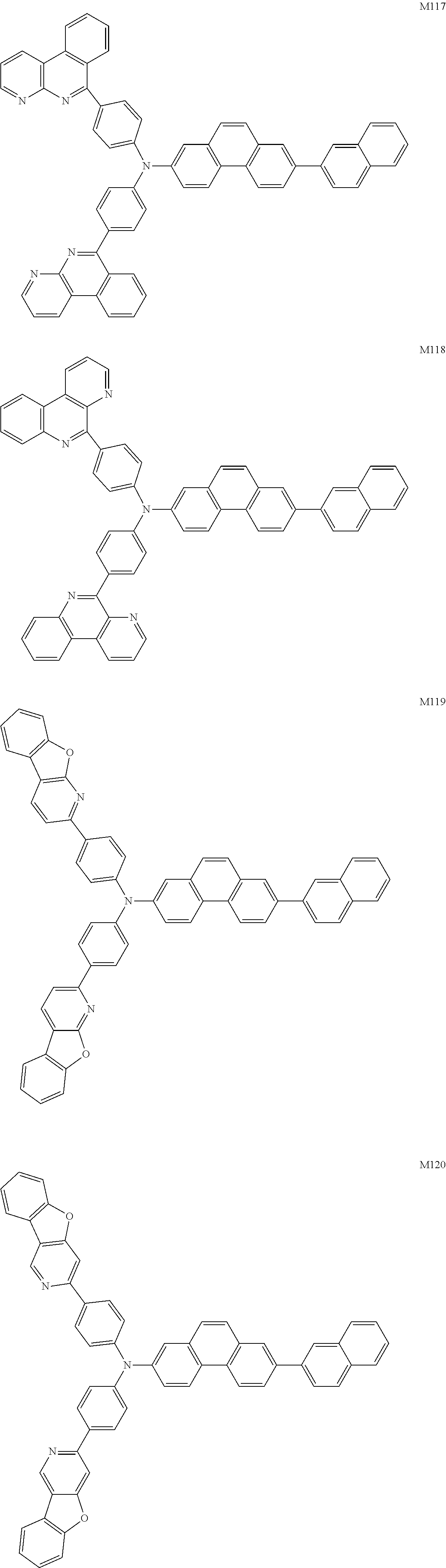 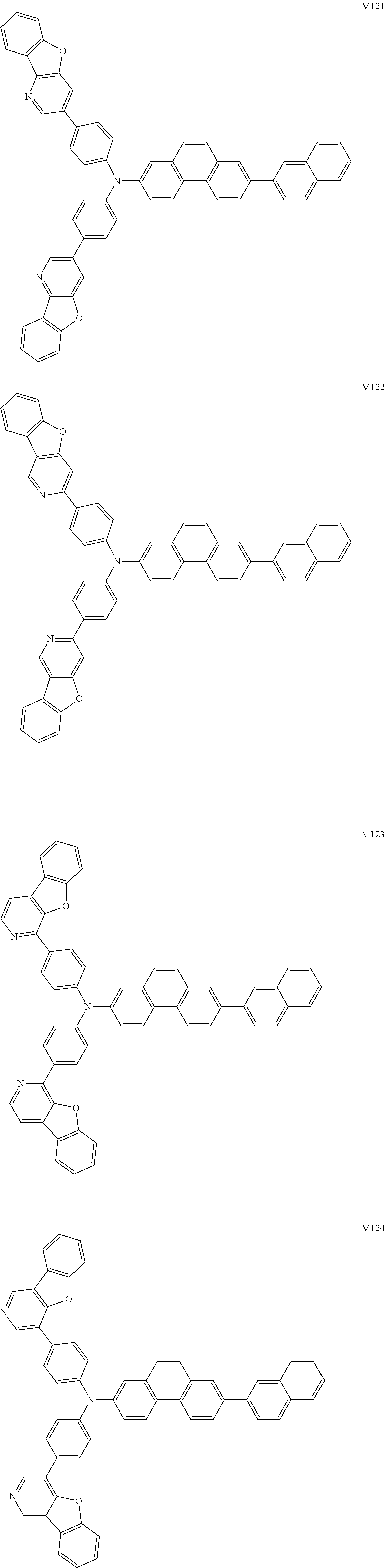 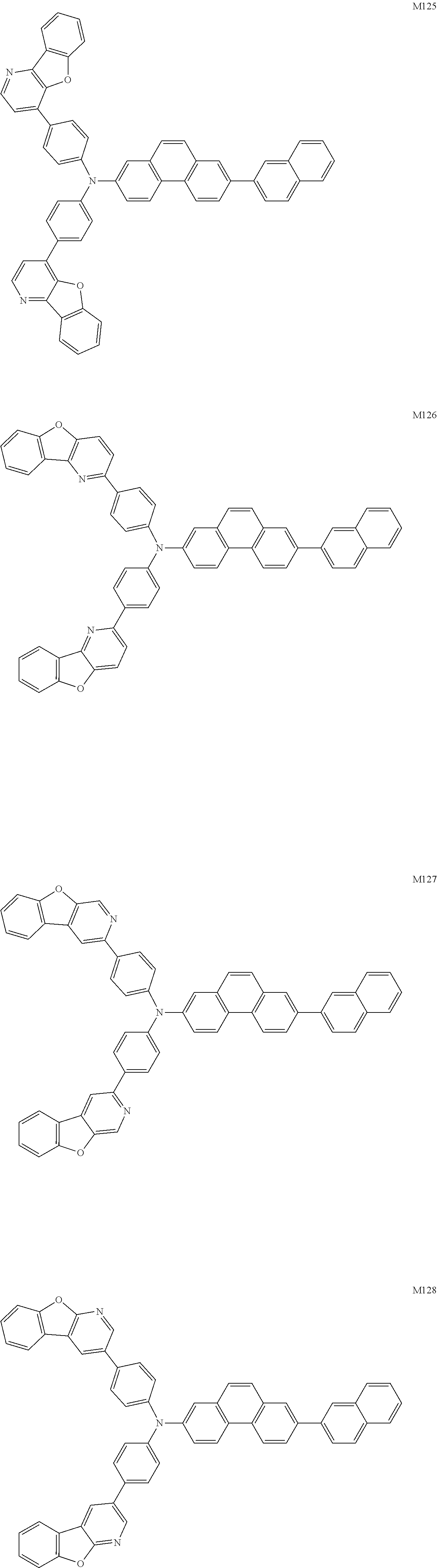 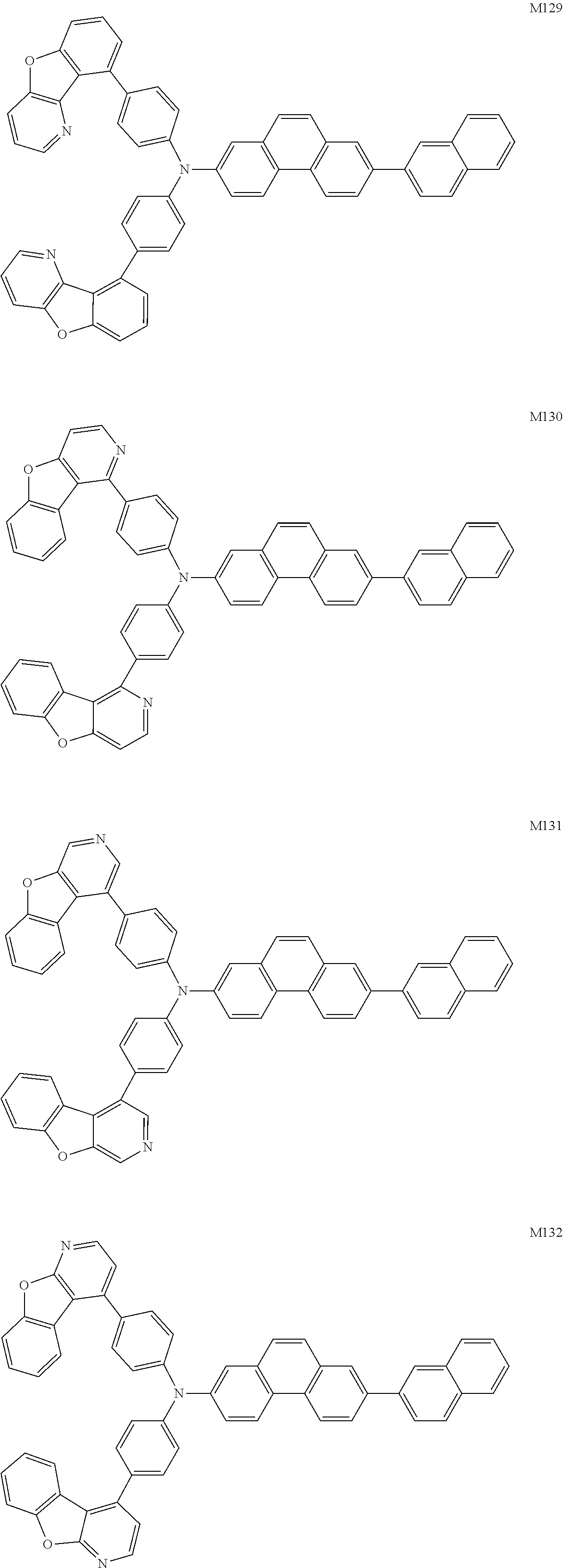 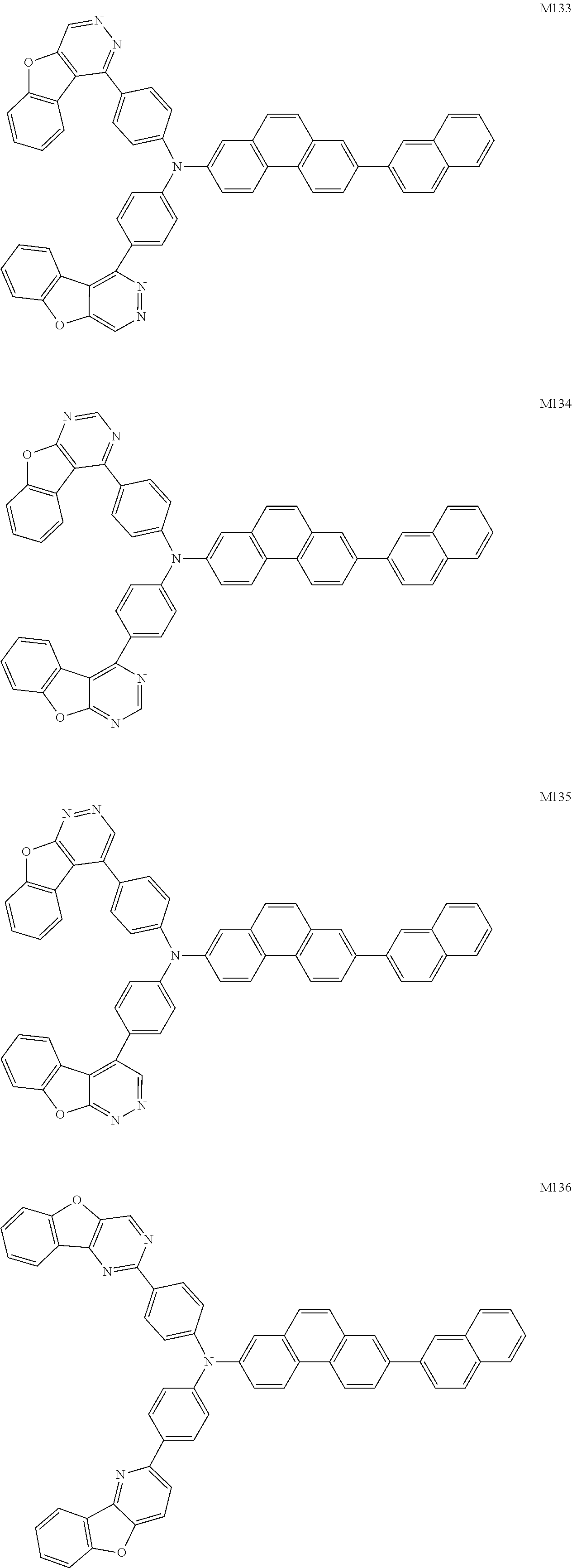 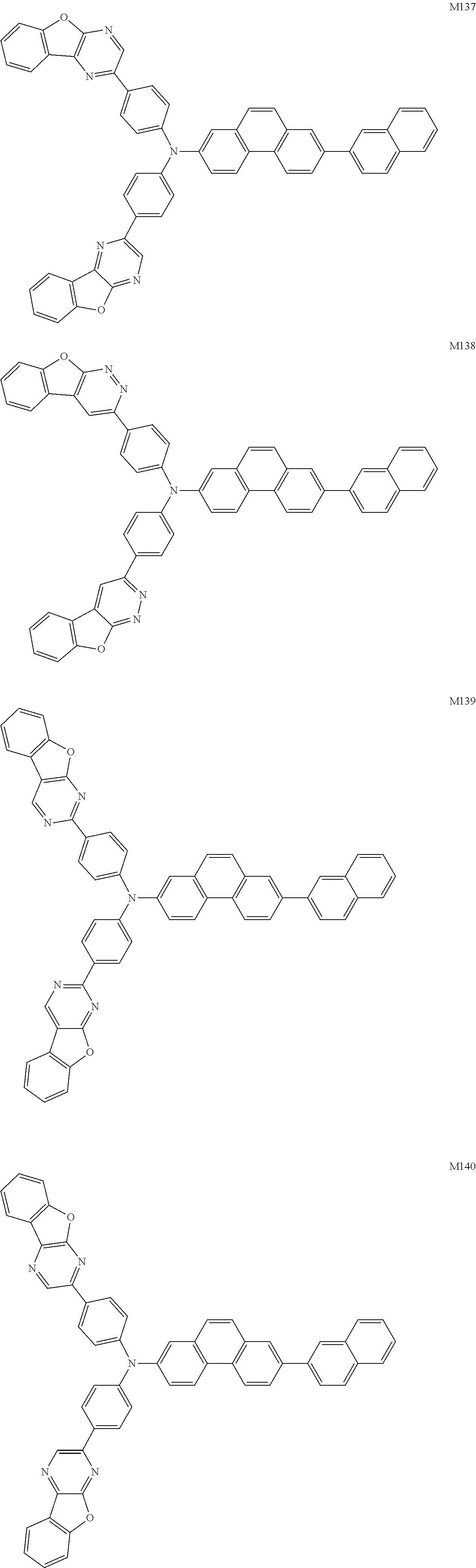 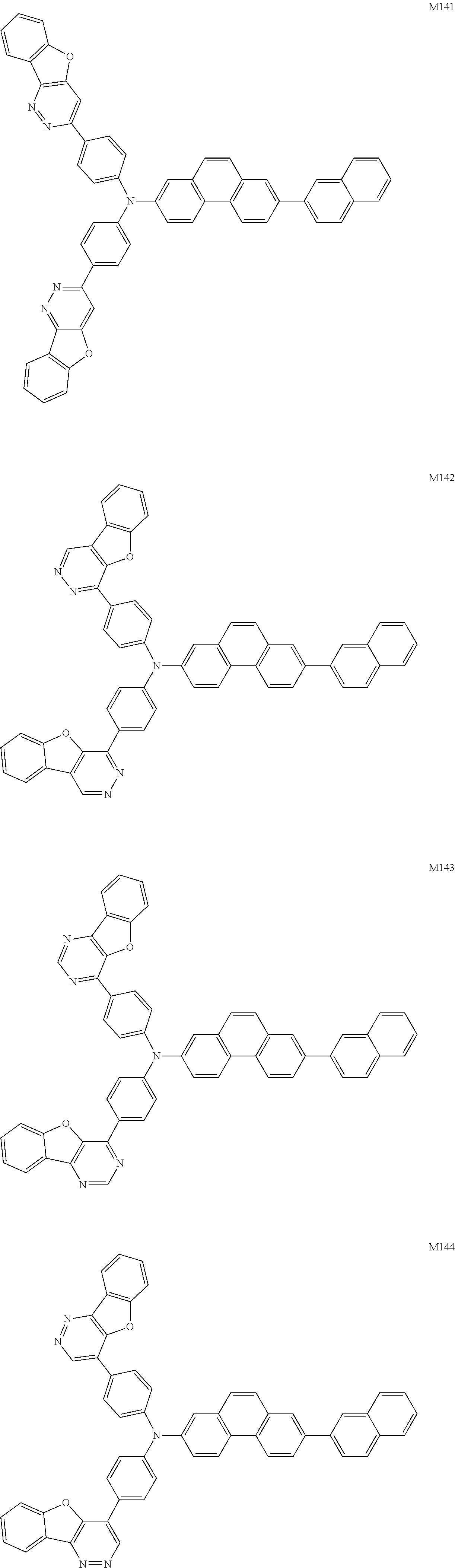 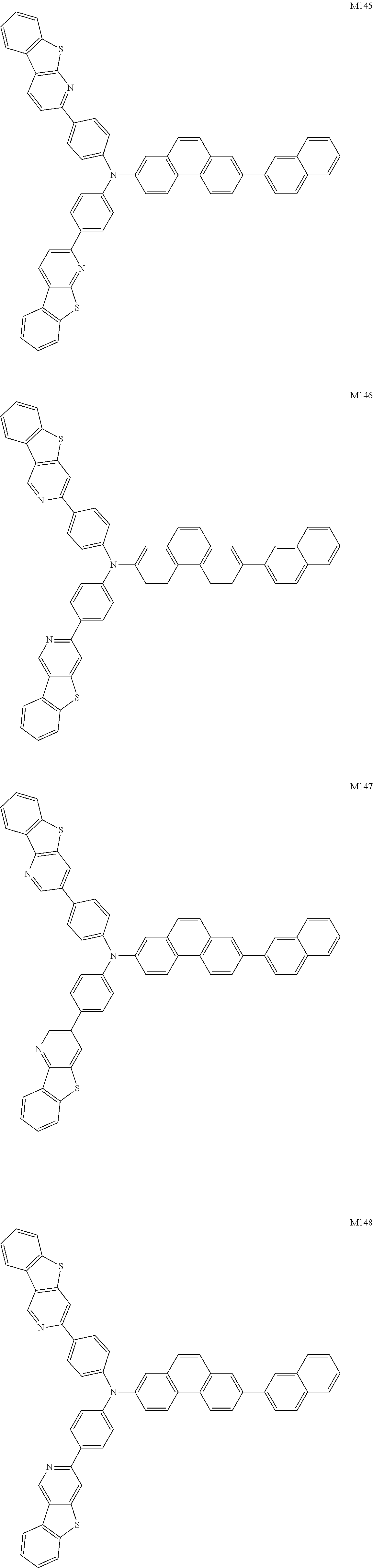 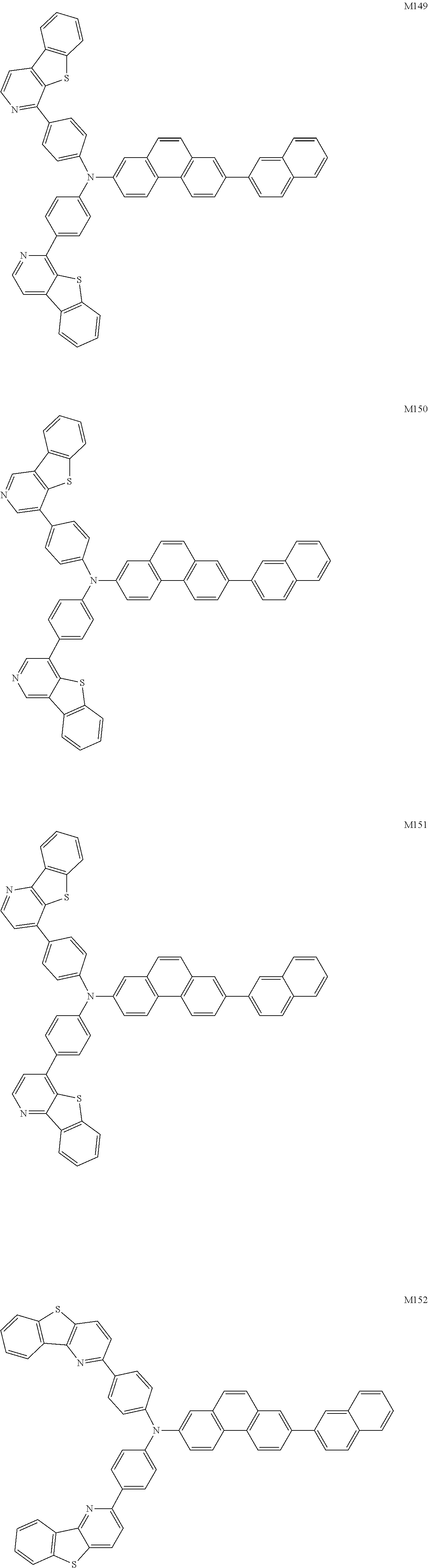 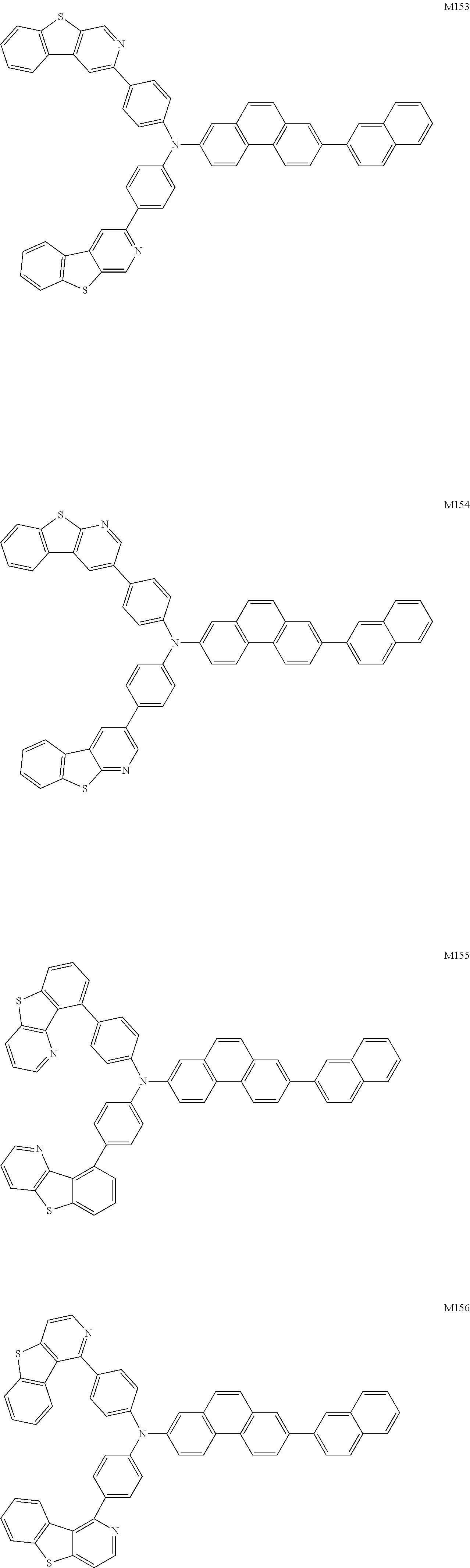 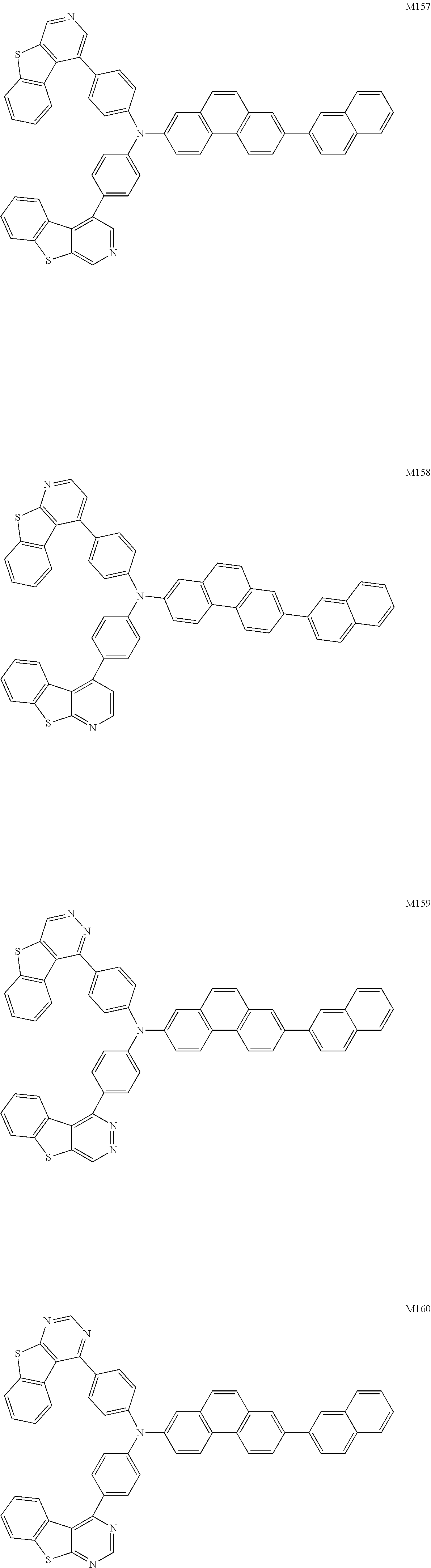 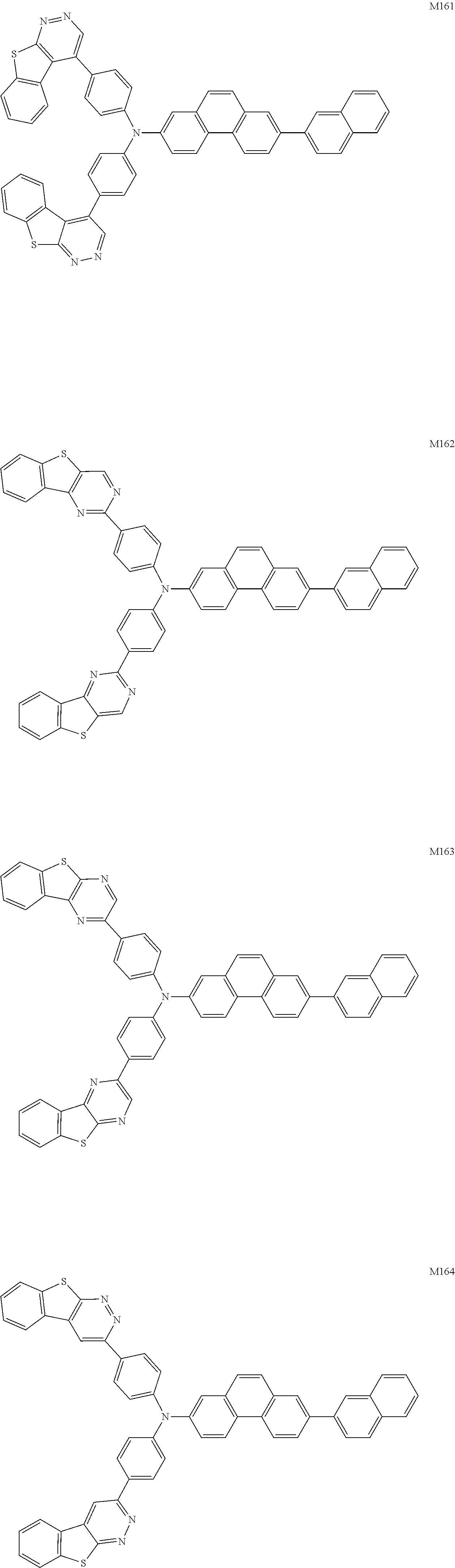 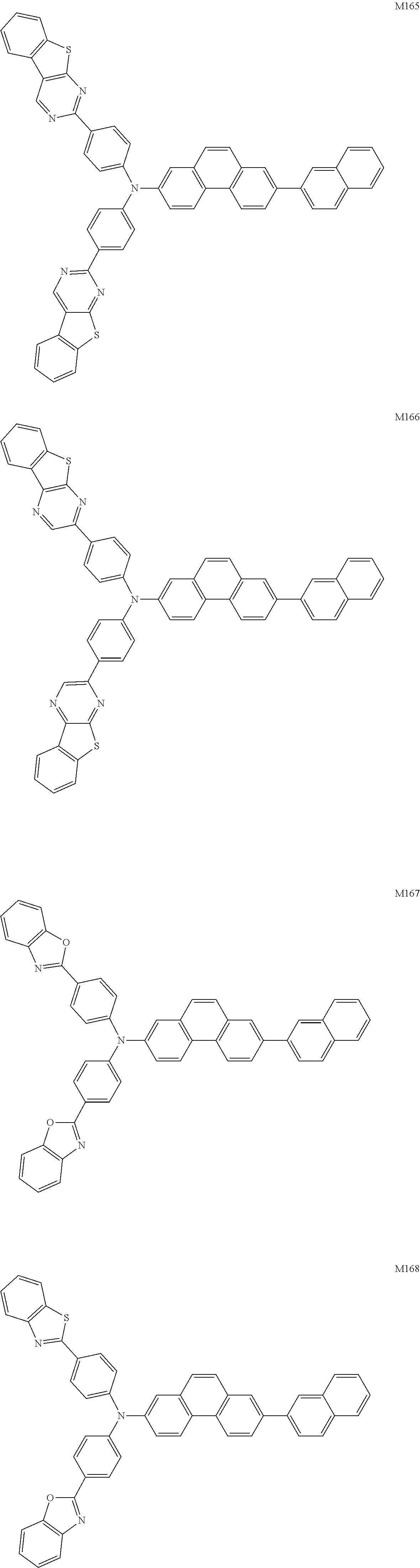 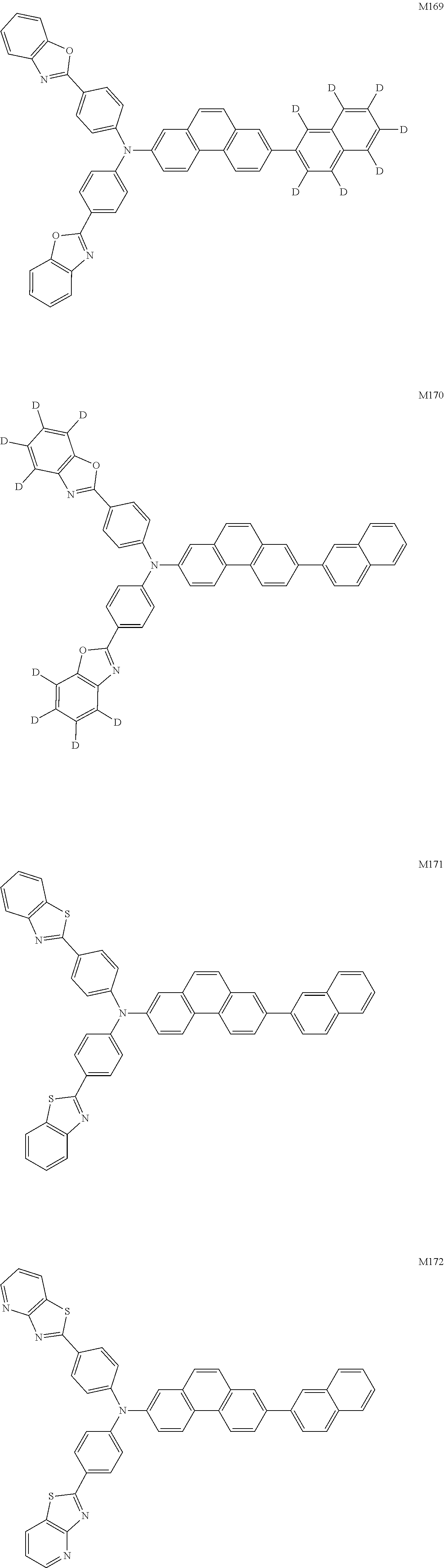 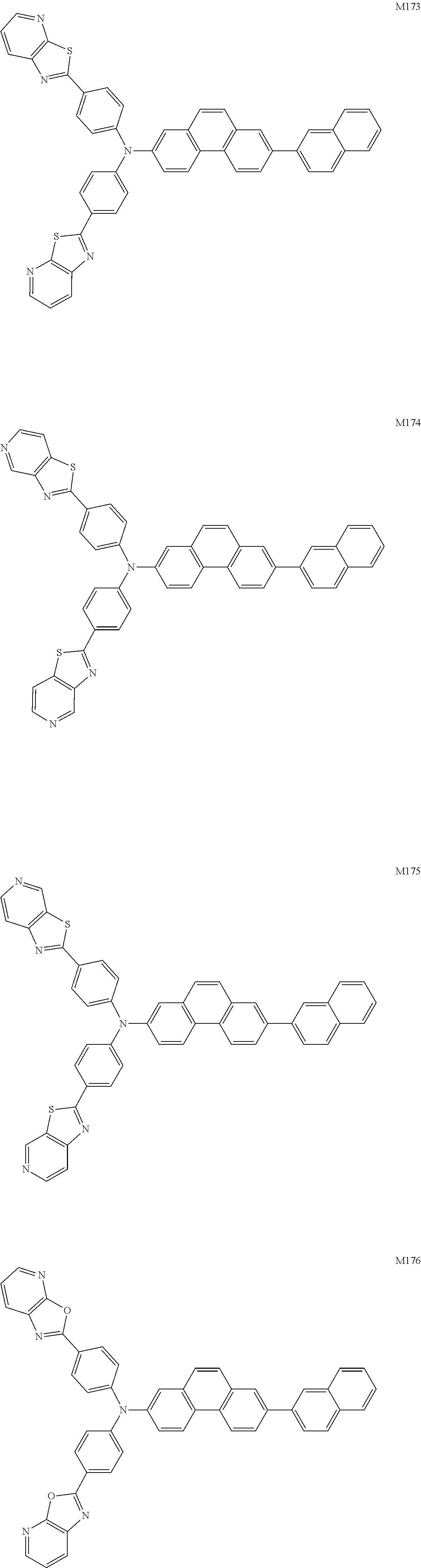 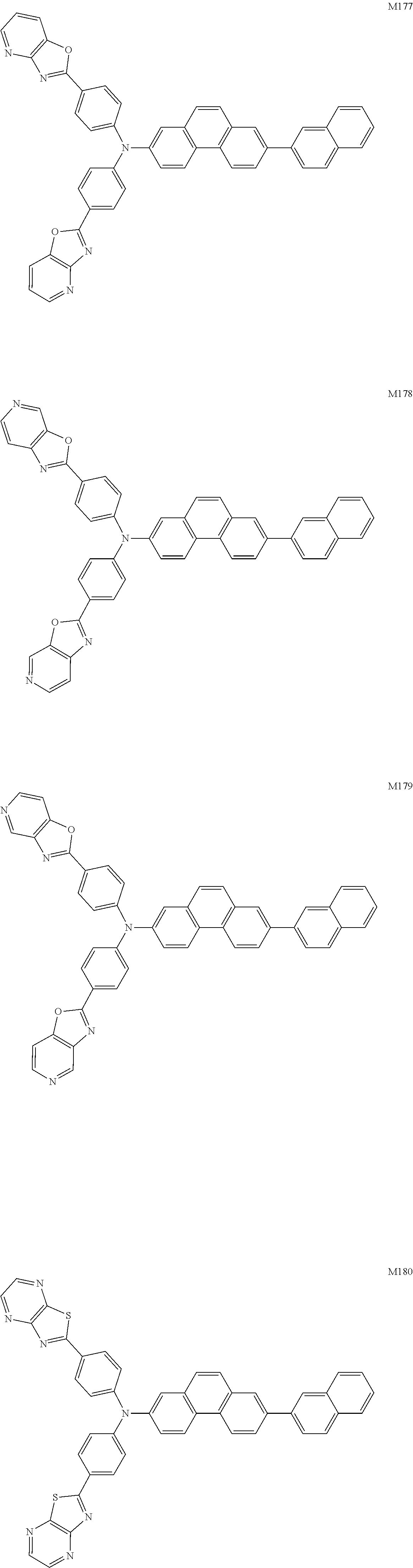 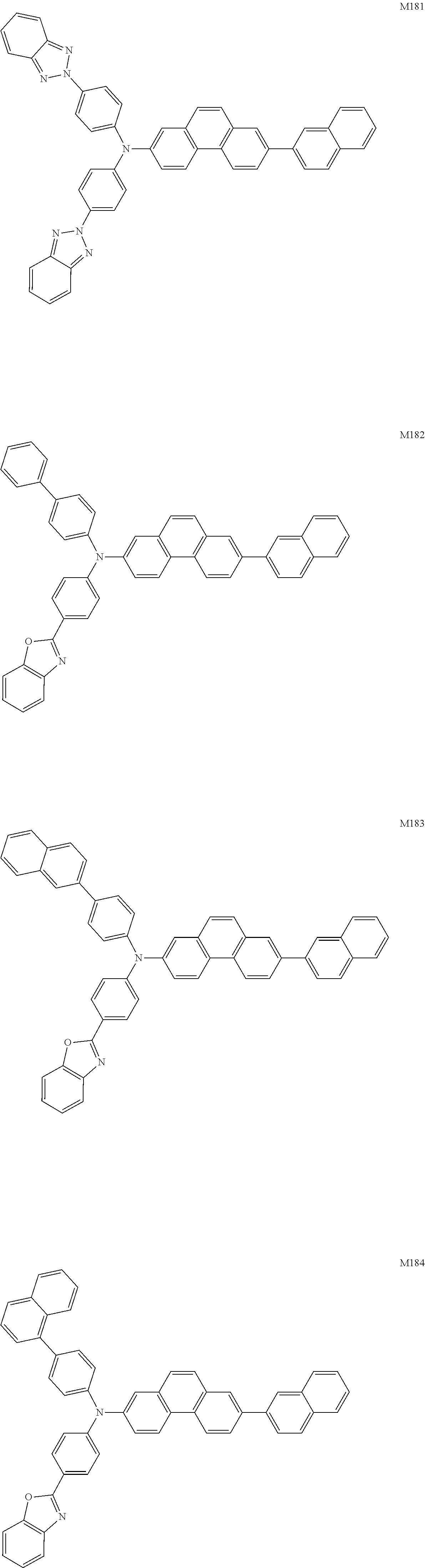 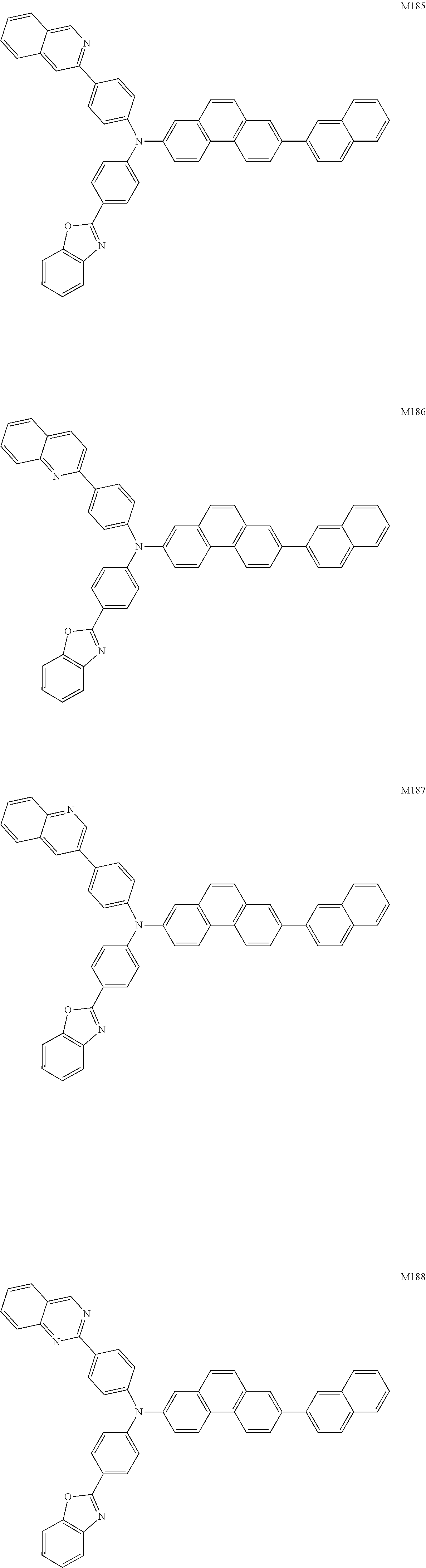 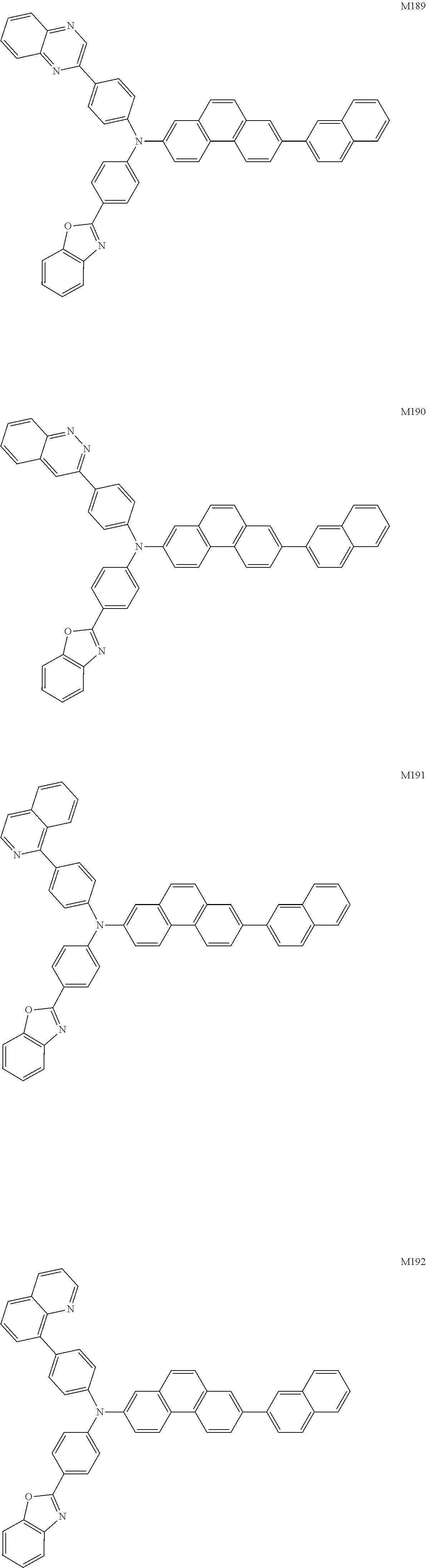 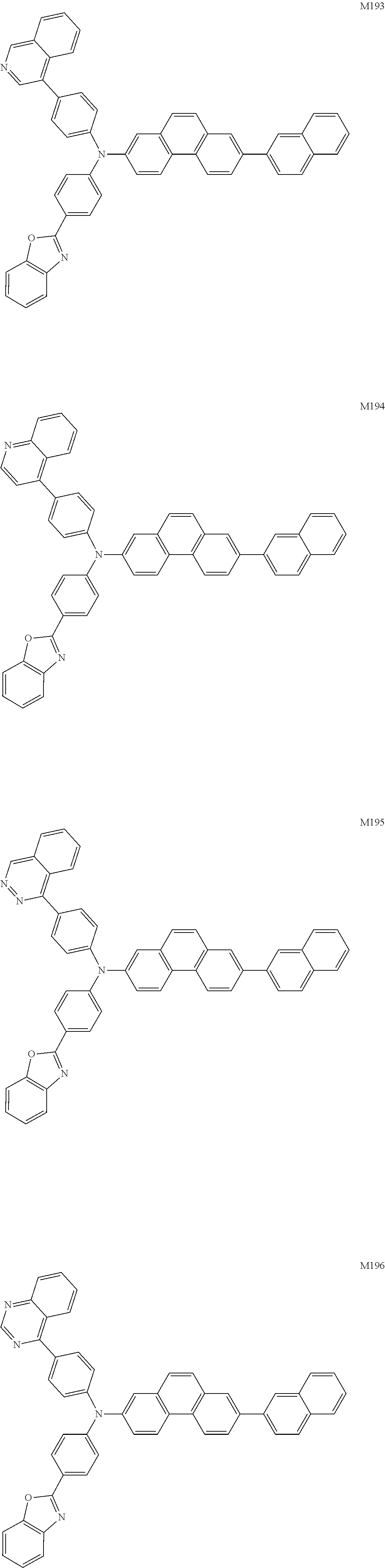 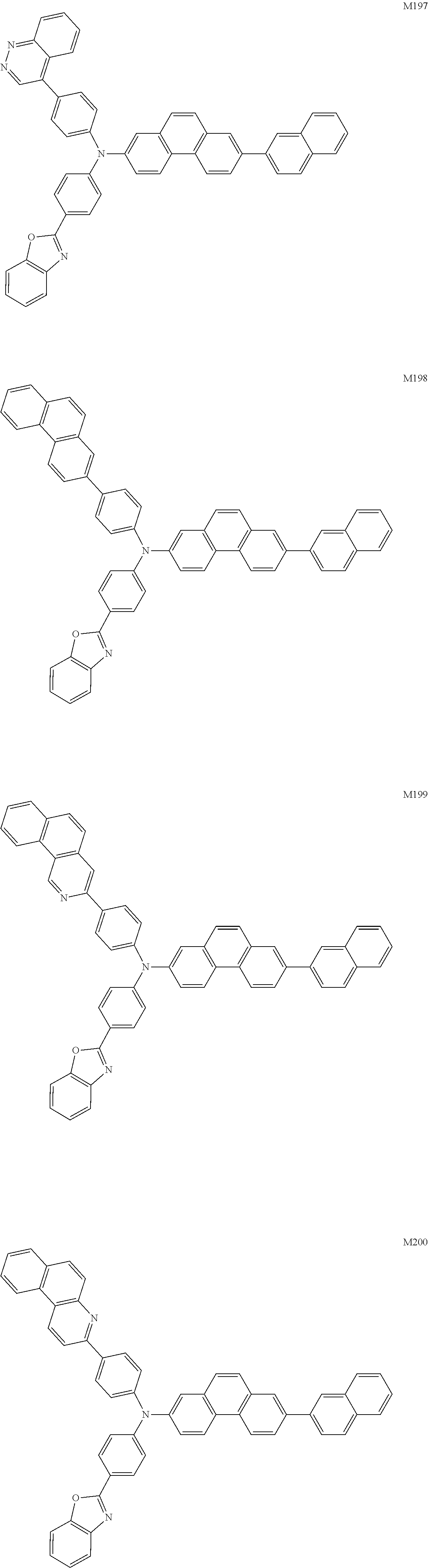 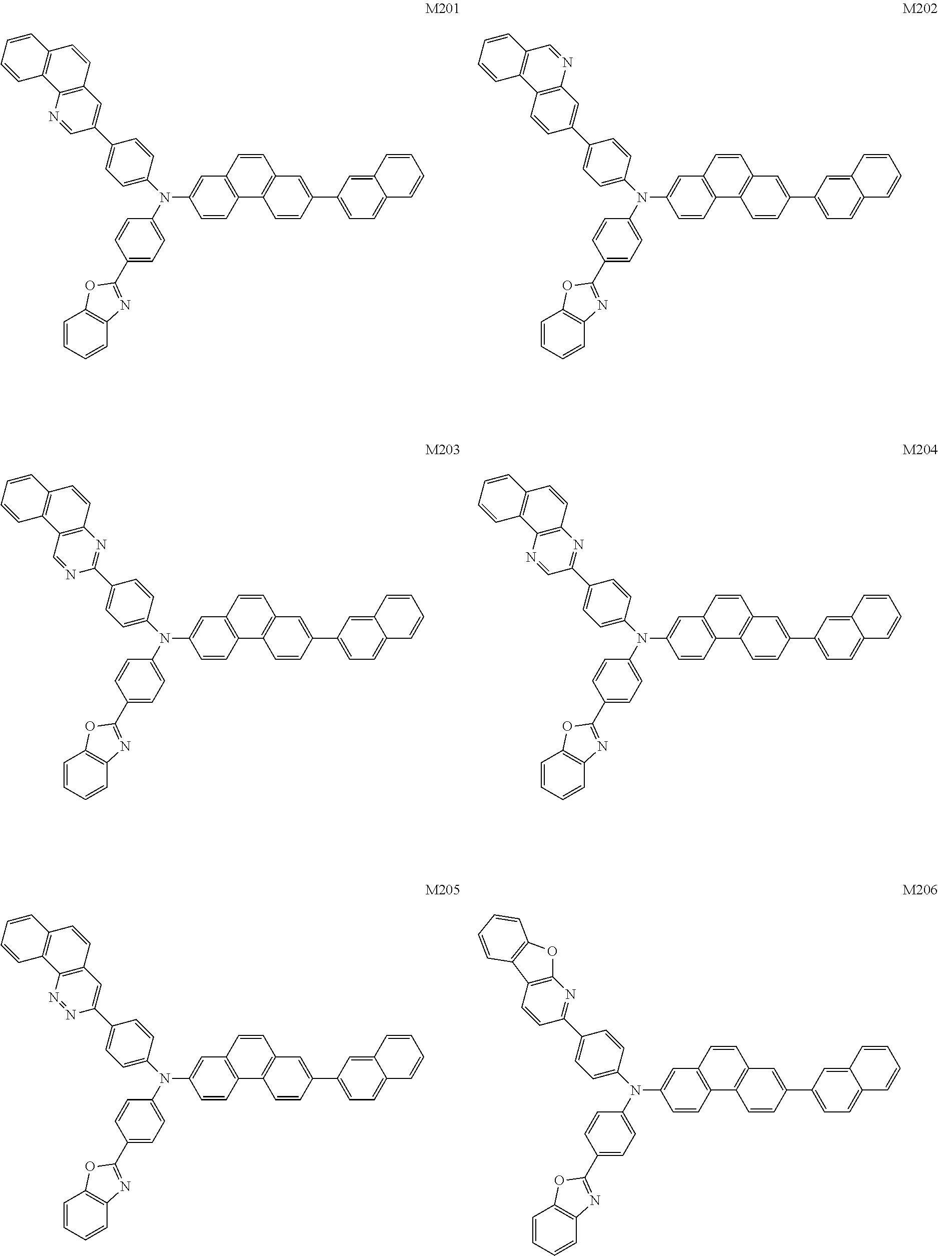 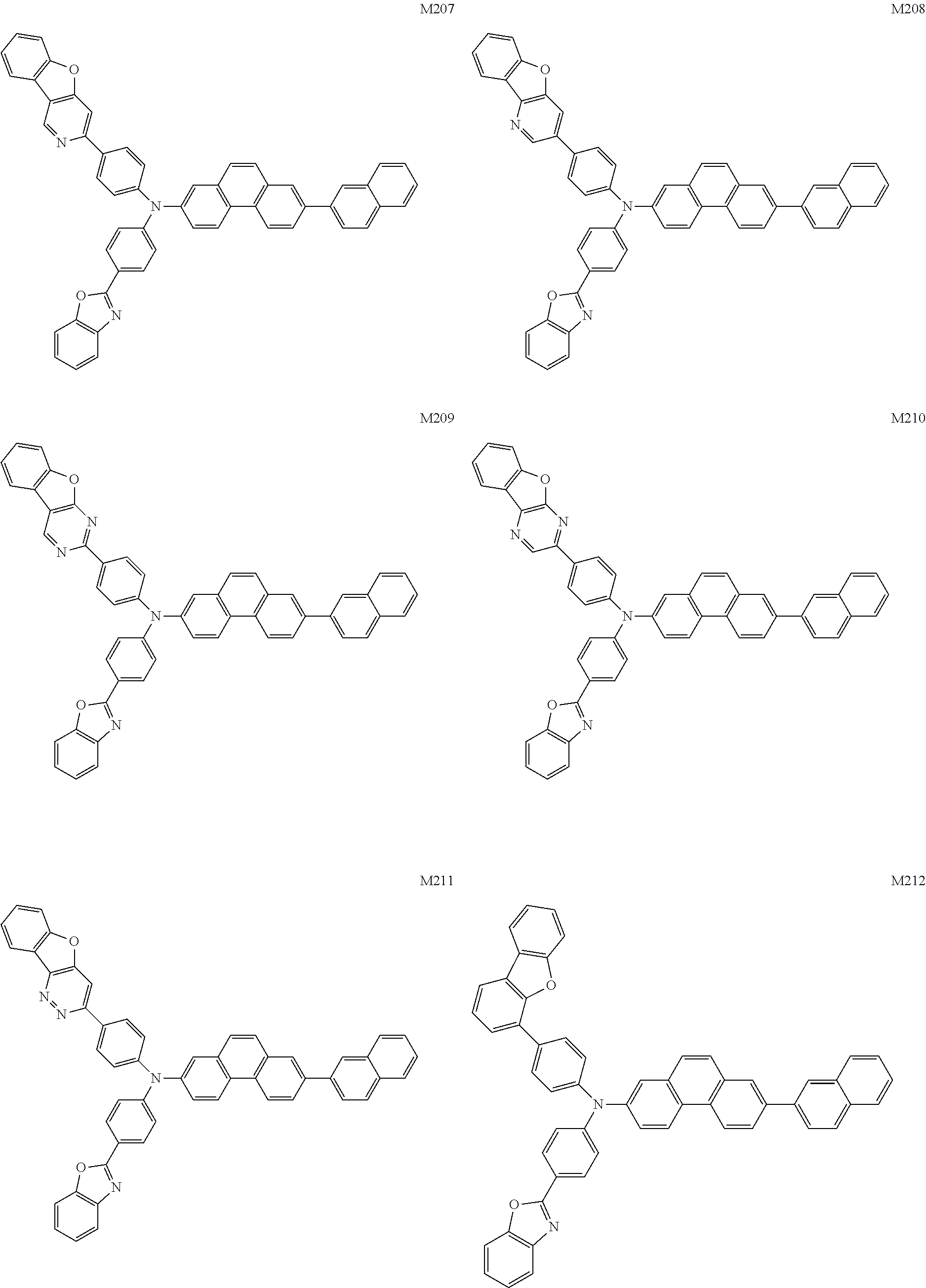 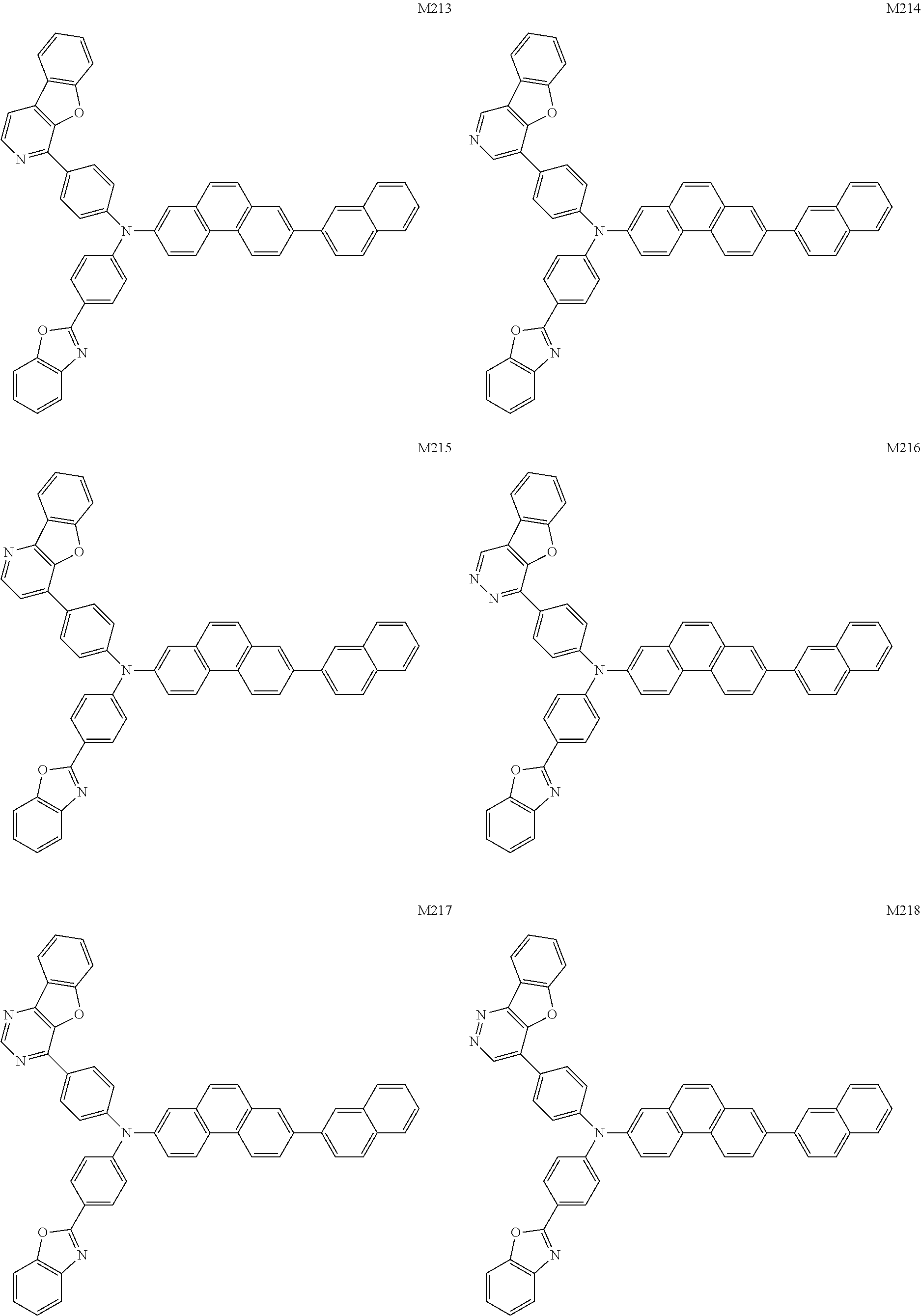 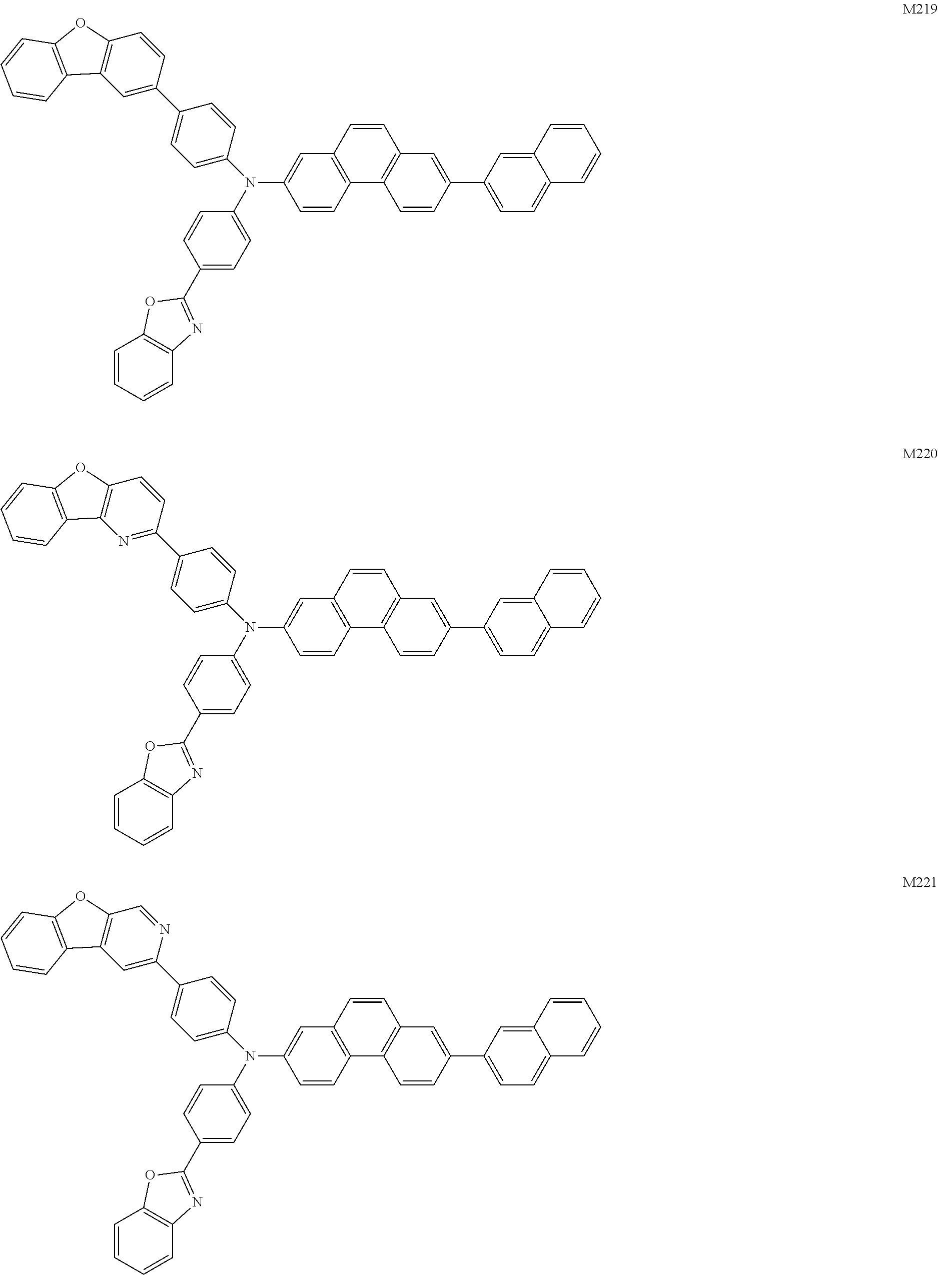 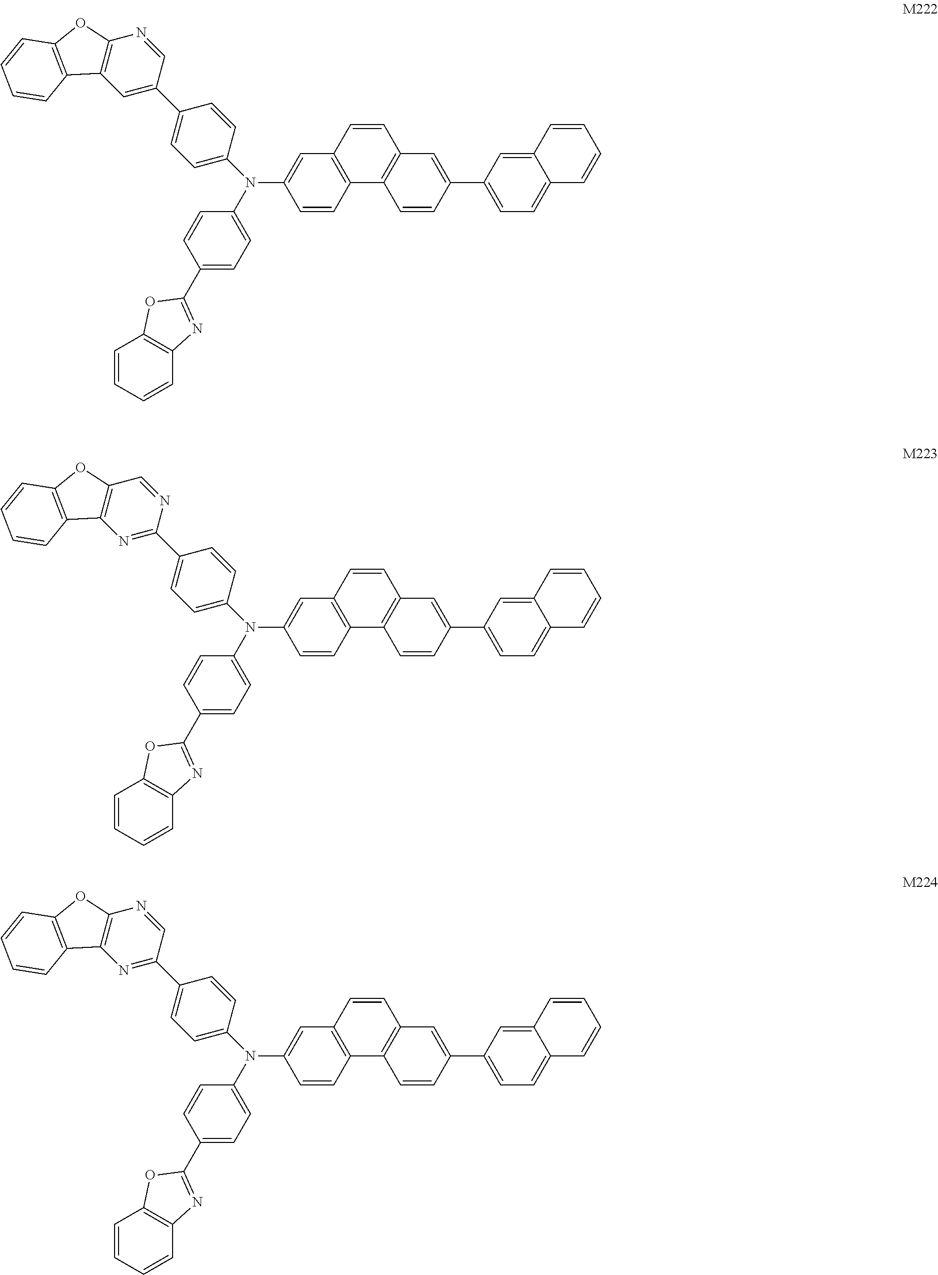 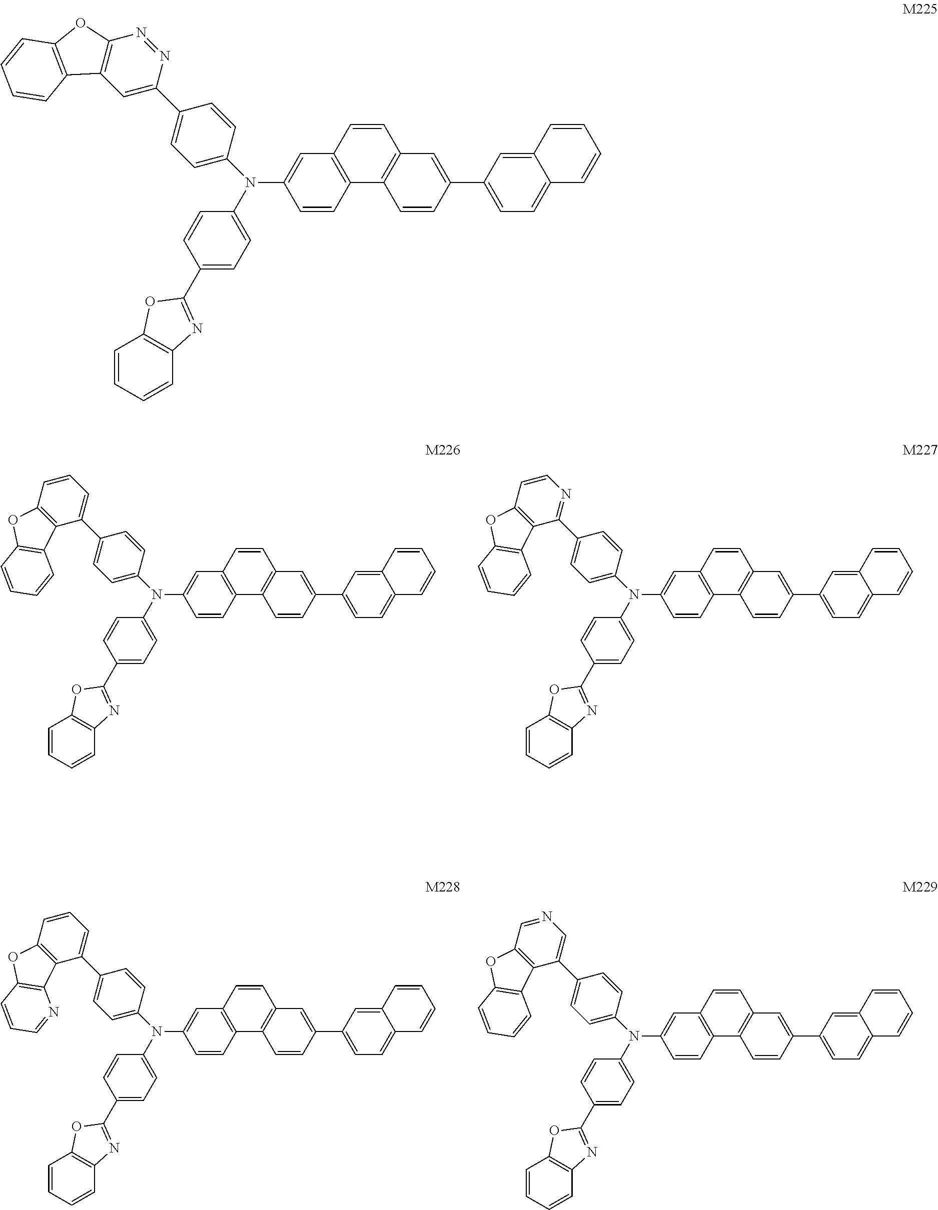 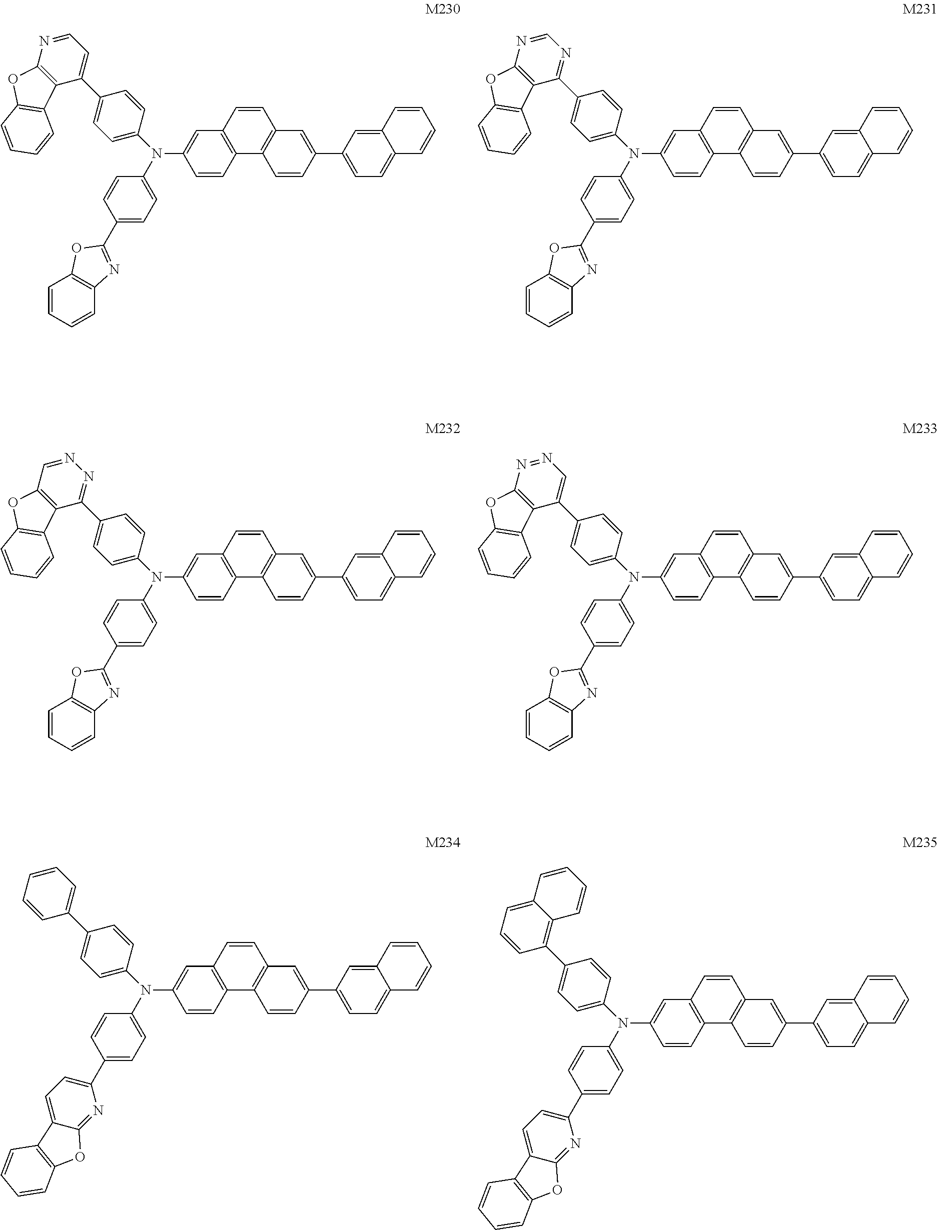 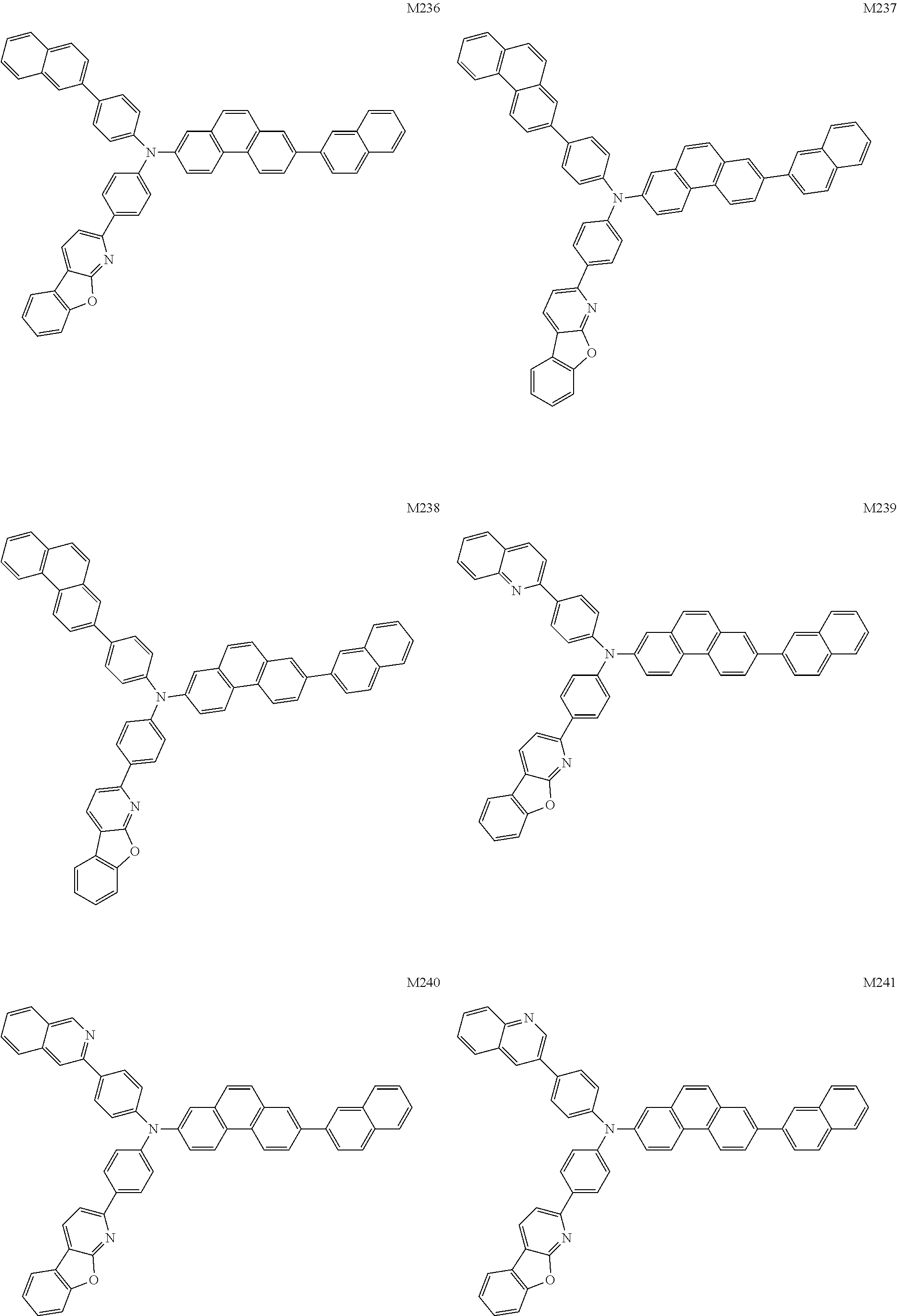 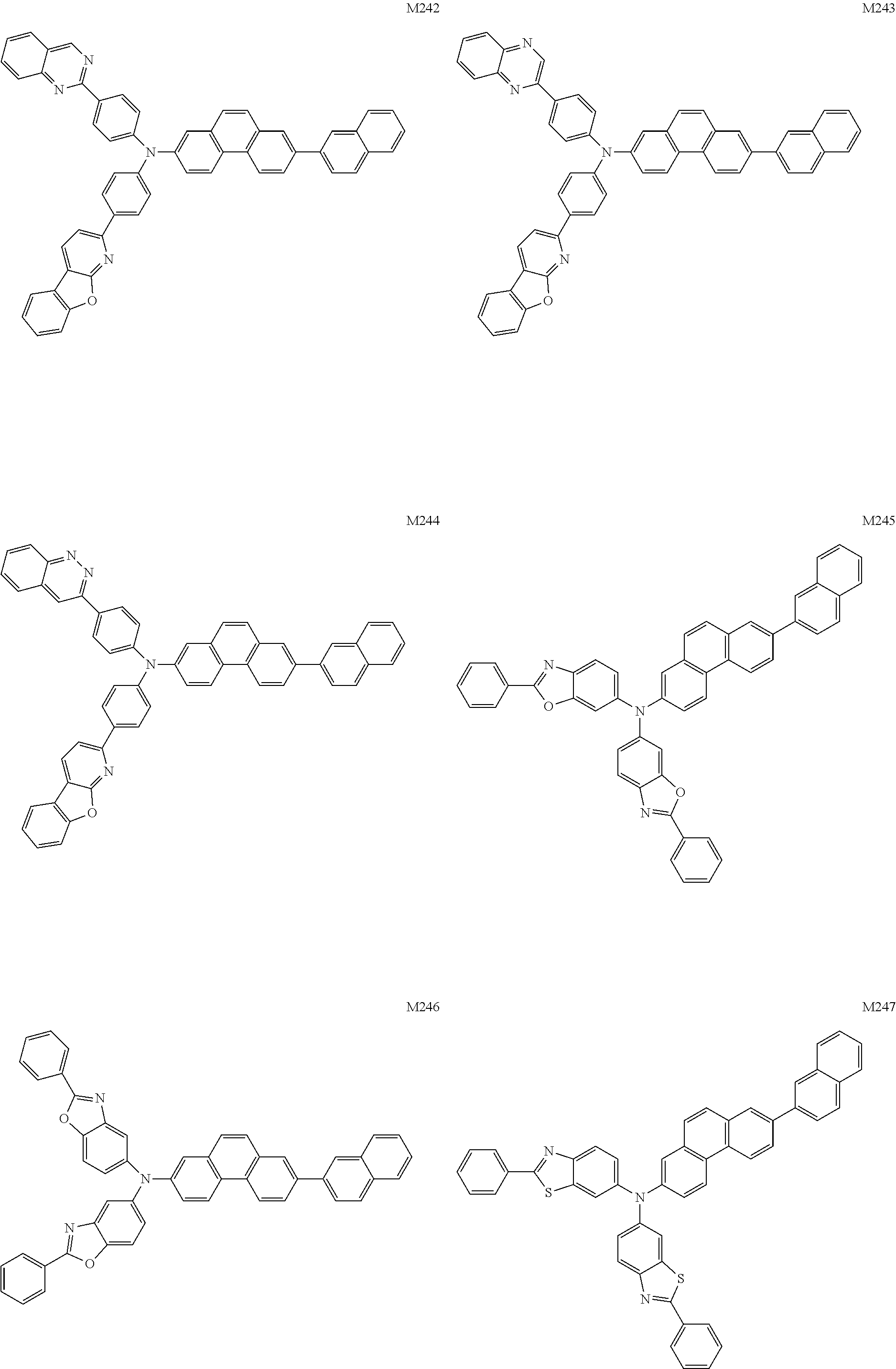 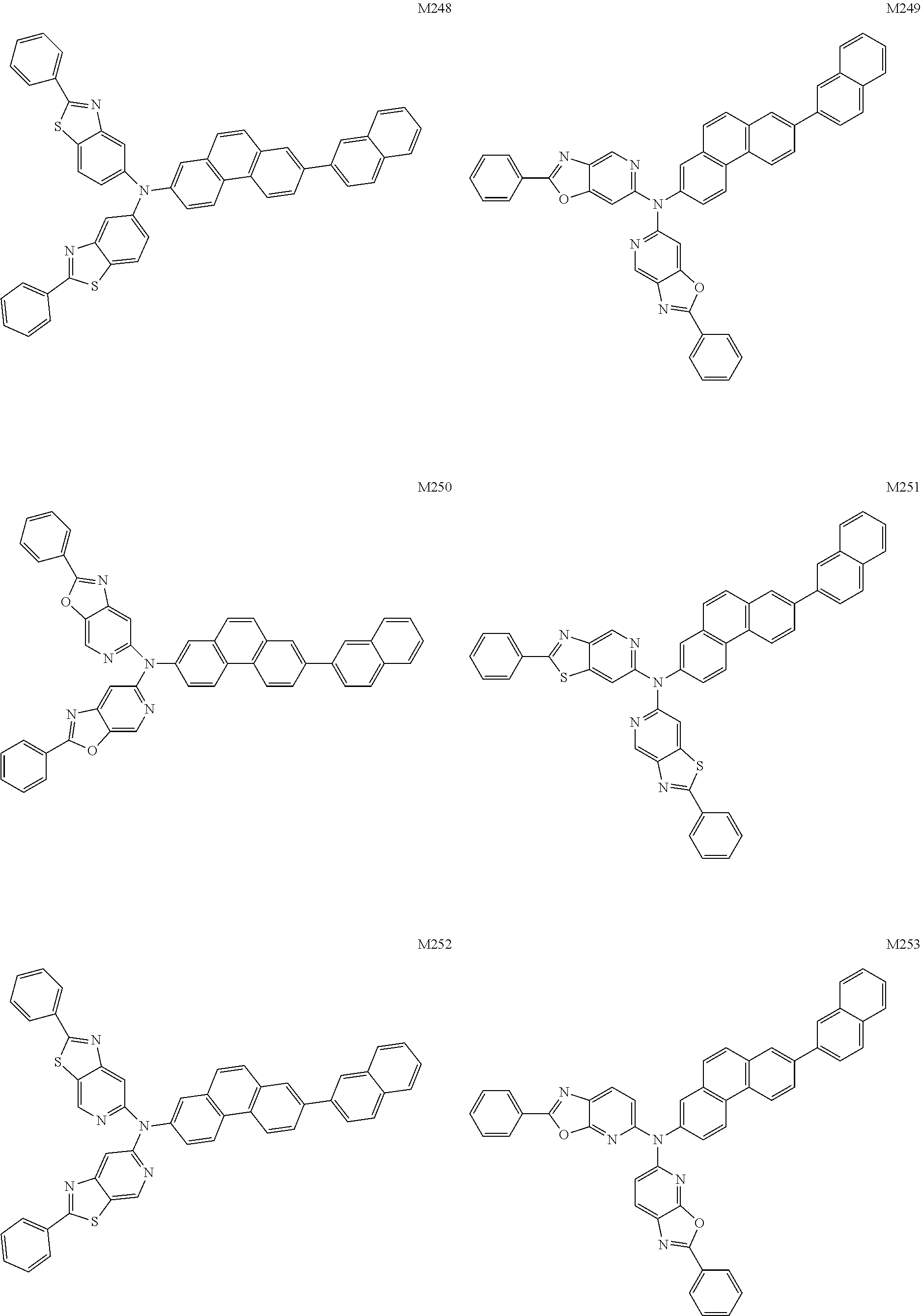 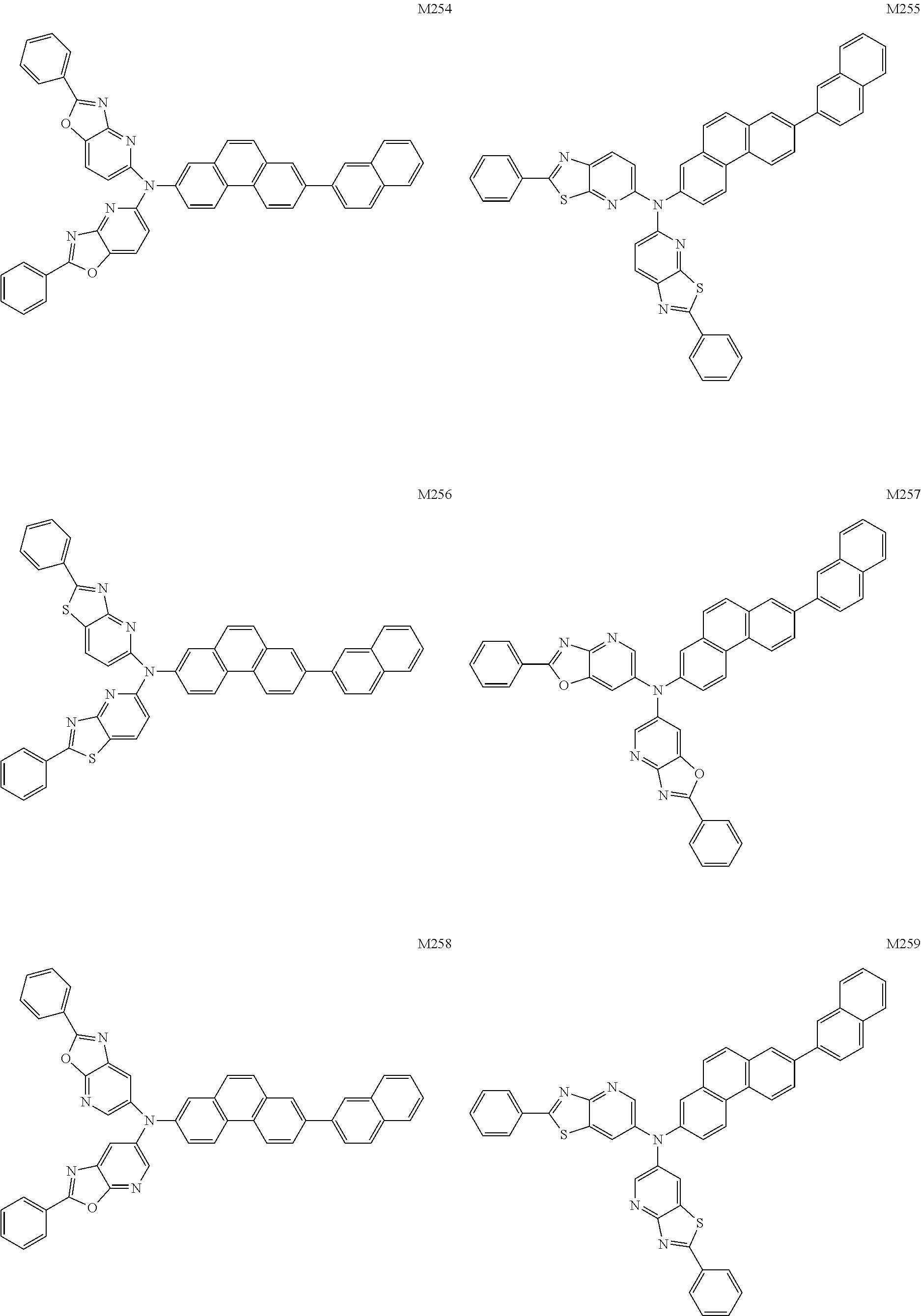 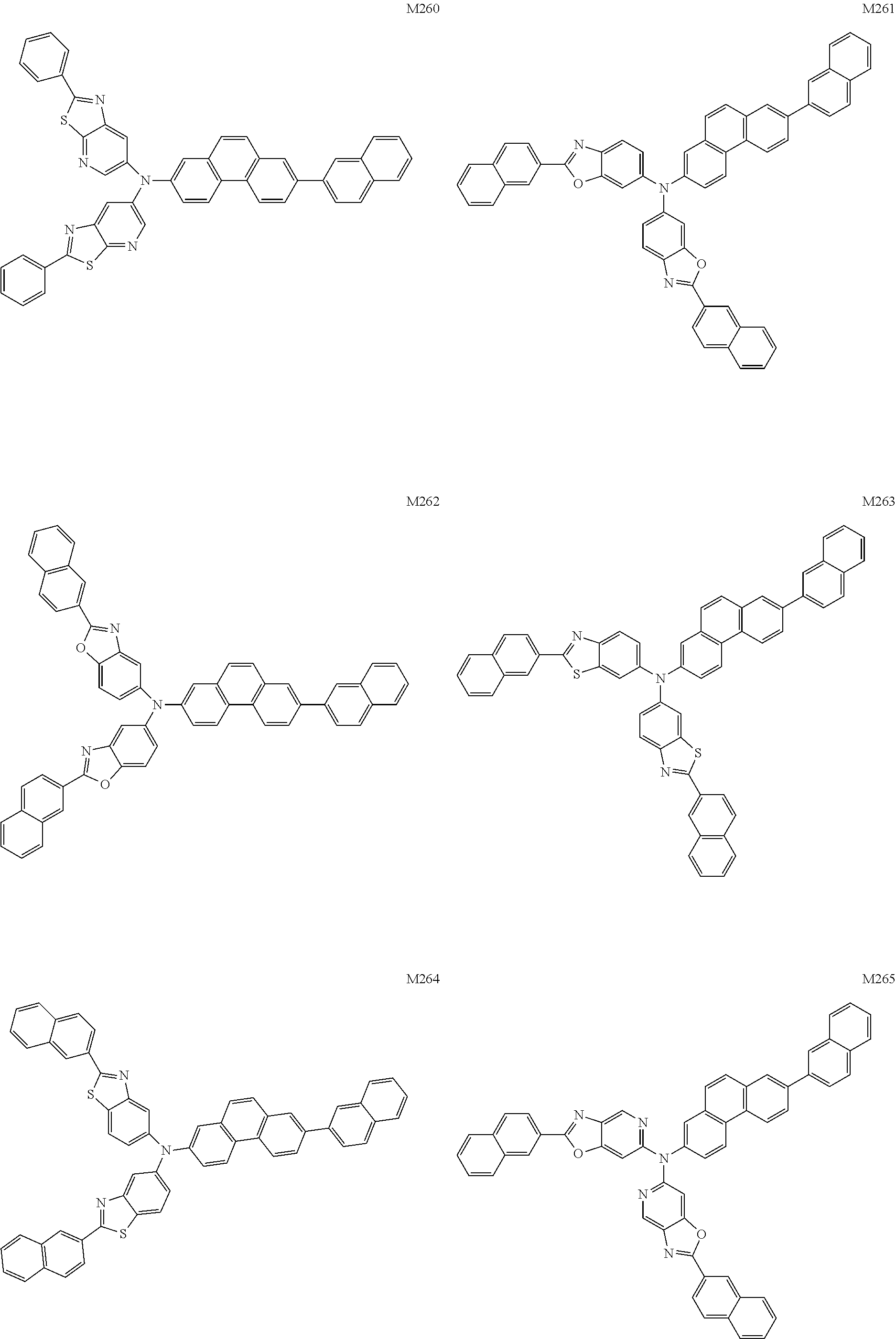 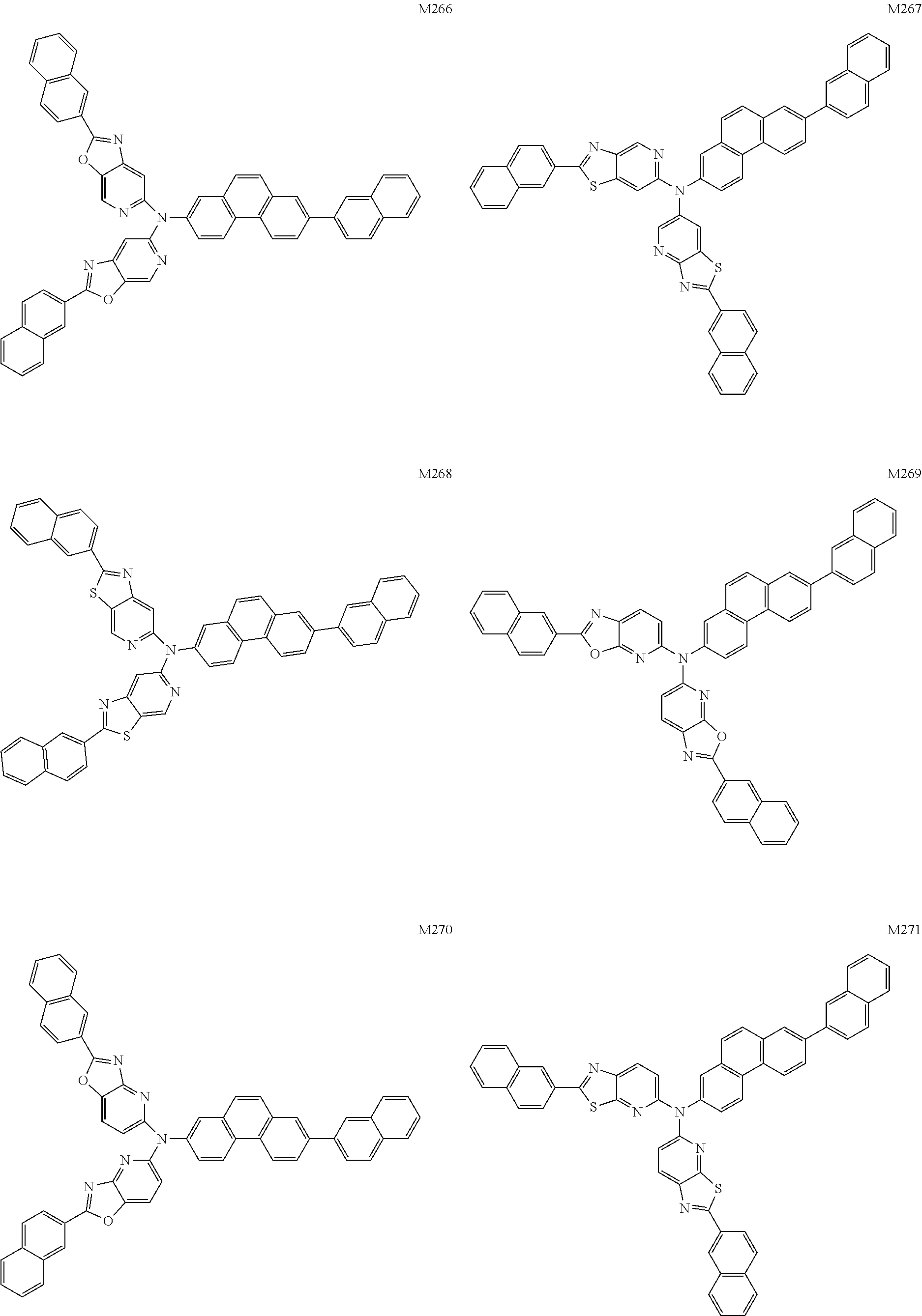 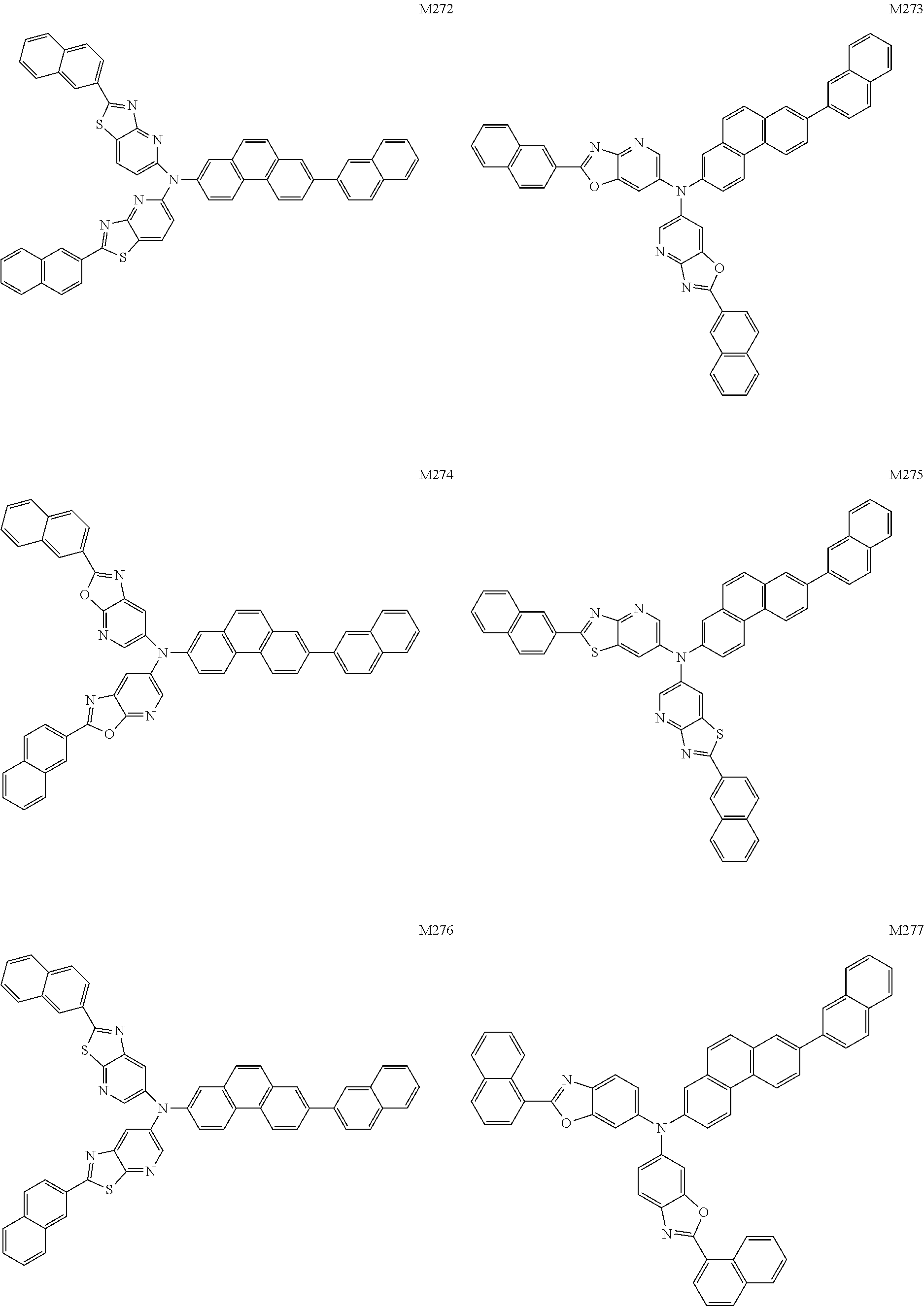 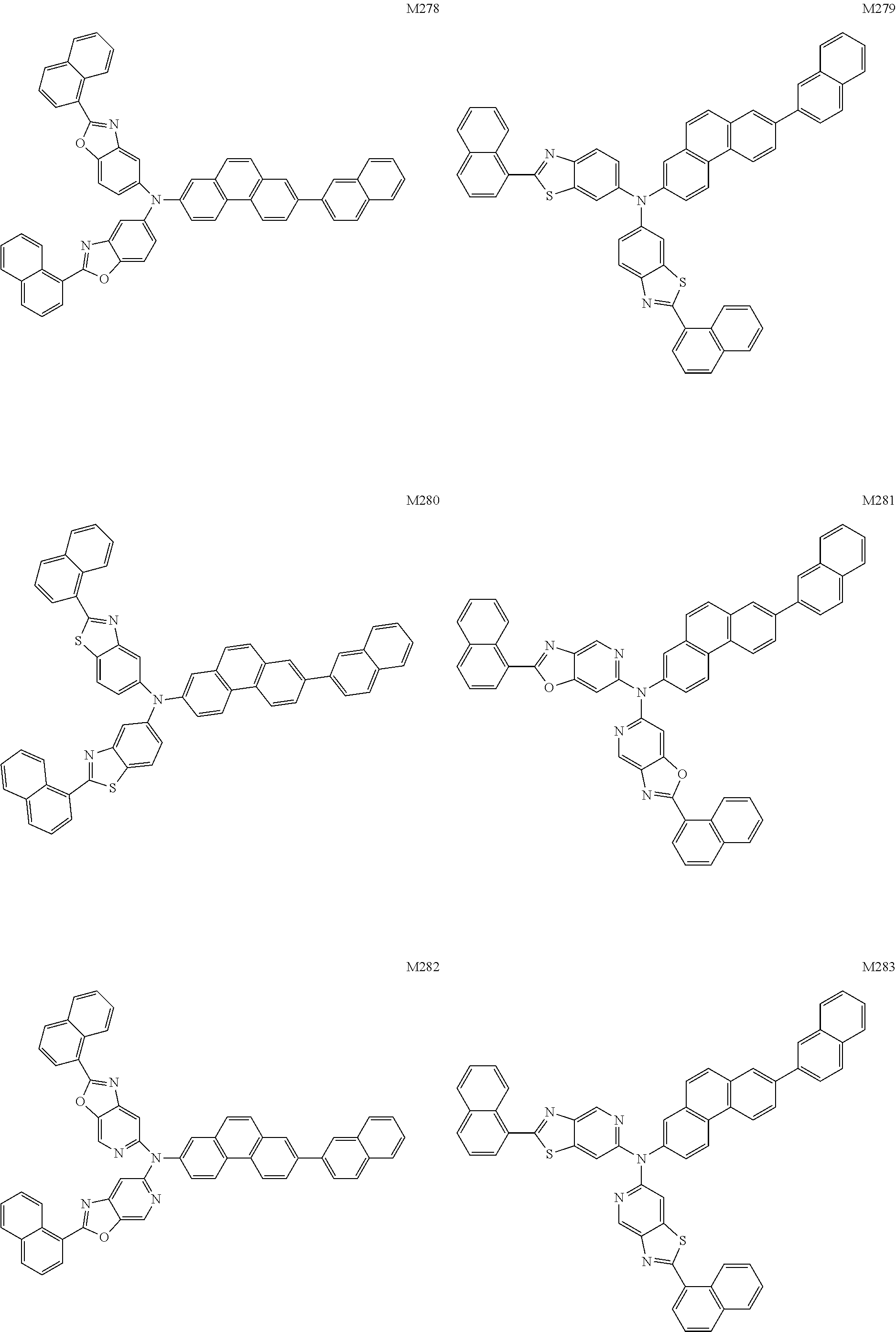 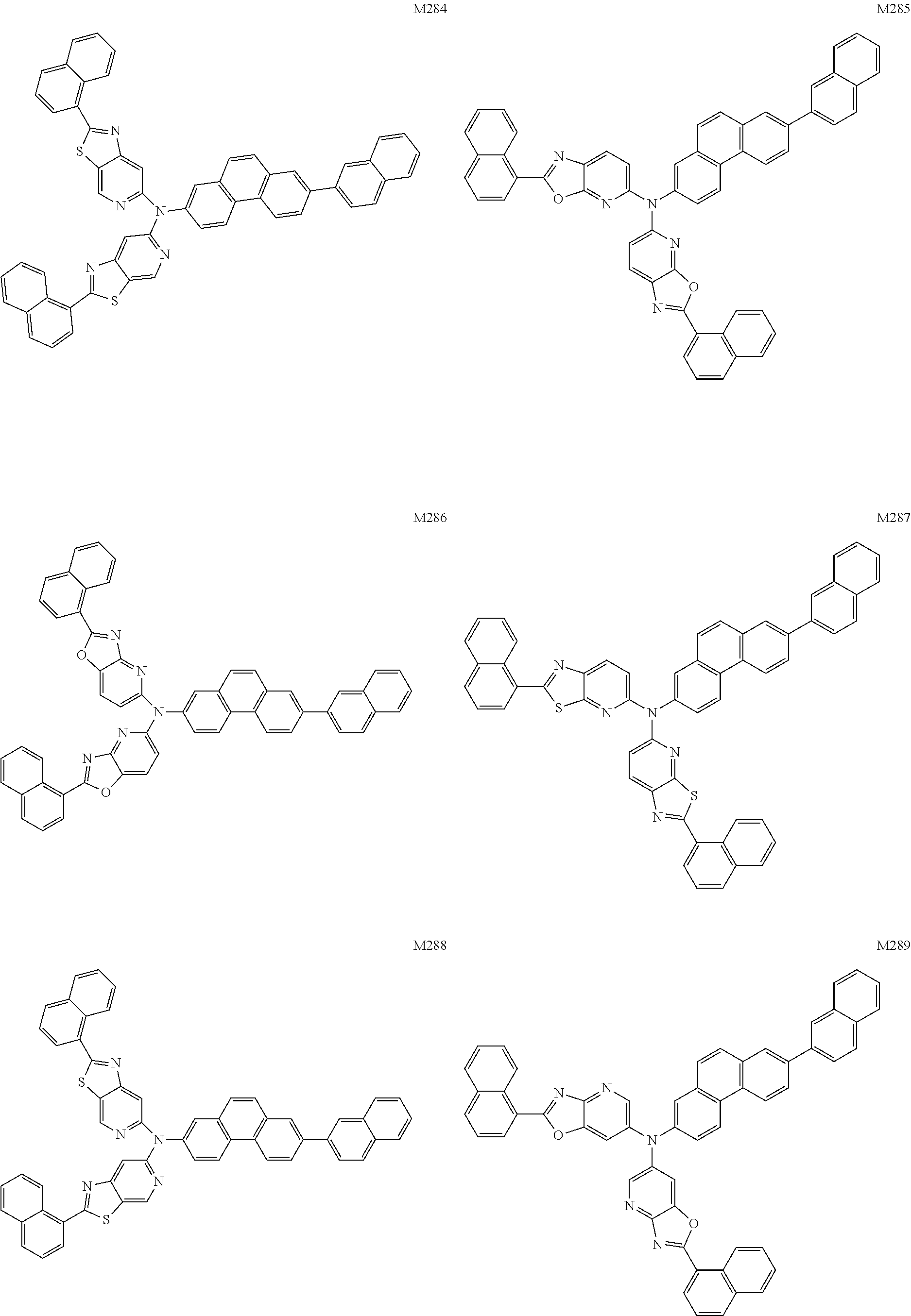 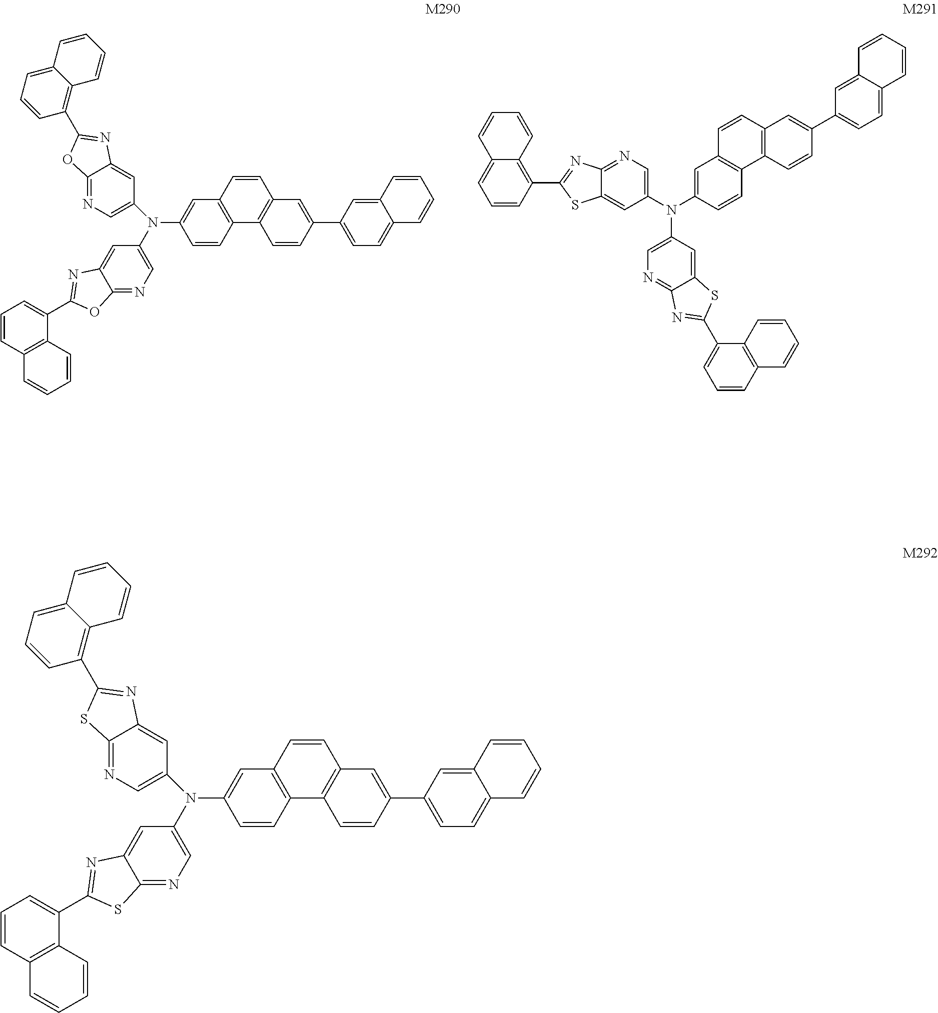 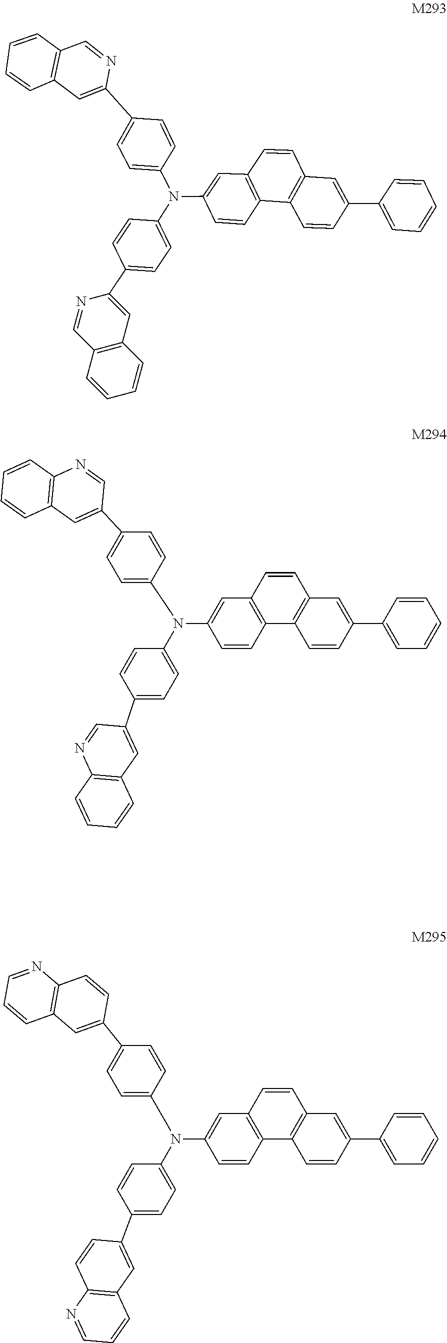 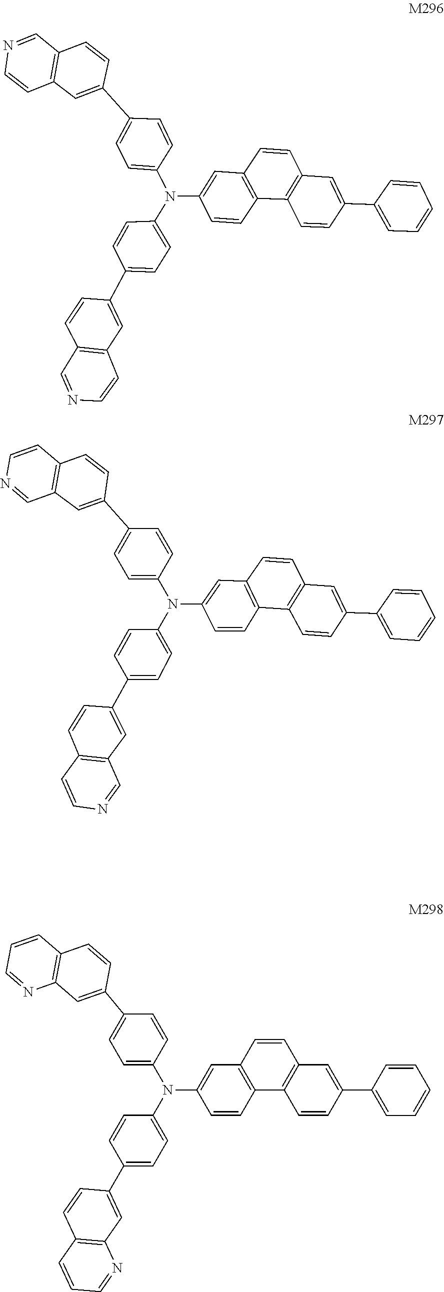 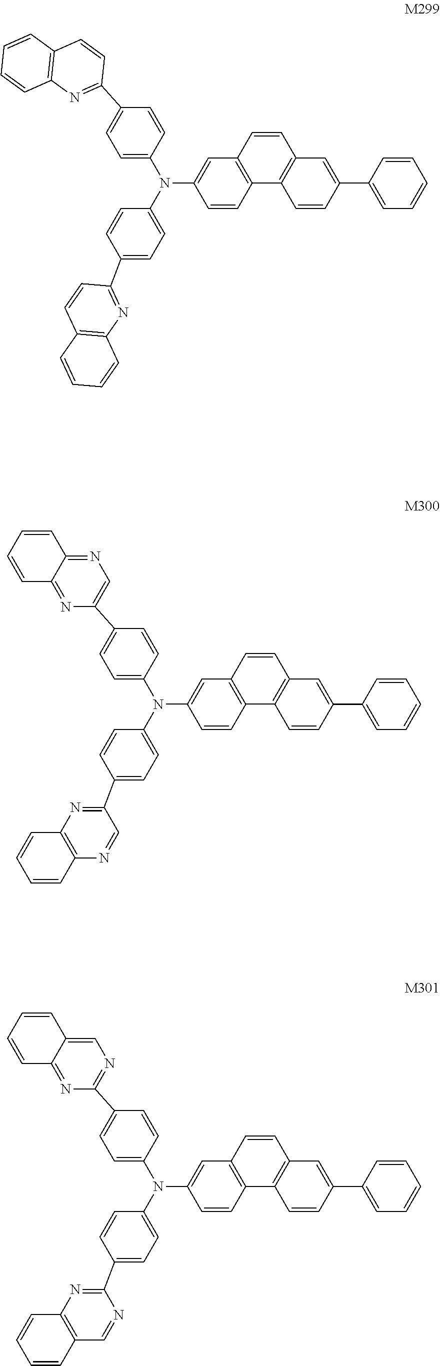 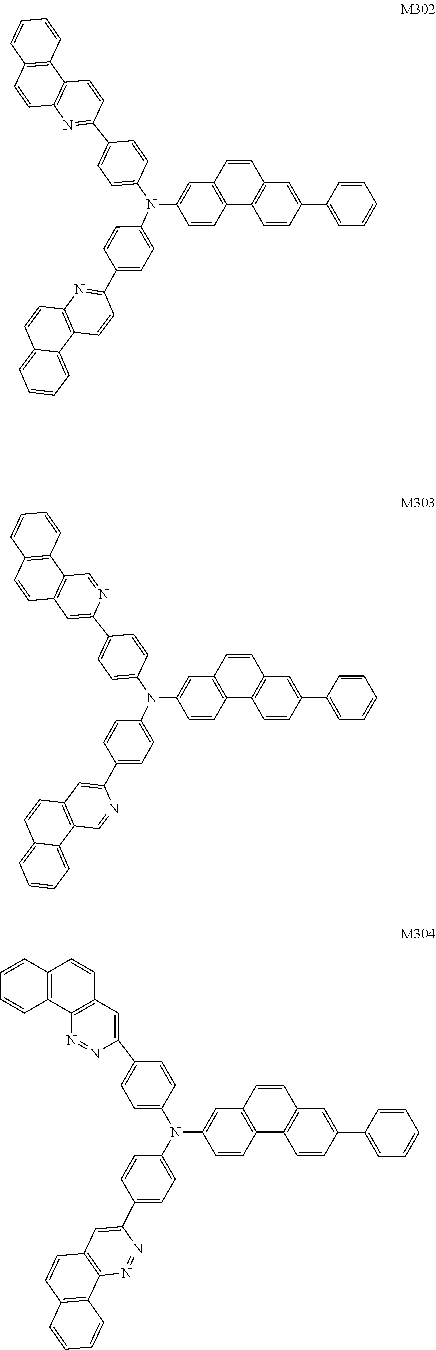 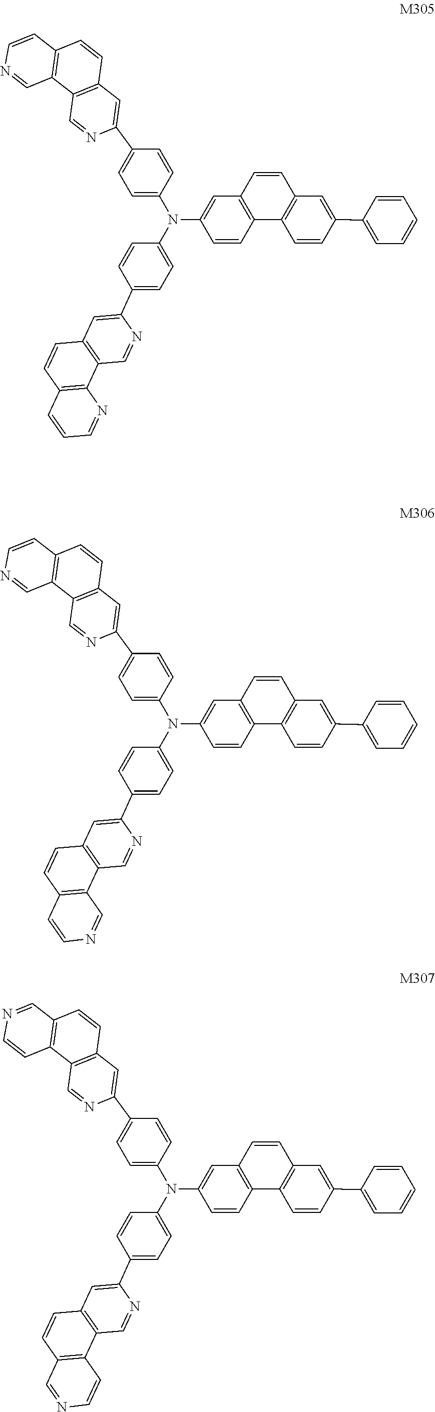 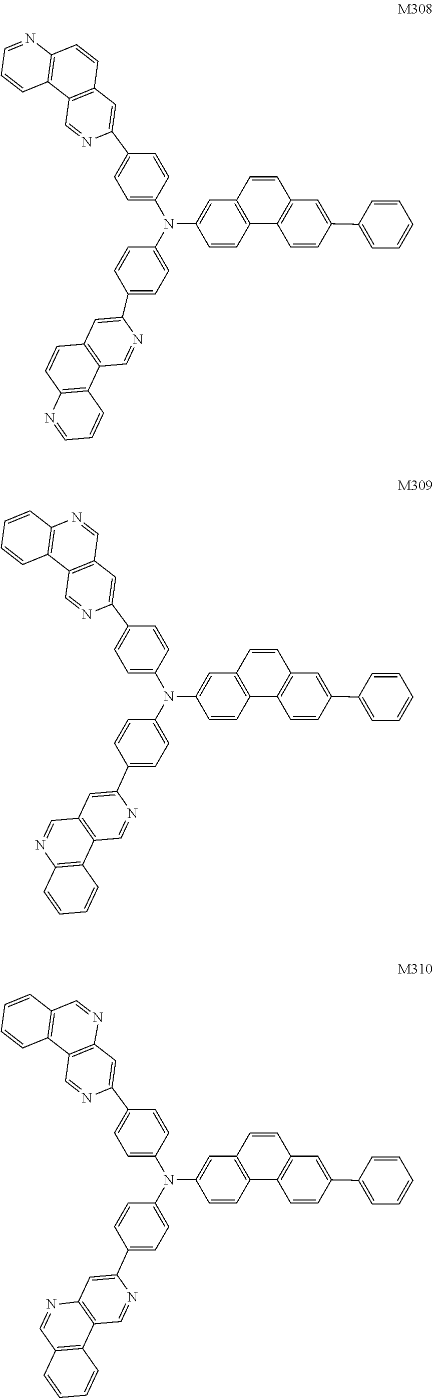 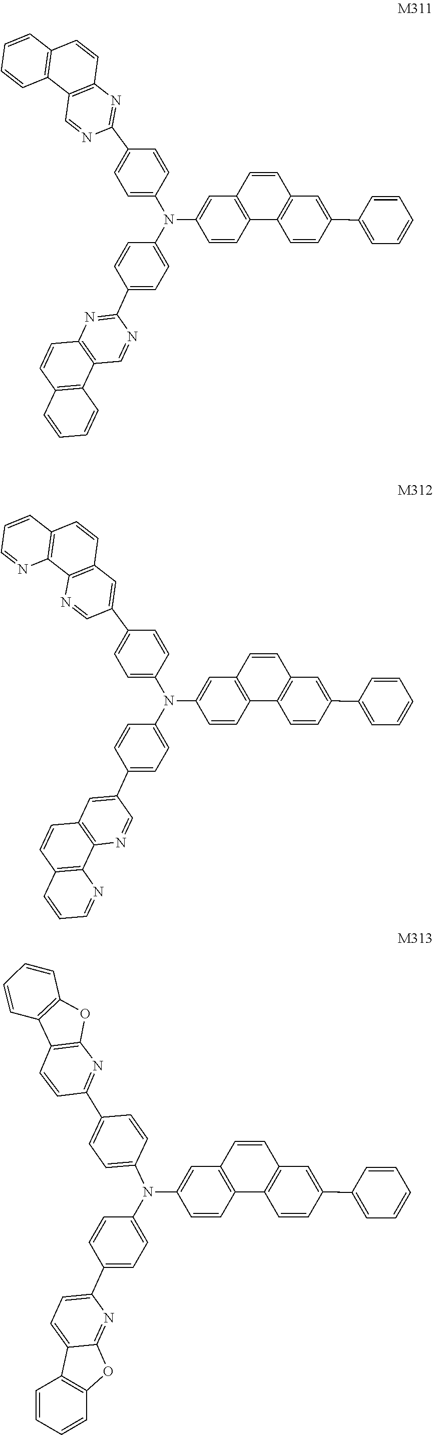 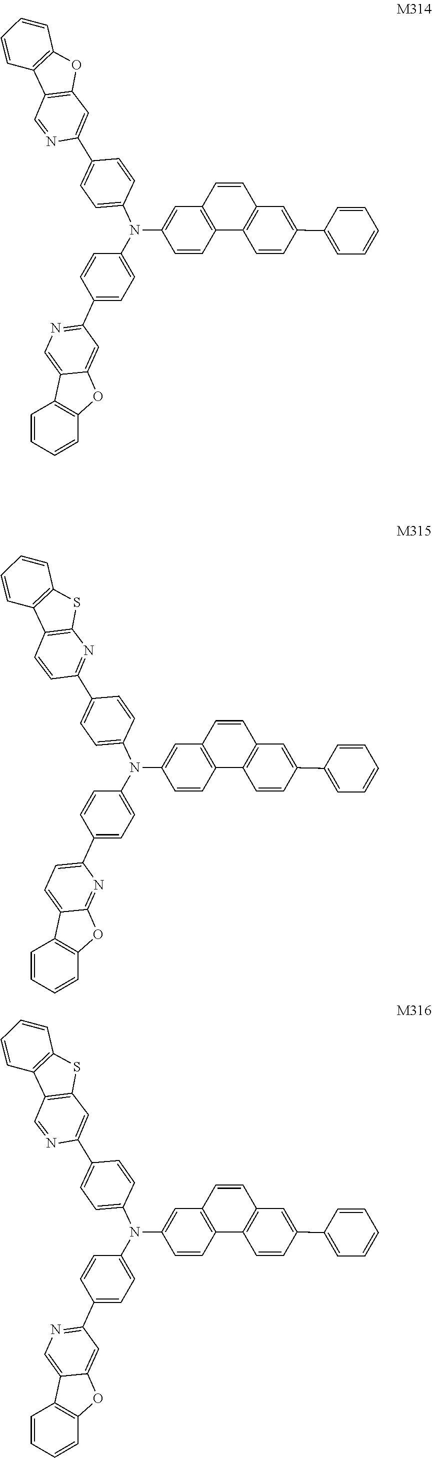 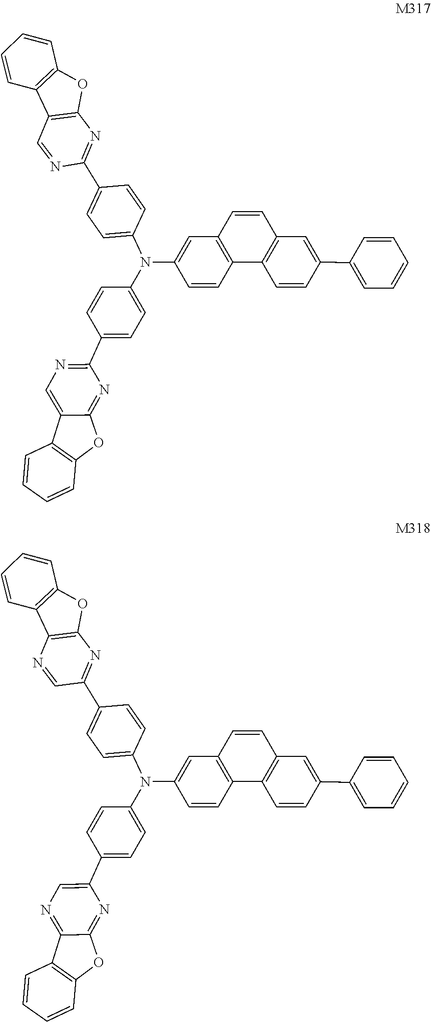 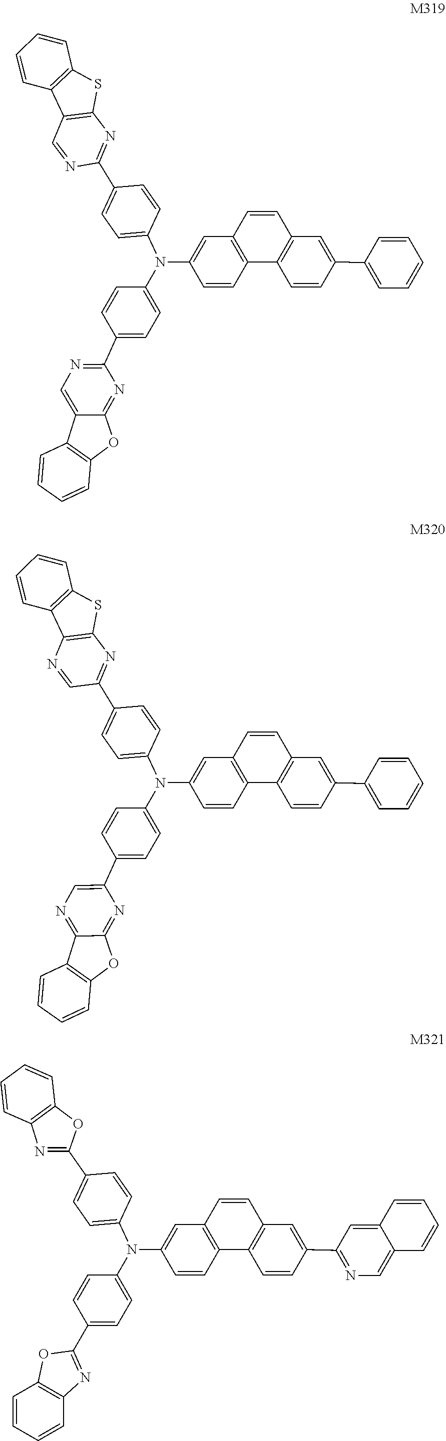 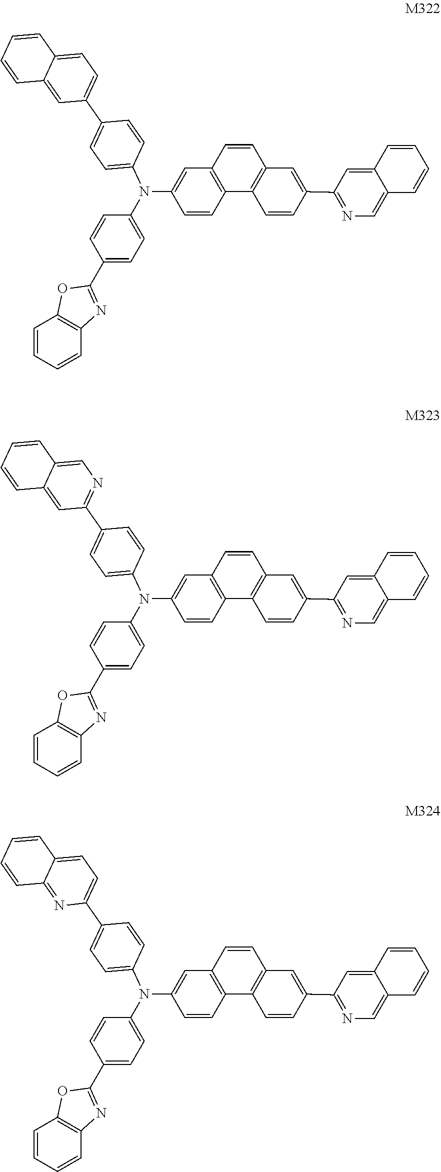 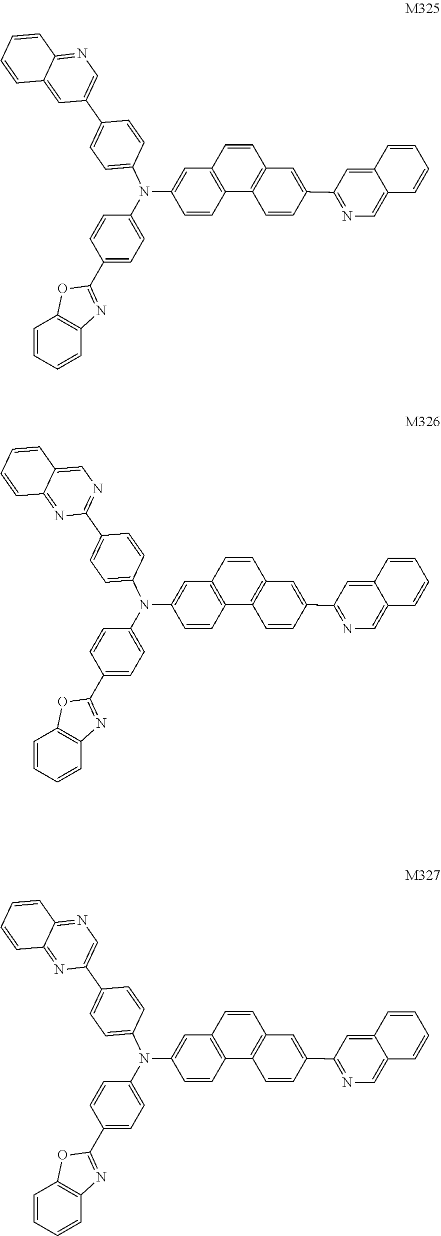 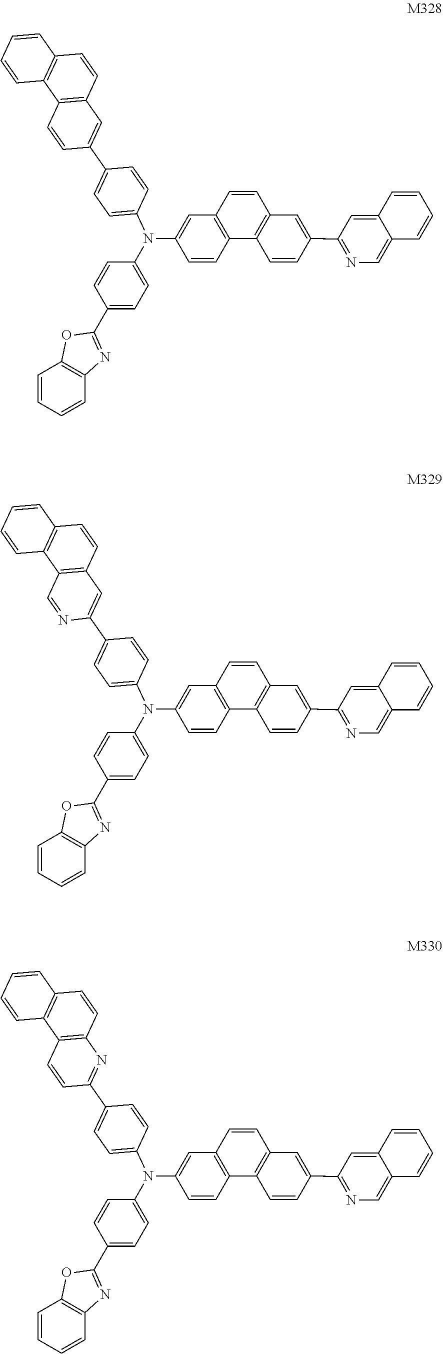 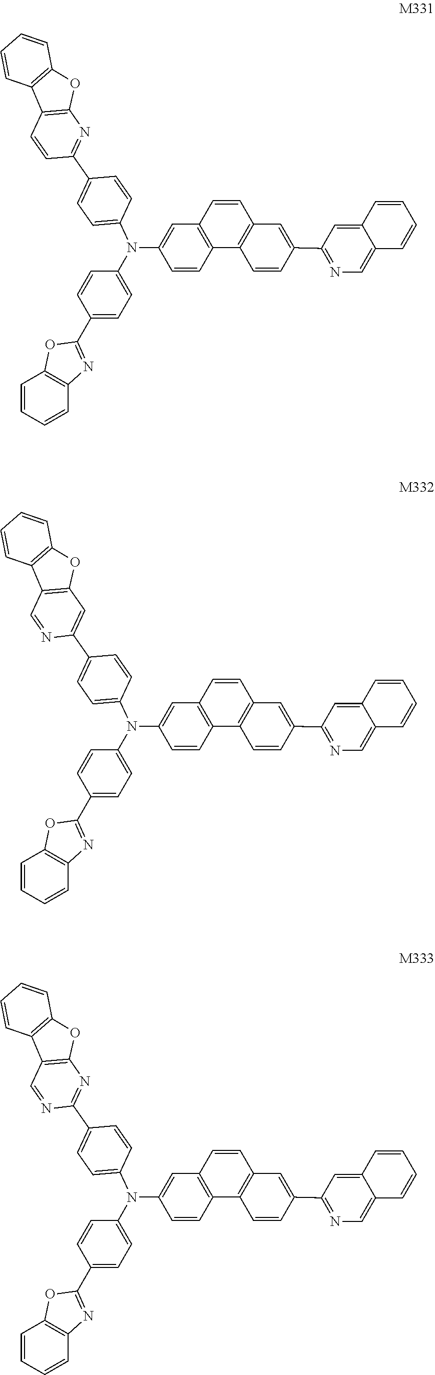 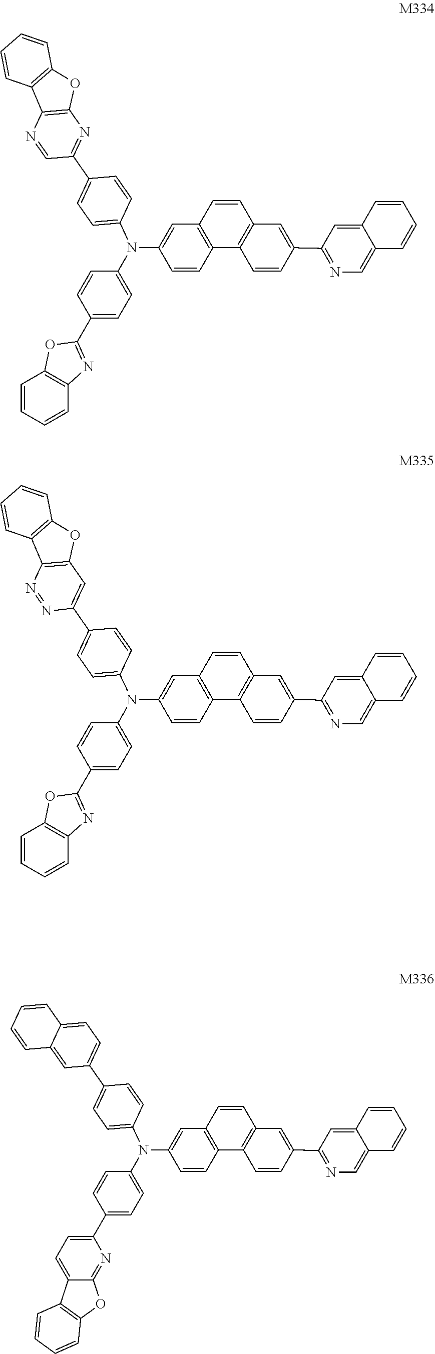 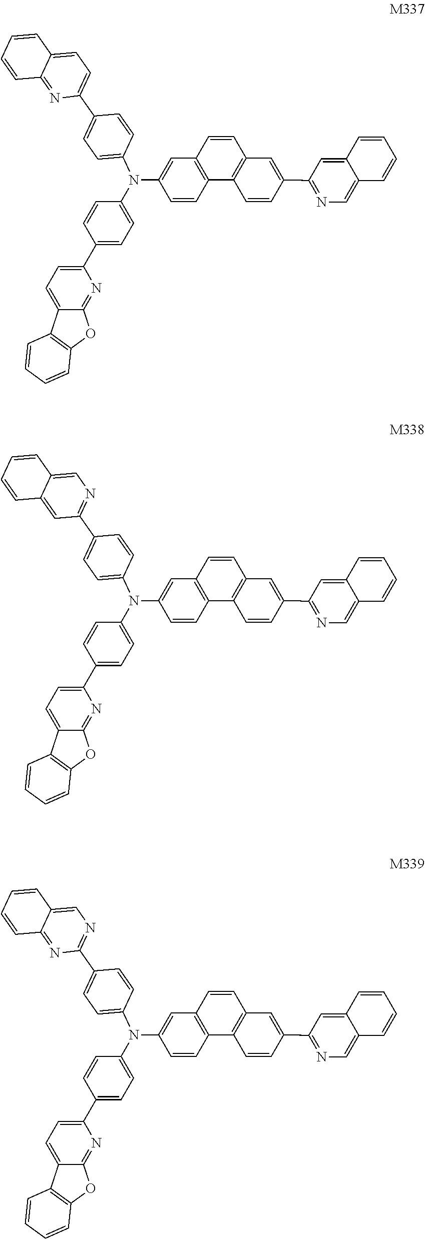 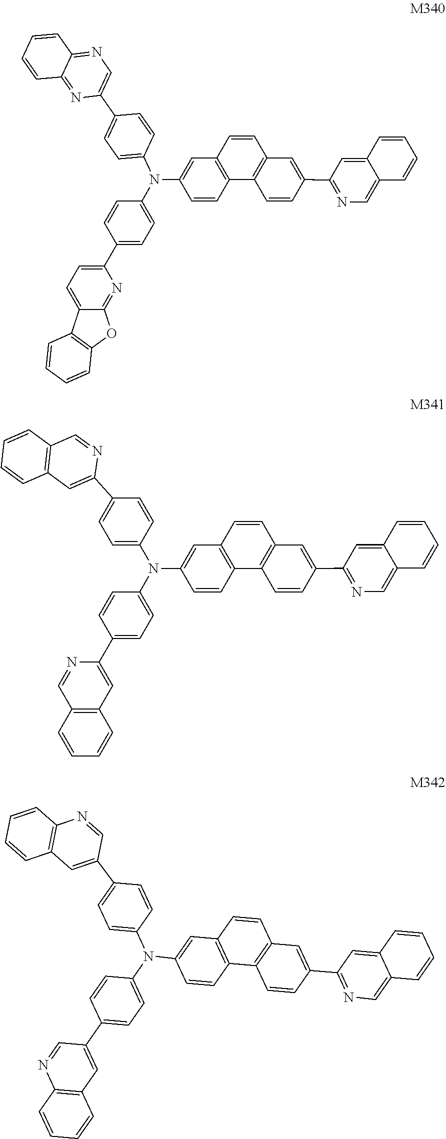 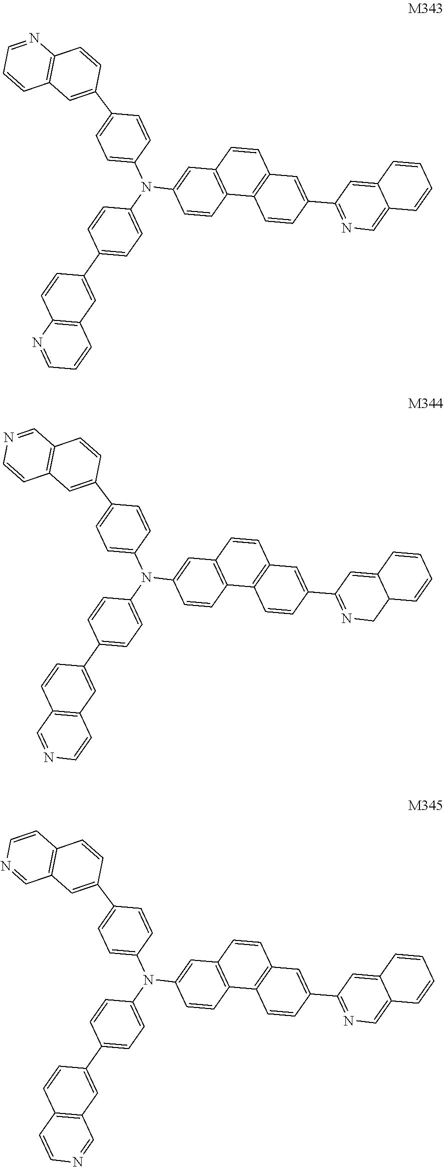 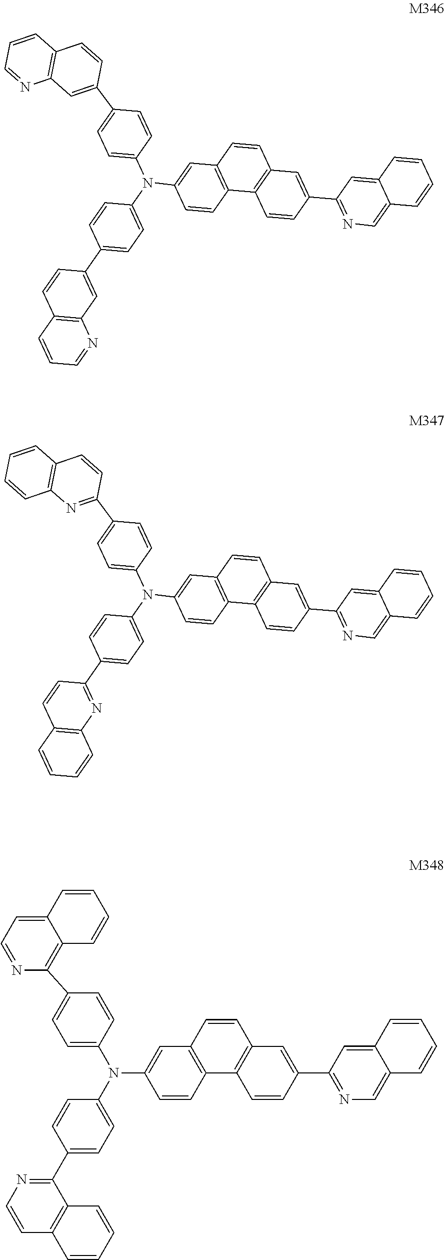 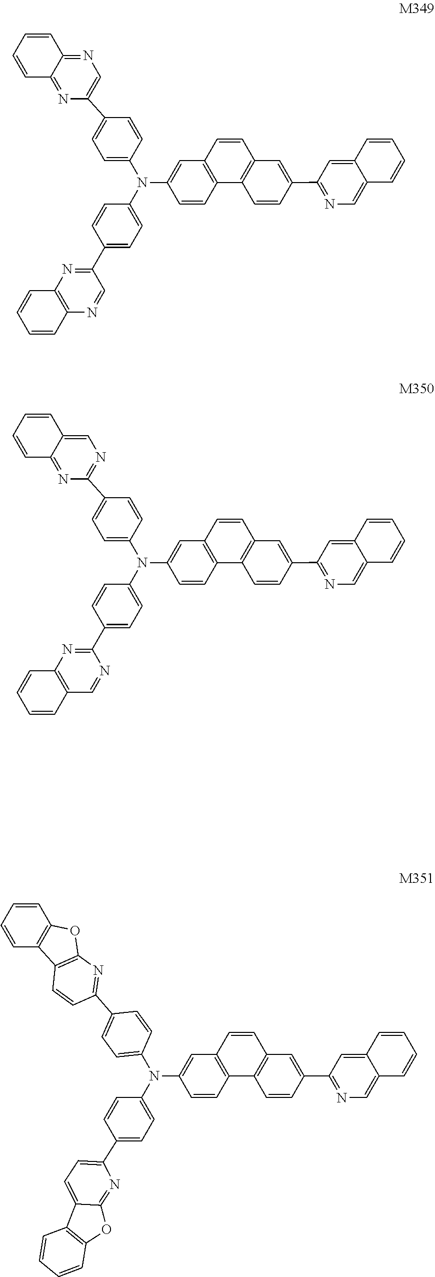 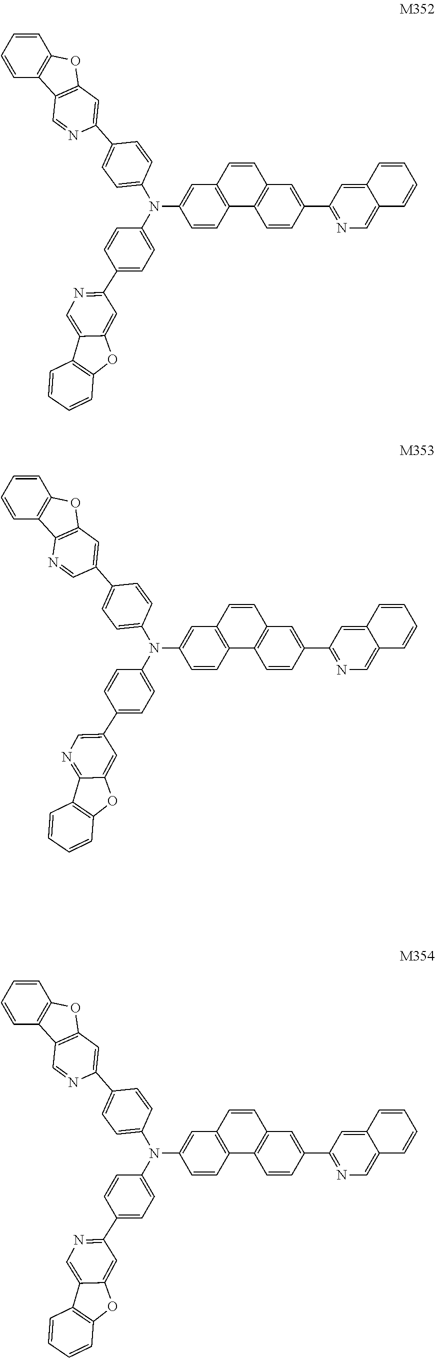 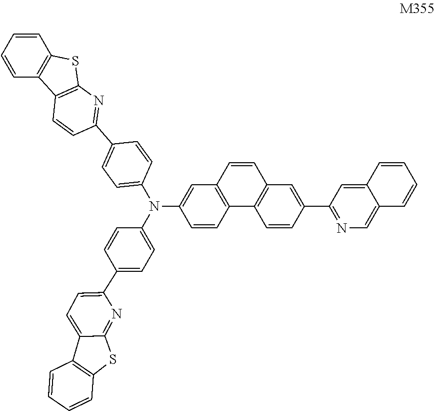 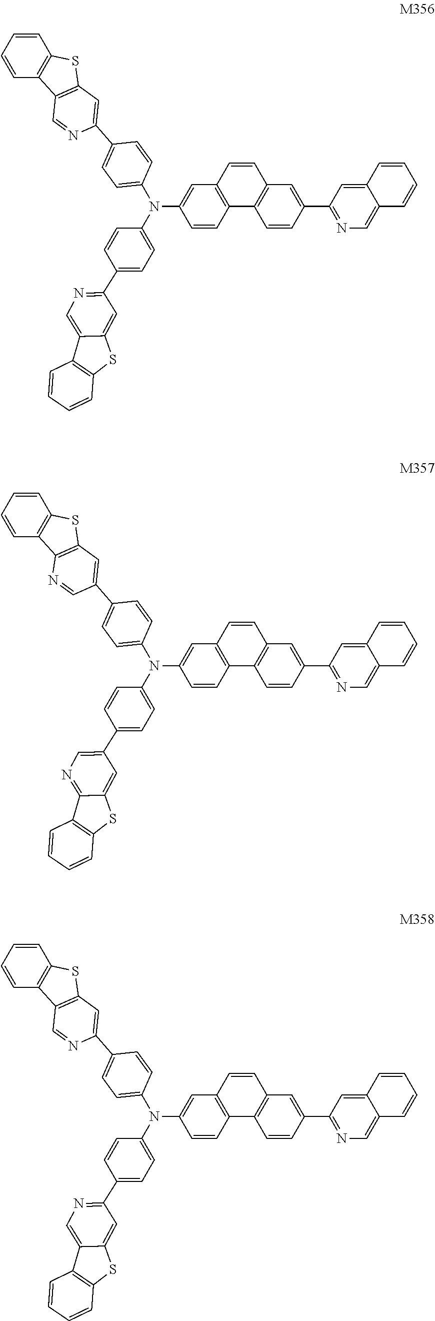 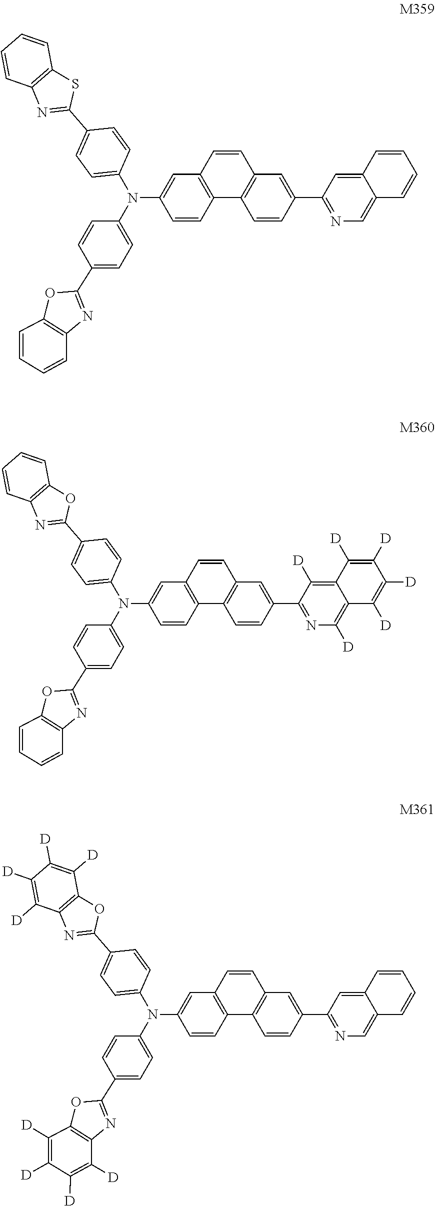 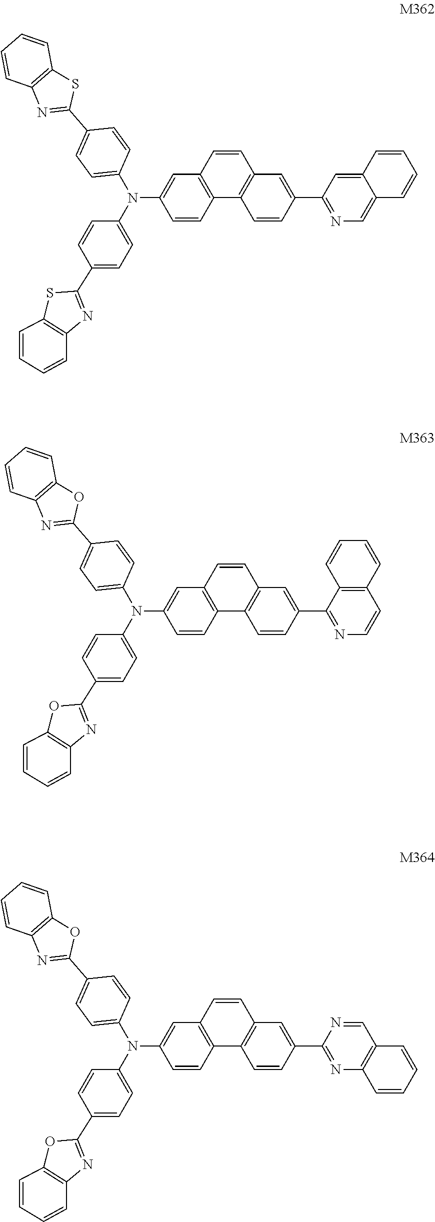 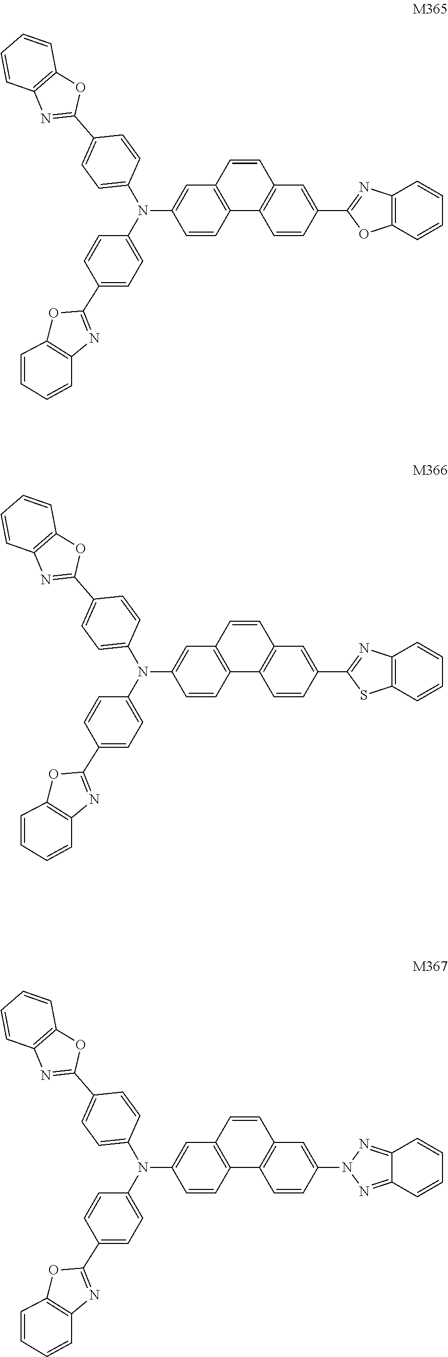 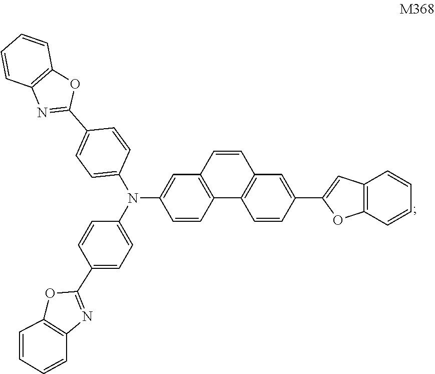
wherein D represents deuterium.

16. An organic electroluminescent device, comprising an anode, a cathode, and an organic thin-film layer disposed between the anode and the cathode, wherein the cathode is covered with a capping layer, and the capping layer comprises the phenanthrene organic compound according to claim 1.

17. An organic electroluminescent device, comprising an anode, a cathode, and an organic thin-film layer disposed between the anode and the cathode, wherein the organic thin-film layer comprises the phenanthrene organic compound according to claim 1.

18. The organic electroluminescent device according to claim 16, wherein the organic thin-film layer comprises an electron blocking layer, and the electron blocking layer comprises the phenanthrene organic compound.

19. A display panel, comprising the organic electroluminescent device according to claim 16.

20. An electronic device, comprising the display panel according to claim 19.
